SEQUENCE LISTING (250 sequences)

SEQ ID NO: 1 - protein, 119 aa, synthetic construct
QVQLQESGPGLVKPSQTLSLTCTVSGGSISRGGHQWTWIRQHPGKGLEWIGYIHHSGSTYYNPSLNDRLTILVDTPKNQFSLKLSSVTAADTAVYYCARWNLGSYGDYWGQGTLVTVSS

SEQ ID NO: 2 - protein, 113 aa, synthetic construct
AIVMTQSPDSLAVSLGERATINCKSSQSVLYSPNNKNYLAWYLQKPGQPPKLLIYWASTRESGVPDRFSGSGSGTDFTLTISSLQAEDVAVYYCQQYYTTPLTFGGGTKVEIK

SEQ ID NO: 3 - protein, 7 aa, synthetic construct
RGGHQWT

SEQ ID NO: 4 - protein, 16 aa, synthetic construct
YIHHSGSTYYNPSLND

SEQ ID NO: 5 - protein, 9 aa, synthetic construct
WNLGSYGDY

SEQ ID NO: 6 - protein, 17 aa, synthetic construct
KSSQSVLYSPNNKNYLA

SEQ ID NO: 7 - protein, 7 aa, synthetic construct
WASTRES

SEQ ID NO: 8 - protein, 9 aa, synthetic construct
QQYYTTPLT

SEQ ID NO: 9 - protein, 119 aa, synthetic construct
QVQLQESGPGLVKPSQTLSLTCTVSGGSISRGGHQWSWIRQNPEKGLEWIGYIHHSGSTYYNPSLNNRLTILVDTPKNQFSLKLSSVTAADTAVYYCARWNLGSYGDYWGQGTLVTVSS

SEQ ID NO: 10 - protein, 113 aa, synthetic construct
DIVMTQSPDSLAVSLGERATINCKSSQSVLYSPNNQNFLAWYLQKPGQPPKLLIYWASTRESGVPDRFSGSGSGTDFTLTISSLQAEDVAVYYCQQYYTTPLTFGGGTKVEIK

SEQ ID NO: 11 - protein, 7 aa, synthetic construct
RGGHQWS

SEQ ID NO: 12 - protein, 16 aa, synthetic construct
YIHHSGSTYYNPSLNN

SEQ ID NO: 13 - protein, 9 aa, synthetic construct
WNLGSYGDY

SEQ ID NO: 14 - protein, 17 aa, synthetic construct
KSSQSVLYSPNNQNFLA

SEQ ID NO: 15 - protein, 7 aa, synthetic construct
WASTRES

SEQ ID NO: 16 - protein, 9 aa, synthetic construct
QQYYTTPLT

SEQ ID NO: 17 - protein, 122 aa, synthetic construct
EVQLLESGGGLVQPGGSLRLSCAASGFTFRHYAMSWVRQAPGKGLEWVSLISGSGASTFYADSVKGRFTISRDNSENMLYLQMNSLRAEDTAVYYCAKDSLAVAGSEYFQHWGQGTLVTVSS

SEQ ID NO: 18 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDTSSDHWVFGGGTKLTVL

SEQ ID NO: 19 - protein, 5 aa, synthetic construct
HYAMS

SEQ ID NO: 20 - protein, 17 aa, synthetic construct
LISGSGASTFYADSVKG

SEQ ID NO: 21 - protein, 13 aa, synthetic construct
DSLAVAGSEYFQH

SEQ ID NO: 22 - protein, 11 aa, synthetic construct
GGNYFGGKSVH

SEQ ID NO: 23 - protein, 7 aa, synthetic construct
DDSDRPS

SEQ ID NO: 24 - protein, 11 aa, synthetic construct
QVYDTSSDHWV

SEQ ID NO: 25 - protein, 122 aa, synthetic construct
EVQLLESGGGLVQPGGSLSLSCTASGFTFSHYAMSWVRQAPGKGLEWVSVISGSGASTFYADSVKGRFTISRDNSENTLYLQMNSLRAEDTAVYYCAKDSIAVAGSEYFQHWGQGTLVTVSS

SEQ ID NO: 26 - protein, 108 aa, synthetic construct
SYVLTQAPSVSVAPGQTARITCGGNNIGGKTVHWYQQKPGQAPMLVVYDDSDRPSGIPERFSGSKSGNMAILTISRVEAGDEADYYCQLYDIYSDHWVFGGGTKLTVL

SEQ ID NO: 27 - protein, 5 aa, synthetic construct
HYAMS

SEQ ID NO: 28 - protein, 17 aa, synthetic construct
VISGSGASTFYADSVKG

SEQ ID NO: 29 - protein, 13 aa, synthetic construct
DSIAVAGSEYFQH

SEQ ID NO: 30 - protein, 11 aa, synthetic construct
GGNNIGGKTVH

SEQ ID NO: 31 - protein, 7 aa, synthetic construct
DDSDRPS

SEQ ID NO: 32 - protein, 11 aa, synthetic construct
QLYDIYSDHWV

SEQ ID NO: 33 - protein, 122 aa, synthetic construct
EVQLLESGGGLVQPGGSLRLSCAASGFKFSYYAMSWVRQAPGKGLEWVSLISGSGYSTYFADSVKGRFTISRDNSKNTLYLQMNSLRAEDTAVYYCAKDSIAVAGSEYFQHWGQGTLVTVSS

SEQ ID NO: 34 - protein, 108 aa, synthetic construct
SYVLTQPPSVSVAPGQTARITCGGDNIGGKSVHWYQQKPGQAPVLVVYDDSDRPSGIPERFSGSDSGNTATLTISWVEAGDEADYYCQVWDDSSDHWVFGGGTKLTVL

SEQ ID NO: 35 - protein, 5 aa, synthetic construct
YYAMS

SEQ ID NO: 36 - protein, 17 aa, synthetic construct
LISGSGYSTYFADSVKG

SEQ ID NO: 37 - protein, 13 aa, synthetic construct
DSIAVAGSEYFQH

SEQ ID NO: 38 - protein, 11 aa, synthetic construct
GGDNIGGKSVH

SEQ ID NO: 39 - protein, 7 aa, synthetic construct
DDSDRPS

SEQ ID NO: 40 - protein, 11 aa, synthetic construct
QVWDDSSDHWV

SEQ ID NO: 41 - protein, 122 aa, synthetic construct
QLQLQESGPGLVKTSETLSLTCTVSGGSITSGVYYWGWIRQPPGKGLEWIGSIYHGGNTYNNPSLKSRVTIFIDTSKNQFSLKLSSVTAADTAVYYCAEGVSSGWSYYFDYWGQGTLVTVSS

SEQ ID NO: 42 - protein, 108 aa, synthetic construct
SSELTQDPAVSVALGQTVRITCQGDSLRRFYASWYQQKPGQAPVLVGYGKNNRPSGIPDRFSGSTSGNAASLTITGSQAEDEADYYCNSRDSSGDHLIFGGGTKLTVL

SEQ ID NO: 43 - protein, 7 aa, synthetic construct
SGVYYWG

SEQ ID NO: 44 - protein, 16 aa, synthetic construct
SIYHGGNTYNNPSLKS

SEQ ID NO: 45 - protein, 12 aa, synthetic construct
GVSSGWSYYFDY

SEQ ID NO: 46 - protein, 11 aa, synthetic construct
QGDSLRRFYAS

SEQ ID NO: 47 - protein, 7 aa, synthetic construct
GKNNRPS

SEQ ID NO: 48 - protein, 11 aa, synthetic construct
NSRDSSGDHLI

SEQ ID NO: 49 - protein, 119 aa, synthetic construct
QVQLVQSGAEVKKPGASVKVSCKASGYSFTTYYMYWVRQAPGQGLEWMGIITPSGGSTTYAQKFQDRVTMTRDASTTTVYMELSSLRSEDTAVYYCARGYEGSYGFDHWGQGTLVTVSS

SEQ ID NO: 50 - protein, 106 aa, synthetic construct
SYELTQPPSVSLSPGQTASITCSGDKLGNKYTCWYQQKPGQSPVLVIYQDNKRPSGIPERFSGSNSGNTATLTISGTQAMDEADYYCQTWDSSTAVFGGGTKLTVL

SEQ ID NO: 51 - protein, 5 aa, synthetic construct
TYYMY

SEQ ID NO: 52 - protein, 17 aa, synthetic construct
IITPSGGSTTYAQKFQD

SEQ ID NO: 53 - protein, 10 aa, synthetic construct
GYEGSYGFDH

SEQ ID NO: 54 - protein, 11 aa, synthetic construct
SGDKLGNKYTC

SEQ ID NO: 55 - protein, 7 aa, synthetic construct
QDNKRPS

SEQ ID NO: 56 - protein, 9 aa, synthetic construct
QTWDSSTAV

SEQ ID NO: 57 - protein, 118 aa, synthetic construct
QLQLQESGPGLVKSSETLSLTCTVSGGPISSSSYYWGWIRQPPGKGLEWIGTIYYSGRTYYNPSLKSRGTISVGTSKNQFSLKLSSVTAADTAVFYCAGGVGVTWDYWGQGTLVTVSS

SEQ ID NO: 58 - protein, 106 aa, synthetic construct
SYDLSQPLSVSVALGQTARITCGGNDIESKNVHWYQQRPGQAPVLVIYRNSNRPSGIPERFSGSNSGNTATLTISRGQGGDEADYYCQVWDSSTVIFGGGTKLTVL

SEQ ID NO: 59 - protein, 7 aa, synthetic construct
SSSYYWG

SEQ ID NO: 60 - protein, 16 aa, synthetic construct
TIYYSGRTYYNPSLKS

SEQ ID NO: 61 - protein, 8 aa, synthetic construct
GVGVTWDY

SEQ ID NO: 62 - protein, 11 aa, synthetic construct
GGNDIESKNVH

SEQ ID NO: 63 - protein, 7 aa, synthetic construct
RNSNRPS

SEQ ID NO: 64 - protein, 9 aa, synthetic construct
QVWDSSTVI

SEQ ID NO: 65 - protein, 122 aa, synthetic construct
QVQLRESGPGLMKPSETLSLTCTVSGGSVSSGVYYWTWIRQPPGKGLEWIGSMFYSGNADYNPSLKSRVTISVDTSKNQFSLRLNSVSAADMAVYYCARGYASGWSYYFDYWGQATLVTVSS

SEQ ID NO: 66 - protein, 108 aa, synthetic construct
SSELTQDPTVSVALGQTVRITCQGDSLRTYYASWYQQKPGQAPVLVLYGKNNRPSGIPDRFSGSSSGNTASLTITGAQAEDEADYYCNSRYSSGTHVVFGGGTKLTVL

SEQ ID NO: 67 - protein, 7 aa, synthetic construct
SGVYYWT

SEQ ID NO: 68 - protein, 15 aa, synthetic construct
SMFYSGNADYNPSLK

SEQ ID NO: 69 - protein, 12 aa, synthetic construct
GYASGWSYYFDY

SEQ ID NO: 70 - protein, 11 aa, synthetic construct
QGDSLRTYYAS

SEQ ID NO: 71 - protein, 7 aa, synthetic construct
GKNNRPS

SEQ ID NO: 72 - protein, 11 aa, synthetic construct
NSRYSSGTHVV

SEQ ID NO: 73 - protein, 125 aa, synthetic construct
QVQLQQSGPGLVKPSQPLSLTCAISGDSVSSNSATWSWIRQSPSRGLEWLGRTYYRSKWYHDYAVFVKSRMTINPDTSKNQFSLQLNSVTPEDTAVYYCARGGMPASDIPHFDNWGQGILVTVSS

SEQ ID NO: 74 - protein, 106 aa, synthetic construct
SYELTQPPSLSVSPGQTASITCSGHKLGDKYACWYQQKPGQSPVLVIYQDNKRPSGIPERFSGSNSGNTATLTISGTQALDEADYYCQAWDSITYVFGTGTKVTVL

SEQ ID NO: 75 - protein, 7 aa, synthetic construct
SNSATWS

SEQ ID NO: 76 - protein, 18 aa, synthetic construct
RTYYRSKWYHDYAVFVKS

SEQ ID NO: 77 - protein, 13 aa, synthetic construct
GGMPASDIPHFDN

SEQ ID NO: 78 - protein, 11 aa, synthetic construct
SGHKLGDKYAC

SEQ ID NO: 79 - protein, 7 aa, synthetic construct
QDNKRPS

SEQ ID NO: 80 - protein, 9 aa, synthetic construct
QAWDSITYV

SEQ ID NO: 81 - protein, 129 aa, synthetic construct
EVQLVESGGGLVKPRGSLRLSCGASGFTFTNAWMTWVRQAPGKGLEWVGRIKSKTDGGTTDYAAPVKGRFTISRDDSKNTLYLQMNSLKTEDTAVYYCNRERELGRGPWDYFYYGMDVWGQGTTVTVSS

SEQ ID NO: 82 - protein, 108 aa, synthetic construct
SYVLTQPPSVSVAPGQTARITCGGNDIGSKSVHWYQQKPGQAPVLVVYDDNDRSSGIPERFSGSNSGNTAALTISRVEAGDEADYYCQVWDSSSDHWVFGGGTKLTVL

SEQ ID NO: 83 - protein, 5 aa, synthetic construct
NAWMT

SEQ ID NO: 84 - protein, 19 aa, synthetic construct
RIKSKTDGGTTDYAAPVKG

SEQ ID NO: 85 - protein, 18 aa, synthetic construct
ERELGRGPWDYFYYGMDV

SEQ ID NO: 86 - protein, 11 aa, synthetic construct
GGNDIGSKSVH

SEQ ID NO: 87 - protein, 7 aa, synthetic construct
DDNDRSS

SEQ ID NO: 88 - protein, 11 aa, synthetic construct
QVWDSSSDHWV

SEQ ID NO: 89 - protein, 122 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSS

SEQ ID NO: 90 - protein, 108 aa, synthetic construct; identical to SEQ ID NO: 18

SEQ ID NO: 91 - protein, 5 aa, synthetic construct
HYAMS

SEQ ID NO: 92 - protein, 17 aa, synthetic construct
LISGSGASTHYADSVKG

SEQ ID NO: 93 - protein, 13 aa, synthetic construct
DSLAVAGSEYFQH

SEQ ID NO: 94 - protein, 11 aa, synthetic construct
GGNYFGGKSVH

SEQ ID NO: 95 - protein, 7 aa, synthetic construct
DDSDRPS

SEQ ID NO: 96 - protein, 11 aa, synthetic construct
QVYDTSSDHWV

SEQ ID NO: 97 - protein, 108 aa, synthetic construct
SYVLTQAPSVSVAPGQTARITCGHNYFGGKSVHWYQQRPGQAPVLVVYDDSDRPSGIPERFSGSKSGNTATLAISRVEAGDEAAYYCQVYDTSSDHWVFGGGTKLTVL

SEQ ID NO: 98 - protein, 11 aa, synthetic construct
GHNYFGGKSVH

SEQ ID NO: 99 - protein, 7 aa, synthetic construct
DDSDRPS

SEQ ID NO: 100 - protein, 11 aa, synthetic construct
QVYDTSSDHWV

SEQ ID NO: 101 - protein, 108 aa, synthetic construct
SYVLTQAPSVSVAPGQTARITCGGNHFGGKSVHWYQQRPGQAPVLVVYDDSDRPSGIPERFSGSKSGNTATLAISRVEAGDEAAYYCQVYDTSSDHWVFGGGTKLTVL

SEQ ID NO: 102 - protein, 11 aa, synthetic construct
GGNHFGGKSVH

SEQ ID NO: 103 - protein, 7 aa, synthetic construct
DDSDRPS

SEQ ID NO: 104 - protein, 11 aa, synthetic construct
QVYDTSSDHWV

SEQ ID NO: 105 - protein, 108 aa, synthetic construct
SYVLTQAPSVSVAPGQTARITCGGNYHGGKSVHWYQQRPGQAPVLVVYDDSDRPSGIPERFSGSKSGNTATLAISRVEAGDEAAYYCQVYDTSSDHWVFGGGTKLTVL

SEQ ID NO: 106 - protein, 11 aa, synthetic construct
GGNYHGGKSVH

SEQ ID NO: 107 - protein, 7 aa, synthetic construct
DDSDRPS

SEQ ID NO: 108 - protein, 11 aa, synthetic construct
QVYDTSSDHWV

SEQ ID NO: 109 - protein, 108 aa, synthetic construct
SYVLTQAPSVSVAPGQTARITCGGNYFGHKSVHWYQQRPGQAPVLVVYDDSDRPSGIPERFSGSKSGNTATLAISRVEAGDEAAYYCQVYDTSSDHWVFGGGTKLTVL

SEQ ID NO: 110 - protein, 11 aa, synthetic construct
GGNYFGHKSVH

SEQ ID NO: 111 - protein, 7 aa, synthetic construct
DDSDRPS

SEQ ID NO: 112 - protein, 11 aa, synthetic construct
QVYDTSSDHWV

SEQ ID NO: 113 - protein, 108 aa, synthetic construct
SYVLTQAPSVSVAPGQTARITCGGNYFGGHSVHWYQQRPGQAPVLVVYDDSDRPSGIPERFSGSKSGNTATLAISRVEAGDEAAYYCQVYDTSSDHWVFGGGTKLTVL

SEQ ID NO: 114 - protein, 11 aa, synthetic construct
GGNYFGGHSVH

SEQ ID NO: 115 - protein, 7 aa, synthetic construct
DDSDRPS

SEQ ID NO: 116 - protein, 11 aa, synthetic construct
QVYDTSSDHWV

SEQ ID NO: 117 - protein, 108 aa, synthetic construct
SYVLTQAPSVSVAPGQTARITCGGNYFGGKSHHWYQQRPGQAPVLVVYDDSDRPSGIPERFSGSKSGNTATLAISRVEAGDEAAYYCQVYDTSSDHWVFGGGTKLTVL

SEQ ID NO: 118 - protein, 11 aa, synthetic construct
GGNYFGGKSHH

SEQ ID NO: 119 - protein, 7 aa, synthetic construct
DDSDRPS

SEQ ID NO: 120 - protein, 11 aa, synthetic construct
QVYDTSSDHWV

SEQ ID NO: 121 - protein, 108 aa, synthetic construct
SYVLTQAPSVSVAPGQTARITCGGNYFGGKSVHWYQQRPGQAPVLVVYHDSDRPSGIPERFSGSKSGNTATLAISRVEAGDEAAYYCQVYDTSSDHWVFGGGTKLTVL

SEQ ID NO: 122 - protein, 11 aa, synthetic construct
GGNYFGGKSVH

SEQ ID NO: 123 - protein, 7 aa, synthetic construct
HDSDRPS

SEQ ID NO: 124 - protein, 11 aa, synthetic construct
QVYDTSSDHWV

SEQ ID NO: 125 - protein, 108 aa, synthetic construct
SYVLTQAPSVSVAPGQTARITCGGNYFGGKSVHWYQQRPGQAPVLVVYDDHDRPSGIPERFSGSKSGNTATLAISRVEAGDEAAYYCQVYDTSSDHWVFGGGTKLTVL

SEQ ID NO: 126 - protein, 11 aa, synthetic construct
GGNYFGGKSVH

SEQ ID NO: 127 - protein, 7 aa, synthetic construct
DDHDRPS

SEQ ID NO: 128 - protein, 11 aa, synthetic construct
QVYDTSSDHWV

SEQ ID NO: 129 - protein, 108 aa, synthetic construct
SYVLTQAPSVSVAPGQTARITCGGNYFGGKSVHWYQQRPGQAPVLVVYDDSDHPSGIPERFSGSKSGNTATLAISRVEAGDEAAYYCQVYDTSSDHWVFGGGTKLTVL

SEQ ID NO: 130 - protein, 11 aa, synthetic construct
GGNYFGGKSVH

SEQ ID NO: 131 - protein, 7 aa, synthetic construct
DDSDHPS

SEQ ID NO: 132 - protein, 11 aa, synthetic construct
QVYDTSSDHWV

SEQ ID NO: 133 - protein, 108 aa, synthetic construct
SYVLTQAPSVSVAPGQTARITCGGNYFGGKSVHWYQQRPGQAPVLVVYDDSDRHSGIPERFSGSKSGNTATLAISRVEAGDEAAYYCQVYDTSSDHWVFGGGTKLTVL

SEQ ID NO: 134 - protein, 11 aa, synthetic construct
GGNYFGGKSVH

SEQ ID NO: 135 - protein, 7 aa, synthetic construct
DDSDRHS

SEQ ID NO: 136 - protein, 11 aa, synthetic construct
QVYDTSSDHWV

SEQ ID NO: 137 - protein, 108 aa, synthetic construct
SYVLTQAPSVSVAPGQTARITCGGNYFGGKSVHWYQQRPGQAPVLVVYDDSDRPHGIPERFSGSKSGNTATLAISRVEAGDEAAYYCQVYDTSSDHWVFGGGTKLTVL

SEQ ID NO: 138 - protein, 11 aa, synthetic construct
GGNYFGGKSVH

SEQ ID NO: 139 - protein, 7 aa, synthetic construct
DDSDRPH

SEQ ID NO: 140 - protein, 11 aa, synthetic construct
QVYDTSSDHWV

SEQ ID NO: 141 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYHTSSDHWVFGGGTKLTVL

SEQ ID NO: 142 - protein, 11 aa, synthetic construct
GGNYFGGKSVH

SEQ ID NO: 143 - protein, 7 aa, synthetic construct
DDSDRPS

SEQ ID NO: 144 - protein, 11 aa, synthetic construct
QVYHTSSDHWV

SEQ ID NO: 145 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDHSSDHWVFGGGTKLTVL

SEQ ID NO: 146 - protein, 11 aa, synthetic construct
GGNYFGGKSVH

SEQ ID NO: 147 - protein, 7 aa, synthetic construct
DDSDRPS

SEQ ID NO: 148 - protein, 11 aa, synthetic construct
QVYDHSSDHWV

SEQ ID NO: 149 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDTHSDHWVFGGGTKLTVL

SEQ ID NO: 150 - protein, 11 aa, synthetic construct
GGNYFGGKSVH

SEQ ID NO: 151 - protein, 7 aa, synthetic construct
DDSDRPS

SEQ ID NO: 152 - protein, 11 aa, synthetic construct
QVYDTHSDHWV

SEQ ID NO: 153 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDTSHDHWVFGGGTKLTVL

SEQ ID NO: 154 - protein, 11 aa, synthetic construct
GGNYFGGKSVH

SEQ ID NO: 155 - protein, 7 aa, synthetic construct
DDSDRPS

SEQ ID NO: 156 - protein, 11 aa, synthetic construct
QVYDTSHDHWV

SEQ ID NO: 157 - protein, 108 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDTSSDHWHFGGGTKLTVL

SEQ ID NO: 158 - protein, 11 aa, synthetic construct
GGNYFGGKSVH

SEQ ID NO: 159 - protein, 7 aa, synthetic construct
DDSDRPS

SEQ ID NO: 160 - protein, 11 aa, synthetic construct
QVYDTSSDHWH

SEQ ID NO: 161 - DNA, 357 bp, synthetic construct
  source 1..357: mol_type = other DNA
caggtgcagctgcaggagtcgggcccaggactggtgaagccgtcacagaccctgtccctcacctgcactgtctctggtggctccatcagcaggggtggtcaccagtggacttggatccgccagcacccagggaagggcctggagtggattgggtacatccaccacagtgggagcacctactacaacccgtccctcaacgaccgacttaccatattggtagacacgcctaagaaccagttttccctgaagctgagctctgtgactgccgcggacacggccgtgtattactgtgcgagatggaatttggggagttatggggactactggggccagggaaccctggtcaccgtctcctca

SEQ ID NO: 162 - DNA, 339 bp, synthetic construct
  source 1..339: mol_type = other DNA
gccatcgtgatgacccagtctccagactccctggctgtgtctctgggcgagagggccaccatcaactgtaagtccagccagagtgttttatacagccccaacaataagaactacttggcttggtacctgcagaaaccagggcagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatactactcctctcactttcggcggagggaccaaggtggagatcaaa

SEQ ID NO: 163 - DNA, 357 bp, synthetic construct
  source 1..357: mol_type = other DNA
caggtgcagctgcaggagtcgggcccaggactggtgaagccgtcccagaccctgtccctcacttgcactgtctctggtggctccatcagcaggggtggtcaccagtggagttggatccgccagaacccagagaagggcctggagtggattgggtacatccaccacagtgggagcacctactacaacccgtccctcaacaatcgacttaccattttggtagacacgcctaagaaccagttttccctgaagctgagctctgtgactgccgcggacacggccgtgtattactgtgcgagatggaatttggggagttatggggactactggggccagggaaccctggtcaccgtctcctca

SEQ ID NO: 164 - DNA, 339 bp, synthetic construct
  source 1..339: mol_type = other DNA
gacatcgtgatgacccagtctccagactccctggctgtgtctctgggcgagagggccaccatcaactgcaagtccagccagagtgttttatacagccccaacaatcagaacttcttagcttggtacctgcagaaaccaggtcagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatactactcctctcactttcggcggagggaccaaggtggagatcaaa

SEQ ID NO: 165 - DNA, 366 bp, synthetic construct
  source 1..366: mol_type = other DNA
gaggtgcagctgttggagtctgggggaggcctggtacagcctggggggtccctgagactctcctgtgcagcctctggattcacctttaggcactatgccatgagctgggtccgccaggctccagggaaggggctggagtgggtctcacttatcagtggtagtggtgccagcacattctacgcagactccgtgaagggccggttcaccatctccagagacaattccgagaacatgctgtatctgcaaatgaacagcctgagagccgaggacacggccgtctattactgtgcgaaagatagtttagcagtagctggttctgaatacttccagcactggggccagggcaccctggtcaccgtctcctca

SEQ ID NO: 166 - DNA, 324 bp, synthetic construct
  source 1..324: mol_type = other DNA
tcctatgtgctgactcaggcgccctcggtgtcagtggccccaggacagacggccaggattacctgtgggggaaactattttggaggtaaaagtgtacactggtaccagcagaggccaggccaggcccctgtcctggtcgtctatgatgatagcgaccggccctcagggatccctgagcgattctctggctccaagtctgggaacacggccaccctggccatcagcagggtcgaagccggggatgaggccgcctattactgtcaggtgtatgatactagtagtgatcattgggtgttcggcggagggaccaagctgaccgtccta

SEQ ID NO: 167 - DNA, 366 bp, synthetic construct
  source 1..366: mol_type = other DNA
gaggtgcagctgttggagtctgggggaggcttggtacagcctggggggtccctgagtctctcctgtacagcctctggattcacctttagccactatgccatgagctgggtccgccaggctccagggaaggggctggagtgggtctcagttatcagtggtagtggtgccagcacattctacgcagactccgtgaagggccggttcaccatctccagagacaattccgagaacacgctgtatctgcaaatgaacagcctgagagccgaggacacggccgtatattactgtgcgaaagatagtatagcagtagctggttctgaatacttccagcactggggccagggcaccctggtcaccgtctcctca

SEQ ID NO: 168 - DNA, 324 bp, synthetic construct
  source 1..324: mol_type = other DNA
tcctatgtgctgactcaggcgccctcggtgtcagtggccccaggacagacggccaggattacctgtgggggaaacaatattggaggtaaaactgtacactggtaccagcagaagccaggccaggcccctatgctggtcgtctatgatgatagcgaccggccctcagggatccctgagcgattctctggctccaagtctgggaatatggccatcctgaccatcagtagggtcgaagccggggatgaggccgactattactgtcaactgtatgatatttatagtgatcattgggtgttcggcggagggaccaagctgaccgtccta

SEQ ID NO: 169 - DNA, 366 bp, synthetic construct
  source 1..366: mol_type = other DNA
gaggtgcagctgttggagtctgggggaggtttggtacagcctggggggtccctgagactctcctgtgcagcctctggattcaaatttagctactatgccatgagctgggtccgccaggctccagggaaggggctggagtgggtctcacttattagtggtagtggttatagcacatacttcgcagactccgtgaagggccggttcaccatctccagagacaattccaagaacacgctgtatcttcaaatgaacagcctgagagccgaggacacggccgtatattactgtgcgaaagattcgatagcagtggctggtagtgaatacttccagcactggggccagggcaccctggtcaccgtctcctca

SEQ ID NO: 170 - DNA, 324 bp, synthetic construct
  source 1..324: mol_type = other DNA
tcctatgtgctgactcagccaccctcggtgtcagtggccccaggacagacggccaggattacctgtgggggagacaacattggaggtaaaagtgtgcactggtaccaacagaagccaggccaggcccctgtgctggtcgtctatgatgatagcgaccggccctcaggtatccctgagcgcttctctggctccgactctgggaacacggccaccctgaccatcagttgggtcgaagccggggatgaggccgactattactgtcaggtgtgggatgatagtagtgatcattgggtgttcggcggagggaccaagctgaccgtccta

SEQ ID NO: 171 - DNA, 366 bp, synthetic construct
  source 1..366: mol_type = other DNA
cagctgcagttgcaggagtcgggcccaggactggtgaagacttcggagaccctgtccctcacctgcactgtctctggtggttccatcaccagtggtgtttactactggggctggatccgccagcccccagggaaggggctggagtggattgggagtatctatcatggtgggaacacctacaacaacccgtccctcaagagtcgagtcaccatattcatagacacgtccaagaaccagttctccctgaagctgagttctgtgaccgccgctgacacggctgtgtattactgtgcggagggagttagcagtggctggtcctactactttgactattggggccagggaaccctggtcaccgtctcctca

SEQ ID NO: 172 - DNA, 324 bp, synthetic construct
  source 1..324: mol_type = other DNA
tcttctgaactgactcaggaccctgctgtgtctgtggccttgggacagacagtcaggatcacatgccaaggagacagcctcagacgcttttatgctagctggtaccagcagaagccaggacaggcccctgttcttgtcggctatggtaaaaataaccggccctcagggatccccgaccgattctccggctccacctcgggaaacgcagcttctttgaccatcactgggtctcaggcggaagatgaggctgactattactgtaactcccgggacagcagtggtgaccatctgatattcggcggaggtaccaagttgaccgtccta

SEQ ID NO: 173 - DNA, 357 bp, synthetic construct
  source 1..357: mol_type = other DNA
caggtgcagctggtgcagtctggggctgaggtgaagaagcctggggcctcagtgaaggtttcctgtaaggcatctggatacagtttcaccacttactatatgtactgggtgcgacaggcccctggacaagggcttgagtggatgggaatcatcacccctagcggtggtagtacaacctacgcacagaagttccaggacagagtcaccatgacaagggacgcgtccacgactacagtctacatggagctgagcagcctgagatctgaggacacggccgtgtattactgtgcgagagggtatgaggggagctacgggtttgaccactggggccagggaaccctggtcaccgtctcctca

SEQ ID NO: 174 - DNA, 318 bp, synthetic construct
  source 1..318: mol_type = other DNA
tcctatgaactgactcagccaccctcagtgtccctgtccccaggacagacagccagcatcacctgctctggagataaattgggaaataaatatacttgctggtatcagcagaagccaggccagtcccctgtcctggtcatctatcaagataacaagcggccctcggggatccctgagcgattctctggctccaactctgggaacacagccactctgaccatcagcgggacccaggctatggatgaggctgactattactgtcagacgtgggacagcagcactgcggtgttcggcggagggaccaagctgaccgtccta

SEQ ID NO: 175 - DNA, 354 bp, synthetic construct
  source 1..354: mol_type = other DNA
cagctgcagctgcaggagtcgggcccaggattagtgaagtcttcggagaccctgtccctcacctgcactgtctctggtggccccatcagcagtagtagttactactggggctggatccgccagcccccagggaaggggctggagtggattgggactatttattatagtgggaggacctactacaatccgtccctcaagagtcgaggcaccatatccgtaggaacgtccaagaaccagttctccctgaagctgagctctgtgaccgccgcagacacggcagtgttttactgcgcggggggagtgggagttacttgggactactggggccagggaaccctggtcaccgtctcctca

SEQ ID NO: 176 - DNA, 318 bp, synthetic construct
  source 1..318: mol_type = other DNA
tcctatgacctgtctcagccactctcagtgtcagtggccctgggacagacggccaggattacctgtgggggaaacgacattgaaagtaaaaatgtgcactggtaccagcagaggccaggccaggcccctgtgctggtcatctataggaatagcaaccggccctctgggatccctgagcgattctctggctccaactcggggaacacggccaccctgaccatcagcagaggccaaggcggggatgaggctgactattactgtcaggtgtgggacagcagcactgtgattttcggcggagggaccaagctgaccgtccta

SEQ ID NO: 177 - DNA, 366 bp, synthetic construct
  source 1..366: mol_type = other DNA
caggtgcagctgcgggagtcgggcccaggactgatgaagccttcggagaccctgtccctcacctgcactgtctctggtggctccgtcagcagtggcgtttactactggacctggatccggcagcccccagggaagggactggaatggattgggtctatgttttacagtgggaacgccgactacaacccctccctcaagagtcgagtcaccatatcagtagacacgtccaagaaccagttctccctgaggctgaactctgtgtccgctgcggacatggccgtttattactgtgcgagaggatatgccagtgggtggtcctactactttgactactggggccaggcaaccctggtcaccgtctcctca

SEQ ID NO: 178 - DNA, 324 bp, synthetic construct
  source 1..324: mol_type = other DNA
tcttctgagctgactcaggaccctactgtgtctgtggccttgggacagacagtcaggatcacatgccaaggagacagcctcagaacctattatgcaagctggtaccagcagaagccaggacaggcccctgtacttgtcctctatggtaaaaacaaccggccctcagggatcccagaccgcttctctggctccagctcaggaaacacagcttccttgaccatcactggggctcaggcggaagatgaggctgactattactgtaactcccgatacagcagtggtacccatgtggtattcggcggagggaccaagctgaccgtccta

SEQ ID NO: 179 - DNA, 375 bp, synthetic construct
  source 1..375: mol_type = other DNA
caggtacagctgcagcagtcaggtccaggactggtgaagccctcgcagcccctctcactcacctgtgccatctccggggacagtgtctctagcaacagtgctacttggagctggatcaggcagtccccatcgagaggccttgagtggctgggaaggacatactacaggtccaagtggtatcatgattatgcagtatttgtgaagagtcgaatgaccatcaacccagacacatccaagaaccagttctccctgcagctgaactctgtgactcccgaggacacggctgtgtattactgtgcaagagggggtatgccagccagtgatataccccactttgacaactggggccagggtattctggtcaccgtctcttca

SEQ ID NO: 180 - DNA, 318 bp, synthetic construct
  source 1..318: mol_type = other DNA
tcctatgagctgacccagccaccctctttgtccgtgtccccaggacagacagccagcatcacctgctctggacataagttgggggataaatatgcttgttggtatcagcagaagccaggccagtcccctgtgttggtcatctatcaagataacaagcggccctcagggatccctgagcgattctctggctccaactctgggaacacagccactctgaccatcagcgggacccaggctttggatgaggctgactattactgtcaggcgtgggacagcatcacttatgtcttcggaactgggaccaaggtcaccgtccta

SEQ ID NO: 181 - DNA, 387 bp, synthetic construct
  source 1..387: mol_type = other DNA
gaggtgcagctggtggagtctgggggaggcttggtaaagcctagggggtcccttagactctcctgtggagcctctggattcactttcactaatgcctggatgacctgggtccgccaggctccagggaaggggctggagtgggttggccgtattaaaagcaaaactgatggtgggacaacagactacgctgcacccgtgaaaggcagattcaccatctcaagagatgactcaaaaaacacgctgtatctgcaaatgaacagcctgaaaaccgaggacactgccgtgtattactgtaatagagagagggaactggggaggggcccgtgggactacttctactacggtatggacgtctggggccaagggaccacggtcaccgtctcctca

SEQ ID NO: 182 - DNA, 324 bp, synthetic construct
  source 1..324: mol_type = other DNA
tcctatgtgctgactcagccaccctcggtgtcagtggccccaggacagacggccaggattacctgtgggggaaacgacattggaagtaaaagtgtgcactggtaccagcagaagccaggccaggcccctgtgctggtcgtctatgatgataacgaccggtcctcagggatccctgagcgattttctggctccaactctgggaacacggccgccctgaccatcagcagggtcgaagccggggatgaggccgactattactgtcaggtgtgggatagtagtagtgatcattgggtgttcggcggagggaccaagctgaccgtccta

SEQ ID NO: 183 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 184 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGK

SEQ ID NO: 185 - protein, 752 aa, Homo sapiens
MGPLMVLFCLLFLYPGLADSAPSCPQNVNISGGTFTLSHGWAPGSLLTYSCPQGLYPSPASRLCKSSGQWQTPGATRSLSKAVCKPVRCPAPVSFENGIYTPRLGSYPVGGNVSFECEDGFILRGSPVRQCRPNGMWDGETAVCDNGAGHCPNPGISLGAVRTGFRFGHGDKVRYRCSSNLVLTGSSERECQGNGVWSGTEPICRQPYSYDFPEDVAPALGTSFSHMLGATNPTQKTKESLGRKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 186 - protein, 509 aa, Homo sapiens
KIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 187 - protein, 752 aa, Macaca fascicularis
MDPLMVLFCLLFLYPGPADSATSCPQNVNISGGTFTLSHGWAPGSLLIYSCPQGLYPSPASRLCKSSGQWQTPRATRSLTKAVCKPVRCPAPVSFENGIYTPRLGSYPVGGNVSFECEDGFILRGSPVRQCRPNGMWDGETAVCDNGAGHCPNPGISLGAVRTGSRFGHGDKVRYRCSSNLVLTGSAERECQGNGVWSGTEPICRQPYSYDFPEDVAPALGTSFSHMLGATNPTQRTKESLGRKIQIQRSGHLNLYLLLDSSQSVSENDFLIFKESASLMVDRIFSFEINVSVAIITFASEPKVLMSVLNVNSRDITEVISSLENAKYKDHENGTGTNTYAALNSVYLMMNNQMQLLGMKTMAWQEIRHAIILLTDGKSNMGGSPKTAVDQIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 188 - protein, 509 aa, Macaca fascicularis
KIQIQRSGHLNLYLLLDSSQSVSENDFLIFKESASLMVDRIFSFEINVSVAIITFASEPKVLMSVLNVNSRDITEVISSLENAKYKDHENGTGTNTYAALNSVYLMMNNQMQLLGMKTMAWQEIRHAIILLTDGKSNMGGSPKTAVDQIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 189 - DNA, 366 bp, synthetic construct
  source 1..366: mol_type = other DNA
gaggtgcagctgctggaatccggcggcggactcgtccaacccggcggatctctgcggctgtcctgtgctgcttctggcttcaccttcagacactacgccatgtcctgggtgcggcaggcccctggcaaaggcctggaatgggtcagcctgatctccggatctggcgcctctacccattatgccgattccgtgaagggcagatttaccatctctcgcgacaacagcgagaacatgctgtacctgcagatgaactccctgagagccgaggacaccgccgtgtactactgcgccaaggactccctggctgtggctggctccgagtacttccagcactggggccagggcacactggtgaccgtgtccagc

SEQ ID NO: 190 - DNA, 324 bp, synthetic construct
  source 1..324: mol_type = other DNA
agctatgtgctgacccaagccccttctgtgtctgtggcccctggacagacagctagaatcacctgtggacacaactactttggaggcaagagcgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccactgggtgtttggaggcggtaccaagctgaccgtgctg

SEQ ID NO: 191 - DNA, 324 bp, synthetic construct
  source 1..324: mol_type = other DNA
agctatgtgctgacccaagccccttctgtgtctgtggcccctggacagacagctagaatcacctgtggaggcaactaccatggaggcaagagcgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccactgggtgtttggaggcggtaccaagctgaccgtgctg

SEQ ID NO: 192 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagagccaccactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccactgggtgtttggaggcggtaccaagctgaccgtgctg

SEQ ID NO: 193 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagagcgtgcactggtatcagcagagacctggccaagcccctgtgctggtggtgtatcatgactctgacagaccttctggcatccctgagagattctctggcagcaagtctggcaacacagccaccctggccatcagcagagtggaggctggagatgaggctgcctactactgccaagtgtatgacacaagctctgaccactgggtgtttggaggcggtaccaagctgaccgtgctg

SEQ ID NO: 194 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtaccacacaagctctgaccactgggtgtttggaggcggtaccaagctgaccgtgctg

SEQ ID NO: 195 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgaccacagctctgaccactgggtgtttggaggcggtaccaagctgaccgtgctg

SEQ ID NO: 196 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacacactctgaccactgggtgtttggaggcggtaccaagctgaccgtgctg

SEQ ID NO: 197 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagccatgaccactgggtgtttggaggcggtaccaagctgaccgtgctg

SEQ ID NO: 198 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccaccatgtgtttggaggcggtaccaagctgaccgtgctg

SEQ ID NO: 199 - DNA, 324 bp, synthetic construct
  source 1..324: mol_type = other DNA
agctatgtgctgacccaagccccttctgtgtctgtggcccctggacagacagctagaatcacctgtggaggcaactactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccactggcactttggaggcggtaccaagctgaccgtgctg

SEQ ID NO: 200 - DNA, 324 bp, synthetic construct
  source 1..324: mol_type = other DNA
agctatgtgctgacccaagccccttctgtgtctgtggcccctggacagacagctagaatcacctgtggaggcaaccactttggaggcaagtctgtgcactggtatcagcagagacctggccaagcccctgtgctggtggtgtatgatgactctgacagaccttctggcatccctgagagattctctggcagcaagtctggcaacacagccaccctggccatcagcagagtggaggctggagatgaggctgcctactactgccaagtgtatgacacaagctctgaccactgggtgtttggaggcggtaccaagctgaccgtgctg

SEQ ID NO: 201 - protein, 306 aa, synthetic construct
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 202 - protein, 633 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 203 - protein, 633 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 204 - protein, 752 aa, synthetic construct
QVQLVQSGAEVKKPGASVKVSCKASGYSFTTYYMYWVRQAPGQGLEWMGIITPSGGSTTYAQKFQDRVTMTRDASTTTVYMELSSLRSEDTAVYYCARGYEGSYGFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 205 - protein, 212 aa, synthetic construct
SYELTQPPSVSLSPGQTASITCSGDKLGNKYTCWYQQKPGQSPVLVIYQDNKRPSGIPERFSGSNSGNTATLTISGTQAMDEADYYCQTWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 206 - protein, 755 aa, synthetic construct
EVQLLESGGGLVQPGGSLRLSCAASGFTFRHYAMSWVRQAPGKGLEWVSLISGSGASTFYADSVKGRFTISRDNSENMLYLQMNSLRAEDTAVYYCAKDSLAVAGSEYFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 207 - protein, 214 aa, synthetic construct
SYVLTQAPSVSVAPGQTARITCGGNYFGGKSVHWYQQRPGQAPVLVVYDDSDRPSGIPERFSGSKSGNTATLAISRVEAGDEAAYYCQVYDTSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 208 - DNA, 2259 bp, synthetic construct
  source 1..2259: mol_type = other DNA
caggtgcagctggtgcagtctggggctgaggtgaagaagcctggggcctcagtgaaggtttcctgtaaggcatctggatacagtttcaccacttactatatgtactgggtgcgacaggcccctggacaagggcttgagtggatgggaatcatcacccctagcggtggtagtacaacctacgcacagaagttccaggacagagtcaccatgacaagggacgcgtccacgactacagtctacatggagctgagcagcctgagatctgaggacacggccgtgtattactgtgcgagagggtatgaggggagctacgggtttgaccactggggccagggaaccctggtcaccgtctcctcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgctgcatgaggctctgcacgcccactacacacagaagagcctctccctgtctctgggtaaagaggattgtaacgagctgccacccagaagaaacacagagatcctgaccggaagctggagcgatcagacataccccgagggcacccaggccatttacaagtgcagacccggatatagaagcctgggcaacattatcatggtgtgcagaaaaggagagtgggtggccctgaaccctctgagaaagtgccagaaaagaccatgcggccacccaggcgacacccccttcggaaccttcacactgacaggcggcaacgtgtttgagtacggcgtgaaagctgtgtatacctgcaacgagggctaccagctgctgggagaaatcaactacagagagtgcgacaccgacggctggaccaacgacatccctatttgcgaggtggtgaagtgcctgcccgtgactgcccccgagaacggaaagatcgtgagcagcgccatggagcccgacagagagtaccactttggacaggccgtgagatttgtgtgcaacagcggctataagatcgagggcgacgaggaaatgcactgcagcgacgacggcttttggtcaaaggagaagcccaaatgcgtggagatcagctgcaaatctcccgacgtgatcaacggcagccctatcagccagaagatcatttacaaggagaacgagagattccagtacaagtgcaacatgggatacgagtactccgagagaggcgacgccgtgtgcaccgagagcggctggagaccactgcccagctgcgaggagaagagctgcgacaacccctacattccaaacggcgactacagcccactgaggatcaagcacagaacaggagatgagattacataccagtgtagaaacggcttctaccccgccaccagaggaaacaccgccaaatgcaccagcaccggctggattcctgccccccggtgcaccctgaaaccatga

SEQ ID NO: 209 - DNA, 639 bp, synthetic construct
  source 1..639: mol_type = other DNA
tcctatgaactgactcagccaccctcagtgtccctgtccccaggacagacagccagcatcacctgctctggagataaattgggaaataaatatacttgctggtatcagcagaagccaggccagtcccctgtcctggtcatctatcaagataacaagcggccctcggggatccctgagcgattctctggctccaactctgggaacacagccactctgaccatcagcgggacccaggctatggatgaggctgactattactgtcagacgtgggacagcagcactgcggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 210 - protein, 330 aa, synthetic construct [sequence omitted]

SEQ ID NO: 211 - protein, 636 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 212 - protein, 755 aa, synthetic construct
QVQLVQSGAEVKKPGASVKVSCKASGYSFTTYYMYWVRQAPGQGLEWMGIITPSGGSTTYAQKFQDRVTMTRDASTTTVYMELSSLRSEDTAVYYCARGYEGSYGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 213 - protein, 758 aa, synthetic construct
EVQLLESGGGLVQPGGSLRLSCAASGFTFRHYAMSWVRQAPGKGLEWVSLISGSGASTFYADSVKGRFTISRDNSENMLYLQMNSLRAEDTAVYYCAKDSLAVAGSEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 214 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 215 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 216 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 217 - DNA, 993 bp, synthetic construct
  source 1..993: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggatccttcttcctctacagcaagctcaccgtggacaagagcaggtggcagcaggggaacgtcttctcatgctccgtgctgcatgaggctctgcactcccactacacgcagaagagcctctccctgtctccgggtaaatag

SEQ ID NO: 218 - DNA, 993 bp, synthetic construct
  source 1..993: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagcagcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggatccttcttcctctacagcaagctcaccgtggacaagagcaggtggcagcaggggaacgtcttctcatgctccgtgctgcatgaggctctgcacgcccactacacgcagaagagcctctccctgtctccgggtaaatag

SEQ ID NO: 219 - DNA, 984 bp, synthetic construct
  source 1..984: mol_type = other DNA
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgctgcatgaggctctgcactcccactacacacagaagagcctctccctgtctctgggtaaatag

SEQ ID NO: 220 - protein, 636 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 221 - protein, 636 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 222 - protein, 633 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 223 - protein, 757 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 224 - protein, 757 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 225 - protein, 757 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 226 - protein, 754 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 227 - protein, 754 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHAHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 228 - protein, 754 aa, synthetic construct
EVQLLESGGGLVQPGGSLRLSCAASGFTFRHYAMSWVRQAPGKGLEWVSLISGSGASTHYADSVKGRFTISRDNSENMLYLQMNSLRAEDTAVYYCAKDSLAVAGSEYFQHWGQGTLVTVSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 229 - protein, 108 aa, synthetic construct; identical to SEQ ID NO: 18

SEQ ID NO: 230 - protein, 108 aa, synthetic construct; identical to SEQ ID NO: 97

SEQ ID NO: 231 - protein, 108 aa, synthetic construct; identical to SEQ ID NO: 101

SEQ ID NO: 232 - protein, 108 aa, synthetic construct; identical to SEQ ID NO: 105

SEQ ID NO: 233 - protein, 108 aa, synthetic construct; identical to SEQ ID NO: 109

SEQ ID NO: 234 - protein, 108 aa, synthetic construct; identical to SEQ ID NO: 113

SEQ ID NO: 235 - protein, 108 aa, synthetic construct; identical to SEQ ID NO: 117

SEQ ID NO: 236 - protein, 108 aa, synthetic construct; identical to SEQ ID NO: 121

SEQ ID NO: 237 - protein, 108 aa, synthetic construct; identical to SEQ ID NO: 125

SEQ ID NO: 238 - protein, 108 aa, synthetic construct; identical to SEQ ID NO: 129

SEQ ID NO: 239 - protein, 108 aa, synthetic construct; identical to SEQ ID NO: 133

SEQ ID NO: 240 - protein, 108 aa, synthetic construct; identical to SEQ ID NO: 137

SEQ ID NO: 241 - protein, 108 aa, synthetic construct; identical to SEQ ID NO: 141

SEQ ID NO: 242 - protein, 108 aa, synthetic construct; identical to SEQ ID NO: 145

SEQ ID NO: 243 - protein, 108 aa, synthetic construct; identical to SEQ ID NO: 149

SEQ ID NO: 244 - protein, 108 aa, synthetic construct; identical to SEQ ID NO: 153

SEQ ID NO: 245 - protein, 108 aa, synthetic construct; identical to SEQ ID NO: 157

SEQ ID NO: 246 - protein, 122 aa, synthetic construct
EVQLLESGGGLVQPGGSLRLSCAASGFTFRHYAMSWVRQAPGKGLEWVSLISGSGASTHYADSVKGRFTISRDNSKNTLYLQMNSLRAEDTAVYYCAKDSLAVAGSEYFQHWGQGTLVTVSS

SEQ ID NO: 247 - protein, 108 aa, synthetic construct
SYVLTQPPSVSVAPGQTARITCGHNYFGGKSVHWYQQKPGQAPVLVVYDDSDRPSGIPERFSGSNSGNTATLTISRVEAGDEADYYCQVYDTSSDHWVFGGGTKLTVL

SEQ ID NO: 248 - protein, 758 aa, synthetic construct
EVQLLESGGGLVQPGGSLRLSCAASGFTFRHYAMSWVRQAPGKGLEWVSLISGSGASTHYADSVKGRFTISRDNSKNTLYLQMNSLRAEDTAVYYCAKDSLAVAGSEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKP

SEQ ID NO: 249 - protein, 214 aa, synthetic construct
SYVLTQPPSVSVAPGQTARITCGHNYFGGKSVHWYQQKPGQAPVLVVYDDSDRPSGIPERFSGSNSGNTATLTISRVEAGDEADYYCQVYDTSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 250 - protein, 14 aa, synthetic construct
DSLAVAGSEYFQHW

(19) United States
(12) Patent Application Publication
(10) Pub. No.: US 2025/0376539 A1
(43) Pub. Date: Dec. 11, 2025

(54) ANTI-C2 ANTIBODIES AND USES THEREOF

(71) Applicants: Kira Pharmaceuticals (Suzhou) Ltd., Suzhou, Jiangsu (CN); The Trustees of the University of Pennsylvania, Philadelphia, PA (US)

(72) Inventors: Ping Tsui, North Potomac, MD (US); Bingbing Jiang, Suzhou (CN); Jianjun Zhang, Suzhou (CN); Yingying Xu, Suzhou (CN); Xiaoxia Hu, Suzhou (CN); Dongqiong Fei, Suzhou, Jiangsu (CN); Wenchao Song, Bryn Mawr, PA (US); Takashi Miwa, Bala Cynwyd, PA (US); Damodar Gullipalli, Mount Laurel, NJ (US); Sayaka Sato, Philadelphia, PA (US)

(21) Appl. No.: 18/833,612

(22) PCT Filed: Jan. 29, 2023
(86) PCT No.: PCT/CN2023/073730

§ 371 Date: Jul. 26, 2024

(51) Int. Cl. C07K 16/40; C12N 15/11; C12N 15/86

(52) U.S. Cl. CPC C07K 16/40; C12N 15/11; C12N 15/86; C07K 2317/52; C07K 2317/54; C07K 2317/55; C07K 2317/565; C07K 2317/622; C07K 2317/94; C07K 2319/00

(57) ABSTRACT

This invention relates to inhibition of the complement signaling using an anti-C2 antibody or a fragment thereof. Specifically, the invention relates to methods of treating a complement-mediated disease or complement-mediated disorder in an individual by contacting the individual with the anti-C2 antibody or a fragment thereof.

### CROSS REFERENCE TO RELATED APPLICATIONS

This application claims priority benefits of International Patent Application No. PCT/CN2022/074995, filed on Jan. 29, 2022, the content of which is incorporated herein by reference in its entirety.

### SUBMISSION OF SEQUENCE LISTING ON ASCII TEXT FILE

The contents of the electronic sequence listing (792252001041SEQLIST.xml; Size: 258,424 bytes; and Date of Creation: Jan. 27, 2023) is herein incorporated by reference in its entirety.

### FIELD OF THE INVENTION

This invention relates to anti-C2 antibodies, antibody derivative constructs, and uses thereof.

### BACKGROUND OF THE INVENTION

The complement system is part of innate immunity that plays a key role in host defense. However, activated complement also has the potential to cause significant tissue injury and destruction and dysregulated complement activity has been found to be associated with a number of rare and common diseases such as paroxysmal nocturnal hemoglobinuria (PNH), atypical hemolytic uremic syndrome, rheumatoid arthritis, age-related macular degeneration, and a number of autoantibody-mediated immunological disorders such as myasthenia gravis and neuromyelitis optica spectrum disorder etc. Thus, anti-complement therapy is a promising way of treating these human disorders.

The complement system can be activated via three pathways. The classical pathway is triggered by antibody-antigen complexes which recruit C1, composed of C1q, C1r and C1s, to cleave C4 and C2. The lectin pathway is initiated by pattern recognition molecules such as mannose-binding lectins and ficolins that activate mannose-binding lectin-associated serine proteases 1 and 2 (MASP-1 and MASP-2) which in turn cleave C4 and C2. The alternative pathway is constitutively active due to spontaneous hydrolysis of C3 which forms an initial C3 convertase C3(H2O)Bb, followed by the amplifying C3 convertase C3bBb, with participation of factor D, factor B and properdin, on susceptible surfaces. The three pathways converge at the C3 activation step which leads to C5 activation with the generation of C5a and C5b, the latter will initiate the formation of membrane attack complex (MAC) after combining with C6, C7, C8 and multiple C9. Activated complement achieves its biological functions through multiple mechanisms, opsonization of target surface with C3b/iC3b to facilitate phagocytosis, promotion of inflammatory response by the anaphylatoxins C3a and C5a to help clear invading pathogens or damaged tissue, and direct cell lysis through formation of MAC.

Complement component 2 (C2) is a critical part of the classical and the lectin pathways of the complement system, acting as a multi-domain serine protease. Activation of the classical and the lectin pathways requires binding of C2 to an activated surface-bound C4b in the presence of Ca²⁺. While bound to C4b, C2 can be cleaved by C1s or MASP2 into C2a and C2b. While cleaved C2b is released into the circulation, the C4b bound C2a forms a C3 convertase, C4bC2a, commonly referred to as the classical pathway C3 convertase. Addition of C3b into this complex during subsequent complement activation forms the C4bC2aC3b complex which serves as the classical/lectin pathway C5-convertase. C2a carries the catalytic activity in the classical/lectin pathway C3 and C5 convertases.

Successful immune surveillance by complement relies on the balance between activation and regulation as well as on discrimination between self and non-self surfaces. Any disruption of this balance may have severe adverse clinical consequences. Over-activation and/or under-regulation of the classical and lectin pathways, of which C2 is a key component, can lead to abnormality of this balance and contribute to autoimmune diseases affecting multiple organ systems such as neuromuscular junction and skin, resulting in neurological and skin diseases.

All references cited herein, including patent applications, patent publications, and Genbank Accession numbers are herein incorporated by reference, as if each individual reference were specifically and individually indicated to be incorporated by reference in its entirety.

### BRIEF SUMMARY OF THE INVENTION

The present application provides anti-C2 antibodies, constructs, and fragments thereof, including anti-C2 antibodies having pH-dependent binding to C2 and C2a, anti-C2 antibody fusion proteins, and fragments thereof.

In some embodiments, there is provided an anti-C2 antibody, a fragment, or a fusion protein thereof comprising a heavy chain variable region (VH) comprising an H-CDR1, an H-CDR2, and an H-CDR3 of an VH comprising the amino acid sequence of any one of SEQ ID NOs: 1, 9, 17, 25, 33, 41, 49, 57, 65, 73, 81, 89, and 246; and a light chain variable region (VL) comprising an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247.

In some embodiments, there is provided an anti-C2 antibody, a fragment, or a fusion protein thereof comprising an VH and VL, wherein:
a) the VH comprises i) an H-CDR1 comprising the amino acid sequence of SEQ ID NO: 3, ii) an H-CDR2 comprising the amino acid sequence of SEQ ID NO: 4, and iii) an H-CDR3 comprising the amino acid sequence of SEQ ID NO: 5, or a variant thereof comprising up to 3, 2, or 1 amino acid substitutions in the H-CDRs; and the VL comprises i) an L-CDR1 comprising the amino acid sequence of SEQ ID NO: 6, an L-CDR2 comprising the amino acid sequence of SEQ ID NO: 7, and iii) an L-CDR3 comprising the amino acid sequence of SEQ ID NO: 8, or a variant thereof comprising up to 3, 2, or 1 amino acid substitutions in the L-CDRs;
b) the VH comprises i) an H-CDR 1 comprising the amino acid sequence of SEQ ID NO: 11, ii) an H-CDR2 comprising the amino acid sequence of SEQ ID NO: 12, and iii) an H-CDR3 comprising the amino acid sequence of SEQ ID NO: 13, or a variant thereof comprising up to 3, 2, or 1 amino acid substitutions in the H-CDRs; and the VL comprises i) an L-CDR1 comprising the amino acid sequence of SEQ ID NO: 14, an L-CDR2 comprising the amino acid sequence of SEQ ID NO: 15, and iii) an L-CDR3 comprising the amino acid sequence of SEQ ID NO: 16, or a variant thereof comprising up to 3, 2, or 1 amino acid substitutions in the L-CDRs;
c) the VH comprises i) an H-CDR1 comprising the amino acid sequence of SEQ ID NO: 19, ii) an H-CDR2 comprising the amino acid sequence of SEQ ID NO: 20. and iii) an H-CDR3 comprising the amino acid sequence of SEQ ID NO: 21, or a variant thereof comprising up to 3, 2, or 1 amino acid substitutions in the H-CDRs; and the VL comprises i) an L-CDR1 comprising the amino acid sequence of SEQ ID NO: 22, an L-CDR2 comprising the amino acid sequence of SEQ ID NO: 23, and iii) an L-CDR3 comprising the amino acid sequence of SEQ ID NO: 24, or a variant thereof comprising up to 3, 2, or 1 amino acid substitutions in the L-CDRs;
d) the VH comprises i) an H-CDR1 comprising the amino acid sequence of SEQ ID NO: 27, ii) an H-CDR2 comprising the amino acid sequence of SEQ ID NO: 28, and iii) an H-CDR3 comprising the amino acid sequence of SEQ ID NO: 29, or a variant thereof comprising up to 3, 2, or 1 amino acid substitutions in the H-CDRs; and the VL comprises i) an L-CDR1 comprising the amino acid sequence of SEQ ID NO: 30, an L-CDR2 comprising the amino acid sequence of SEQ ID NO: 31, and iii) an L-CDR3 comprising the amino acid sequence of SEQ ID NO: 32, or a variant thereof comprising up to 3, 2, or 1 amino acid substitutions in the L-CDRs;
e) the VH comprises i) an H-CDR1 comprising the amino acid sequence of SEQ ID NO: 35. ii) an H-CDR2 comprising the amino acid sequence of SEQ ID NO: 36, and iii) an H-CDR3 comprising the amino acid sequence of SEQ ID NO: 37, or a variant thereof comprising up to 3, 2, or 1 amino acid substitutions in the H-CDRs; and the VL comprises i) an L-CDR1 comprising the amino acid sequence of SEQ ID NO: 38, an L-CDR2 comprising the amino acid sequence of SEQ ID NO: 39, and iii) an L-CDR3 comprising the amino acid sequence of SEQ ID NO: 40, or a variant thereof comprising up to 3, 2, or 1 amino acid substitutions in the L-CDRs;
f) the VH comprises i) an H-CDR1 comprising the amino acid sequence of SEQ ID NO: 43, ii) an H-CDR2 comprising the amino acid sequence of SEQ ID NO: 44, and iii) an H-CDR3 comprising the amino acid sequence of SEQ ID NO: 45, or a variant thereof comprising up to 3, 2, or 1 amino acid substitutions in the H-CDRs; and the VL comprises i) an L-CDR 1 comprising the amino acid sequence of SEQ ID NO: 46, an L-CDR2 comprising the amino acid sequence of SEQ ID NO: 47, and iii) an L-CDR3 comprising the amino acid sequence of SEQ ID NO: 48, or a variant thereof comprising up to 3, 2, or 1 amino acid substitutions in the L-CDRs;
g) the VH comprises i) an H-CDR1 comprising the amino acid sequence of SEQ ID NO: 51, ii) an H-CDR2 comprising the amino acid sequence of SEQ ID NO: 52, and iii) an H-CDR3 comprising the amino acid sequence of SEQ ID NO: 53, or a variant thereof comprising up to 3, 2, or 1 amino acid substitutions in the H-CDRs; and the VL comprises i) an L-CDR1 comprising the amino acid sequence of SEQ ID NO: 54, an L-CDR2 comprising the amino acid sequence of SEQ ID NO: 55, and iii) an L-CDR3 comprising the amino acid sequence of SEQ ID NO: 56, or a variant thereof comprising up to 3, 2, or 1 amino acid substitutions in the L-CDRs;
h) the VH comprises i) an H-CDR1 comprising the amino acid sequence of SEQ ID NO: 59, ii) an H-CDR2 comprising the amino acid sequence of SEQ ID NO: 60, and iii) an H-CDR3 comprising the amino acid sequence of SEQ ID NO: 61, or a variant thereof comprising up to 3, 2, or 1 amino acid substitutions in the H-CDRs; and the VL comprises i) an L-CDR 1 comprising the amino acid sequence of SEQ ID NO: 62, an L-CDR2 comprising the amino acid sequence of SEQ ID NO: 63, and iii) an L-CDR3 comprising the amino acid sequence of SEQ ID NO: 64, or a variant thereof comprising up to 3, 2, or 1 amino acid substitutions in the L-CDRs;
i) the VH comprises i) an H-CDR1 comprising the amino acid sequence of SEQ ID NO: 67, ii) an H-CDR2 comprising the amino acid sequence of SEQ ID NO: 68, and iii) an H-CDR3 comprising the amino acid sequence of SEQ ID NO: 69, or a variant thereof comprising up to 3, 2, or 1 amino acid substitutions in the H-CDRs; and the VL comprises i) an L-CDR1 comprising the amino acid sequence of SEQ ID NO: 70, an L-CDR2 comprising the amino acid sequence of SEQ ID NO: 71, and iii) an L-CDR3 comprising the amino acid sequence of SEQ ID NO: 72, or a variant thereof comprising up to 3, 2, or 1 amino acid substitutions in the L-CDRs;
j) the VH comprises i) an H-CDR1 comprising the amino acid sequence of SEQ ID NO: 75, ii) an H-CDR2 comprising the amino acid sequence of SEQ ID NO: 76, and iii) an H-CDR3 comprising the amino acid sequence of SEQ ID NO: 77, or a variant thereof comprising up to 3, 2, or 1 amino acid substitutions in the H-CDRs; and the VL comprises i) an L-CDR1 comprising the amino acid sequence of SEQ ID NO: 78, an L-CDR2 comprising the amino acid sequence of SEQ ID NO: 79, and iii) an L-CDR3 comprising the amino acid sequence of SEQ ID NO: 80, or a variant thereof comprising up to 3, 2, or 1 amino acid substitutions in the L-CDRs;
k) the VH comprises i) an H-CDR1 comprising the amino acid sequence of SEQ ID NO: 83, ii) an H-CDR2 comprising the amino acid sequence of SEQ ID NO: 84, and iii) an H-CDR3 comprising the amino acid sequence of SEQ ID NO: 85, or a variant thereof comprising up to 3, 2, or 1 amino acid substitutions in the H-CDRs; and the VL comprises i) an L-CDR1 comprising the amino acid sequence of SEQ ID NO: 86, an L-CDR2 comprising the amino acid sequence of SEQ ID NO: 87, and iii) an L-CDR3 comprising the amino acid sequence of SEQ ID NO: 88, or a variant thereof comprising up to 3, 2, or 1 amino acid substitutions in the L-CDRs; or
l) the VH comprises i) an H-CDR1 comprising the amino acid sequence of SEQ ID NO: 19, ii) an H-CDR2 comprising the amino acid sequence of SEQ ID NO: 92, and iii) an H-CDR3 comprising the amino acid sequence of SEQ ID NO: 250, or a variant thereof comprising up to 3, 2, or 1 amino acid substitutions in the H-CDRs; and the VL comprises i) an L-CDR1 comprising the amino acid sequence of SEQ ID NO: 98, an L-CDR2 comprising the amino acid sequence of SEQ ID NO: 23, and iii) an L-CDR3 comprising the amino acid sequence of SEQ ID NO: 24, or a variant thereof comprising up to 3, 2, or 1 amino acid substitutions in the L-CDRs.

In some embodiments, there is provided a pH-dependent, anti-C2 antibody, a fragment, or a fusion protein thereof comprising an VH and VL, wherein:
l) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 94, an L-CDR2 comprising the amino acid sequence of SEQ ID NO: 95, and iii) an L-CDR3 comprising the amino acid sequence of SEQ ID NO: 96;
m) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 98, an L-CDR2 comprising the amino acid sequence of SEQ ID NO: 99, and iii) an L-CDR3 comprising the amino acid sequence of SEQ ID NO: 100;
n) the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 102, an L-CDR2 comprising the amino acid sequence of SEQ ID NO: 103, and iii) an L-CDR3 comprising the amino acid sequence of SEQ ID NO: 104;
o) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 106, an L-CDR2 comprising the amino acid sequence of SEQ ID NO: 107, and iii) an L-CDR3 comprising the amino acid sequence of SEQ ID NO: 108;
p) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 110, an L-CDR2 comprising the amino acid sequence of SEQ ID NO: 111, and iii) an L-CDR3 comprising the amino acid sequence of SEQ ID NO: 112;
q) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 114, an L-CDR2 comprising the amino acid sequence of SEQ ID NO: 115, and iii) an L-CDR3 comprising the amino acid sequence of SEQ ID NO: 116;
r) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 118, an L-CDR2 comprising the amino acid sequence of SEQ ID NO: 119, and iii) an L-CDR3 comprising the amino acid sequence of SEQ ID NO: 120;
s) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 122, an L-CDR2 comprising the amino acid sequence of SEQ ID NO: 123, and iii) an L-CDR3 comprising the amino acid sequence of SEQ ID NO: 124;
t) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 126, an L-CDR2 comprising the amino acid sequence of SEQ ID NO: 127, and iii) an L-CDR3 comprising the amino acid sequence of SEQ ID NO: 128;
u) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 130, an L-CDR2 comprising the amino acid sequence of SEQ ID NO: 131, and iii) an L-CDR3 comprising the amino acid sequence of SEQ ID NO: 132;
v) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 134, ii) an L-CDR2 comprising the amino acid sequence of SEQ ID NO: 135, and iii) an L-CDR3 comprising the amino acid sequence of SEQ ID NO: 136;
w) the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 138, ii) an L-CDR2 comprising the amino acid sequence of SEQ ID NO: 139, and iii) an L-CDR3 comprising the amino acid sequence of SEQ ID NO: 140;
x) the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 142, ii) an L-CDR2 comprising the amino acid sequence of SEQ ID NO: 143, and iii) an L-CDR3 comprising the amino acid sequence of SEQ ID NO: 144;
y) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 146, ii) an L-CDR2 comprising the amino acid sequence of SEQ ID NO: 147, and iii) an L-CDR3 comprising the amino acid sequence of SEQ ID NO: 148;
z) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 150, ii) an L-CDR2 comprising the amino acid sequence of SEQ ID NO: 151, and iii) an L-CDR3 comprising the amino acid sequence of SEQ ID NO: 152;
aa) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 154, ii) an L-CDR2 comprising the amino acid sequence of SEQ ID NO: 155, and iii) an L-CDR3 comprising the amino acid sequence of SEQ ID NO: 156;
ab) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 158, ii) an L-CDR2 comprising the amino acid sequence of SEQ ID NO: 159, and iii) an L-CDR3 comprising the amino acid sequence of SEQ ID NO: 160; or
ac) the VH comprises i) an H-CDR1 comprising the amino acid sequence of SEQ ID NO: 19, ii) an H-CDR2 comprising the amino acid sequence of SEQ ID NO: 92, and iii) an H-CDR3 comprising the amino acid sequence of SEQ ID NO: 250; and the VL comprises i) an L-CDR1 comprising the amino acid sequence of SEQ ID NO: 98, ii) an L-CDR2 comprising the amino acid sequence of SEQ ID NO: 23, and iii) an L-CDR3 comprising the amino acid sequence of SEQ ID NO: 24.

In some embodiments according to any one of the anti-C2 antibodies or fragments thereof provided herein, the VH comprises i) an H-CDR1 comprising the amino acid sequence of SEQ ID NO: 3, ii) an H-CDR2 comprising the amino acid sequence of SEQ ID NO: 4, and iii) an H-CDR3 comprising the amino acid sequence of SEQ ID NO: 5, or a variant thereof comprising up to 3, 2, or 1 amino acid substitutions in the H-CDRs; and the VL comprises i) an L-CDR1 comprising the amino acid sequence of SEQ ID NO: 6, an L-CDR2 comprising the amino acid sequence of SEQ ID NO: 7, and iii) an L-CDR3 comprising the amino acid sequence of SEQ ID NO: 8, or a variant thereof comprising up to 3, 2, or 1 amino acid substitutions in the L-CDRs.

In some embodiments according to any one of the anti-C2 antibody or fragments thereof provided herein, the VH comprises i) an H-CDR1 comprising the amino acid sequence of SEQ ID NO: 11, ii) an H-CDR2 comprising the amino acid sequence of SEQ ID NO: 12, and iii) an H-CDR3 comprising the amino acid sequence of SEQ ID NO: 13, or a variant thereof comprising up to 3, 2, or 1 amino acid substitutions in the H-CDRs; and the VL comprises i) an L-CDR 1 comprising the amino acid sequence of SEQ ID NO: 14, an L-CDR2 comprising the amino acid sequence of SEQ ID NO: 15, and iii) an L-CDR3 comprising the amino acid sequence of SEQ ID NO: 16, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 19, ii) an H-CDR2 comprising the amino acid sequence of SEQ ID NO: 20, and iii) an H-CDR3 comprising the amino acid sequence of SEQ ID NO: 21, or a variant thereof comprising up to 3, 2, or 1 amino acid substitutions in the H-CDRs; and the VL comprises i) an L-CDR 1 comprising the amino acid sequence of SEQ ID NO: 22, an L-CDR2 comprising the amino acid sequence of SEQ ID NO: 23, and iii) an L-CDR3 comprising the amino acid sequence of SEQ ID NO: 24, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 27, ii) an H-CDR2 comprising the amino acid sequence of SEQ ID NO: 28, and iii) an H-CDR3 comprising the amino acid sequence of SEQ ID NO: 29, or a variant thereof comprising up to 3, 2, or 1 amino acid substitutions in the H-CDRs; and the VL comprises i) an L-CDR 1 comprising the amino acid sequence of SEQ ID NO: 30, an L-CDR2 comprising the amino acid sequence of SEQ ID NO: 31, and iii) an L-CDR3 comprising the amino acid sequence of SEQ ID NO: 32, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 35, ii) an H-CDR2 comprising the amino acid sequence of SEQ ID NO: 36, and iii) an H-CDR3 comprising the amino acid sequence of SEQ ID NO: 37, or a variant thereof comprising up to 3, 2, or 1 amino acid substitutions in the H-CDRs; and the VL comprises i) an L-CDR1 comprising the amino acid sequence of SEQ ID NO: 38, an L-CDR2 comprising the amino acid sequence of SEQ ID NO: 39, and iii) an L-CDR3 comprising the amino acid sequence of SEQ ID NO: 40, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 43, ii) an H-CDR2 comprising the amino acid sequence of SEQ ID NO: 44, and iii) an H-CDR3 comprising the amino acid sequence of SEQ ID NO: 45, or a variant thereof comprising up to 3, 2, or 1 amino acid substitutions in the H-CDRs; and the VL comprises i) an L-CDR1 comprising the amino acid sequence of SEQ ID NO: 46, an L-CDR2 comprising the amino acid sequence of SEQ ID NO: 47, and iii) an L-CDR3 comprising the amino acid sequence of SEQ ID NO: 48, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 51, ii) an H-CDR2 comprising the amino acid sequence of SEQ ID NO: 52, and iii) an H-CDR3 comprising the amino acid sequence of SEQ ID NO: 53, or a variant thereof comprising up to 3, 2, or 1 amino acid substitutions in the H-CDRs; and the VL comprises i) an L-CDR1 comprising the amino acid sequence of SEQ ID NO: 54, an L-CDR2 comprising the amino acid sequence of SEQ ID NO: 55, and iii) an L-CDR3 comprising the amino acid sequence of SEQ ID NO: 56, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 59, ii) an H-CDR2 comprising the amino acid sequence of SEQ ID NO: 60, and iii) an H-CDR3 comprising the amino acid sequence of SEQ ID NO: 61, or a variant thereof comprising up to 3, 2, or 1 amino acid substitutions in the H-CDRs; and the VL comprises i) an L-CDR1 comprising the amino acid sequence of SEQ ID NO: 62, an L-CDR2 comprising the amino acid sequence of SEQ ID NO: 63, and iii) an L-CDR3 comprising the amino acid sequence of SEQ ID NO: 64, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 67, ii) an H-CDR2 comprising the amino acid sequence of SEQ ID NO: 68, and iii) an H-CDR3 comprising the amino acid sequence of SEQ ID NO: 69, or a variant thereof comprising up to 3, 2, or 1 amino acid substitutions in the H-CDRs; and the VL comprises i) an L-CDR1 comprising the amino acid sequence of SEQ ID NO: 70, an L-CDR2 comprising the amino acid sequence of SEQ ID NO: 71, and iii) an L-CDR3 comprising the amino acid sequence of SEQ ID NO: 72, or a variant thereof comprising up to 3, 2, or 1 amino acid substitutions in the L-CDRs.

In some embodiments according to any one of the anti-C2 antibodies or fragments thereof provided herein, the VH comprises i) an H-CDR 1 comprising the amino acid sequence of SEQ ID NO: 75, ii) an H-CDR2 comprising the amino acid sequence of SEQ ID NO: 76, and iii) an H-CDR3 comprising the amino acid sequence of SEQ ID NO: 77, or a variant thereof comprising up to 3, 2, or 1 amino acid substitutions in the H-CDRs; and the VL comprises i) an L-CDR1 comprising the amino acid sequence of SEQ ID NO: 78, an L-CDR2 comprising the amino acid sequence of SEQ ID NO: 79, and iii) an L-CDR3 comprising the amino acid sequence of SEQ ID NO: 80, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 83, ii) an H-CDR2 comprising the amino acid sequence of SEQ ID NO: 84, and iii) an H-CDR3 comprising the amino acid sequence of SEQ ID NO: 85, or a variant thereof comprising up to 3, 2, or 1 amino acid substitutions in the H-CDRs; and the VL comprises i) an L-CDR1 comprising the amino acid sequence of SEQ ID NO: 86, an L-CDR2 comprising the amino acid sequence of SEQ ID NO: 87, and iii) an L-CDR3 comprising the amino acid sequence of SEQ ID NO: 88, or a variant thereof comprising up to 3, 2, or 1 amino acid substitutions in the L-CDRs.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 94, an L-CDR2 comprising the amino acid sequence of SEQ ID NO: 95, and iii) an L-CDR3 comprising the amino acid sequence of SEQ ID NO: 96.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 98, an L-CDR2 comprising the amino acid sequence of SEQ ID NO: 99, and iii) an L-CDR3 comprising the amino acid sequence of SEQ ID NO: 100.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 102, an L-CDR2 comprising the amino acid sequence of SEQ ID NO: 103, and iii) an L-CDR3 comprising the amino acid sequence of SEQ ID NO: 104.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 106, an L-CDR2 comprising the amino acid sequence of SEQ ID NO: 107, and iii) an L-CDR3 comprising the amino acid sequence of SEQ ID NO: 108.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 110, an L-CDR2 comprising the amino acid sequence of SEQ ID NO: 111, and iii) an L-CDR3 comprising the amino acid sequence of SEQ ID NO: 112.

In some embodiments according to any one of the anti-C2 antibodies or fragments thereof provided herein, the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 114, an L-CDR2 comprising the amino acid sequence of SEQ ID NO: 115, and iii) an L-CDR3 comprising the amino acid sequence of SEQ ID NO: 116.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 118, an L-CDR2 comprising the amino acid sequence of SEQ ID NO: 119, and iii) an L-CDR3 comprising the amino acid sequence of SEQ ID NO: 120.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 122, an L-CDR2 comprising the amino acid sequence of SEQ ID NO: 123, and iii) an L-CDR3 comprising the amino acid sequence of SEQ ID NO: 124.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 126, an L-CDR2 comprising the amino acid sequence of SEQ ID NO: 127, and iii) an L-CDR3 comprising the amino acid sequence of SEQ ID NO: 128.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 130, an L-CDR2 comprising the amino acid sequence of SEQ ID NO: 131, and iii) an L-CDR3 comprising the amino acid sequence of SEQ ID NO: 132.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 134, ii) an L-CDR2 comprising the amino acid sequence of SEQ ID NO: 135, and iii) an L-CDR3 comprising the amino acid sequence of SEQ ID NO: 136.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 138, ii) an L-CDR2 comprising the amino acid sequence of SEQ ID NO: 139, and iii) an L-CDR3 comprising the amino acid sequence of SEQ ID NO: 140.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 142, ii) an L-CDR2 comprising the amino acid sequence of SEQ ID NO: 143, and iii) an L-CDR3 comprising the amino acid sequence of SEQ ID NO: 144.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 146, ii) an L-CDR2 comprising the amino acid sequence of SEQ ID NO: 147, and iii) an L-CDR3 comprising the amino acid sequence of SEQ ID NO: 148.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 150, ii) an L-CDR2 comprising the amino acid sequence of SEQ ID NO: 151, and iii) an L-CDR3 comprising the amino acid sequence of SEQ ID NO: 152.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 154, ii) an L-CDR2 comprising the amino acid sequence of SEQ ID NO: 155, and iii) an L-CDR3 comprising the amino acid sequence of SEQ ID NO: 156.

In some embodiments according to any one of the anti-C2 antibodies or fragments thereof provided herein, the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 158, ii) an L-CDR2 comprising the amino acid sequence of SEQ ID NO: 159, and iii) an L-CDR3 comprising the amino acid sequence of SEQ ID NO: 160.

In some embodiments according to any one of the anti-C2 antibodies or fragments thereof provided herein, the VH comprises an amino acid sequence of any one of SEQ ID NOs: 1, 9, 17, 25, 33, 41, 49, 57, 65, 73, 81, 89, and 246 or a variant comprising an amino acid consequence having at least about 80% sequence identity; and/or wherein the VL comprises an amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247 or a variant comprising an amino acid consequence having at least about 80% sequence identity.

In some embodiments according to any one of the anti-C2 antibodies or fragments thereof provided herein,
a) the VH comprises an amino acid sequence of SEQ ID NO: 1, or a variant comprising an amino acid sequence having at least about 80% sequence identity; and the VL comprises an amino acid sequence of SEQ ID NO: 2, or a variant comprising an amino acid sequence having at least about 80% sequence identity;
b) the VH comprises an amino acid sequence of SEQ ID NO: 9, or a variant comprising an amino acid sequence having at least about 80% sequence identity; and the VL comprises an amino acid sequence of SEQ ID NO: 10, or a variant comprising an amino acid sequence having at least about 80% sequence identity;
c) the VH comprises an amino acid sequence of SEQ ID NO: 17, or a variant comprising an amino acid sequence having at least about 80% sequence identity; and the VL comprises an amino acid sequence of SEQ ID NO: 18, or a variant comprising an amino acid sequence having at least about 80% sequence identity;
d) the VH comprises an amino acid sequence of SEQ ID NO: 25, or a variant comprising an amino acid sequence having at least about 80% sequence identity; and the VL comprises an amino acid sequence of SEQ ID NO: 26, or a variant comprising an amino acid sequence having at least about 80% sequence identity;
e) the VH comprises an amino acid sequence of SEQ ID NO: 33, or a variant comprising an amino acid sequence having at least about 80% sequence identity; and the VL comprises an amino acid sequence of SEQ ID NO: 34, or a variant comprising an amino acid sequence having at least about 80% sequence identity;
f) the VH comprises an amino acid sequence of SEQ ID NO: 41, or a variant comprising an amino acid sequence having at least about 80% sequence identity; and the VL comprises an amino acid sequence of SEQ ID NO: 42, or a variant comprising an amino acid sequence having at least about 80% sequence identity;
g) the VH comprises an amino acid sequence of SEQ ID NO: 49, or a variant comprising an amino acid sequence having at least about 80% sequence identity; and the VL comprises an amino acid sequence of SEQ ID NO: 50, or a variant comprising an amino acid sequence having at least about 80% sequence identity;
h) the VH comprises an amino acid sequence of SEQ ID NO: 57, or a variant comprising an amino acid sequence having at least about 80% sequence identity; and the VL comprises an amino acid sequence of SEQ ID NO: 58, or a variant comprising an amino acid sequence having at least about 80% sequence identity;
i) the V H comprises an amino acid sequence of SEQ ID NO: 65, or a variant comprising an amino acid sequence having at least about 80% sequence identity; and the VL comprises an amino acid sequence of SEQ ID NO: 66, or a variant comprising an amino acid sequence having at least about 80% sequence identity;
j) the VH comprises an amino acid sequence of SEQ ID NO: 73, or a variant comprising an amino acid sequence having at least about 80% sequence identity; and the VL comprises an amino acid sequence of SEQ ID NO: 74, or a variant comprising an amino acid sequence having at least about 80% sequence identity;
k) the VH comprises an amino acid sequence of SEQ ID NO: 81, or a variant comprising an amino acid sequence having at least about 80% sequence identity; and the VL comprises an amino acid sequence of SEQ ID NO: 82, or a variant comprising an amino acid sequence having at least about 80% sequence identity;
l) the VH comprises an amino acid sequence of SEQ ID NO: 89, or a variant comprising an amino acid sequence having at least about 80% sequence identity; and the VL comprises an amino acid sequence of SEQ ID NO: 90, or a variant comprising an amino acid sequence having at least about 80% sequence identity;
m) the VH comprises an amino acid sequence of SEQ ID NO: 89, or a variant comprising an amino acid sequence having at least about 80% sequence identity; and the VL comprises an amino acid sequence of SEQ ID NO: 97, or a variant comprising an amino acid sequence having at least about 80% sequence identity;
n) the VH comprises an amino acid sequence of SEQ ID NO: 89, or a variant comprising an amino acid sequence having at least about 80% sequence identity; and the VL comprises an amino acid sequence of SEQ ID NO: 101, or a variant comprising an amino acid sequence having at least about 80% sequence identity;
o) the VH comprises an amino acid sequence of SEQ ID NO: 89, or a variant comprising an amino acid sequence having at least about 80% sequence identity; and the VL comprises an amino acid sequence of SEQ ID NO: 105, or a variant comprising an amino acid sequence having at least about 80% sequence identity;
p) the VH comprises an amino acid sequence of SEQ ID NO: 89, or a variant comprising an amino acid sequence having at least about 80% sequence identity; and the VL comprises an amino acid sequence of SEQ ID NO: 109, or a variant comprising an amino acid sequence having at least about 80% sequence identity;
q) the VH comprises an amino acid sequence of SEQ ID NO: 89, or a variant comprising an amino acid sequence having at least about 80% sequence identity; and the VL comprises an amino acid sequence of SEQ ID NO: 113, or a variant comprising an amino acid sequence having at least about 80% sequence identity;
r) the VH comprises an amino acid sequence of SEQ ID NO: 89, or a variant comprising an amino acid sequence having at least about 80% sequence identity; and the VL comprises an amino acid sequence of SEQ ID NO: 117, or a variant comprising an amino acid sequence having at least about 80% sequence identity;
s) the VH comprises an amino acid sequence of SEQ ID NO: 89, or a variant comprising an amino acid sequence having at least about 80% sequence identity; and the VL comprises an amino acid sequence of SEQ ID NO: 121, or a variant comprising an amino acid sequence having at least about 80% sequence identity;
t) the V H comprises an amino acid sequence of SEQ ID NO: 89, or a variant comprising an amino acid sequence having at least about 80% sequence identity; and the VL comprises an amino acid sequence of SEQ ID NO: 125, or a variant comprising an amino acid sequence having at least about 80% sequence identity;
u) the VH comprises an amino acid sequence of SEQ ID NO: 89, or a variant comprising an amino acid sequence having at least about 80% sequence identity; and the VL comprises an amino acid sequence of SEQ ID NO: 129, or a variant comprising an amino acid sequence having at least about 80% sequence identity;
v) the VH comprises an amino acid sequence of SEQ ID NO: 89, or a variant comprising an amino acid sequence having at least about 80% sequence identity; and the VL comprises an amino acid sequence of SEQ ID NO: 133, or a variant comprising an amino acid sequence having at least about 80% sequence identity;
w) the VH comprises an amino acid sequence of SEQ ID NO: 89, or a variant comprising an amino acid sequence having at least about 80% sequence identity; and the VL comprises an amino acid sequence of SEQ ID NO: 137, or a variant comprising an amino acid sequence having at least about 80% sequence identity;
x) the VH comprises an amino acid sequence of SEQ ID NO: 89, or a variant comprising an amino acid sequence having at least about 80% sequence identity; and the VL comprises an amino acid sequence of SEQ ID NO: 141, or a variant comprising an amino acid sequence having at least about 80% sequence identity;
y) the VH comprises an amino acid sequence of SEQ ID NO: 89, or a variant comprising an amino acid sequence having at least about 80% sequence identity; and the VL comprises an amino acid sequence of SEQ ID NO: 145, or a variant comprising an amino acid sequence having at least about 80% sequence identity;
z) the VH comprises an amino acid sequence of SEQ ID NO: 89, or a variant comprising an amino acid sequence having at least about 80% sequence identity; and the VL comprises an amino acid sequence of SEQ ID NO: 149, or a variant comprising an amino acid sequence having at least about 80% sequence identity;
aa) the VH comprises an amino acid sequence of SEQ ID NO: 89, or a variant comprising an amino acid sequence having at least about 80% sequence identity; and the VL comprises an amino acid sequence of SEQ ID NO: 153, or a variant comprising an amino acid sequence having at least about 80% sequence identity;
ab) the VH comprises an amino acid sequence of SEQ ID NO: 89, or a variant comprising an amino acid sequence having at least about 80% sequence identity; and the VL comprises an amino acid sequence of SEQ ID NO: 157, or a variant comprising an amino acid sequence having at least about 80% sequence identity; or
ac) the VH comprises an amino acid sequence of SEQ ID NO: 246, or a variant comprising an amino acid sequence having at least about 80% sequence identity; and the VL comprises an amino acid sequence of SEQ ID NO: 247, or a variant comprising an amino acid sequence having at least about 80% sequence identity.

In some embodiments, there is provided an anti-C2 antibody, a fragment, or a fusion protein thereof that binds to C2 or C2a competitively with any one of the anti-C2 antibodies or fragments thereof provided herein.

In some embodiments according to any one of the anti-C2 antibodies or fragments thereof provided herein, the antibody or a fragment thereof is selected from the group consisting of: a full-length antibody, Fab, Fab′, F(ab)2, F(ab′)2, and scFv.

In some embodiments according to any one of the anti-C2 antibodies or fragments thereof provided herein, the antibody or a fragment thereof further comprises an Fc region. In some embodiments, the Fc region comprises an IgG4 sequence. In some embodiments, the Fc region comprises the amino acid sequence of SEQ ID NO: 183 or a variant thereof. In some embodiments, the Fc region comprises one or more mutations selected from the group consisting of S228P, M428L, N434A and N434S, wherein the mutations are relative to SEQ ID NO: 183 under the EU numbering system. In some embodiments, the Fc region comprises mutations S228P, M428L and N434A. In some embodiments, the Fc region comprises mutations S228P, M428L and N434S. In some embodiments, the Fc region comprises amino acid sequence of SEQ ID NO: 184. In some embodiments, the Fc region comprises amino acid sequence of SEQ ID NO: 216. In some embodiments, the Fc region comprises an IgG1 sequence. In some embodiments, the Fc region comprises the amino acid sequence of SEQ ID NO: 210 or a variant thereof. In some embodiments, the Fc region comprises one or more mutations selected from the group consisting of L234A, L235A, M428L, N434A and N434S, wherein the mutations are relative to SEQ ID NO: 210 under the EU numbering system. In some embodiments, the Fc region comprises mutations L234A, L235A, M428L and N434A. In some embodiments, the Fc region comprises mutations L234A, L235A, M428L and N434S. In some embodiments, the Fc region comprises the amino acid sequence of SEQ ID NO: 214. In some embodiments, the Fc region comprises the amino acid sequence of SEQ ID NO: 215.

In some embodiments according to any one of the anti-C2 antibodies or fragments thereof provided herein, the low-pH dissociation factor of the antibody or a fragment thereof dissociating from human C2 is between about 40% to about 95%.

In some embodiments according to any one of the anti-C2 antibodies or fragments thereof provided herein, the neutral-pH dissociation factor of the antibody or a fragment thereof dissociating from human C2 is between about 0% to about 15%.

In some embodiments according to any one of the anti-C2 antibodies or fragments thereof provided herein, the ratio of low-pH dissociation to neutral-pH dissociation from human C2 is 5 or more.

In some embodiments according to any one of the anti-C2 antibodies or fragments thereof provided herein, the low-pH dissociation factor of the antibody or a fragment thereof dissociating from cynomolgus C2 is between about 40% to about 80%.

In some embodiments according to any one of the anti-C2 antibodies or fragments thereof provided herein, the neutral-pH dissociation factor of the antibody or a fragment thereof dissociating from cynomolgus C2 is between about 0% to about 10%.

In some embodiments according to any one of the anti-C2 antibodies or fragments thereof provided herein, the ratio of low-pH dissociation to neutral-pH dissociation from cynomolgus C2 is 7 or more.

In some embodiments according to any one of the anti-C2 antibodies or fragments thereof provided herein, the antibody or a fragment thereof inhibits the cleavage of human C2 into fragments C2a and C2b.

In some embodiments according to any one of the anti-C2 antibodies or fragments thereof provided herein, the antibody or a fragment thereof binds to human C2a. In some embodiments, the antibody or a fragment thereof cross-reacts with a cynomolgus monkey C2. In some embodiment, the antibody or a fragment thereof cross-reacts with a cynomolgus monkey C2a.

In some embodiments according to any one of the anti-C2 antibodies or fragments thereof provided herein, the antibody or a fragment thereof has a serum half-life in humans that is at least about 2 days.

In some embodiments according to any one of the anti-C2 antibodies or fragments thereof provided herein, the antibody or a fragment thereof is manufactured in CHO cells.

In some embodiments, there is provided a nucleic acid encoding any one of the anti-C2 antibodies or fragments thereof provided herein. In some embodiments, there is provided a vector comprising the nucleic acid encoding any one of the anti-C2 antibodies or fragments thereof provided herein. In some embodiments, there is provided a host cell comprising said vector.

In some embodiments, there is provided a method of producing any one of the anti-C2 antibodies or fragments thereof provided herein by allowing expression of the antibody or a fragment thereof by cell under a sufficient condition.

In some embodiments, there is provided a pharmaceutical composition comprising any one of the anti-C2 antibodies or fragments thereof provided herein and a pharmaceutically acceptable carrier. In some embodiments, there is provided a method of treating an individual having a complement-associated disease or condition, comprising administering to the individual an effective amount of the pharmaceutical composition provided herein.

In some embodiments, the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, Hidradenitis Suppurativa, bullous pemphigoid, Myelin oligodendrocyte glycoprotein antibody-associated disease (MOGAD), sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD) or any combinations thereof.

In some embodiments, there is provided a method of reducing the activity of a complement system in an individual, comprising administering to the individual an effective amount of the pharmaceutical composition provided herein.

In some embodiments according to any one of the anti-C2 antibodies or fragments thereof provided herein, the antibody or a fragment thereof is an isolated anti-C2 antibody or a fragment thereof.

In some embodiments, there is provided a fusion protein comprising any one of the anti-C2 antibodies or fragments thereof provided herein. In some embodiments, the fusion protein further comprises a factor H protein or a fragment thereof.

In some embodiments according to any one of the anti-C2 antibodies or fragments thereof provided herein, the antibody or a fragment thereof is a fully human antibody or a fragment thereof.

In some embodiments, there is provided an anti-C2 antibody fusion protein comprising a heavy chain-factor H fusion polypeptide and a light chain, wherein the heavy chain-factor H fusion comprises an amino acid sequence of any one of SEQ ID NOs: 202-204, 206, 211-213, and 220-228, and the light chain comprises an amino acid sequence of any one of SEQ ID NOs: 205, 207, and 229-245.

In some embodiments, the anti-C2 antibody fusion protein provided herein is capable of overcoming the C2 bypass phenomenon.

In some embodiments, the anti-C2 antibody fusion protein comprises a VL sequence selected from any of SEQ ID NOs: 229-245, and a heavy-chain FH fusion sequence selected from any of SEQ ID NOs: 223-228.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIGS. 1A and 1B show the human C2 binding activities of 10 recombinant anti-C2 antibodies (34.5, 199.1, 39.4, 3.2, 48.14, 83.16, 139.14, 149.46, 59.45, and 69.44).

FIGS. 2A and 2B show the cyno C2 binding activities of 10 recombinant anti-C2 antibodies (34.5, 199.1, 39.4, 3.2, 48.14, 83.16, 139.14, 149.46, 59.45, and 69.44).

FIG. 3 shows the human C2a binding activities of 10 recombinant anti-C2 antibodies (34.5, 199.1, 39.4, 3.2, 48.14, 83.16, 139.14, 149.46, 59.45, and 69.44).

FIG. 4 shows the cyno C2a binding activities of 10 recombinant anti-C2 antibodies (34.5, 199.1, 39.4, 3.2, 48.14, 83.16, 139.14, 149.46, 59.45, and 69.44).

FIG. 5 shows classical pathway inhibitory activities of top 5 selections of recombinant anti-C2 antibodies (34.5, 199.1, 3.2, 149.46, and 69.44) in 5% normal human serum, determined by sheep RBC lysis assay.

FIG. 6 shows classical pathway inhibitory activities of top 5 selections of recombinant anti-C2 antibodies (34.5, 199.1, 3.2, 149.46, and 69.44) in 1% normal human serum, determined by IgM-mediated C3b deposition ELISA.

FIG. 7 shows lectin pathway inhibitory activities of top 5 selections of recombinant anti-C2 antibodies (34.5, 199.1, 3.2, 149.46, and 69.44) in 1% normal human serum, determined by mannan-mediated C3b deposition ELISA.

FIG. 8 shows the binding affinity measurement of anti-C2 mAb 69.44, 3.2 and 3.2 H2-10 to human C2 under neutral pH.

FIG. 9 shows the human C2 binding affinity measurements of mAb 69.44-FH1-5 and mAb 3.2 H2-10 FH1-5.

FIG. 10 shows pH-dependent human C2 binding affinity measurements of selected FH1-5 fusion constructs in comparison to non-FH fusion anti-C2 constructs.

FIGS. 11A and 11B show the classical pathway inhibitory activities of recombinant anti-C2 antibodies 69.44 and 3.2 in 5% and 50% normal human serum, determined by sheep RBC lysis assay.

FIGS. 12A and 12B show the classical pathway inhibitory activities of recombinant anti-C2 antibodies 69.44 and 3.2 H2-10 with or without FH1-5 fusion in 1% and 50% normal human serum, determined by sheep RBC lysis assay.

FIGS. 13A and 13B show the classical pathway inhibitory activities of recombinant anti-C2-FH fusion antibodies 69.44FH1-5 and 3.2 H2-10 FH1-5 in 1% and 50% normal human serum, determined by IgM-mediated C3b deposition ELISA.

FIGS. 14A and 14B show the lectin pathway inhibitory activities of recombinant anti-C2-FH fusion antibodies 69.44FH1-5 and 3.2 H2-10 FH1-5 in 1% and 50% normal human serum, determined by mannan-mediated C3b deposition ELISA.

FIGS. 15A and 15B show the alternative pathway inhibitory activities of recombinant anti-C2-FH fusion antibodies 69.44FH1-5 and 3.2 H2-10 FH1-5 in 10% and 50% normal human serum, determined by LPS-mediated C3b deposition ELISA.

FIGS. 16A and 16B show the classical pathway inhibitory activities of recombinant anti-C2-FH fusion antibodies 69.44FH1-5 and 3.2 H2-10 FH1-5 in 1% and 50% normal human serum, determined by IgM-mediated C5b-9 complex deposition ELISA.

FIGS. 17A and 17B show the alternative pathway inhibitory activities of recombinant anti-C2-FH fusion antibodies 69.44FH1-5 and 3.2 H2-10 I FH1-5 in 10% and 50% normal human serum, determined by LPS-mediated C5b-9 complex deposition ELISA.

FIG. 18 shows elements of an AAV vector containing the human C2 cDNA.

FIG. 19 shows that while the 69.44 antibody is detected, there is no detection of the 3.2 antibody in a human C2 ELISA.

FIG. 20 shows different combinations of capture and detection antibodies for a human C2 ELISA.

FIG. 21 shows the ELISA results for different combinations of capture and detection antibodies.

FIGS. 22A and 22B show the pharmacokinetic profiles of anti-C2 antibodies in a human-C2 mouse model and a table of the corresponding raw data of the human IgG4 ELISA, respectively.

FIG. 23 shows detection of human C2 protein in mice plasma at various time points following a single intravenous (i.v.) injection of the anti-human C2 antibodies at 40 mg/kg.

FIGS. 24A-24C show the pharmacodynamic properties of the anti-human C2 antibodies using a sheep red blood cell lysis assay and tables of the corresponding raw data.

FIG. 25 shows the pharmacokinetic profile of wild type (3.2) and pH-dependent (3.2 pH) anti-human C2 antibodies.

FIG. 26A shows the detection of human C2 protein in mice plasma at various time points following a single i.v. injection of 40 mg/kg wild type (3.2) or pH-dependent (3.2 pH) anti-human C2 antibodies. FIG. 26B shows a table of the corresponding raw data of the human C2 ELISA in FIG. 26A. FIG. 26C shows the fold increase of control for the human C2 ELISA results.

FIG. 27A shows the pharmacokinetic profiles of a wild type anti-human C2 antibody (3.2WT), pH-dependent anti-human C2 antibody (3.2pHGL), and anti-human C2 fusion proteins (69.44-FH and 3.2pHGL-FH). FIG. 27B shows a table of the corresponding raw data of the IgG4 ELISAs.

FIG. 28A shows the detection of human C2 protein in mice plasma at various time points following a single i.v. injection of 40 mg/kg wild type anti-human C2 antibody (3.2WT), pH-dependent anti-human C2 antibody (3.2pHGL), and anti-human C2 fusion proteins (69.44-FH and 3.2pHGL-FH). FIG. 28B shows a table of the corresponding raw data of human C2 ELISA in FIG. 28A. FIG. 28C shows the fold increase of control for the human C2 ELISA results.

FIG. 29A shows a pharmacokinetic analysis of recombinant anti-C2 antibodies (34.5, 199.1, 149.46, and 69.44) from mice expressing low or high levels of human C2. FIG. 29B shows a table of the corresponding raw data.

FIG. 30 show the pharmacokinetic profiles of IgG1 versus IgG4 constructs for 69.44 and 3.2 pH anti-human C2 antibodies.

FIG. 31 show the detection of human C2 protein in mice plasma at various time points following a single i.v. injection of 40 mg/kg 3.2 pH-IgG1, 3.2pH-IgG4, 69.44-IgG1, and 69.44-IgG4.

FIGS. 32A and 32B show the pharmacokinetic profiles of various anti-human C2 fusion proteins (69.44-IgG4-FH and 3.2 pH-IgG4-FH).

FIG. 33 shows the k_{off}, kₒₙ, and KD for 3.2 pH and germlined 3.2 pH anti-C2 antibodies.

FIG. 34 shows the detection of IgG1/IgG4 in mice plasma at various time points following a single i.v. injection of 40 mg/kg 3.2 pH IgG4 PLA, 3.2 pH IgG4 PLS, 3.2 pH IgG1 LALALA, and 3.2 pH IgG1 LALALS.

FIG. 35 shows the detection of human C2 protein in mice plasma at various time points following a single i.v. injection of 40 mg/kg 3.2 pH IgG4 PLA, 3.2 pH IgG4 PLS, 3.2 pH IgG1 LALALA, and 3.2 pH IgG1 LALALS.

FIG. 36 shows the fold change of C2 accumulation relative to pre-injection control of 3.2 pH IgG4 PLA, 3.2 pH IgG4 PLS, 3.2 pH IgG1 LALALA, and 3.2 pH IgG1 LALALS.

FIG. 37 shows the detection of IgG1/IgG4 in mice plasma at various time points following a single i.v. injection of 40 mg/kg 69 IgG4 PLA-FH, 3.2 pH IgG4 PLA-FH, 3.2pHGL-IgG1 LALA-LS-FH (germline), and 3.2 pH-IgG1 LALA-LS-FH anti-C2 fusion proteins.

FIG. 38 show the percent change of 2 hours for 69 IgG4 PLA-FH, 3.2 pH IgG4 PLA-FH, 3.2pHGL-IgG1 LALA-LS-FH (germline), and 3.2 pH-IgG1 LALA-LS-FH anti-C2 fusion proteins detection.

FIG. show the detection of human C2 protein in mice plasma at various time points following a single i.v. injection of 40 mg/kg 69 IgG4 PLA-FH, 3.2 pH IgG4 PLA-FH, 3.2pHGL-IgG1 LALA-LS-FH (germline), and 3.2 pH-IgG1 LALA-LS-FH anti-C2 fusion proteins.

FIG. 40 shows the fold change of C2 accumulation relative to pre-injection control of 40 mg/kg 69 IgG4 PLA-FH, 3.2 pH IgG4 PLA-FH, 3.2pHGL-IgG1 LALA-LS-FH (germline), and 3.2 pH-IgG1 LALA-LS-FH anti-C2 fusion proteins.

FIG. 41 shows the wildtype and germline-derived 3.2 pH (3.2 pH and 3.2pHGL, respectively) have identical binding affinities.

FIG. 42 shows the biological activities of anti-C2 antibodies 3.2 pH-IgG1-LALA-LS and 3.2pHGL-IgG1-LALA-LS in 20% normal human serum, determined by a sheep RBC lysis assay.

FIG. 43 shows the biological activities of anti-C2 antibodies 3.2 pH-IgG1-LALA-LS and 3.2pHGL-IgG1-LALA-LS in 20% cynomolgus serum, determined by a sheep RBC lysis assay.

FIG. 44 shows 3.2 pH-FH-IgG1-LALA-LS, 3.2 pH-FH- IgG4-PLA, and 3.2pHGL-FH-IgG1-LALA-LS fusions demonstrate identical biological activities in in vitro assays.

### DETAILED DESCRIPTION OF THE INVENTION

The present application provides novel anti-C2 antibodies and antibody constructs that specifically binds to C2 and C2a and comprises a heavy chain variable domain (VH) and a light chain variable domain (VL). In some aspects, the anti-C2 antibodies and antibody constructs contain mutations in its VH and/or VL domains. In one aspect, the mutations in the VH and the VL render the antibodies and antibody constructs pH sensitive in antigen binding. These antibodies and antibody constructs are useful for treating complement-medicated diseases or complement-mediated disorder by effectively and specifically targeting and inhibiting target key components of classical and lectin pathway over-activation.

In some embodiments, there is provided a method of reducing the activity of a complement system by targeting C2 and C2a proteins. In some embodiments, there is provided a method of inhibiting the complement signaling cascade by targeting C2 and C2a proteins. In one embodiment, there is provided a method of treating and preventing inflammation and autoimmune diseases and disorders mediated by unwanted, uncontrolled, or excessive complement activation. In one embodiment, there is provided a method of treatment of a complement-mediated disease or a complement-mediated disorder in an individual by contacting the individual with an anti-C2 antibody or an anti-C2 antibody construct, including fusion proteins or a fragment thereof.

In various embodiments, there is provided a method of compositions and methods for treating a complement-mediated disease or complement-mediated disorder in an individual by contacting the individual with an anti-C2 antibody or an antibody construct. The complement-mediated diseases and disorders that can be treated with the compositions and methods of the invention include, but are not limited to, macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, Hidradenitis Suppurativa, bullous pemphigoid, Myelin oligodendrocyte glycoprotein antibody-associated disease (MOGAD), sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD) or any combinations thereof.

In another aspect, there are provided methods for inhibiting complement activation and/or methods of treating diseases (such as complement associated diseases) by administering any one or more of the anti-C2 antibodies or constructs thereof.

In another aspect, there are provided exemplary nucleic acids encoding any one or more of the anti-C2 antibodies or constructs thereof, as well as vectors or host cells comprising such nucleic acids. Methods of making the anti-C2 antibodies are also described.

### 1. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “effective amount” and “pharmaceutically effective amount” as used herein refer to a sufficient amount of an agent to provide the desired biological result. That result can be reduction and/or alleviation of the signs, symptoms, or causes of a disease or disorder, or any other desired alteration of a biological system.

As used herein, the terms “patient,” “subject,” “individual,” and the like are used interchangeably herein, and refer to any animal, in some embodiments a mammal, and in some embodiments a human, having a complement system, including a human in need of therapy for, or susceptible to, a condition or its sequelae. The individual may include, for example, dogs, cats, pigs, cows, sheep, goats, horses, rats, monkeys, mice and humans. In some embodiments, the individual is a human.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody” may refer an immunoglobulin molecule or a fragment thereof which is able to specifically bind to a specific epitope of an antigen (including the basic 4-chain antibody unit). Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), antigen-binding fragments (such as Fv, Fab, Fab′, F(ab)2 and F(ab′)2), as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the VH and V_{L} antibody domains connected into a single polypeptide chain. In some embodiments, the scFv polypeptide further comprises a polypeptide linker between the VH and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the VH and the C_{L} is aligned with the first constant domain of the heavy chain (C_{H}1). Particular amino acid residues are believed to form an interface between the light chain and heavy chain variable domains. The pairing of an VH and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

An “isolated” antibody is one that has been identified, separated and/or recovered from a component of its production environment (E.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant cells since at least one component of the antibody's natural environment will not be present. Ordinarily, however, an isolated polypeptide or antibody will be prepared by at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelidae species have a single heavy chain variable region, which is referred to as “VHH”. VHH is thus a special type of VH.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present application may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody,” “intact antibody” or “whole antibody” are used interchangeably to refer to an antibody in its substantially intact form, as opposed to an antibody fragment. Specifically full-length 4-chain antibodies include those with heavy and light chains including an Fc region. The constant domains may be native sequence constant domains (e.g., human native sequence constant domains) or amino acid sequence variants thereof. In some cases, the intact antibody may have one or more effector functions.

The term “diabodies” refers to small antibody fragments prepared by constructing sFv fragments (see preceding paragraph) with short linkers (about 5-10) residues) between the VH and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the VH and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATTZFD®, antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. A suitable human acceptor antibody may be one selected from a conventional database, e.g., the KABAT database, Los Alamos database, the AbM, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see, for example, EP-A-0239400 and EP-A-054951). For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

The term “attach” or “attached” as used herein, refers to connecting or uniting by a bond, link, force or tie in order to keep two or more components together, which encompasses either direct or indirect attachment such that, for example, where a first polypeptide is directly bound to a second polypeptide or material, and, for example, where one or more intermediate compounds (e.g., amino acids, peptides, polypeptides, etc.) are disposed between the first polypeptide and the second polypeptide or material.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, an “immunoassay” refers to any binding assay that uses an antibody capable of binding specifically to a target molecule to detect and quantify the target molecule.

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991)

As use herein, the term “specifically binds” or is “specific for” refers to measurable and reproducible interactions such as binding between a target and an antibody, which is determinative of the presence of the target in the presence of a heterogeneous population of molecules including biological molecules. For example, an antibody that specifically binds a target (which can be an epitope) is an antibody that binds this target with greater affinity, avidity, more readily, and/or with greater duration than it binds other targets. In certain embodiments, an antibody that specifically binds a target has a dissociation constant (Kd) of ≤1 μM, ≤100 nM, ≤10 nM, ≤1 nM, or ≤0.1 nM. In certain embodiments, an antibody specifically binds an epitope on a protein that is conserved among the protein from different species. In another embodiment, specific binding can include, but does not require exclusive binding.

The term “specificity” refers to selective recognition of an antigen binding protein or antibody for a particular epitope of an antigen. Natural antibodies, for example, are monospecific. The term “multispecific” as used herein denotes that an antigen binding protein or an antibody has two or more antigen-binding sites of which at least two bind a different antigen or a different epitope of the same antigen. “Bispecific” as used herein denotes that an antigen binding protein or an antibody has two different antigen-binding specificities. The term “monospecific” antibody as used herein denotes an antibody that has one or more binding sites each of which bind the same epitope of the same antigen.

“Effector cells” are leukocytes which express one or more FcRs and perform effector functions. In one aspect, the effector cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils. The effector cells may be isolated from a native source, e.g., blood. Effector cells generally are lymphocytes associated with the effector phase, and function to produce cytokines (helper T cells), killing cells in infected with pathogens (cytotoxic T cells) or secreting antibodies (differentiated B cells).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present application. Specific illustrative and exemplary embodiments for measuring binding affinity are described in the following.

An “on-rate,” “rate of association,” “association rate,” or “ko,” as used herein can also be determined as described above using methods such as biolayer interferometry and surface plasmon resonance.

A “low-pH dissociation factor” as used herein is defined as the percentage of antibody dissociated at pH 5.8 from the antigen at 25° C., wherein the antibody is pre-bound to the antigen at pH 7.4. The low-pH dissociation factor may be measured by associating an antibody and an antigen (e.g. the anti-C2 antibody and any one of: human C2, human C2a, cynomolgus C2, cynomolgus C2a) at pH 7.4 for 600 seconds, followed by a dissociation period of 600 seconds in a buffer at pH 5.8, and calculation of the percentage of antibody dissociated at pH 5.8 from the antigen. A “neutral-pH dissociation factor” is defined as the percentage of antibody dissociated at pH 7.4 from the antigen at 25° C., wherein the antibody is pre-bound to the antigen at pH 7.4. The neutral-pH dissociation factor may be measured by associating antibody and antigen (e.g. the anti-C2 antibody and any one of: human C2, human C2a, cynomolgus C2, cynomolgus C2a) at pH 7.4 for 600 seconds, followed by a dissociation period of 600 seconds in a buffer at pH 7.4, and calculation of the percentage of antibody dissociated at pH 7.4 from the antigen. The antibody-antigen association and dissociation may be measured in various ways that are with the skill in the art, for instance, using biolayer interferometry.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “hybridoma,” as used herein refers to a cell resulting from the fusion of a B-lymphocyte and a fusion partner such as a myeloma cell. A hybridoma can be cloned and maintained indefinitely in cell culture and is able to produce monoclonal antibodies. A hybridoma can also be considered to be a hybrid cell.

The terms “nucleic acid molecule”, “nucleic acid” and “polynucleotide” may be used interchangeably, and refer to a polymer of nucleotides. Such polymers of nucleotides may contain natural and/or unnatural nucleotides, and include, but are not limited to, DNA, RNA, and PNA. “Nucleic acid sequence” refers to the linear sequence of nucleotides that comprise the nucleic acid molecule or polynucleotide. An “isolated nucleic acid” refers to a nucleic acid segment or fragment which has been separated from sequences which flank it in a naturally occurring state. i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and or at least about 75%, or at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide” and “peptide” are used interchangeably to refer to a polymer of amino acid residues, and are not limited to a minimum length. Such polymers of amino acid residues may contain natural or unnatural amino acid residues. Both full-length proteins and fragments thereof are encompassed by the definition. The terms also include post-expression modifications of the polypeptide, for example, glycosylation, sialylation, acetylation, phosphorylation, and the like. Furthermore, a “polypeptide” includes modifications, such as deletions, additions, and substitutions (generally conservative in nature), to the native sequence, as long as the polypeptide maintains the desired activity. These modifications may be deliberate, as through site-directed mutagenesis, or may be accidental, such as through mutations of hosts which produce the proteins or errors due to PCR amplification.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In various embodiments, the variant sequence is at least 99%, at least 98%, at least 97%, at least 96%, at least 95%, at least 94%, at least 93%, at least 92%, at least 91%, at least 90%, at least 89%, at least 88%, at least 87%, at least 86%, at least 85% identical to the reference sequence.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Reference to “about” a value or parameter herein includes (and describes) variations that are directed to that value or parameter per se. For example, description referring to “about X” includes description of “X”. As used herein, reference to “not” a value or parameter generally means and describes “other than” a value or parameter. For example, the method is not used to treat cancer of type X means the method is used to treat cancer of types other than X. The term “about X-Y” used herein has the same meaning as “about X to about Y.”

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “sterile” formulation is aseptic or essentially free from living microorganisms and their spores.

A “stable” formulation is one in which the protein therein essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month and/or stable at 2-8° C. for at least 2 years. Where the formulation is to be stored at 30° C., generally the formulation should be stable for at least 2 years at 30° C. and/or stable at 40° C. for at least 6 months. For example, the extent of aggregation during storage can be used as an indicator of protein stability. Thus, a “stable” formulation may be one wherein less than about 10% and preferably less than about 5% of the protein are present as an aggregate in the formulation. In other embodiments, any increase in aggregate formation during storage of the formulation can be determined.

A “reconstituted” formulation is one which has been prepared by dissolving a lyophilized protein or antibody formulation in a diluent such that the protein is dispersed throughout. The reconstituted formulation is suitable for administration (e.g. subcutaneous administration) to a patient to be treated with the protein of interest and, in certain embodiments, may be one which is suitable for parenteral or intravenous administration.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example. The formulations of the present application can be hypertonic as a result of the addition of salt and/or buffer.

It is understood that embodiments described herein include “consisting” and/or “consisting essentially of” embodiments.

As used herein and in the appended claims, the singular forms “a,” “or,” and “the” include plural referents unless the context clearly dictates otherwise.

### II. Anti-C2 Antibodies

The present application provides novel, anti-C2 antibodies and antibody constructs comprising such antibodies. In some embodiments, the antibody has mutations in its VH and/or VL domains. In some embodiments, the mutations render the antibodies pH sensitive in antigen binding. In some embodiments, the anti-C2 antibodies harbor certain mutations that render binding of the antibody to C2 and/or C2a stronger at a neutral pH (e.g., about pH 7.4; such as that found in the blood) than at a more acidic pH (e.g., about pH 5.8; such as that found in the endosome). In some embodiments, the mutations are histidine mutations, for example the F58H mutation in the VH domain.

The anti-C2 antibodies of the present application comprise at least one antigen binding portion comprising a heavy chain variable domain (VH) and a light chain variable domain (VL). Exemplary antigen binding fragments contemplated herein include, but are not limited to, Fab, Fab′, F(ab′)₂, and Fv fragments; diabodies; linear antibodies; single-chain antibody molecules (such as scFv); and multispecific antibodies formed from antibody fragments. Such antigen binding portion can be a full-length conventional antibody consisting of two heavy chains and two light chains, or an antigen binding fragment derived therefrom.

In some embodiments, the anti-C2 antibody comprises an H-CDR1, an H-CDR2, and an H-CDR3 of an VH comprising the amino acid sequence of any one of SEQ ID NOs: 1, 9, 17, 25, 33, 41, 49, 57, 65, 73, 81, 89, and 246. In some embodiments, the anti-C2 antibody comprises an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247.

In some embodiment, the anti-C2 antibody further comprises one or more additional mutations (such as histidine mutations) in the variable domain (VH) of heavy chain. In some embodiments, the anti-C2 antibody comprises an F58H mutation in the VH, wherein the VH mutation is in reference to SEQ ID NO: 17 under the Kabat numbering system:

(SEQ ID NO: 17)
EVQLLESGGGLVQPGGSLRLSCAASGFTFRHYAMSWVRQAPGKGLEWVSL
ISGSGASTFYADSVKGRFTISRDNSENMLYLQMNSLRAEDTAVYYCAKDS
LAVAGSEYFQHWGQGTLVTVSS.

In some embodiment, the anti-C2 antibody further comprises one or more additional mutations (such as histidine mutations) in the variable domain (VL) of light chain, wherein the VL mutation is in reference to SEQ ID NO: 18 under the Kabat numbering system:

(SEQ ID NO: 18)
SYVLTQAPSVSVAPGQTARITCGGNYFGGKSVHWYQQRPGQAPVLVVYDD
SDRPSGIPERFSGSKSGNTATLAISRVEAGDEAAYYCQVYDTSSDHWVFG
GGTKLTVL

In some embodiments, the anti-C2 antibody comprises an Fc region, such as a human Fc region. In some embodiments, the Fc region is derived from an IgG molecule, such as any one of the IgG1, IgG2, IgG3, or IgG4 subclass. In some embodiments, the Fc region is capable of mediating an antibody effector function, such as ADCC (antibody-dependent cell-mediated cytotoxicity) and/or CDC (complement-dependent cytotoxicity). For example, antibodies of subclass IgG1, IgG2, and IgG3 with wild-type Fc sequences usually show complement activation including CIq and C3 binding, whereas IgG4 does not activate the complement system and does not bind CIq and/or C3. In some embodiments, the Fc region comprises a modification that reduces binding affinity of the Fc region to an Fc receptor. In some embodiments, the Fc region is an IgG4 Fc. In some embodiments, the IgG4 Fc region comprises the amino acid sequence of SEQ ID NO: 183. In some embodiments, the IgG4 Fc comprises mutations. See, for example, Armour K L et al., Eur J. Immunol. 1999; 29: 2613; and Shields R L et al., J. Biol. Chem. 2001; 276: 6591. In some embodiments, the Fc region comprises one or more mutations selected from the group consisting of S228P, M428L, N434A and N434S, wherein the mutations are relative to SEQ ID NO: 183 under the EU numbering system. In some embodiments, the Fc region comprises mutations S228P, M428L, and N434A. In some embodiments, the Fc region comprises mutations S228P, M428L, and N434S. In some embodiments, the IgG4 Fc region comprises the amino acid sequence of SEQ ID NO: 184. In some embodiments, the IgG4 Fc region comprises the amino acid sequence of SEQ ID NO: 216. In some embodiments, the Fc region is an IgG1 Fc. In some embodiments, the IgG1 Fc region comprises the amino acid sequence of SEQ ID NO: 210. In some embodiments, the Fc region comprises one or more mutations selected from the group consisting of L234A, L235A, M428L, N434A and N434S, wherein the mutations are relative to SEQ ID NO: 210 under the EU numbering system. In some embodiments, the Fc region comprises mutations L234A, L235A, M428L and N434A. In some embodiments, the Fc region comprises mutations L234A, L235A, M428L and N434S. In some embodiments, the IgG1 Fc region comprises the amino acid sequence of SEQ ID NO: 214. In some embodiments, the IgG1 Fc region comprises the amino acid sequence of SEQ ID NO: 215.

In some embodiments, the anti-C2 antibody comprises a VH CDR 1 (“H-CDR 1”) comprising the amino acid sequence of SEQ ID NO: 3 or a variant thereof comprising 1, 2, or 3 amino acid substitutions; a VH CDR2 (“H-CDR2”) comprising the amino acid sequence of SEQ ID NO: 4 or a variant thereof comprising 1, 2, or 3 amino acid substitutions: a VH CDR3 (“H-CDR3”) comprising the amino acid sequence of SEQ ID NO: 5 or a variant thereof comprising 1, 2, or 3 amino acid substitutions; a VL CDR1 (“L-CDR1”) comprising the amino acid sequence of SEQ ID NO: 6 or a variant thereof comprising 1, 2, or 3 amino acid substitutions; a VL CDR2 (“L-CDR2”) comprising the amino acid sequence of SEQ ID NO: 7 or a variant thereof comprising 1, 2, or 3 amino acid substitutions; and a VL CDR3 (“L-CDR3”) comprising the amino acid sequence of SEQ ID NO: 8 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 3; a VH CDR2 (“H-CDR2”) comprising the amino acid sequence of SEQ ID NO: 4; a VH CDR3 (“H-CDR3”) comprising the amino acid sequence of SEQ ID NO: 5; a VL CDR1 (“L-CDR 1”) comprising the amino acid sequence of SEQ ID NO: 6; a VL CDR2 (“L-CDR2”) comprising the amino acid sequence of SEQ ID NO: 7; and a VL CDR3 (“L-CDR3”) comprising the amino acid sequence of SEQ ID NO: 8.

In some embodiments (independent of or in addition to the CDR sequences described here), the anti-C2 antibody comprises an VH comprising the amino acid sequence SEQ ID NO: 1 or a variant thereof that is at least about any one of 60%, 70%, 80%, 85%, 90%, 95%, 96%, 97%, 98%, 99% or more of amino acid sequence homology to SEQ ID NO: 1; and an VL comprising the amino acid sequence of SEQ ID NO: 2 or a variant thereof that is at least about any one of 60%, 70%, 80%, 85%, 90%, 95%, 96%, 97%, 98%, 99% or more of amino acid sequence homology to SEQ ID NO: 2. In some embodiments, the anti-C2 antibody comprises an VH comprising the amino acid sequence SEQ ID NO: 1 and an VL comprising the amino acid sequence of SEQ ID NO: 2. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 11 or a variant thereof comprising 1, 2, or 3 amino acid substitutions; a VH CDR2 (“H-CDR2”) comprising the amino acid sequence of SEQ ID NO: 12 or a variant thereof comprising 1, 2, or 3 amino acid substitutions; a VH CDR3 (“H-CDR3”) comprising the amino acid sequence of SEQ ID NO: 13 or a variant thereof comprising 1, 2, or 3 amino acid substitutions; a VL CDR1 (“L-CDR 1”) comprising the amino acid sequence of SEQ ID NO: 14 or a variant thereof comprising 1, 2, or 3 amino acid substitutions; a VL CDR2 (“L-CDR2”) comprising the amino acid sequence of SEQ ID NO: 15 or a variant thereof comprising 1, 2, or 3 amino acid substitutions; and a VL CDR3 (“L-CDR3”) comprising the amino acid sequence of SEQ ID NO: 16 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 11; a VH CDR2 (“H-CDR2”) comprising the amino acid sequence of SEQ ID NO: 12; a VH CDR3 (“H-CDR3”) comprising the amino acid sequence of SEQ ID NO: 13; a VL CDR1 (“L-CDR1”) comprising the amino acid sequence of SEQ ID NO: 14; a VL CDR2 (“L-CDR2”) comprising the amino acid sequence of SEQ ID NO: 15; and a VL CDR3 (“L-CDR3”) comprising the amino acid sequence of SEQ ID NO: 16.

In some embodiments (independent of or in addition to the CDR sequences described here), the anti-C2 antibody comprises an VH comprising the amino acid sequence SEQ ID NO: 9 or a variant thereof that is at least about any one of 60%, 70%, 80%, 85%, 90%, 95%, 96%, 97%, 98%, 99% or more of amino acid sequence homology to SEQ ID NO: 9; and an VL comprising the amino acid sequence of SEQ ID NO: 10 or a variant thereof that is at least about any one of 60%, 70%, 80%, 85%, 90%, 95%, 96%, 97%, 98%, 99% or more of amino acid sequence homology to SEQ ID NO: 10. In some embodiments, the anti-C2 antibody comprises an VH comprising the amino acid sequence SEQ ID NO: 9 and an VL comprising the amino acid sequence of SEQ ID NO: 10. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 19 or a variant thereof comprising 1, 2, or 3 amino acid substitutions; a VH CDR2 (“H-CDR2”) comprising the amino acid sequence of SEQ ID NO: 20 or a variant thereof comprising 1, 2, or 3 amino acid substitutions; a VH CDR3 (“H-CDR3”) comprising the amino acid sequence of SEQ ID NO: 21 or a variant thereof comprising 1, 2, or 3 amino acid substitutions; a VL CDR1 (“L-CDR1”) comprising the amino acid sequence of SEQ ID NO: 22 or a variant thereof comprising 1, 2, or 3 amino acid substitutions; a VL CDR2 (“L-CDR2”) comprising the amino acid sequence of SEQ ID NO: 23 or a variant thereof comprising 1, 2, or 3 amino acid substitutions; and a VL CDR3 (“L-CDR3”) comprising the amino acid sequence of SEQ ID NO: 24 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 19; a VH CDR2 (“H-CDR2”) comprising the amino acid sequence of SEQ ID NO: 20; a VH CDR3 (“H-CDR3”) comprising the amino acid sequence of SEQ ID NO: 21; a VL CDR 1 (“L-CDR1”) comprising the amino acid sequence of SEQ ID NO: 22; a VL CDR2 (“L-CDR2”) comprising the amino acid sequence of SEQ ID NO: 23; and a VL CDR3 (“L-CDR3”) comprising the amino acid sequence of SEQ ID NO: 24.

In some embodiments (independent of or in addition to the CDR sequences described here), the anti-C2 antibody comprises an VH comprising the amino acid sequence SEQ ID NO: 17 or a variant thereof that is at least about any one of 60%, 70%, 80%, 85%, 90%, 95%, 96%, 97%, 98%, 99% or more of amino acid sequence homology to SEQ ID NO: 17; and an VL comprising the amino acid sequence of SEQ ID NO: 18 or a variant thereof that is at least about any one of 60%, 70%, 80%, 85%, 90%, 95%, 96%, 97%, 98%, 99% or more of amino acid sequence homology to SEQ ID NO: 18. In some embodiments, the anti-C2 antibody comprises an VH comprising the amino acid sequence SEQ ID NO: 17 and an VL comprising the amino acid sequence of SEQ ID NO: 18. In some embodiments, the anti-C2 antibody further comprises an Fc region (such as the Fc region of an IgG4 or an IgG1).

In some embodiments, the anti-C2 antibody comprises a VH CDR 1 (“H-CDR 1”) comprising the amino acid sequence of SEQ ID NO: 27 or a variant thereof comprising 1, 2, or 3 amino acid substitutions; a VH CDR2 (“H-CDR2”) comprising the amino acid sequence of SEQ ID NO: 28 or a variant thereof comprising 1, 2, or 3 amino acid substitutions; a VH CDR3 (“H-CDR3”) comprising the amino acid sequence of SEQ ID NO: 29 or a variant thereof comprising 1, 2, or 3 amino acid substitutions; a VL CDR1 (“L-CDR 1”) comprising the amino acid sequence of SEQ ID NO: 30 or a variant thereof comprising 1, 2, or 3 amino acid substitutions; a VL CDR2 (“L-CDR2”) comprising the amino acid sequence of SEQ ID NO: 31 or a variant thereof comprising 1, 2, or 3 amino acid substitutions; and a VL CDR3 (“L-CDR3”) comprising the amino acid sequence of SEQ ID NO: 32 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR 1 (“H-CDR 1”) comprising the amino acid sequence of SEQ ID NO: 27; a VH CDR2 (“H-CDR2”) comprising the amino acid sequence of SEQ ID NO: 28; a VH CDR3 (“H-CDR3”) comprising the amino acid sequence of SEQ ID NO: 29; a VL CDR1 (“L-CDR 1”) comprising the amino acid sequence of SEQ ID NO: 30; a VL CDR2 (“L-CDR2”) comprising the amino acid sequence of SEQ ID NO: 31; and a VL CDR3 (“L-CDR3”) comprising the amino acid sequence of SEQ ID NO: 32.

In some embodiments (independent of or in addition to the CDR sequences described here), the anti-C2 antibody comprises an VH comprising the amino acid sequence SEQ ID NO: 25 or a variant thereof that is at least about any one of 60%, 70%, 80%, 85%, 90%, 95%, 96%, 97%, 98%, 99% or more of amino acid sequence homology to SEQ ID NO: 25; and an V_{L} comprising the amino acid sequence of SEQ ID NO: 26 or a variant thereof that is at least about any one of 60%, 70%, 80%, 85%, 90%, 95%, 96%, 97%, 98%, 99% or more of amino acid sequence homology to SEQ ID NO: 26. In some embodiments, the anti-C2 antibody comprises an VH comprising the amino acid sequence SEQ ID NO: 25 and an VL comprising the amino acid sequence of SEQ ID NO: 26. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 35 or a variant thereof comprising 1, 2, or 3 amino acid substitutions; a VH CDR2 (“H-CDR2”) comprising the amino acid sequence of SEQ ID NO: 36 or a variant thereof comprising 1, 2, or 3 amino acid substitutions; a VH CDR3 (“H-CDR3”) comprising the amino acid sequence of SEQ ID NO: 37 or a variant thereof comprising 1, 2, or 3 amino acid substitutions; a VL CDR1 (“L-CDR1”) comprising the amino acid sequence of SEQ ID NO: 38 or a variant thereof comprising 1, 2, or 3 amino acid substitutions; a VL CDR2 (“L-CDR2”) comprising the amino acid sequence of SEQ ID NO: 39 or a variant thereof comprising 1, 2, or 3 amino acid substitutions; and a VL CDR3 (“L-CDR3”) comprising the amino acid sequence of SEQ ID NO: 40 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 35; a VH CDR2 (“H-CDR2”) comprising the amino acid sequence of SEQ ID NO: 36; a VH CDR3 (“H-CDR3”) comprising the amino acid sequence of SEQ ID NO: 37; a VL CDR 1 (“L-CDR 1”) comprising the amino acid sequence of SEQ ID NO: 38; a VL CDR2 (“L-CDR2”) comprising the amino acid sequence of SEQ ID NO: 39; and a VL CDR3 (“L-CDR3”) comprising the amino acid sequence of SEQ ID NO: 40.

In some embodiments (independent of or in addition to the CDR sequences described here), the anti-C2 antibody comprises an VH comprising the amino acid sequence SEQ ID NO: 33 or a variant thereof that is at least about any one of 60%, 70%, 80%, 85%, 90%, 95%, 96%, 97%, 98%, 99% or more of amino acid sequence homology to SEQ ID NO: 33; and an VL comprising the amino acid sequence of SEQ ID NO: 34 or a variant thereof that is at least about any one of 60%, 70%, 80%, 85%, 90%, 95%, 96%, 97%, 98%, 99% or more of amino acid sequence homology to SEQ ID NO: 34. In some embodiments, the anti-C2 antibody comprises an VH comprising the amino acid sequence SEQ ID NO: 33 and an VL comprising the amino acid sequence of SEQ ID NO: 34. In some embodiments, the anti-C2 antibody further comprises an Fc region (such as the Fc region of an IgG4 or an IgG1).

In some embodiments, the anti-C2 antibody comprises a VH CDR1 (“H-CDR 1”) comprising the amino acid sequence of SEQ ID NO: 43 or a variant thereof comprising 1, 2, or 3 amino acid substitutions; a VH CDR2 (“H-CDR2”) comprising the amino acid sequence of SEQ ID NO: 44 or a variant thereof comprising 1, 2, or 3 amino acid substitutions; a VH CDR3 (“H-CDR3”) comprising the amino acid sequence of SEQ ID NO: 45 or a variant thereof comprising 1, 2, or 3 amino acid substitutions; a VL CDR1 (“L-CDR1”) comprising the amino acid sequence of SEQ ID NO: 46 or a variant thereof comprising 1, 2, or 3 amino acid substitutions; a VL CDR2 (“L-CDR2”) comprising the amino acid sequence of SEQ ID NO: 47 or a variant thereof comprising 1, 2, or 3 amino acid substitutions; and a VL CDR3 (“L-CDR3”) comprising the amino acid sequence of SEQ ID NO: 48 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 43; a VH CDR2 (“H-CDR2”) comprising the amino acid sequence of SEQ ID NO: 44; a VH CDR3 (“H-CDR3”) comprising the amino acid sequence of SEQ ID NO: 45; a VL CDR1 (“L-CDR 1”) comprising the amino acid sequence of SEQ ID NO: 46; a VL CDR2 (“L-CDR2”) comprising the amino acid sequence of SEQ ID NO: 47; and a VL CDR3 (“L-CDR3”) comprising the amino acid sequence of SEQ ID NO: 48.

In some embodiments (independent of or in addition to the CDR sequences described here), the anti-C2 antibody comprises an VH comprising the amino acid sequence SEQ ID NO: 41 or a variant thereof that is at least about any one of 60%, 70%, 80%, 85%, 90%, 95%, 96%, 97%, 98%, 99% or more of amino acid sequence homology to SEQ ID NO: 41; and an V_{L} comprising the amino acid sequence of SEQ ID NO: 42 or a variant thereof that is at least about any one of 60%, 70%, 80%, 85%, 90%, 95%, 96%, 97%, 98%, 99% or more of amino acid sequence homology to SEQ ID NO: 42. In some embodiments, the anti-C2 antibody comprises an VH comprising the amino acid sequence SEQ ID NO: 41 and an VL comprising the amino acid sequence of SEQ ID NO: 42. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 51 or a variant thereof comprising 1, 2, or 3 amino acid substitutions; a VH CDR2 (“H-CDR2”) comprising the amino acid sequence of SEQ ID NO: 52 or a variant thereof comprising 1, 2, or 3 amino acid substitutions; a VH CDR3 (“H-CDR3”) comprising the amino acid sequence of SEQ ID NO: 53 or a variant thereof comprising 1, 2, or 3 amino acid substitutions; a VL CDR1 (“L-CDR1”) comprising the amino acid sequence of SEQ ID NO: 54 or a variant thereof comprising 1, 2, or 3 amino acid substitutions; a VL CDR2 (“L-CDR2”) comprising the amino acid sequence of SEQ ID NO: 55 or a variant thereof comprising 1, 2, or 3 amino acid substitutions; and a VL CDR3 (“L-CDR3”) comprising the amino acid sequence of SEQ ID NO: 56 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 51; a VH CDR2 (“H-CDR2”) comprising the amino acid sequence of SEQ ID NO: 52; a VH CDR3 (“H-CDR3”) comprising the amino acid sequence of SEQ ID NO: 53; a VL CDR 1 (“L-CDR1”) comprising the amino acid sequence of SEQ ID NO: 54; a VL CDR2 (“L-CDR2”) comprising the amino acid sequence of SEQ ID NO: 55; and a VL CDR3 (“L-CDR3”) comprising the amino acid sequence of SEQ ID NO: 56.

In some embodiments (independent of or in addition to the CDR sequences described here), the anti-C2 antibody comprises an VH comprising the amino acid sequence SEQ ID NO: 49 or a variant thereof that is at least about any one of 60%, 70%, 80%, 85%, 90%, 95%, 96%, 97%, 98%, 99% or more of amino acid sequence homology to SEQ ID NO: 49; and an VL comprising the amino acid sequence of SEQ ID NO: 50 or a variant thereof that is at least about any one of 60%, 70%, 80%, 85%, 90%, 95%, 96%, 97%, 98%, 99% or more of amino acid sequence homology to SEQ ID NO: 50. In some embodiments, the anti-C2 antibody comprises an VH comprising the amino acid sequence SEQ ID NO: 49 and an VL comprising the amino acid sequence of SEQ ID NO: 50. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 59 or a variant thereof comprising 1, 2, or 3 amino acid substitutions; a VH CDR2 (“H-CDR2”) comprising the amino acid sequence of SEQ ID NO: 60 or a variant thereof comprising 1, 2, or 3 amino acid substitutions; a VH CDR3 (“H-CDR3”) comprising the amino acid sequence of SEQ ID NO: 61 or a variant thereof comprising 1, 2, or 3 amino acid substitutions; a VL CDR 1 (“L-CDR 1”) comprising the amino acid sequence of SEQ ID NO: 62 or a variant thereof comprising 1, 2, or 3 amino acid substitutions; a VL CDR2 (“L-CDR2”) comprising the amino acid sequence of SEQ ID NO: 63 or a variant thereof comprising 1, 2, or 3 amino acid substitutions; and a VL CDR3 (“L-CDR3”) comprising the amino acid sequence of SEQ ID NO: 64 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 59; a VH CDR2 (“H-CDR2”) comprising the amino acid sequence of SEQ ID NO: 60; a VH CDR3 (“H-CDR3”) comprising the amino acid sequence of SEQ ID NO: 61; a VL CDR1 (“L-CDR1”) comprising the amino acid sequence of SEQ ID NO: 62; a VL CDR2 (“L-CDR2”) comprising the amino acid sequence of SEQ ID NO: 63; and a VL CDR3 (“L-CDR3”) comprising the amino acid sequence of SEQ ID NO: 64.

In some embodiments (independent of or in addition to the CDR sequences described here), the anti-C2 antibody comprises an VH comprising the amino acid sequence SEQ ID NO: 57 or a variant thereof that is at least about any one of 60%, 70%, 80%, 85%, 90%, 95%, 96%, 97%, 98%, 99% or more of amino acid sequence homology to SEQ ID NO: 57; and an VL comprising the amino acid sequence of SEQ ID NO: 58 or a variant thereof that is at least about any one of 60%, 70%, 80%, 85%, 90%, 95%, 96%, 97%, 98%, 99% or more of amino acid sequence homology to SEQ ID NO: 58. In some embodiments, the anti-C2 antibody comprises an VH comprising the amino acid sequence SEQ ID NO: 57 and an VL comprising the amino acid sequence of SEQ ID NO: 58. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 67 or a variant thereof comprising 1, 2, or 3 amino acid substitutions; a VH CDR2 (“H-CDR2”) comprising the amino acid sequence of SEQ ID NO: 68 or a variant thereof comprising 1, 2, or 3 amino acid substitutions; a VH CDR3 (“H-CDR3”) comprising the amino acid sequence of SEQ ID NO: 69 or a variant thereof comprising 1, 2, or 3 amino acid substitutions; a VL CDR1 (“L-CDR1”) comprising the amino acid sequence of SEQ ID NO: 70 or a variant thereof comprising 1, 2, or 3 amino acid substitutions; a VL CDR2 (“L-CDR2”) comprising the amino acid sequence of SEQ ID NO: 71 or a variant thereof comprising 1, 2, or 3 amino acid substitutions; and a VL CDR3 (“L-CDR3”) comprising the amino acid sequence of SEQ ID NO: 72 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 67; a VH CDR2 (“H-CDR2”) comprising the amino acid sequence of SEQ ID NO: 68; a VH CDR3 (“H-CDR3”) comprising the amino acid sequence of SEQ ID NO: 69; a VL CDR1 (“L-CDR1”) comprising the amino acid sequence of SEQ ID NO: 70; a VL CDR2 (“L-CDR2”) comprising the amino acid sequence of SEQ ID NO: 71; and a VL CDR3 (“L-CDR3”) comprising the amino acid sequence of SEQ ID NO: 72.

In some embodiments (independent of or in addition to the CDR sequences described here), the anti-C2 antibody comprises an VH comprising the amino acid sequence SEQ ID NO: 65 or a variant thereof that is at least about any one of 60%, 70%, 80%, 85%, 90%, 95%, 96%, 97%, 98%, 99% or more of amino acid sequence homology to SEQ ID NO: 66; and an VL comprising the amino acid sequence of SEQ ID NO: 50 or a variant thereof that is at least about any one of 60%, 70%, 80%, 85%, 90%, 95%, 96%, 97%, 98%, 99% or more of amino acid sequence homology to SEQ ID NO: 65. In some embodiments, the anti-C2 antibody comprises an VH comprising the amino acid sequence SEQ ID NO: 66 and an VL comprising the amino acid sequence of SEQ ID NO: 50. In some embodiments, the anti-C2 antibody further comprises an Fc region (such as the Fc region of an IgG4 or an IgG1).

In some embodiments, the anti-C2 antibody comprises a VH CDR 1 (“H-CDR 1”) comprising the amino acid sequence of SEQ ID NO: 75 or a variant thereof comprising 1, 2, or 3 amino acid substitutions; a VH CDR2 (“H-CDR2”) comprising the amino acid sequence of SEQ ID NO: 76 or a variant thereof comprising 1, 2, or 3 amino acid substitutions; a VH CDR3 (“H-CDR3”) comprising the amino acid sequence of SEQ ID NO: 77 or a variant thereof comprising 1, 2, or 3 amino acid substitutions; a VL CDR1 (“L-CDR1”) comprising the amino acid sequence of SEQ ID NO: 78 or a variant thereof comprising 1, 2, or 3 amino acid substitutions; a VL CDR2 (“L-CDR2”) comprising the amino acid sequence of SEQ ID NO: 79 or a variant thereof comprising 1, 2, or 3 amino acid substitutions; and a VL CDR3 (“L-CDR3”) comprising the amino acid sequence of SEQ ID NO: 80 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 75; a VH CDR2 (“H-CDR2”) comprising the amino acid sequence of SEQ ID NO: 76; a VH CDR3 (“H-CDR3”) comprising the amino acid sequence of SEQ ID NO: 77; a VL CDR1 (“L-CDR1”) comprising the amino acid sequence of SEQ ID NO: 78; a VL CDR2 (“L-CDR2”) comprising the amino acid sequence of SEQ ID NO: 79; and a VL CDR3 (“L-CDR3”) comprising the amino acid sequence of SEQ ID NO: 80.

In some embodiments (independent of or in addition to the CDR sequences described here), the anti-C2 antibody comprises an VH comprising the amino acid sequence SEQ ID NO: 73 or a variant thereof that is at least about any one of 60%, 70%, 80%, 85%, 90%, 95%, 96%, 97%, 98%, 99% or more of amino acid sequence homology to SEQ ID NO: 73; and an VL comprising the amino acid sequence of SEQ ID NO: 74 or a variant thereof that is at least about any one of 60%, 70%, 80%, 85%, 90%, 95%, 96%, 97%, 98%, 99% or more of amino acid sequence homology to SEQ ID NO: 74. In some embodiments, the anti-C2 antibody comprises an VH comprising the amino acid sequence SEQ ID NO: 73 and an VL comprising the amino acid sequence of SEQ ID NO: 74. In some embodiments, the anti-C2 antibody further comprises an Fc region (such as the Fc region of an IgG4 or an IgG1).

In some embodiments, the anti-C2 antibody comprises a VH CDR 1 (“H-CDR1”) comprising the amino acid sequence of SEQ ID NO: 83 or a variant thereof comprising 1, 2, or 3 amino acid substitutions; a VH CDR2 (“H-CDR2”) comprising the amino acid sequence of SEQ ID NO: 84 or a variant thereof comprising 1, 2, or 3 amino acid substitutions; a VH CDR3 (“H-CDR3”) comprising the amino acid sequence of SEQ ID NO: 85 or a variant thereof comprising 1, 2, or 3 amino acid substitutions; a VL CDR1 (“L-CDR 1”) comprising the amino acid sequence of SEQ ID NO: 86 or a variant thereof comprising 1, 2, or 3 amino acid substitutions; a VL CDR2 (“L-CDR2”) comprising the amino acid sequence of SEQ ID NO: 87 or a variant thereof comprising 1, 2, or 3 amino acid substitutions; and a VL CDR3 (“L-CDR3”) comprising the amino acid sequence of SEQ ID NO: 88 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 83; a VH CDR2 (“H-CDR2”) comprising the amino acid sequence of SEQ ID NO: 84; a VH CDR3 (“H-CDR3”) comprising the amino acid sequence of SEQ ID NO: 85; a VL CDR1 (“L-CDR1”) comprising the amino acid sequence of SEQ ID NO: 86; a VL CDR2 (“L-CDR2”) comprising the amino acid sequence of SEQ ID NO: 87; and a VL CDR3 (“L-CDR3”) comprising the amino acid sequence of SEQ ID NO: 88.

In some embodiments (independent of or in addition to the CDR sequences described here), the anti-C2 antibody comprises an VH comprising the amino acid sequence SEQ ID NO: 81 or a variant thereof that is at least about any one of 60%, 70%, 80%, 85%, 90%, 95%, 96%, 97%, 98%, 99% or more of amino acid sequence homology to SEQ ID NO: 81; and an VL comprising the amino acid sequence of SEQ ID NO: 82 or a variant thereof that is at least about any one of 60%, 70%, 80%, 85%, 90%, 95%, 96%, 97%, 98%, 99% or more of amino acid sequence homology to SEQ ID NO: 82. In some embodiments, the anti-C2 antibody comprises an VH comprising the amino acid sequence SEQ ID NO: 81 and an VL comprising the amino acid sequence of SEQ ID NO: 82. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 94 or a variant thereof comprising 1, 2, or 3 amino acid substitutions; a VL CDR2 (“L-CDR2”) comprising the amino acid sequence of SEQ ID NO: 95 or a variant thereof comprising 1, 2, or 3 amino acid substitutions; and a VL CDR3 (“L-CDR3”) comprising the amino acid sequence of SEQ ID NO: 96 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 94; a VL CDR2 (“L-CDR2”) comprising the amino acid sequence of SEQ ID NO: 95; and a VL CDR3 (“L-CDR3”) comprising the amino acid sequence of SEQ ID NO: 96.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 90 or a variant thereof that is at least about any one of 60%, 70%, 80%, 85%, 90%, 95%, 96%, 97%, 98%, 99% or more of amino acid sequence homology to SEQ ID NO: 90. In some embodiments, the anti-C2 antibody comprises an VH comprising the amino acid sequence SEQ ID NO: 89 and an VL comprising the amino acid sequence of SEQ ID NO: 90. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 98 or a variant thereof comprising 1, 2, or 3 amino acid substitutions; a VL CDR2 (“L-CDR2”) comprising the amino acid sequence of SEQ ID NO: 99 or a variant thereof comprising 1, 2, or 3 amino acid substitutions; and a VL CDR3 (“L-CDR3”) comprising the amino acid sequence of SEQ ID NO: 100 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR 1 (“H-CDR 1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 98; a VL CDR2 (“L-CDR2”) comprising the amino acid sequence of SEQ ID NO: 99; and a VL CDR3 (“L-CDR3”) comprising the amino acid sequence of SEQ ID NO: 100.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 97 or a variant thereof that is at least about any one of 60%, 70%, 80%, 85%, 90%, 95%, 96%, 97%, 98%, 99% or more of amino acid sequence homology to SEQ ID NO: 97. In some embodiments, the anti-C2 antibody comprises an VH comprising the amino acid sequence SEQ ID NO: 89 and an VL comprising the amino acid sequence of SEQ ID NO: 97. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 102 or a variant thereof comprising 1, 2, or 3 amino acid substitutions; a VL CDR2 (“L-CDR2”) comprising the amino acid sequence of SEQ ID NO: 103 or a variant thereof comprising 1, 2, or 3 amino acid substitutions; and a VL CDR3 (“L-CDR3”) comprising the amino acid sequence of SEQ ID NO: 104 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR 1 (“L-CDR 1”) comprising the amino acid sequence of SEQ ID NO: 102; a VL CDR2 (“L-CDR2”) comprising the amino acid sequence of SEQ ID NO: 103; and a VL CDR3 (“L-CDR3”) comprising the amino acid sequence of SEQ ID NO: 104.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 97 or a variant thereof that is at least about any one of 60%, 70%, 80%, 85%, 90%, 95%, 96%, 97%, 98%, 99% or more of amino acid sequence homology to SEQ ID NO: 101. In some embodiments, the anti-C2 antibody comprises an VH comprising the amino acid sequence SEQ ID NO: 89 and an VL comprising the amino acid sequence of SEQ ID NO: 101. In some embodiments, the anti-C2 antibody further comprises an Fc region (such as the Fc region of an IgG4 or an IgG1).

In some embodiments, the anti-C2 antibody comprises a VH CDR 1 (“H-CDR 1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR 1”) comprising the amino acid sequence of SEQ ID NO: 106 or a variant thereof comprising 1, 2, or 3 amino acid substitutions; a VL CDR2 (“L-CDR2”) comprising the amino acid sequence of SEQ ID NO: 107 or a variant thereof comprising 1, 2, or 3 amino acid substitutions; and a VL CDR3 (“L-CDR3”) comprising the amino acid sequence of SEQ ID NO: 108 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR 1”) comprising the amino acid sequence of SEQ ID NO: 106; a VL CDR2 (“L-CDR2”) comprising the amino acid sequence of SEQ ID NO: 107; and a VL CDR3 (“L-CDR3”) comprising the amino acid sequence of SEQ ID NO: 108.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 105 or a variant thereof that is at least about any one of 60%, 70%, 80%, 85%, 90%, 95%, 96%, 97%, 98%, 99% or more of amino acid sequence homology to SEQ ID NO: 105. In some embodiments, the anti-C2 antibody comprises an VH comprising the amino acid sequence SEQ ID NO: 89 and an VL comprising the amino acid sequence of SEQ ID NO: 105. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 110 or a variant thereof comprising 1, 2, or 3 amino acid substitutions; a VL CDR2 (“L-CDR2”) comprising the amino acid sequence of SEQ ID NO: 111 or a variant thereof comprising 1, 2, or 3 amino acid substitutions; and a VL CDR3 (“L-CDR3”) comprising the amino acid sequence of SEQ ID NO: 112 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR 1 (“L-CDR1”) comprising the amino acid sequence of SEQ ID NO: 110; a VL CDR2 (“L-CDR2”) comprising the amino acid sequence of SEQ ID NO: 111; and a VL CDR3 (“L-CDR3”) comprising the amino acid sequence of SEQ ID NO: 112.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 109 or a variant thereof that is at least about any one of 60%, 70%, 80%, 85%, 90%, 95%, 96%, 97%, 98%, 99% or more of amino acid sequence homology to SEQ ID NO: 109. In some embodiments, the anti-C2 antibody comprises an VH comprising the amino acid sequence SEQ ID NO: 89 and an VL comprising the amino acid sequence of SEQ ID NO: 109. In some embodiments, the anti-C2 antibody further comprises an Fc region (such as the Fc region of an IgG4 or an IgG1).

In some embodiments, the anti-C2 antibody comprises a VH CDR1 (“H-CDR 1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 114 or a variant thereof comprising 1, 2, or 3 amino acid substitutions; a VL CDR2 (“L-CDR2”) comprising the amino acid sequence of SEQ ID NO: 115 or a variant thereof comprising 1, 2, or 3 amino acid substitutions; and a VL CDR3 (“L-CDR3”) comprising the amino acid sequence of SEQ ID NO: 116 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR 1”) comprising the amino acid sequence of SEQ ID NO: 114; a VL CDR2 (“L-CDR2”) comprising the amino acid sequence of SEQ ID NO: 115; and a VL CDR3 (“L-CDR3”) comprising the amino acid sequence of SEQ ID NO: 116.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 113 or a variant thereof that is at least about any one of 60%, 70%, 80%, 85%, 90%, 95%, 96%, 97%, 98%, 99% or more of amino acid sequence homology to SEQ ID NO: 113. In some embodiments, the anti-C2 antibody comprises an VH comprising the amino acid sequence SEQ ID NO: 89 and an VL comprising the amino acid sequence of SEQ ID NO: 113. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 118 or a variant thereof comprising 1, 2, or 3 amino acid substitutions; a VL CDR2 (“L-CDR2”) comprising the amino acid sequence of SEQ ID NO: 119 or a variant thereof comprising 1, 2, or 3 amino acid substitutions; and a VL CDR3 (“L-CDR3”) comprising the amino acid sequence of SEQ ID NO: 120 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 118; a VL CDR2 (“L-CDR2”) comprising the amino acid sequence of SEQ ID NO: 119; and a VL CDR3 (“L-CDR3”) comprising the amino acid sequence of SEQ ID NO: 120.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 117 or a variant thereof that is at least about any one of 60%, 70%, 80%, 85%, 90%, 95%, 96%, 97%, 98%, 99% or more of amino acid sequence homology to SEQ ID NO: 117. In some embodiments, the anti-C2 antibody comprises an VH comprising the amino acid sequence SEQ ID NO: 89 and an VL comprising the amino acid sequence of SEQ ID NO: 117. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR 1 (“L-CDR 1”) comprising the amino acid sequence of SEQ ID NO: 122 or a variant thereof comprising 1, 2, or 3 amino acid substitutions; a VL CDR2 (“L-CDR2”) comprising the amino acid sequence of SEQ ID NO: 123 or a variant thereof comprising 1, 2, or 3 amino acid substitutions; and a VL CDR3 (“L-CDR3”) comprising the amino acid sequence of SEQ ID NO: 124 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 122; a VL CDR2 (“L-CDR2”) comprising the amino acid sequence of SEQ ID NO: 123; and a VL CDR3 (“L-CDR3”) comprising the amino acid sequence of SEQ ID NO: 124.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 121 or a variant thereof that is at least about any one of 60%, 70%, 80%, 85%, 90%, 95%, 96%, 97%, 98%, 99% or more of amino acid sequence homology to SEQ ID NO: 121. In some embodiments, the anti-C2 antibody comprises an VH comprising the amino acid sequence SEQ ID NO: 89 and an VL comprising the amino acid sequence of SEQ ID NO: 121. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 126 or a variant thereof comprising 1, 2, or 3 amino acid substitutions; a VL CDR2 (“L-CDR2”) comprising the amino acid sequence of SEQ ID NO: 127 or a variant thereof comprising 1, 2, or 3 amino acid substitutions; and a VL CDR3 (“L-CDR3”) comprising the amino acid sequence of SEQ ID NO: 128 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 126; a VL CDR2 (“L-CDR2”) comprising the amino acid sequence of SEQ ID NO: 127; and a VL CDR3 (“L-CDR3”) comprising the amino acid sequence of SEQ ID NO: 128.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 125 or a variant thereof that is at least about any one of 60%, 70%, 80%, 85%, 90%, 95%, 96%, 97%, 98%, 99% or more of amino acid sequence homology to SEQ ID NO: 125. In some embodiments, the anti-C2 antibody comprises an VH comprising the amino acid sequence SEQ ID NO: 89 and an VL comprising the amino acid sequence of SEQ ID NO: 125. In some embodiments, the anti-C2 antibody further comprises an Fc region (such as the Fc region of an IgG4 or an IgG1).

In some embodiments, the anti-C2 antibody comprises a VH CDR 1 (“H-CDR 1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 130 or a variant thereof comprising 1, 2, or 3 amino acid substitutions; a VL CDR2 (“L-CDR2”) comprising the amino acid sequence of SEQ ID NO: 131 or a variant thereof comprising 1, 2, or 3 amino acid substitutions; and a VL CDR3 (“L-CDR3”) comprising the amino acid sequence of SEQ ID NO: 132 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 130; a VL CDR2 (“L-CDR2”) comprising the amino acid sequence of SEQ ID NO: 131; and a VL CDR3 (“L-CDR3”) comprising the amino acid sequence of SEQ ID NO: 132.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 129 or a variant thereof that is at least about any one of 60%, 70%, 80%, 85%, 90%, 95%, 96%, 97%, 98%, 99% or more of amino acid sequence homology to SEQ ID NO: 129. In some embodiments, the anti-C2 antibody comprises an VH comprising the amino acid sequence SEQ ID NO: 89 and an VL comprising the amino acid sequence of SEQ ID NO: 129. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 134 or a variant thereof comprising 1, 2, or 3 amino acid substitutions; a VL CDR2 (“L-CDR2”) comprising the amino acid sequence of SEQ ID NO: 135 or a variant thereof comprising 1, 2, or 3 amino acid substitutions; and a VL CDR3 (“L-CDR3”) comprising the amino acid sequence of SEQ ID NO: 136 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR 1 (“L-CDR1”) comprising the amino acid sequence of SEQ ID NO: 134; a VL CDR2 (“L-CDR2”) comprising the amino acid sequence of SEQ ID NO: 135; and a VL CDR3 (“L-CDR3”) comprising the amino acid sequence of SEQ ID NO: 136.

In s some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 133 or a variant thereof that is at least about any one of 60%, 70%, 80%, 85%, 90%, 95%, 96%, 97%, 98%, 99% or more of amino acid sequence homology to SEQ ID NO: 133. In some embodiments, the anti-C2 antibody comprises an VH comprising the amino acid sequence SEQ ID NO: 89 and an VL comprising the amino acid sequence of SEQ ID NO: 133. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR 1 (“L-CDR 1”) comprising the amino acid sequence of SEQ ID NO: 138 or a variant thereof comprising 1, 2, or 3 amino acid substitutions; a VL CDR2 (“L-CDR2”) comprising the amino acid sequence of SEQ ID NO: 139 or a variant thereof comprising 1, 2, or 3 amino acid substitutions; and a VL CDR3 (“L-CDR3”) comprising the amino acid sequence of SEQ ID NO: 140 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR 1”) comprising the amino acid sequence of SEQ ID NO: 138; a VL CDR2 (“L-CDR2”) comprising the amino acid sequence of SEQ ID NO: 139; and a VL CDR3 (“L-CDR3”) comprising the amino acid sequence of SEQ ID NO: 140.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 137 or a variant thereof that is at least about any one of 60%, 70%, 80%, 85%, 90%, 95%, 96%, 97%, 98%, 99% or more of amino acid sequence homology to SEQ ID NO: 137. In some embodiments, the anti-C2 antibody comprises an VH comprising the amino acid sequence SEQ ID NO: 89 and an VL comprising the amino acid sequence of SEQ ID NO: 137. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 142 or a variant thereof comprising 1, 2, or 3 amino acid substitutions; a VL CDR2 (“L-CDR2”) comprising the amino acid sequence of SEQ ID NO: 143 or a variant thereof comprising 1, 2, or 3 amino acid substitutions; and a VL CDR3 (“L-CDR3”) comprising the amino acid sequence of SEQ ID NO: 144 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 142; a VL CDR2 (“L-CDR2”) comprising the amino acid sequence of SEQ ID NO: 143; and a VL CDR3 (“L-CDR3”) comprising the amino acid sequence of SEQ ID NO: 144.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 141 or a variant thereof that is at least about any one of 60%, 70%, 80%, 85%, 90%, 95%, 96%, 97%, 98%, 99% or more of amino acid sequence homology to SEQ ID NO: 141. In some embodiments, the anti-C2 antibody comprises an VH comprising the amino acid sequence SEQ ID NO: 89 and an VL comprising the amino acid sequence of SEQ ID NO: 141. In some embodiments, the anti-C2 antibody further comprises an Fc region (such as the Fc region of an IgG4 or an IgG1).

In some embodiments, the anti-C2 antibody comprises a VH CDR 1 (“H-CDR 1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 146 or a variant thereof comprising 1, 2, or 3 amino acid substitutions; a VL CDR2 (“L-CDR2”) comprising the amino acid sequence of SEQ ID NO: 147 or a variant thereof comprising 1, 2, or 3 amino acid substitutions; and a VL CDR3 (“L-CDR3”) comprising the amino acid sequence of SEQ ID NO: 148 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 146; a VL CDR2 (“L-CDR2”) comprising the amino acid sequence of SEQ ID NO: 147; and a VL CDR3 (“L-CDR3”) comprising the amino acid sequence of SEQ ID NO: 148.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 145 or a variant thereof that is at least about any one of 60%, 70%, 80%, 85%, 90%, 95%, 96%, 97%, 98%, 99% or more of amino acid sequence homology to SEQ ID NO: 145. In some embodiments, the anti-C2 antibody comprises an VH comprising the amino acid sequence SEQ ID NO: 89 and an VL comprising the amino acid sequence of SEQ ID NO: 145. In some embodiments, the anti-C2 antibody further comprises an Fc region (such as the Fc region of an IgG4 or an IgG1).

In some embodiments, the anti-C2 antibody comprises a VH CDR 1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR 1”) comprising the amino acid sequence of SEQ ID NO: 150 or a variant thereof comprising 1, 2, or 3 amino acid substitutions; a VL CDR2 (“L-CDR2”) comprising the amino acid sequence of SEQ ID NO: 151 or a variant thereof comprising 1, 2, or 3 amino acid substitutions; and a VL CDR3 (“L-CDR3”) comprising the amino acid sequence of SEQ ID NO: 152 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 150; a VL CDR2 (“L-CDR2”) comprising the amino acid sequence of SEQ ID NO: 151; and a VL CDR3 (“L-CDR3”) comprising the amino acid sequence of SEQ ID NO: 152.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 149 or a variant thereof that is at least about any one of 60%, 70%, 80%, 85%, 90%, 95%, 96%, 97%, 98%, 99% or more of amino acid sequence homology to SEQ ID NO: 149. In some embodiments, the anti-C2 antibody comprises an VH comprising the amino acid sequence SEQ ID NO: 89 and an VL comprising the amino acid sequence of SEQ ID NO: 149. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions: a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 154 or a variant thereof comprising 1, 2, or 3 amino acid substitutions; a VL CDR2 (“L-CDR2”) comprising the amino acid sequence of SEQ ID NO: 155 or a variant thereof comprising 1, 2, or 3 amino acid substitutions; and a VL CDR3 (“L-CDR3”) comprising the amino acid sequence of SEQ ID NO: 156 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR1”) comprising the amino acid sequence of SEQ ID NO: 91; a VH CDR2 (“H-CDR2”) comprising the amino acid sequence of SEQ ID NO: 92: a VH CDR3 (“H-CDR3”) comprising the amino acid sequence of SEQ ID NO: 93; a VL CDR1 (“L-CDR1”) comprising the amino acid sequence of SEQ ID NO: 154; a VL CDR2 (“L-CDR2”) comprising the amino acid sequence of SEQ ID NO: 155; and a VL CDR3 (“L-CDR3”) comprising the amino acid sequence of SEQ ID NO: 156.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 153 or a variant thereof that is at least about any one of 60%, 70%, 80%, 85%, 90%, 95%, 96%, 97%, 98%, 99% or more of amino acid sequence homology to SEQ ID NO: 153. In some embodiments, the anti-C2 antibody comprises an VH comprising the amino acid sequence SEQ ID NO: 89 and an VL comprising the amino acid sequence of SEQ ID NO: 153. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 91 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 93 or a variant thereof comprising 1, 2, or 3 amino acid substitutions; a VL CDR1 (“L-CDR1”) comprising the amino acid sequence of SEQ ID NO: 158 or a variant thereof comprising 1, 2, or 3 amino acid substitutions; a VL CDR2 (“L-CDR2”) comprising the amino acid sequence of SEQ ID NO: 159 or a variant thereof comprising 1, 2, or 3 amino acid substitutions; and a VL CDR3 (“L-CDR3”) comprising the amino acid sequence of SEQ ID NO: 160 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR 1 (“H-CDR 1”) comprising the amino acid sequence of SEQ ID NO: 91; a VH CDR2 (“H-CDR2”) comprising the amino acid sequence of SEQ ID NO: 92; a VH CDR3 (“H-CDR3”) comprising the amino acid sequence of SEQ ID NO: 93; a VL CDR1 (“L-CDR1”) comprising the amino acid sequence of SEQ ID NO: 158; a VL CDR2 (“L-CDR2”) comprising the amino acid sequence of SEQ ID NO: 159; and a VL CDR3 (“L-CDR3”) comprising the amino acid sequence of SEQ ID NO: 160.

In some embodiments (independent of or in addition to the CDR sequences described here), the anti-C2 antibody comprises an VH comprising the amino acid sequence SEQ ID NO: 89 or a variant thereof that is at least about any one of 60%, 70%, 80%, 85%, 90%, 95%, 96%, 97%, 98%, 99% or more of amino acid sequence homology to SEQ ID NO: 89; and an VL comprising the amino acid sequence of SEQ ID NO: 157 or a variant thereof that is at least about any one of 60%, 70%, 80%, 85%, 90%, 95%, 96%, 97%, 98%, 99% or more of amino acid sequence homology to SEQ ID NO: 157. In some embodiments, the anti-C2 antibody comprises an VH comprising the amino acid sequence SEQ ID NO: 89 and an VL comprising the amino acid sequence of SEQ ID NO: 157. In some embodiments, the anti-C2 antibody further comprises an Fc region (such as the Fc region of an IgG4 or an IgG1).

In some embodiments, the anti-C2 antibody comprises a VH CDR1 (“H-CDR1”) comprising the amino acid sequence of SEQ ID NO: 19 or a variant thereof comprising 1, 2, or 3 amino acid substitutions; a VH CDR2 (“H-CDR2”) comprising the amino acid sequence of SEQ ID NO: 92 or a variant thereof comprising 1, 2, or 3 amino acid substitutions; a VH CDR3 (“H-CDR3”) comprising the amino acid sequence of SEQ ID NO: 250 or a variant thereof comprising 1, 2, or 3 amino acid substitutions; a VL CDR1 (“L-CDR1”) comprising the amino acid sequence of SEQ ID NO: 98 or a variant thereof comprising 1, 2, or 3 amino acid substitutions; a VL CDR2 (“L-CDR2”) comprising the amino acid sequence of SEQ ID NO: 23 or a variant thereof comprising 1, 2, or 3 amino acid substitutions; and a VL CDR3 (“L-CDR3”) comprising the amino acid sequence of SEQ ID NO: 24 or a variant thereof comprising 1, 2, or 3 amino acid substitutions. In some embodiments, the anti-C2 antibody comprises up to 3, 2, or 1 amino acid substitutions in the H-CDRs. In some embodiments, the anti-C2 antibody comprises up to 3, 2, or 1 amino acid substitutions in the L-CDRs. In some embodiments, the anti-C2 antibody comprises a VH CDR1 (“H-CDR 1”) comprising the amino acid sequence of SEQ ID NO: 19; a VH CDR2 (“H-CDR2”) comprising the amino acid sequence of SEQ ID NO: 92; a VH CDR3 (“H-CDR3”) comprising the amino acid sequence of SEQ ID NO: 250; a VL CDR 1 (“L-CDR1”) comprising the amino acid sequence of SEQ ID NO: 98; a VL CDR2 (“L-CDR2”) comprising the amino acid sequence of SEQ ID NO: 23; and a VL CDR3 (“L-CDR3”) comprising the amino acid sequence of SEQ ID NO: 24.

In some embodiments (independent of or in addition to the CDR sequences described here), the anti-C2 antibody comprises an VH comprising the amino acid sequence SEQ ID NO: 246 or a variant thereof that is at least about any one of 60%, 70%, 80%, 85%, 90%, 95%, 96%, 97%, 98%, 99% or more of amino acid sequence homology to SEQ ID NO: 246; and an VL comprising the amino acid sequence of SEQ ID NO: 247 or a variant thereof that is at least about any one of 60%, 70%, 80%, 85%, 90%, 95%, 96%, 97%, 98%, 99% or more of amino acid sequence homology to SEQ ID NO: 247. In some embodiments, the anti-C2 antibody comprises an VH comprising the amino acid sequence SEQ ID NO: 246 and an VL comprising the amino acid sequence of SEQ ID NO: 247. In some embodiments, the anti-C2 antibody further comprises an Fc region (such as the Fc region of an IgG4 or an IgG1).

### C2, C2a Proteins and C2/C2a Binding Analysis

The complement system, also known as the complement cascade, is a part of the innate immune system that enhances (complements) the ability of antibodies and phagocytic cells to clear microbes and damaged cells from an organism, promote inflammation, and attack the pathogen's cell membrane. The complement system is not adaptable and does not change during an individual's lifetime. The complement system can, however, be recruited and brought into action by antibodies generated by the adaptive immune system. The complement system plays an important role in the pathology of many autoimmune, inflammatory and ischemic diseases. Inappropriate complement activation and deposition on host cells can lead to complement-mediated lysis and/or injury of cells and target tissues, as well as tissue destruction due to the generation of powerful mediators of inflammation.

There are three known complement pathways: the alternative complement pathway (AP), the classical pathway (CP), and the lectin pathway (LP). Generally, the CP is initiated by antigen-antibody complexes, the LP is activated by binding of lectins to sugar molecules on microbial surfaces, while the AP is constitutively active at a low level but can be quickly amplified on bacterial, viral, and parasitic cell surfaces due to the lack of regulatory proteins. Host cells are usually protected from AP complement activation by regulatory proteins. But in some situations, such as when the regulatory proteins are defective or missing, the AP can also be activated uncontrollably on host cells, leading to complement-mediated disease or disorder. The CP consists of components C1, C2, C4 and converges with the AP at the C3 activation step. The LP consists of mannose-binding lectins (MBLs) and MBL-associated serine proteases (MASPs) and shares with the CP the components C4 and C2. The AP consists of components C3 and several factors, such as factor B, factor D, properdin, and the fluid phase regulator factor H. Complement activation consists of three stages: (a) recognition, (b) enzymatic activation, and (c) membrane attack leading to cell death. The first phase of CP complement activation begins with C1. C1 is made up of three distinct proteins: a recognition subunit, C1q, and the serine protease subcomponents, C1r and C1s, which are bound together in a calcium-dependent tetrameric complex, C1r2s2. An intact C1 complex is necessary for physiological activation of C1 to result. Activation occurs when the intact C1 complex binds to immunoglobulin complexed with antigen. This binding activates C1s which then cleaves both the C4 and C2 proteins to generate C4a and C4b, as well as C2a and C2b. The C4b and C2a fragments combine to form the C3 convertase, C4b2a, which in turn cleaves C3 to form C3a and C3b. Activation of the LP is initiated by MBL binding to certain sugars on the target surface and this triggers the activation of MBL-associated serine proteases (MASPs) which then cleave C4 and C2 in a manner analogous to the activity of C1s of the CP, resulting in the generation of the C3 convertase, C4b2a. Thus, the CP and LP are activated by different mechanisms but they share the same components C4 and C2 and both pathways lead to the generation of the same C3 convertase, C4b2a. The cleavage of C3 by C4b2a into C3b and C3a is a central event of the complement pathway for two reasons. It initiates the AP amplification loop because surface deposited C3b is a central intermediate of the AP C3 convertase C3bBb. Both C3a and C3b are biologically important. C3a is proinflammatory and together with C5a are referred to as anaphylatoxins. C3b and its further cleavage products also bind to complement receptors present on neutrophils, eosinophils, monocytes and macrophages, thereby facilitating phagocytosis and clearance of C3b-opsonized particles. Finally, C3b can associate with C4b2a or C3bBb to form the C5 convertase of the CP and LP, and AP, respectively, to activate the terminal complement sequence, leading to the production of C5a, a potent proinflammatory mediator, and the assembly of the lytic membrane attack complex (MAC), C5-C9.

Successful immune surveillance by complement relies on the balance between activation and regulation as well as on discrimination between self and non-self surfaces. Disruption of this balance may have severe adverse clinical consequences. See, Ricklin D, Reis E S, Lambris J D, Nat Rev Nephrol. 2016; 12:383-401: Merle N S, Church S E. Fremeaux-Bacchi V, Roumenina L T, Front Immunol. 2015; 6:262.

Dysregulation or excessive activation of complement system is now recognized as a key pathogenic driver in many immune-mediated and inflammatory diseases. Some cells and organs, including the eyes, kidneys, and neuromuscular junctions, are particularly affected by complement-mediated damage. Disorders with complement involvement cover a broad range, including but are not limited to the inflammatory disorders affecting the kidney, hematological pathologies, ocular pathologies, cancer, ageing-related neuro-inflammatory and neurodegenerative disorders, and organ transplant rejection. Defective complement action is a cause of several human glomerular diseases including atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, renal ischemic reperfusion injury, lupus nephritis, membranous nephropathy, and chronic transplant mediated glomerulopathy. Aberrant complement component activation has been proposed as markers in various types of cancers and their clinical outcomes. Lung cancer patients show significantly higher plasma levels of complement proteins and activation fragments than do control donors, and elevated complement levels are correlated with lung tumor size. Complement-related proteins are also elevated in biological fluids from patients with other types of tumor. See, for example, Pio et al. Semin Immunol. 2013 February; 25(1): 54-64. Inhibition of the complement cascade has been proposed for glomerular diseases and cancer treatment.

Human C2 is a 83 kDa protein (100 kDa glycosylated) that is produced as an inactive, heavily glycosylated zymogen consisting of five domains: three N-terminal complement-control-protein (CCP) domains, a von Willebrand factor A-type (VWA) domain, and a C-terminal trypsin-like serine proteinase (SP) domain. The last two domains, VWA and SP, form the C2a fragment (residues 224-732, 70 kDa) that is produced in the proteolytic activation cascade of the classical and lectin pathways. The larger fragment C2a is 57.4 kDa (70 kDa glycosylated) and provides the catalytic center to the convertase complexes of the classical and lectin-binding pathways of complement activation. Exemplary human and cynomolgus C2 and C2a sequences are shown in any of SEQ ID NOs: 185-188.

In some embodiments, the anti-C2 antibody described herein inhibits the activity of the classical and lectin complement pathways. In some embodiments, the activity of the complement pathway that is inhibited using the anti-C2 antibody is a complement pathway activation induced by at least one of the group selected from a lipopolysacchride (LPS), lipooligosaccharide (LOS), pathogen-associated molecular patterns (PAMPs) and danger-associated molecular patterns (DAMPs), and auto-antibodies from autoimmune and inflammatory diseases. In some embodiments, the activity of complement signaling that is inhibited using a method of the invention is the formation of MAC. In another embodiment, the activity of complement signaling that is inhibited using a method of invention is the availability of C2 protein and/or C2a protein.

Binding affinity and specificity of the anti-C2 antibody described herein can be determined experimentally by methods known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), Bio-Layer Interferometry (BLI) (e.g. Octet system, ForteBio), RIA, ECL, IRMA, EIA, peptide scans, and enzyme-linked immunosorbent assay (ELISA). See, e.g., Benny K. C. Lo (2004) “Antibody Engineering: Methods and Protocols.” Humana Press (ISBN: 1588290921); Borrebaek (1992) “Antibody Engineering, A Practical Guide.” W. H. Freeman and Co., N.Y.; Borrebaek (1995) “Antibody Engineering.” 2nd Edition, Oxford University Press, N.Y., Oxford; Johne et al. (1993). J Immunol Meth 160:191-198; Jonssonetal. (1993) Ann Biol Clin 51:19-26; and Jonsson et al. (1991) Biotechniques 11:620-627. In addition, methods for measuring the affinity (e.g., dissociation and association constants by BLI) are set forth in the working examples.

Methods for determining whether a particular antibody described herein inhibits C2 cleavage are known in the art. Inhibition of human complement component C2 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay in chicken erythrocyte hemolysis method as described in, e.g., Hillmen et al. (2004) N Engl. J Med 350(6):552. In order to determine the effect of the anti-C2 antibody on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor.

IgM-mediated C3b formation assays can be used to determine the inhibitory activity of the anti-C2 antibodies on classical pathway induced C3b formation. Plate-bound and immobilized IgM can resemble immune complex and activate the classical pathway complement. In this assay, human IgM is coated onto ELISA plate, and after washing with PBS buffer, normal human serum (1% or 50%) in GVB++ buffer is added to the plate and incubated at room temperature for 60 min. After washing, the amount of C3b generated and bound to the plate is detected by anti-C3b/iC3b antibodies.

Inhibition of the lectin pathway by the anti-C2 antibodies may be assessed by assays targeting the mannan-binding lectin (MBL) pathway of complement activation. MBL is a carbohydrate-binding serum protein, which circulates in complex with serine proteases known as mannan-binding lectin associated serine proteases (MASPs). When bound to microorganisms, the MBL complex activates, through activities of MASP-1 and MASP-2, the complement components C4 and C2, thereby generating the C3 convertase and leading to opsonisation by the deposition of C4b and C3b fragments. See, for example. Petersen et al. J Immunol Methods. 2001 Nov. 1; 257(1-2): 107-16.

Methods for determining whether a candidate compound inhibits the cleavage of human C2 into forms C2a and C2b are known in the art and described in, e.g., Thomas et al. (1996) Mol Immunol 33(17-18): 1389-401; and Evans et al. (1995) Mol Immunol 32(16): 1183-95. For example, the concentration and/or physiologic activity of C2a and C2b in a body fluid can be measured by methods well known in the art. Methods for measuring C2a concentration or activity include, e.g., biolayer interferometry, RIAs, or ELISAs (see, e.g., Wurzner et al. (1991) Complement Inflamm 8:328-340). Using assays of these or other suitable types, candidate agents capable of inhibiting human complement component C2 can be screened.

### pH Dependent Anti-C2 Antibodies

In some embodiments, the anti-C2 antibodies described herein possess pH-dependent dissociation from C2. Such pH-dependent binding provides for greater persistence of administered antibody or antibody fusion protein molecules, because immune complexes (i.e., the anti-C2 antibody bound to C2 and/or C2a) taken up by cells will dissociate in the acidic environment of the endosome and allow the freed antibody or antibody fusion protein to be recycled back out of the cell through the neonatal Fc receptor (FcRn) where it is available to bind to a new C2 and/or C2a molecule. It is to be understood that discussion about pH-dependent anti-C2 antibodies in this section also applies to pH-dependent anti-C2 antibody fusion proteins (such as pH-dependent anti-C2 factor H fusion proteins).

In some embodiments, the anti-C2 antibody described possesses pH-dependent binding to C2 and/or C2a. As used herein, the expression “pH-dependent binding” means that the antibody exhibits reduced binding to C2 and/or C2a at acidic pH (e.g. about pH 5.8; such as in the endosome) as compared to its binding at neutral pH (e.g. about pH 7.4; such as in the blood).

pH-dependency of the anti-C2 antibody described herein can be determined experimentally by methods known in the art, such as in U.S. Pat. No. 9,079,949, and WO2016/098356. pH-dependency may be reflected in the differences in binding properties such as binding affinity (e.g. dissociation constant), kinetic parameters (e.g. association rate and dissociation rate), and percentage dissociation, at different pH levels. In some embodiments, the pH-dependency of the anti-C2 antibody described herein may be expressed in terms of the ratio of the percentage dissociation. In some embodiments, the percentage dissociation may be expressed in terms of the low-pH dissociation factor and the neutral-pH dissociation factor.

The pH dependence of the anti-C2 antibody can be assessed based on the dissociation of a C2-bound or a C2a-bound antibody at an acidic pH (e.g., pH 5.8) or at a neutral pH (e.g., pH 7.4). Low-pH dissociation factor, namely, the percentage of antibody dissociated at pH 5.8 from the antigen at 25° C., wherein the antibody is pre-bound to the antigen at pH 7.4, can be used to determine the dissociation of a C2-bound antibody at an acidic pH. The low-pH dissociation factor may be measured by associating an antibody and an antigen (e.g. the anti-C2 antibody and any one of: human C2, human C2a, cyno C2, cyno C2a) at pH 7.4 for 600 seconds, followed by a dissociation period of 600 seconds in a buffer at pH 5.8, and calculation of the percentage of antibody dissociated at pH 5.8 from the antigen. In some embodiments, the low-pH dissociation factor of the anti-C2 antibody of the present invention is in the range of any one of about 5% to about 95%, about 10% to about 90%, about 15% to about 85%, about 20% to about 80%, about 20% to about 75%, about 20% to about 70%, about 20% to about 65%, about 20% to about 60%, about 25% to about 75%, about 25% to about 70%, about 25% to about 65%, about 25% to about 60%, about 30% to about 75%, about 30% to about 70%, about 30% to about 65%, about 30% to about 60%, about 35% to about 75%, about 35% to about 70%, about 35% to about 65%, about 35% to about 60%, about 40% to about 75%, about 40% to about 70%, about 40% to about 65%, about 40% to about 60%. In some embodiments, the low-pH dissociation factor of the anti-C2 antibody is no less than about any of 95%, 90%, 85%, 80%, 75%, 70%, 65%, 60%, 55%, 50%, 45%, 40%, 35%, 30%, 25%, 20%, 15%, 10%, or 5%.

Neutral-pH dissociation factor, namely, the percentage of antibody dissociated at pH 7.4 from the antigen at 25° C., wherein the antibody is pre-bound to the antigen at pH 7.4 and can be used to determine the dissociation of a C2-bound antibody at a neutral pH. The neutral-pH dissociation factor may be measured by associating an antibody and an antigen (e.g. the anti-C2 antibody and any one of: human C2, human C2a, cyno C2, cyno C2a) at pH 7.4 for 600 seconds, followed by a dissociation period of 600 seconds in a buffer at pH 7.4, and calculation of the percentage of antibody dissociated at pH 7.4 from the antigen. In some embodiments, the neutral-pH dissociation factor of the anti-C2 antibody of the present invention is no more than about any of 20%, 18%, 16%, 14%, 12%, 10%, 9%, 8%, 7%, 6%, 5%, 4%, 3%, 2%, or 1%.

In some embodiments, the anti-C2 antibody binds to antigens (such as the human C2/human C2a/cyno C2/cyno C2a) more strongly at a neutral pH (such as pH 7.4) than it does at an acidic pH (such as pH 5.8). In some embodiments, the low-pH dissociation factor of the anti-C2 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C2 antibody is no more than about any of 20%, 18%, 16%, 14%, 12%, 10%, 9%, 8%, 7%, 6%, 5%, 4%. 3%, 2%, or 1%. In some embodiments, the ratio of the low-pH dissociation factor over the neutral-pH dissociation factor of the anti-C2 antibody is about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the anti-C2 antibody is no less than about any of 1.5, 2, 2.5, 3, 3.5, 4, 4.5, 5, 5.5, 6, 6.5, 7, 7.5, 8, 8.5, 9, 9.5, or 10. In some embodiments, the ratio of the low-pH dissociation factor over the neutral-pH dissociation factor of the anti-C2 antibody is no less than 4. In some embodiments, the ratio of the low-pH dissociation factor over the neutral-pH dissociation factor of the anti-C2 antibody is no less than 5. In some embodiments, the ratio of the low-pH dissociation factor over the neutral-pH dissociation factor of the anti-C2 antibody is no less than 6.

### Anti-C2 Antibody Fusion Protein

Activation of C2 by Cis to generate C2a and C2b is required to form the classical pathway C3 convertase C4b2a which cleaves C3. Previous genetic studies in animals and humans have revealed a ‘C2-bypass’ phenomenon such that in the total absence of C2, the classical pathway is not totally abolished and still functional (see, for example, Molecular Immunology, Vol. 27, No. 11, pp. 1155-1161, 1990; J Immunol 1989; 143:2256-2261; J Immunol 1999; 163:3549-3558). The mechanism of this ‘C2-bypass’ phenomenon is thought to involve a collaboration and participation of the alternative pathway complement proteins, e.g. by forming a hybrid C3 convertase C4bBb. Accordingly, while C2 antibodies may bind C2 and completely block its function, they may not completely block classical pathway complement activity due to this ‘C2-bypass’ phenomenon.

The anti-C2 antibodies described herein can be conjugated (e.g., fused) to another moiety. In some embodiments, the other moiety is a targeting moiety (such as an antigen binding polypeptide). In some embodiments, the other moiety is an effector moiety (e.g., a drug, a toxin, etc.). In some embodiments, the other moiety is a complement associated protein or functional fragment thereof.

The complement system is tightly regulated by a network of proteins known as regulators of complement activation that help distinguish target cells as self or non-self. A subset of this family of proteins, complement control proteins (also known as sushi domains) are characterized by domains of conserved repeats that direct interaction with components of the complement system. Most complement control proteins prevent activation of the complement system on the surface of host cells and protect host tissues against damage caused by autoimmunity. Because of this, these proteins play important roles in autoimmune disorders and cancers. Exemplary complement control proteins include, but are not limited to, membrane cofactor protein, MCP (CD46); decay accelerating factor, DAF (CD55); protectin (CD59); complement C3b/C4b receptor 1, CR1 (CD35); complement regulator of the immunoglobulin superfamily, CRIg; factor H; and C4-binding protein (C4 bp). The anti-C2 antibody described herein can be fused to any one of these complement associated proteins or fragments thereof. In some embodiments, the anti-C2 antibody fusion proteins have activities that overcome the “C2-bypass” phenomenon and blocks the classical pathway and lectin pathway completely.

In some embodiments, the anti-C2 antibody is fused to factor H or a functional fragment thereof. The present application thus in some embodiments provides a fusion protein comprising an anti-C2 antibody moiety (such as, but not necessarily limited to, any one of the anti-C2 antibodies described herein) fused to a factor H or a fragment thereof. Factor H (fH) is a single polypeptide chain plasma glycoprotein. The protein is composed of 20 conserved short consensus repeat (SCR) domains of approximately 60 amino acids, arranged in a continuous fashion like a string of beads, separated by short linker sequences of 2-6 amino acids each. Factor H binds to C3b, accelerates the decay of the alternative pathway C3-convertase (C3bBb), and acts as a cofactor for the proteolytic inactivation of C3b by factor I. Alternative pathway amplification is initiated when circulating factor B binds to activated C3b. This complex is then cleaved by circulating factor D to yield an enzymatically active C3 convertase complex, C3bBb. C3bBb cleaves additional C3 generating C3b, which drives inflammation and also further amplifies the activation process, generating a positive feedback loop. Factor H is a key regulator (inhibitor) of the alternative complement pathway activation and initiation mechanisms that competes with factor B for binding to conformationally altered C3 (hydrolyzed form of C3 or C3(H2O)) in the tick-over mechanism and to C3b in the amplification loop. Binding of C3b to Factor H also leads to degradation of C3b by factor I to the inactive form iC3b (also designated C3bi), thus exerting a further check on complement activation. Factor H regulates complement in the fluid phase, circulating at a plasma concentration of approximately 500 μg/ml, but its binding to cells is a regulated phenomenon enhanced by the presence of a negatively charged surface as well as fixed C3b, iC3b, C3dg or C3d. See, for example, Jozsi et al., Histopathol. (2004) 19: 251-258.

In some embodiments, the complement associated protein described herein comprises at factor-H (FH) protein or a fragment thereof. In one embodiment, the fragment of factor H comprises at least one SCR domains of the factor H protein. In one embodiment, the fragment of factor H comprises at least two SCR domains of the factor H protein. In one embodiment, the fragment of factor H comprises at least three SCR domains of the factor H protein. In one embodiment, the fragment of factor H comprises at least four SCR domains of the factor H protein. In one embodiment, the fragment of factor H comprises at least five SCR domains of the factor H protein. In one embodiment, the fragment of factor H comprises at least six SCR domains of the factor H protein. In one embodiment, the fragment of factor H comprises at least seven SCR domains of the factor H protein. In one embodiment, the fragment of factor H comprises at least eight SCR domains of the factor H protein. In one embodiment, the fragment of factor H comprises at least nine SCR domains of the factor H protein. In one embodiment, the fragment of factor H comprises at least ten SCR domains of the factor H protein. In one embodiment, the fragment of factor H comprises at least eleven SCR domains of the factor H protein. In one embodiment, the fragment of factor H comprises at least twelve SCR domains of the factor H protein. In one embodiment, the fragment of factor H comprises at least thirteen SCR domains of the factor H protein. In one embodiment, the fragment of factor H comprises at least fourteen SCR domains of the factor H protein. In one embodiment, the fragment of factor H comprises at least fifteen SCR domains of the factor H protein. In one embodiment, the fragment of factor H comprises at least sixteen SCR domains of the factor H protein. In one embodiment, the fragment of factor H comprises at least seventeen SCR domains of the factor H protein. In one embodiment, the fragment of factor H comprises at least eighteen SCR domains of the factor H protein. In one embodiment, the fragment of factor H comprises at least nineteen SCR domains of the factor H protein. In one embodiment, the fragment of factor H comprises at least twenty SCR domains of the factor H protein.

In one embodiment, the fragment of factor H comprises SCR domains 1-20 of the factor H protein. In one embodiment, the fragment of factor H comprises SCR domains 1-5 of the factor H protein. In one embodiment, the fragment of factor H comprises SCR domains 1-4 of the factor H protein. In one embodiment, the fragment of factor H comprises SCR domains 2-5 of the factor H protein. In one embodiment, the fragment of factor H comprises SCR domains 3-5 of the factor H protein. In one embodiment, the fragment of factor H comprises SCR domains 4 and 5 of the factor H protein. In one embodiment, the fragment of factor H comprises SCR domain 5 of the factor H protein.

In some embodiments, there is provided a fusion protein comprising a fusion protein partner linked to an anti-C2 antibody (such as any one of the anti-C2 antibodies described herein). In some embodiments, there is provided a fusion protein comprising an anti-C2 antibody comprising a heavy chain variable region (VH) comprising an H-CDR1, an H-CDR2, and an H-CDR3 of an VH comprising the amino acid sequence of any one of SEQ ID NOs: 1, 9, 17, 25, 33, 41, 49, 57, 65, 73, 81, 89, and 246; and a light chain variable region (VL) comprising an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247.

In some embodiments, the fusion protein comprises a fusion protein partner bound to any one of the anti-C2 antibodies described herein with at least one linker. In some embodiments, the fusion protein comprises a fusion protein partner bound to any one of the anti-C2 antibodies described herein without a linker. In some embodiments, the fusion protein comprises a fusion protein partner bound to C-terminal of the heavy chain of any one of the anti-C2 antibodies described herein. In some embodiments, the fusion protein comprises a fusion protein partner bound to N-terminal of light or heavy chain of any one of the anti-C2 antibodies described herein.

In some embodiments, the anti-C2 antibody fusion protein further comprises a complement control protein or a fragment of a complement control protein. In one embodiment, the complement control protein or fragment of complement control protein is an inhibitor of C3 convertase. In one embodiment, the C3 convertase is the alternative pathway C3 convertase C3bBb. In one embodiment, the C3 convertase is the classical pathway C3 convertase C4b2a. In one embodiment, the complement control protein or fragment of complement control protein is an inhibitor of complement activation steps other than C3 or C2 activation. In various embodiments, the fusion protein comprises a complement receptor 1 (CR1) or a fragment thereof, a membrane cofactor protein (MCP) or a factor thereof, a C4b-binding protein (C4BP) or a fragment thereof, a decay-accelerating factor (DAF) or a fragment thereof, an Apolipoprotein E (ApoE) or a fragment thereof, a factor H protein or a fragment thereof, a human IgG4 or a fragment thereof, a linker, or any combination thereof. In some embodiments, the fragment of factor H comprises SCR domains 1-5 of the factor H protein. In some embodiments, the fragment of decay-accelerating factor (DAF) is the extracellular domain of DAF. In some embodiments, the fragment of CR1 is selected SCRs of the extracellular domain of CR1. In some embodiments, the anti-C2 antibodies described herein are fused (i.e., covalently linked) to factor H or a fragment thereof.

In some embodiments, the anti-C2 antibody fusion protein is an anti-C2 antibody or an antigen binding fragment thereof linked to factor H or a fragment thereof. In some embodiments, the anti-C2 antibody factor H fusion protein comprises any one of the anti-C2 antibody V_{L} sequences described herein. In some embodiments, the anti-C2 antibody is any one of the anti-C2 antibodies described herein. In some embodiments, the factor H is human factor H. In some embodiments, the anti-C2 antibody is fused to the factor H polypeptide or fragment thereof comprises SCR1-5 domains of factor H. In some embodiments, the fragment of factor H comprises SCR domains 1-5 of the factor H protein. In some embodiments, the fragment of factor H comprising the amino acid sequence of SEQ ID NO: 201.

In some embodiments, the anti-C2 antibody factor H fusion protein or a fragment thereof exhibits pH-dependent binding to C2. In some embodiments, the pH-dependent anti-C2 antibody factor H fusion protein or a fragment thereof binds more strongly to C2 at a more neutral pH (e.g., about pH 7.4; such as that found in the blood) than it does at a more acidic pH (e.g., about pH 5.8; such as that found in the endosome).

In some embodiments, the anti-C2 antibody factor H fusion protein or a fragment thereof has comparable pH dependence to the anti-C2 antibody. In some embodiments, the anti-C2 antibody factor H fusion protein or a fragment thereof bound to human C2 at pH 7.4 and 25° C. dissociates from human C2 at pH 5.8 and 25° C. over a time window of 600 seconds in the percentage range of any one of about 5% to about 95%, about 10% to about 90%, about 15% to about 85%, about 20% to about 80%, about 20% to about 75%, about 20% to about 70%, about 20% to about 65%, about 20% to about 60%, about 25% to about 75%, about 25% to about 70%, about 25% to about 65%, about 25% to about 60%, about 30% to about 75%, about 30% to about 70%, about 30% to about 65%, about 30% to about 60%, about 35% to about 75%, about 35% to about 70%, about 35% to about 65%, about 35% to about 60%, about 40% to about 75%, about 40% to about 70%, about 40% to about 65%, about 40% to about 60%. In some embodiments, the anti-C2 antibody factor H fusion protein or a fragment thereof bound to human C2 at pH 7.4 and 25° C. dissociates from human C2 at pH 5.8 and 25° C. in the percentage range of 20% to about 80%.

In some embodiments, the anti-C2 antibody factor H fusion protein or a fragment thereof bound to human C2 at pH 7.4 and 25° C. dissociates from human C2 at pH 7.4 and 25° C. over a time window of 600 seconds in the percentage range of any one of about 0% to about 20%, about 0% to about 18%, about 0% to about 16%, about 0% to about 14%, about 0% to about 12%, about 0% to about 10%, about 0% to about 9%, about 0% to about 8%, about 0% to about 7%, about 0% to about 6%, about 0% to about 5%, about 0% to about 4%, about 0% to about 3%, about 0% to about 2%, about 0% to about 1%. In some embodiments, the anti-C2 antibody factor H fusion protein or a fragment thereof bound to human C2 at pH 7.4 and 25′C dissociates from human C2 at pH 7.4 and 25′C in the percentage range of 0% to about 12%.

In some embodiments, the percentage of dissociation of anti-C2 antibody factor H fusion protein or a fragment thereof at pH 5.8 over the percentage of dissociation of the anti-C2 antibody factor H fusion protein or a fragment thereof at pH 7.4 is any one of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the anti-C2 antibody factor H fusion protein or a fragment thereof at pH 5.8 over the percentage of dissociation of the anti-C2 antibody factor H fusion protein or a fragment thereof at pH 7.4 is 4 or more.

In some embodiments, the anti-C2 antibody factor H fusion protein or a fragment thereof inhibits the classical complement pathway. In some embodiments, the anti-C2 antibody factor H fusion protein or a fragment thereof shows potent inhibition of sheep red blood cell lysis in the presence of normal human serum, for example, in 50% normal human serum or in 1% human serum. In some embodiments, the anti-C2 antibody factor H fusion protein or a fragment thereof shows potent inhibition of sheep red blood cell lysis in 1% human serum with an IC₅₀ value of less than 1 nM. In some embodiments, the anti-C2 antibody factor H fusion protein or a fragment thereof shows potent inhibition of sheep red blood cell lysis in 50% human serum with an IC₅₀ value of about 1 nM to about 1000 nM, such as about 10's nM concentration.

The anti-C2 antibody and the factor H may be linked directly by a single chemical bond (such as peptide bond) or via a peptide linker. The peptide linker may have a naturally occurring sequence, or a non-naturally occurring sequence. For example, a sequence derived from the hinge region of heavy chain-only antibodies may be used as the linker. See, for example, WO1996/34103. In some embodiments, the peptide linker is a flexible linker.

In some embodiments, the fusion protein comprises a fusion protein partner bound to an VH sequence of the antibody. In some embodiments, the fusion protein comprises a fusion protein partner bound to C-terminal of an VH sequence of the antibody. In one embodiment, the fusion protein comprises a fusion protein partner bound to N-terminal of an VH sequence of the antibody.

In some embodiments, the fusion protein comprises a fusion protein partner fused to the Fc of the anti-C2 antibody. The anti-C2 antibody, a fragment, or a fusion protein thereof (such as a full-length antibody or an scFv) may be fused to the factor H or a fragment thereof at either the N-terminus or the C-terminus of the factor H or a fragment thereof. In some embodiments, the anti-C2 antibody, a fragment, or a fusion protein thereof (such as a full-length antibody or an scFv) is fused at the N-terminus of the factor H polypeptide or fragment thereof. In some embodiments, the anti-C2 antibody, a fragment, or a fusion protein thereof is fused at the C-terminus of the factor H polypeptide or fragment thereof. In some embodiments, the factor H polypeptide or fragment thereof is fused to the N or C terminus of an IgG or fragment thereof at one or more heavy or light chains.

In some embodiments, the anti-C2 antibody factor H fusion protein comprises a heavy chain-factor H fusion comprising the amino acid sequence of SEQ ID NO: 202, or a variant thereof having at least 80% (such as 80%, 85%, 90%, 95%, 96%, 97%, 98%, 98%, 99%) amino acid sequence homology to SEQ ID NO: 202. In some embodiments, the anti-C2 antibody factor H fusion protein comprises a heavy chain-factor H fusion comprising the amino acid sequence of SEQ ID NO: 203, or a variant thereof having at least 80% (such as 80%, 85%, 90%, 95%, 96%, 97%, 98%, 98%, 99%) amino acid sequence homology to SEQ ID NO: 203. In some embodiments, the anti-C2 antibody factor H fusion protein comprises a heavy chain-factor H fusion comprising the amino acid sequence of SEQ ID NO: 211, or a variant thereof having at least 80% (such as 80%, 85%, 90%, 95%, 96%, 97%, 98%, 98%, 99%) amino acid sequence homology to SEQ ID NO: 211. In some embodiments, the anti-C2 antibody factor H fusion protein comprises a heavy chain-factor H fusion comprising the amino acid sequence of SEQ ID NO: 214, or a variant thereof having at least 80% (such as 80%, 85%, 90%, 95%, 96%, 97%, 98%, 98%, 99%) amino acid sequence homology to SEQ ID NO: 214. In some embodiments, the anti-C2 antibody factor H fusion protein comprises a heavy chain-factor H fusion comprising the amino acid sequence of SEQ ID NO: 215, or a variant thereof having at least 80% (such as 80%, 85%, 90%, 95%, 96%, 97%, 98%. 98%, 99%) amino acid sequence homology to SEQ ID NO: 215. In some embodiments, the anti-C2 antibody factor H fusion protein comprises a heavy chain-factor H fusion comprising the amino acid sequence of SEQ ID NO: 216, or a variant thereof having at least 80% (such as 80%, 85%, 90%, 95%, 96%, 97%, 98%, 98%, 99%) amino acid sequence homology to SEQ ID NO: 216.

In some embodiments, the anti-C2 antibody factor H fusion protein comprises: an VH comprising the amino acid sequence of any one of SEQ ID NOs: 1, 9, 17, 25, 33, 41, 49, 57, 65, 73, 81, 89, and 246; an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247; and a heavy chain constant domain-factor H fusion comprising the amino acid sequence of any of SEQ ID NOs: 202, 203, 211, and 214-216.

In some embodiments, the anti-C2 antibody fusion protein comprises a heavy chain-factor H fusion polypeptide and a light chain, wherein the heavy chain-factor H fusion comprises an amino acid sequence of any of SEQ ID NOs: 202-204, 206, 211-213, and 220-228, and the light chain comprises an amino acid sequence of any of SEQ ID NOs: 205, 207, 229-245, and 249. In some embodiments, the anti-C2 antibody fusion protein comprises a VL sequence selected from any of SEQ ID NOs: 229-245 and 249, and a heavy-chain FH fusion sequence selected from any of SEQ ID NOs: 223-228 and 248.

### Properties of the Anti-C2 Antibodies and Fusion Proteins Thereof

The anti-C2 antibodies and fusion proteins described herein are amenable for development and use as a pharmaceutical composition. It is to be understood that discussion about anti-C2 antibodies (including the pH-dependent anti-C2 antibodies) in this section also applies to anti-C2 antibody fusion proteins (such as anti-C2 factor H fusion proteins, including pH-dependent anti-C2 factor H fusion proteins, wherein the anti-C2 antibody comprise a full length antibody or a fragment thereof, such as scFv).

In some embodiments, the anti-C2 antibody or fusion protein inhibits the classical complement pathway (e.g. inhibiting sheep red blood cell lysis in 5% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at most 5 nM, at most 6 nM, at most 7 nM, at most 8 nM, at most 9 nM, at most 10 nM, at most 20 nM, or at most 30 nM. In some embodiments, there is provided an anti-C2 antibody or fusion protein which inhibits the classical complement pathway (e.g. inhibiting sheep red blood cell lysis in 5% human serum) with an IC₅₀ value of at least 1 nM to at most 5 nM.

In some embodiments, the anti-C2 antibody or fusion protein inhibits the classical complement pathway (e.g. inhibiting sheep red blood cell lysis in 20% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at most 20 nM, or at most 30 nM. In some embodiments, there is provided an anti-C2 antibody or fusion protein which inhibits the classical complement pathway (e.g., inhibiting sheep red blood cell lysis in 20% human serum) with an IC₅₀ value of at least 10 nM to at most 20 nM.

In some embodiments, the anti-C2 antibody or fusion protein inhibits the classical complement pathway (e.g. inhibiting sheep red blood cell lysis in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 200 nM, at most 300 nM, at most 400 nM, or at most 500 nM. In some embodiments, there is provided an anti-C2 antibody or fusion protein which inhibits the classical complement pathway (e.g. inhibiting sheep red blood cell lysis in 50% human serum) with an IC₅₀ value of at least 1 nM to at most 300 nM.

In some embodiments, the anti-C2 antibody or fusion protein inhibits the classical complement pathway (e.g. inhibiting sheep red blood cell lysis in 20% cyno serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at most 20 nM, or at most 30 nM. In some embodiments, there is provided an anti-C2 antibody or fusion protein which inhibits the classical complement pathway (e.g. inhibiting sheep red blood cell lysis in 20% cyno serum) with an IC₅₀ value of at least 10 nM to at most 15 nM.

In some embodiments, the anti-C2 antibody or fusion protein inhibits the classical complement pathway (e.g. using IgM-C3b ELISA in 1% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at most 5 nM, at most 6 nM, at most 7 nM, at most 8 nM, at most 9 nM, at most 10 nM. In some embodiments, there is provided an anti-C2 antibody or fusion protein which inhibits the classical complement pathway (e.g. using IgM-C3b ELISA in 1% human serum) with an IC₅₀ value of at least 0.1 nM to at most 5 nM.

In some embodiments, the anti-C2 antibody or fusion protein inhibits the classical complement pathway (e.g. using IgM-C3b ELISA in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 200 nM, at most 300 nM, at most 400 nM, at most 500 nM. In some embodiments, there is provided an anti-C2 antibody or fusion protein which inhibits the classical complement pathway (e.g., using IgM-C3b ELISA in 50% human serum) with an IC₅₀ value of at least 1 nM to at most 300 nM.

In some embodiments, the anti-C2 antibody or fusion protein inhibits the lectin complement pathway (e.g. using Mannan-C3b ELISA in 1% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, or at most 10 nM. In some embodiments, there is provided an anti-C2 antibody or fusion protein which inhibits the lectin complement pathway (e.g., using Mannan-C3b ELISA in 1% human serum) with an IC₅₀ value of at least 0.1 nM to at most 10 nM.

In some embodiments, the anti-C2 antibody or fusion protein inhibits the lectin complement pathway (e.g. using Mannan-C3b ELISA in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 200 nM, at least 300 nM, at least 400 nM, at most 500 nM, at most 600 nM, at most 700 nM, at most 800 nM, at most 900 nM, or at most 1 μM. In some embodiments, there is provided an anti-C2 antibody or fusion protein which inhibits the lectin complement pathway (e.g., using Mannan-C3b ELISA in 50% human serum) with an IC₅₀ value of at least 10 nM to at most 500 nM.

In some embodiments, the anti-C2 antibody or fusion protein inhibits the alternative complement pathway (e.g. using LPS-C3b ELISA in 10% human serum) with an IC₅₀ value of at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at most 50 nM, at most 60 nM, at most 70 nM, at most 80 nM, at most 90 nM, or at most 100 nM. In some embodiments, there is provided an anti-C2 antibody or fusion protein which inhibits the alternative complement pathway (e.g., using LPS-C3b ELISA in 10% human serum) with an IC₅₀ value of at least 0.5 nM to at most 50 nM.

In some embodiments, the anti-C2 antibody or fusion protein inhibits the alternative complement pathway (e.g. using LPS-C3b ELISA in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 200 nM, at most 300 nM, at most 400 nM, at most 500 nM, at most 600 nM, at most 700 nM, at most 800 nM, at most 900 nM, or at most 1 μM. In some embodiments, there is provided an anti-C2 antibody or fusion protein which inhibits the alternative complement pathway (e.g., using LPS-C3b ELISA in 50% human serum) with an IC₅₀ value of at least 10 nM to at most 300 nM.

In some embodiments, the anti-C2 antibody or fusion protein inhibits the classical/terminal complement pathway (e.g. using IgM-C5b-9 ELISA in 1% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at most 5 nM, at most 6 nM, at most 7 nM, at most 8 nM, at most 9 nM, at most 10 nM, at most 20 nM, or at most 30 nM. In some embodiments, there is provided an anti-C2 antibody or fusion protein which inhibits the classical/terminal complement pathway (e.g. using IgM-C5b-9 ELISA in 1% human serum) with an IC₅₀ value of at least 0.05 nM to at most 5 nM.

In some embodiments, the anti-C2 antibody or fusion protein inhibits the classical/terminal complement pathway (e.g. using IgM-C5b-9 ELISA in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at most 200 nM, at most 300 nM, at most 400 nM, at most 500 nM, at most 600 nM, at most 700 nM, at most 800 nM, at most 900 nM, or at most 1 μM. In some embodiments, there is provided an anti-C2 antibody or fusion protein which inhibits the classical/terminal complement pathway (e.g. using IgM-C5b-9 ELISA in 50% human serum) with an IC₅₀ value of at least 10 nM to at most 200 nM.

In some embodiments, the anti-C2 antibody or fusion protein inhibits the alternative/terminal complement pathway (e.g. using LPS-C5b-9 ELISA in 10% human serum) with an IC₅₀ value of at least 0.01 nM, at least 0.05 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at most 100 nM, at most 200 nM, at most nM, at most 400 nM, or at most 500 nM. In some embodiments, there is provided an anti-C2 antibody or fusion protein which inhibits the alternative/terminal complement pathway (e.g. using LPS-C5b-9 ELISA in 10% human serum) with an IC₅₀ value of at least 1 nM to at most 100 nM.

In some embodiments, the anti-C2 antibody or fusion protein inhibits the alternative/terminal complement pathway (e.g. using LPS-C5b-9 ELISA in 50% human serum) with an IC₅₀ value of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at most 200 nM, at most 300 nM, at most 400 nM, at most 500 nM, at most 600 nM, at most 700 nM, at most 800 nM, at most 900 nM, or at most 1 μM. In some embodiments, there is provided an anti-C2 antibody or fusion protein which inhibits the alternative/terminal complement pathway (e.g. using LPS-C5b-9 ELISA in 50% human serum) with an IC₅₀ value of at least 10 nM to at most 200 nM.

The anti-C2 antibody described herein in some embodiments exhibit prolonged serum half-life in vivo. In some embodiments, the anti-C2 antibody exhibits prolonged serum half-life in mice (including transgenic mice). In some embodiments, the anti-C2 antibody exhibits prolonged serum half-life in other test animals. Exemplary test animals include but are not limited to, rats, cynomolgus monkeys, chickens, rabbits, and sheep. In some embodiments, there is provided an anti-C2 antibody that has a serum half-life in mice of any one of at least about 2 hours, about 3 days, about 5 days, about 7 days, about 9 days, about 11 days, about 13 days, about 15 days, about 17 days, about 19 days, about 21 days, about 23 days, about 25 days. In some embodiments, there is provided an anti-C2 antibody that has a serum half-life in humans that is at least about 7 days.

The pH-dependent anti-C2 antibody described herein in some embodiments exhibit prolonged serum half-life in vivo. In some embodiments, the anti-C2 antibody exhibits prolonged serum half-life in cynomolgus monkeys. In some embodiments, the anti-C2 antibody exhibits prolonged serum half-life in other test animals. Exemplary test animals include but are not limited to, rats, mice (including transgenic mice), chickens, rabbits, and sheep. In some embodiments, the anti-C2 antibody exhibits prolonged serum half-life in humans. In some embodiments, there is provided an anti-C2 antibody that has a serum half-life in humans of any one of at least about 2 hours, about 3 days, about 5 days, about 7 days, about 9 days, about 11 days, about 13 days, about 15 days, about 17 days, about 19 days, about 21 days, about 23 days, about 25 days. In some embodiments, there is provided an anti-C2 antibody that has a serum half-life in humans that is at least about 25 days.

In some embodiments, the pH-dependent anti-C2 antibody has comparable binding affinity to human C2 to benchmark anti-C2 antibodies.

In some embodiments, the KD of the binding between an anti-C2 antibody described herein and C2 is about 10⁻⁹ M to about 10⁻¹¹ M (such as about 10⁻⁹ M to about 10⁻¹¹ M, or about 10⁻¹⁰ M to about 10⁻¹¹ M).

The anti-C2 antibody or fusion protein described herein may have cross-species reactivity to C2 other than human C2, or to C2a other than human C2a. Without being bound by any theory or hypothesis, cross-reactivity occurs when immunoglobulins from different species share conserved sequences and similar quaternary structure. The paratope (antigen-binding site) of an antibody that recognizes immunoglobulin from one species may detect a homologous epitope on immunoglobulin from another species. This is common in closely related species such as mouse and rat, but may also occur in less obvious pairings. Exemplary non-human C2 and C2a include, but are not limited to, mouse C2 and C2a, rat C2 and C2a, rabbit C2 and C2a, sheep C2 and C2a, cyno monkey C2 and C2a. In some embodiments, the anti-C2 antibody cross-reacts with cynomolgus C2 and/or C2a.

### III. Pharmaceutical Compositions

Further provided by the present application are pharmaceutical compositions comprising any one of the anti-C2 antibodies and a pharmaceutically acceptable carrier. Pharmaceutical compositions can be prepared by mixing the anti-C2 antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. It is to be understood that discussion about anti-C2 antibodies in this section also applies to anti-C2 antibody fusion proteins (such as anti-C2 factor H fusion proteins).

In some embodiments, the pharmaceutical composition further comprises additional ingredients. Additional ingredients include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

Additional excipients include agents which can serve as one or more of the following: (1) bulking agents, (2) solubility enhancers, (3) stabilizers and (4) and agents preventing denaturation or adherence to the container wall.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Compositions for sustained release or implantation may comprise pharmaceutically acceptable polymeric or hydrophobic materials such as an emulsion, an ion exchange resin, a sparingly soluble polymer, or a sparingly soluble salt. Suitable examples of sustained-release preparations include semi-permeable matrices of solid hydrophobic polymers containing the antagonist, which matrices are in the form of shaped articles, e.g. films, or microcapsules.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

The formulations of the pharmaceutical compositions may be prepared by any method known or hereafter developed in the art of pharmacology. Preparations include but are not limited to, bringing the active ingredient into association with a carrier or one or more other accessory ingredients, and then, if necessary or desirable, shaping or packaging the product into a desired single- or multi-dose unit. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

### IV. Methods of Use

Also provided herein are methods of inhibiting complement activation and treating diseases (such as complement-mediated diseases or disorders) in an individual by administering an effective amount of the anti-C2 antibody or an antibody construct comprising the anti-C2 antibody (such as any of the fusion proteins described herein) to the individual. In some embodiments, the individual is a human. It is to be understood that discussion about anti-C2 antibodies (including the pH-dependent anti-C2 antibodies) in this section also applies to anti-C2 antibody fusion proteins (such as anti-C2 factor H fusion proteins, including pH-dependent anti-C2 factor H fusion proteins, wherein the anti-C2 antibody comprise a full length antibody or a fragment thereof, such as scFv).

The anti-C2 antibody and antibody construct (such as fusion proteins described herein) can be used in combination with other treatment modalities, such as, for example anti-inflammatory therapies, and the like. Examples of anti-inflammatory therapies that can be used in combination with the methods of the invention include, for example, therapies that employ steroidal drugs, as well as therapies that employ non-steroidal drugs.

In some embodiments, there is provided a method of inhibiting complement activation in an individual, comprising administering (such as systemically administering, for example by subcutaneous or intravenous administration) to the individual an effective amount of an anti-C2 antibody or antibody construct comprising the anti-C2 antibody, a fragment, or a fusion protein thereof (such as factor H fusion protein described herein). In some embodiments, the anti-C2 antibody or fusion protein comprises an H-CDR1, an H-CDR2, and an H-CDR3 of an VH comprising the amino acid sequence of any one of SEQ ID NOs: 1, 9, 17, 25, 33, 41, 49, 57, 65, 73, 81 and 89. In some embodiments, the anti-C2 antibody comprises an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153 and 157. In some embodiments, the anti-C2 antibody further comprises one or more additional mutations (such as histidine mutations) in the variable domain (VL) of light chain. In some embodiments, the anti-C2 antibody comprises an F58H mutation in the VH, wherein the VH mutation is in reference to SEQ ID NO: 17 under the Kabat numbering system.

In some embodiment, the anti-C2 antibody further comprises one or more additional mutations (such as histidine mutations) in the variable domain (VL) of light chain, wherein the VL mutation is in reference to SEQ ID NO: 18 under the Kabat numbering system. In some embodiments, the anti-C2 antibody further comprises an IgG4 Fc region (such as an IgG4 Fc region comprises PLA mutation: S228P, M428L, and N434A). In some embodiments, the anti-C2 antibody further comprises an IgG1 Fc region. In some embodiments, there is provided a method of using an anti-C2 antibody wherein the anti-C2 antibody binds more strongly at a neutral pH (such as pH 7.4) than it does at an acidic pH (such as pH 5.8). In some embodiments, the low-pH dissociation factor of the anti-C2 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C2 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C2 antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the anti-C2 antibody from human C2 at pH 5.8 over the percentage of dissociation of the anti-C2 antibody from human C2 at pH 7.4 is 5 or more. In some embodiments, the percentage of dissociation of the anti-C2 antibody from cyno C2 at pH 5.8 over the percentage of dissociation of the anti-C2 antibody from cyno C2 at pH 7.4 is 7 or more. In some embodiments, the anti-C2 antibody is administered by intravenous administration.

In some embodiments, there is provided a method of using an anti-C2 antibody wherein the anti-C2 antibody inhibits complement activation by at least about any of 10%, 20%, 30%, 40%, 50%, 60%, 70%, 80%, 90%, or more. In some embodiments, there is provided a method of using an anti-C2 antibody wherein the anti-C2 antibody inhibits IgM-mediated C3b formation with an IC₅₀ value of about 0.1 nM to about 10 nM. In some embodiments, there is provided a method of using an anti-C2 antibody wherein the anti-C2 antibody inhibits mannan-mediated C3b formation with an IC₅₀ value of about 0.1 nM to about 10 nM. In some embodiments, there is provided a method of using an anti-C2 antibody wherein the anti-C2 antibody does not inhibit complement activation.

In some embodiments, there is provided a method of inhibiting complement activation in an individual, comprising administering (such as systemically administering, for example by subcutaneous or intravenous administration) to the individual an effective amount of an anti-C2 antibody. In some embodiments, the anti-C2 antibody comprises an H-CDR 1, an H-CDR2, and an H-CDR3 of an VH comprising the amino acid sequence of any one of SEQ ID NOs: 1, 9, 17, 25, 33, 41, 49, 57, 65, 73, 81 and 89. In some embodiments, the anti-C2 antibody comprises an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153 and 157. In some embodiments, the anti-C2 antibody further comprises one or more additional mutations (such as histidine mutations) in the variable domain (VL) of light chain. In some embodiments, the anti-C2 antibody comprises an F58H mutation in the VH, wherein the VH mutation is in reference to SEQ ID NO: 17 under the Kabat numbering system. In some embodiment, the anti-C2 antibody further comprises one or more additional mutations (such as histidine mutations) in the variable domain (VL) of light chain, wherein the VL mutation is in reference to SEQ ID NO: 18 under the Kabat numbering system. In some embodiments, the anti-C2 antibody further comprises an IgG4 Fc region (such as an IgG4 Fc region comprises PLA mutation: S228P, M428L, and N434A). In some embodiments, the anti-C2 antibody further comprises an IgG1 Fc region. In some embodiments, there is provided a method of using an anti-C2 antibody wherein the anti-C2 antibody binds more strongly at a neutral pH (such as pH 7.4) than it does at an acidic pH (such as pH 5.8). In some embodiments, the low-pH dissociation factor of the anti-C2 antibody is no less than about any of 95%, 90%, 85%, 80%, 75%, 70%, 65%, 60%, 55%, 50%, 45%, 40%, 35%, 30%, 25%, 20%, 15%, 10%, or 5%. In some embodiments, the neutral-pH dissociation factor of the anti-C2 antibody is no more than about any of 20%, 18%, 16%, 14%, 12%, 10%, 9%, 8%, 7%, 6%, 5%, 4%, 3%, 2%, or 1%. In some embodiments, the ratio of the low-pH dissociation factor over the neutral-pH dissociation factor of the anti-C2 antibody is about any of 1 or more, 1.5 or more, 2 or more, 2.5 or more, 3 or more, 3.5 or more, 4 or more, 4.5 or more, 5 or more, 6 or more, 7 or more, 8 or more, 9 or more, 10 or more. In some embodiments, the percentage of dissociation of the anti-C2 antibody from human C2 at pH 5.8 over the percentage of dissociation of the anti-C2 antibody from human C2 at pH 7.4 is 5 or more. In some embodiments, the percentage of dissociation of the anti-C2 antibody from cyno C2 at pH 5.8 over the percentage of dissociation of the anti-C2 antibody from cyno C2 at pH 7.4 is 7 or more. In some embodiments, the anti-C2 antibody is administered by intravenous administration.

In some embodiments, the complement-mediated disease or disorder is at least selected from the group consisting of: macular degeneration (MD), age-related macular degeneration (AMD), ischemia reperfusion injury, arthritis, rheumatoid arthritis, asthma, allergic asthma, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, myasthenia gravis, neuromyelitis optica, (NMO), multiple sclerosis, delayed graft function, antibody-mediated rejection, atypical hemolytic uremic (aHUS) syndrome, central retinal vein occlusion (CRVO), central retinal artery occlusion (CRAO), epidermolysis bullosa, Hidradenitis Suppurativa, bullous pemphigoid, Myelin oligodendrocyte glycoprotein antibody-associated disease (MOGAD), sepsis, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, COVID-19, or any combinations thereof.

Among the diseases listed above, aHUS is a rare, severe form of thrombotic microangiopathy (TMA) that is characterized by thrombocytopenia, hemolytic anemia and acute kidney injury with endothelial lesions that often lead to end-stage renal disease (ESRD). The interplay between the complement, coagulation and endothelial barrier systems seems to be particularly important in aHUS pathogenesis. Eculizumab (anti-C5) is currently a treatment of choice. See, for example, Fakhouri F et al. Lancet. 2017; 390:681-696; Roumenina L T et al. Immunol Rev. 2016; 274:307-329.

C3 glomerulopathies are rare spectrum of kidney diseases that are primarily mediated by complement dysregulation are the C3Gs, which include dense deposit disease and C3 glomerulonephritis. C3Gs are primarily driven by excessive complement turnover in the circulation due to convertase-stabilizing autoantibodies, which manifests in massive deposition of C3 activation fragments in the kidney (typically on the glomerular basement membrane in the case of dense deposit disease). Currently, no approved treatment options for C3G exist. See, Zipfel P F, et al. Mol Immunol. 2015; 67:21-30.

Transplant rejection of the kidney is partially related to complement activation. The unavoidable ischemia during organ transplantation and storage leads to endothelial cell damage, which trigger complement activation during reperfusion and contribute to ischemia-reperfusion injury (IRI). Antibody-mediated rejection (AMR) is perhaps the most feared complication after transplantation, which can lead to rapid loss of the transplanted organ, largely as a result of complement-mediated damage upon massive triggering of the classical pathway by anti-ABO or anti-HLA antibodies. Transplant-related complications were among the first indications considered for complement therapeutics. See. Ricklin et al. Nat Rev Nephrol. 2016; 12: 383-401; Sacks et al. Nat Rev Immunol. 2012; 12: 431-442; Fremeaux-Bacchi et al. Kidney Int. 2015; 88: 967-973; Stegall et al. Nat Rev Nephrol. 2012; 8: 670-678.

Complement activation might be a major contributor to diabetic nephropathy, which is the leading cause of end-stage renal disease (ESRD) in developed countries. See, Flyvbjerg A. Nat Rev Nephrol. 2017; 13: 311-318.

The IgA nephropathy is caused by aberrant glycosylation of IgA molecules that are subsequently recognized by anti-glycan autoantibodies. See, Lai K N, et al. IgA nephropathy. Nat Rev Dis Primers. 2016; 2: 16001. Glomerular immune-complex deposits can lead to complement activation, which causes podocyte damage either directly or indirectly via activation of mesangial cells and stimulation of cytokines and other downstream immune mediators.

Lupus nephritis, a common clinical manifestation and major cause of morbidity in SLE, is believed to have a similar mechanism to that of IgA nephropathy. Promising data from case studies suggest a potential benefit of Eculizumab (anti-C5) in lupus nephritis, particularly in patients with TMA. See, Yu et al., Nat Rev Nephrol. 2017; 13: 483-495.

Complement has a pathogenic role in anti-neutrophil cytoplasmic antibody (ANCA)-associated vasculitis (AAV). In this disease, priming of neutrophils induces the translocation of proteins such as myeloblastin or myeloperoxidase to the cell surface. These proteins are then recognized by autoantibodies, leading to the activation of complement and other host defense systems and the release of effector molecules such as C5a. This release in turn activates neutrophils and therefore results in a vicious cycle that exacerbates the disease, with potentially life-threatening consequences. Complement is centrally involved in the pathology of AAV, and blockage of the C5a signaling axis has emerged as a promising treatment option. See, Chen et al. Nat Rev Nephrol. 2017; 13:359-367; Schreiber et al. J Am Soc Nephrol. 2009; 20:289-298.

Glaucoma—An ocular disease characterized by the optic nerve damage and subsequent visual loss. The complement involvement implicated in glaucoma was firstly from studies performed in 2003 using gene microarray analysis on a disease model of glaucoma in cynomolgus monkeys, where retinal C4 and properdin gene transcription was elevated in both mild and severe glaucoma and C3 and C1q gene transcription up-regulated in severe glaucoma. Inhibition of the classical complement pathway in animal models of glaucoma resulted in reduced RGC loss, thus providing a clear link between classical complement activation and accelerated RGC damage in disease. The exact mechanism by which complement contributes to the development of pathology is the subject of several on-going studies. See, Miyahara et al. Investig Ophthalmol Vis Sci 2003; 44: 4347-4356; Williams et al. Mol Neurodegener 2016; 11: 26.

Diabetic retinopathy is a leading cause of preventable blindness around the world; about one-third of the diabetic population suffers from some stage of retinopathy. Diabetes itself is not a complement-mediated disease, but some data imply a link between progression of diabetic retinopathy and complement dysregulation. The deposition of C5b-9 complexes has been observed within the retinal blood vessel walls of both diabetic rats and humans. See, Schreiber et al. J Am Soc Nephrol. 2009; 20: 289-298; Zhang et al. Diabetes 2002; 51: 3499-3504.

Autoimmune uveitis is a group of inflammatory conditions that damage the eye internally. A potential role of complement activation has recently been identified. See, Jha et al, Invest Ophthalmol Vis Sci 2006; 47: 1030-1038; Jha et al. J Immunol 2006; 176: 7221-7231. Complement activation in the eye appears critical for driving the local production of cytokines (IFN-γ and IL-10), chemokines (IP-10), and adhesion molecules (ICAM-1 and LECAM-1) in a rat experimental model of anterior uveitis; depletion of complement in this model resulted in inhibition of disease. See, Caspi R. Drug Discov Today Dis Mech 2006; 3:199-206.

Age-related macular degeneration (AMD) results in progressive destruction of the macula, the central part of the retina which is responsible for high resolution vision. Complement activation and turnover has been demonstrated in the choriocapillaris layer, and increased levels occur in AMD and may precede the condition. See, Whitmore et al. Prog Retin Eye Res 2015; 45: 1-29; Mullins et al. Am J Pathol 2014; 184: 3142-3153; Keenan et al. Investig Ophthalmol Vis Sci 2015; 56: 4870-4879.

Amyotrophic lateral sclerosis (ALS) is a fatal and rapidly progressing motor neuron disease. Currently there is no effective treatment for ALS. In animal models and post-mortal in humans the progression of disease is correlated with a dysregulation of complement in the spinal cord. Complement activation in the spinal cord is concordant with onset of ALS and seems to be located on motor neurons and surrounding microglia, indicating that complement activation exacerbates the motor neuron loss during progression of disease. See, Kawamata T, et al. Am. J. Pathol. 1992; 140: 691-707. Ferraiuolo L, et al. J. Neurosci. 2007; 27: 9201-9219. Sta M, et al. Neurobiol. Dis. 2011; 42: 211-220. Annunziata et al. Acta Neurol. Scand. 1985; 72: 61-64. Ganesalingam J, et al. J. Neurochem. 2011; 117, 528-537. Tsuboi Y et al. J. Neurol. Neurosurg. Psychiatry 1994; 57: 859-861. Heurich B, et al. J. Neuroimmunol. 2011; 235: 104-109. Lee J D, et al. J. Neuroinflammation 2013; 10: 119. Lobsiger C S, et al. Proc. Natl. Acad. Sci. U.S.A 2007; 104: 7319-7326.

Alzheimer's Disease (AD) is characterized by two hallmark pathologies; amyloid-β (Aβ) plaques and neurofibrillary tangles comprising hyperphosphorylated tau. Recent studies have implicated complement involvement in AD pathogenesis. See, Ishii et al. Acta Neuropathol. 1984: 63: 296-300. Rogers J et al. Proc Natl Acad Sci USA. 1992; 89: 10016-20. Veerhuis R et al. Virchows Arch. 1995; 426: 603-10. Cribbs et al. J Neuroinflammation. 2012; 9: 179. Dejanovic et al. Neuron. 2018; 100: 1322-36. Lansita J A et al. Int J Toxicol. 2017; 36: 449-62.

Huntington's Disease (HD) is an inherited neurodegenerative disease characterized by progressive motor symptoms, psychiatric disturbances, and dementia. Expression of mRNA encoding early complement components C1q (c-chain), C1r, C3, and C4, complement regulators C11NH, Clusterin, MCP, DAF and CD59, and complement receptors C3a and C5a was upregulated in the HD striatum. Microarray analysis in HD post-mortem tissue demonstrated increased expression of complement components C4A, C4B and C3, most significantly in the most affected areas, caudate nucleus, and motor cortex. See, Hodges A et al. Hum Mol Genet. 2006; 15: 965-77.

Myasthenia gravis (MG) is a rare chronic autoimmune disorder affecting the neuromuscular junction (NMJ). Approximately 80-90% of cases have antibodies against the nicotinic acetylcholine receptor (AChR). In MG antibodies destroy the normal communication between nerves and muscles, leading to weakness in the skeletal muscles including the muscles responsible for functions involving breathing and moving parts of the body, including the eyes, mouth, throat and limbs. The involvement of complement in anti-AChR antibody-positive (AChR+) MG has been well established. Research findings encouraged the development of novel therapeutic approaches targeting different levels of the complement cascade. Currently, eculizumab (Soliris) that targets complement C5 is approved for AChR+ gMG in USA, for refractory AChR+ gMG in the EU, and for AChR+gMG patients that do not respond to IVIg/PE in Japan. A trial evaluates the efficacy of ravulizumab (Ultomiris), another anti-C5 antibody, in complement-inhibitor-naïve adult gMG patients (NCT03920293) started in 2019 and is currently underway in Europe, United States, Japan, and South Korea. See, Mantegazza et al. Immunotargets Ther. 2020; 9: 317-331. Drachman et al. N Engl J Med. 1978; 298(20): 1116-22.

Guillain-Barre Syndrome (GBS) a paralyzing autoimmune condition affecting the peripheral nervous system. Because of the complement activation observed in GBS patients, recent clinical trials have examined the role of Eculizumab, a humanized monoclonal antibody against C5 in patients with GBS, which showed that Eculizumab may be effective in severe cases. See, Misawa et al. Clin Exp Neuroimmunol. 2020; 11: 90-93.

Lambert-Eaton Myasthenic Syndrome (LEMS) is an autoimmune disorder in which the immune system produces autoantibodies that attacks the neuromuscular junctions, resulting in muscle weakness, a tingling sensation in the affected areas, fatigue, and dry mouth. LEMS is closely associated with cancer, in particular small cell lung cancer. More than half the individuals diagnosed with LEMS also develop small cell lung cancer. LEMS may appear up to 3 years before cancer is diagnosed. There is no cure for LEMS. See, Jayarangaiah et al. 2020 Jul. 15. In: StatPearls [Internet] PMID: 29939668; Cetin et al. Semin Neurol. 2018; 38(3):344-354.

Multiple Sclerosis (MS) is an inflammatory, demyelinating, and neurodegenerative disease of the central nervous system. The complement system has an established role in the pathogenesis of MS. Increased levels of plasma C3 and C4 components have been detected in MS patients in the case of the progressive forms. Mannose-binding lectin (MBL) and MBL-associated serine protease-2 (MASP-2) are increased in the plasma of MS patients in comparison to non-MS patients. See, Tatomir A, Talpos-Caia A, Anselmo F, Kruszewski A M, Boodhoo D, Rus V, Rus H. The complement system as a biomarker of disease activity and response to treatment in multiple sclerosis. Immunol Res. 2017; 65(6):1103-1109. Ingram G et al. Mult Scler. 2012; 18:1401-11. Kwok J Y et al. J Neuroimmunol. 2011; 239:98-100.

### Dosage and Routes of Administration

Dosages and desired drug concentrations of pharmaceutical compositions of the present application may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti. J. and Chappell, W. “The Use of Interspecies Scaling in Toxicokinetics,” In Toxicokinetics and New Drug Development, Yacobi et al., Eds, Pergamon Press, New York 1989, pp. 42-46.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

In some embodiments, there is provided an anti-C2 antibody or antibody construct comprising the anti-C2 antibody or fragment thereof (such as fusion protein) that is administered for a single time. In some embodiments, there is provided an anti-C2 antibody that is administered for multiple times (such as any of 2, 3, 4, 5, 6, or more times). In some embodiments, there is provided an anti-C2 antibody or antibody construct comprising the anti-C2 antibody or fragment thereof (such as fusion protein) that is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly. The frequency of the dose will be readily apparent to the skilled artisan and will depend upon any number of factors, include but not limited to, the type and severity of the disease being treated, the type and age of the subject, etc.

The anti-C2 antibody or antibody construct comprising the anti-C2 antibody or fragment thereof (such as fusion protein) of the present application, including but not limited to reconstituted and liquid formulations, are administered to an individual in need of treatment, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, ophthalmic, rectal, vaginal, parenteral, pulmonary, buccal, intraocular or inhalation routes. Other contemplated formulations include projected nanoparticles, liposomal preparations, resealed erythrocytes containing the active ingredient, and immunologically-based formulations. Parenteral administration of the anti-C2 antibody or antibody construct comprising the anti-C2 antibody or fragment thereof (such as fusion protein) includes any route of administration characterized by physical breaching of a tissue of an individual and administration of the pharmaceutical composition through the breach in the tissue. Parental administration can be local, regional or systemic. Parenteral administration thus includes, but is not limited to, administration of the anti-C2 antibody or antibody construct comprising the anti-C2 antibody or fragment thereof (such as fusion protein) by injection of the composition, by application of the composition through a surgical incision, by application of the anti-C2 antibody or antibody construct comprising the anti-C2 antibody or fragment thereof (such as fusion protein) through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

The anti-C2 antibody or antibody construct comprising the anti-C2 antibody or fragment thereof (such as fusion protein) of the invention may be prepared, packaged, or sold in bulk, as a single unit dose, or as a plurality of single unit doses. A unit dose is discrete amount of the anti-C2 antibody or antibody construct comprising the anti-C2 antibody or fragment thereof (such as fusion protein) comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

### V. Methods of Preparation

The present application also provides isolated nucleic acids encoding the anti-C2 antibodies or antibody construct comprising the anti-C2 antibody or fragment thereof (such as fusion protein), vectors and host cells comprising such isolated nucleic acids, and recombinant methods for the production of the anti-C2 antibodies.

### Expression Vectors and Cells Producing Antibodies

In some embodiments, the invention is a cell or cell line (such as host cells) that produces at least one of the anti-C2 antibodies or antibody construct comprising the anti-C2 antibody or fragment thereof (such as fusion protein) described herein. In one embodiment, the cell or cell line is a genetically modified cell that produces at least one of the anti-C2 antibodies or antibody construct comprising the anti-C2 antibody or fragment thereof (such as fusion protein) described herein. In one embodiment, the cell or cell line is a hybridoma that produces at least one of the anti-C2 antibodies thereof described herein.

Hybrid cells (hybridomas) are generally produced from mass fusions between murine splenocytes, which are highly enriched for B-lymphocytes, and myeloma “fusion partner cells” (Alberts et al., Molecular Biology of the Cell (Garland Publishing, Inc. 1994); Harlow et al., Antibodies. A Laboratory Manual (Cold Spring Harbor Laboratory, Cold Spring Harbor, 1988). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are referred to as monoclonal antibodies.

Also provided are nucleic acids encoding any of the anti-C2 antibodies or antibody constructs comprising the anti-C2 antibody or fragment thereof (such as fusion proteins) thereof disclosed herein, as well as vectors comprising the nucleic acids. Thus, the anti-C2 antibodies of the invention can be generated by expressing the nucleic acid in a cell or a cell line, such as the cell lines typically used for expression of recombinant or humanized immunoglobulins. Thus, the antibodies and fragments of the invention can also be generated by cloning the nucleic acids into one or more expression vectors, and transforming the vector into a cell line such as the cell lines typically used for expression of recombinant or humanized immunoglobulins. In some embodiments, there are provided nucleic acids encoding any of the anti-C2 antibodies or antibody constructs, comprising the sequence of any one of SEQ ID NOs: 161-182, 189-200, 208-209, 217-219, or any combination thereof.

The genes encoding the heavy and light chains of the anti-C2 antibodies thereof can be engineered according to methods, including but not limited to full length chemical gene synthesis, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al., 1992, J. Immunol. 148:1149). For example, genes encoding heavy and light chains, or fragments thereof, can be cloned from an antibody secreting cell's genomic DNA, or cDNA is produced by reverse transcription of the RNA of the cell. Cloning is accomplished by conventional techniques including the use of PCR primers that hybridize to the sequences flanking or overlapping the genes, or segments of genes, to be cloned.

Nucleic acids encoding the anti-C2 antibodies described herein, or the heavy chain or light chain or fragments thereof, can be obtained and used in accordance with recombinant nucleic acid techniques for the production of the specific immunoglobulin, immunoglobulin chain, or a fragment or variant thereof, in a variety of host cells or in an in vitro translation system. For example, the antibody-encoding nucleic acids, or fragments thereof, can be placed into suitable prokaryotic or eukaryotic vectors, e.g., expression vectors, and introduced into a suitable host cell by an appropriate method, e.g., transformation, transfection, electroporation, infection, such that the nucleic acid is operably linked to one or more expression control elements, e.g., in the vector or integrated into the host cell genome.

In some embodiments, the heavy and light chains, or fragments thereof, can be assembled in two different expression vectors that can be used to co-transfect a recipient cell. In some embodiments, each vector can contain two or more selectable genes, one for selection in a bacterial system and one for selection in a eukaryotic system. These vectors allow for the production and amplification of the genes in a bacterial system, and subsequent co-transfection of eukaryotic cells and selection of the co-transfected cells. The selection procedure can be used to select for the expression of antibody nucleic acids introduced on two different DNA vectors into a eukaryotic cell.

Alternatively, the nucleic acids encoding the heavy and light chains, or fragments thereof, may be expressed from one vector. Although the light and heavy chains are coded for by separate genes, they can be joined, using recombinant methods. For example, the two polypeptides can be joined by a synthetic linker that enables them to be made as a single protein chain in which the VL and VH regions pair to form monovalent molecules (known as single chain Fv (scFv); see e.g., Bird et al., 1988, Science 242: 423-426; and Huston et al., 1988, Proc. Natl. Acad. Sci. USA 85:5879-5883).

The invention provides for an isolated nucleic acid molecule comprising a nucleic acid sequence encoding a heavy chain and/or a light chain, as well as fragments thereof. A nucleic acid molecule comprising sequences encoding both the light and heavy chain, or fragments thereof, can be engineered to contain a synthetic signal sequence for secretion of the antibody, or fragment, when produced in a cell. Furthermore, the nucleic acid molecule can contain specific DNA links which allow for the insertion of other antibody sequences and maintain the translational reading frame so to not alter the amino acids normally found in antibody sequences. Exemplary nucleic acids sequences are set for in any of SEQ ID NOs: 33-62.

In accordance with the present invention, antibody-encoding nucleic acid sequences can be inserted into an appropriate expression vector. In various embodiments, the expression vector comprises the necessary elements for transcription and translation of the inserted antibody-encoding nucleic acid so as to generate recombinant DNA molecules that direct the expression of antibody sequences for the formation of an antibody, or a fragment thereof.

The antibody-encoding nucleic acids, or fragments thereof, can be subjected to various recombinant nucleic acid techniques known to those skilled in the art such as site-directed mutagenesis.

A variety of methods can be used to express nucleic acids in a cell. Nucleic acids can be cloned into a number of types of vectors. However, the present invention should not be construed to be limited to any particular vector. Instead, the present invention should be construed to encompass a wide variety of vectors which are readily available and/or known in the art. For example, the nucleic acid of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

In some embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Viral vector technology is well known in the art and is described, for example, in Sambrook et al. (2012), and in Ausubel et al. (1999), and in other virology and molecular biology manuals. Viruses, which are useful as vectors include, but are not limited to, retroviruses, adenoviruses, adeno-associated viruses, herpes viruses, and lentiviruses. In some embodiments, a suitable vector contains an origin of replication functional in at least one organism, a promoter sequence, convenient restriction endonuclease sites, and one or more selectable markers. (See, e.g., WO 01/96584: WO 01/29058; and U.S. Pat. No. 6,326,193).

Additional regulatory elements, e.g., enhancers, can be used modulate the frequency of transcriptional initiation. A promoter may be one naturally associated with a gene or nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

A promoter and/or enhancer that effectively directs the expression of the DNA segment in the cell type, organelle, and organism chosen for expression may be employed. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and fragments thereof. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue-specific promoter or cell-type specific promoter, which is a promoter that is active only in a desired tissue or cell. Tissue-specific promoters are well known in the art and include, but are not limited to, the HER-2 promoter and the PSA associated promoter sequences.

In order to assess the expression of the nucleic acids, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate nucleic acid and used in a co-transfection procedure. Both selectable markers and reporter genes may be flanked with appropriate regulatory sequences to enable expression in the host cells.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells. Suitable expression systems are well known and may be prepared using well known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing nucleic acids into a cell are known in the art. In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, laserporation, biological methods, chemical means, and the like. Methods for producing cells comprising vectors and/or exogenous nucleic acids are well-known in the art. See, for example, Sambrook et al. (2012) and Ausubel et al. (1999).

Regardless of the method used to introduce exogenous nucleic acids into a host cell or otherwise expose a cell to the nucleic acid of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### 1. Vector Construction

Polynucleotide sequences encoding polypeptide components of the anti-C2 antibody or antibody construct comprising the anti-C2 antibody or fragment thereof (such as fusion protein) of the present application can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present application. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. The expression vector described herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter. Promoters suitable for use with prokaryotic hosts include the PhoA promoter, the -galactamase and lactose promoter systems, a tryptophan (trp) promoter system and hybrid promoters such as the tac or the trc promoter. However, other promoters that are functional in bacteria (such as other known bacterial or phage promoters) are suitable as well.

In some embodiments, the production of the anti-C2 antibodies or antibody construct comprising the anti-C2 antibody or fragment thereof (such as fusion protein) can occur in the cytoplasm of the host cell, and therefore does not require the presence of secretion signal sequences within each cistron. In some embodiments, polypeptide components, such as the polypeptide encoding the VH domain of the first antigen binding portion optionally fused to the second antigen binding portion, and the polypeptide encoding the VL domain of the first antigen binding portion optionally fused to the second antigen binding portion, are expressed, folded and assembled to form functional anti-C2 antibodies or antibody construct comprising the anti-C2 antibody or fragment thereof (such as fusion protein) within the cytoplasm.

### 2. Protein Production in Prokarotic Host Cells.

Prokaryotic host cells suitable for expressing the anti-C2 antibodies or antibody constructs comprising the anti-C2 antibody or fragment thereof (such as fusion proteins) of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts. Methods for constructing derivatives of any of the above-mentioned bacteria having defined genotypes are known in the art and described in, for example, Bass et al., Proteins, 8: 309-314 (1990). It is generally necessary to select the appropriate bacteria taking into consideration replicability of the replicon in the cells of a bacterium.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells as described above.

Prokaryotic cells used to produce the anti-C2 antibodies or antibody constructs comprising the anti-C2 antibody or fragment thereof (such as fusion proteins) of the present application are grown at suitable temperatures in media known in the art and suitable for culture of the selected host cells. Examples of suitable media include Luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene. If an inducible promoter is used in the expression vector, protein expression is induced under conditions suitable for the activation of the promoter.

The expressed anti-C2 antibodies or antibody constructs comprising the anti-C2 antibody or fragment thereof (such as fusion proteins) of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale and small-scale fed-batch fermentation procedures are available for production of recombinant proteins.

To improve the production yield and quality of the anti-C2 antibodies or antibody constructs comprising the anti-C2 antibody or fragment thereof (such as fusion proteins) of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present application.

E. coli strains deficient for proteolytic enzymes and transformed with plasmids overexpressing one or more chaperone proteins may be used as host cells in the expression system encoding the anti-C2 antibodies or antibody constructs comprising the anti-C2 antibody or fragment thereof (such as fusion proteins) of the present application.

### 3. Protein Production in Eukaryotic Cells

In some embodiments, the anti-C2 antibodies or antibody constructs comprising the anti-C2 antibody or fragment thereof (such as fusion proteins) described herein can be expressed in eukaryotic cells. For eukaryotic expression, the vector components generally include, but are not limited to, one or more of the following, a signal sequence, an origin of replication, one or more marker genes, and enhancer element, a promoter, and a transcription termination sequence.

### a) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad Sci. 383:44-68 (1982)); MRC 5 cells; FS4 cells; and a human hepatoma line (Hep G2).

Host cells are transformed with the above-described expression or cloning vectors for the anti-C2 antibodies or antibody constructs comprising the anti-C2 antibody or fragment thereof (such as fusion proteins) production and cultured in conventional nutrient media modified as appropriate for inducing promoters, selecting transformants, or amplifying the genes encoding the desired sequences. In some embodiments, the anti-C2 antibodies or antibody constructs comprising the anti-C2 antibody or fragment thereof (such as fusion proteins) are expressed in CHO cells. In some embodiments, the anti-C2 antibodies are expressed in Expi-CHO cells.

### b) Culturing the Host Cells

The host cells used to produce the anti-C2 antibodies or antibody constructs comprising the anti-C2 antibody or fragment thereof (such as fusion proteins) of the present application may be cultured in a variety of media. The culture conditions, such as temperature, pH, and the like, are those previously used with the host cell selected for expression, and will be apparent to the ordinarily skilled artisan.

### c) Protein Purification

The anti-C2 antibodies or antibody constructs comprising the anti-C2 antibody or fragment thereof (such as fusion proteins) produced herein may be further purified to obtain preparations that are substantially homogeneous for further assays and uses. Standard protein purification methods known in the art can be employed.

### 4. Antibody Production and Modification

Components of the anti-C2 antibodies or antibody constructs comprising the anti-C2 antibody or fragment thereof (such as fusion proteins) can be produced using any known methods in the art, including methods described below.

### a) Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Generally either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. s.

Preferred immortalized myeloma cells are those that fuse efficiently, support stable high-level production of antibody by the selected antibody-producing cells, and are sensitive to a medium such as HAT medium. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding affinity and specificity of monoclonal antibodies produced by hybridoma cells is determined by immunoprecipitation or by an in vitro binding assay, such as radioimmunoassay (RIA) or enzyme-linked immunosorbent assay (ELISA).

After hybridoma cells are identified that produce antibodies of the desired specificity, affinity, and/or activity, the clones may be subcloned by limiting dilution procedures and grown by standard methods (Goding, supra), the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5: 256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 4, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### b) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA. 90:2551 (1993); Jakobovits et al., Nature, 362:255-258 (1993); Bruggermann et al., Year in Immuno., 7:33 (1993); U.S. Pat. No. 5,591,669 and WO 97/17852. Transgenic mice or rats capable of producing fully human antibodies are known in the art. See, e.g., US20090307787A1, U.S. Pat. No. 8,754,287, US20150289489A1, US20100122358A1, and WO2004049794.

Alternatively, phage display technology can be used to produce human antibodies and antibody fragments in vitro, from immunoglobulin variable (V) domain gene repertoires from uninmunized donors. McCafferty et al., Nature 348:552-553 (1990); Hoogenboom and Winter. J. Mol. Biol. 227: 381 (1991).

Human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. See, for example, U.S. Pat. Nos. 5,545,807; 5,545,806, 5,569,825, 5,625,126, 5,633,425, 5,661,016. Finally, human antibodies may also be generated in vitro by activated B cells (see U.S. Pat. Nos. 5,567,610 and 5,229,275).

In some embodiments, the anti-C2 antibody or a fragment thereof described herein is a humanized antibody, a human antibody, or a fully human antibody. In some embodiments, the anti-C2 antibody or a fragment thereof is a fully human antibody.

### c) Antibody Fragments

In certain circumstances there are advantages to using antibody fragments, such as antigen binding fragments, rather than whole antibodies. Smaller fragment sizes allow for rapid clearance, and may lead to improved access to solid tumors.

Various techniques have been developed for the production of antibody fragments. Traditionally, these fragments were derived via proteolytic digestion of intact antibodies (see, e.g., Morimoto et al., J Biochem Biophys. Method 24:107-117 (1992); and Brennan et al., Science 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ with increase in vivo half-life is described in U.S. Pat. No. 5,869,046. In other embodiments, the antibody of choice is a single chain Fv fragment (scFv). See WO 93/16185; U.S. Pat. Nos. 5,571,894 and 5,587,458. The antibody fragment may also be a “linear antibody”, e.g., as described in U.S. Pat. No. 5,641,870. Such linear antibody fragments may be monospecific or bispecific.

### d) Effector Function Engineering

It may be desirable to modify the anti-C2 antibodies of the present application with respect to Fc effector function, e.g., so as to modify (e.g., enhance or eliminate) antigen-dependent cell-mediated cytotoxicity (ADCC) and/or complement dependent cytotoxicity (CDC) of the antibody. In a preferred embodiment, Fc effector function of the anti-C2 antibody is reduced or eliminated. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric humanized anti-C2 antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half-life of the antibody, one may incorporate a salvage receptor binding epitope into the ani-C2 antibody as described in U.S. Pat. No. 5,739,277, for example. As used herein, the term “salvage receptor binding epitope” refers to an epitope of the Fe region of an IgG molecule (e.g., IgG1, IgG2, IgG3, or IgG4) that is responsible for increasing the in vivo serum half-life of the IgG molecule.

### e) Other Amino Acid Sequence Modifications

Amino acid sequence modification(s) of the antibodies, such as single chain antibodies or antibody components of the anti-C2 antibodies, described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells in Science, 244:1081-1085 (1989).

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the table below under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1
Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or hlelical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants to the anti-C2 antibodies of the present application are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant versions.

### f) Other Modifications

The anti-C2 antibodies of the present application can be further modified to contain additional nonproteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

### Kits

The invention also includes a kit comprising an anti-C2 antibody of the invention and an instructional material which describes, for instance, administering the anti-C2 antibody to an individual as a therapeutic treatment or a non-treatment use as described elsewhere herein. In an embodiment, this kit further comprises a (optionally sterile) pharmaceutically acceptable carrier suitable for dissolving or suspending therapeutic composition, comprising a humanized anti-C2 antibody, or combinations thereof, of the invention, for instance, prior to administering the antibody to an individual. Optionally, the kit comprises an applicator for administering the antibody. Also provided are unit dosage forms comprising the ani-C2 antibodies.

### EXEMPLARY EMBODIMENTS

The present disclosure may be better understood with reference to the following exemplary embodiments.

Embodiment 1. An anti-C2 antibody, a fragment, or a fusion protein thereof comprising a heavy chain variable region (VH) comprising an H-CDR 1, an H-CDR2, and an H-CDR3 of an VH comprising the amino acid sequence of any one of SEQ II) NOs: 1, 9, 17, 25, 33, 41, 49, 57, 65, 73, 81 and 89; and a light chain variable region (VL) comprising an L-CDR1, an L-CDR2, and an L-CDR3 of an VL comprising the amino acid sequence of any one of SEQ ID NOs: 2, 10, 18, 26, 34, 42, 50, 58, 66, 74, 82, 90, 97, 101, 105,109, 113, 117, 121,125, 129, 133, 137, 141, 145, 149, 153 and 157.

Embodiment 2. An anti-C2 antibody, a fragment, or a fusion protein thereof comprising an VH and VL, wherein:
a) the VH comprises i) an H-CDR1 comprising the amino acid sequence of SEQ ID NO: 3, ii) an H-CDR2 comprising the amino acid sequence of SEQ ID NO: 4, and iii) an H-CDR3 comprising the amino acid sequence of SEQ ID NO: 5, or a variant thereof comprising up to 3, 2, or 1 amino acid substitutions in the H-CDRs; and the VL comprises i) an L-CDR1 comprising the amino acid sequence of SEQ ID NO: 6, an L-CDR2 comprising the amino acid sequence of SEQ ID NO: 7, and iii) an L-CDR3 comprising the amino acid sequence of SEQ ID NO: 8, or a variant thereof comprising up to 3, 2, or 1 amino acid substitutions in the L-CDRs;
b) the VH comprises i) an H-CDR1 comprising the amino acid sequence of SEQ ID NO: 11, ii) an H-CDR2 comprising the amino acid sequence of SEQ ID NO: 12, and iii) an H-CDR3 comprising the amino acid sequence of SEQ ID NO: 13, or a variant thereof comprising up to 3, 2, or 1 amino acid substitutions in the H-CDRs; and the V_{L} comprises i) an L-CDR1 comprising the amino acid sequence of SEQ ID NO: 14, an L-CDR2 comprising the amino acid sequence of SEQ ID NO: 15, and iii) an L-CDR3 comprising the amino acid sequence of SEQ ID NO: 16, or a variant thereof comprising up to 3, 2, or 1 amino acid substitutions in the L-CDRs;
c) the VH comprises i) an H-CDR1 comprising the amino acid sequence of SEQ ID NO: 19, ii) an H-CDR2 comprising the amino acid sequence of SEQ ID NO: 20, and iii) an H-CDR3 comprising the amino acid sequence of SEQ ID NO: 21, or a variant thereof comprising up to 3, 2, or 1 amino acid substitutions in the H-CDRs; and the VL comprises i) an L-CDR1 comprising the amino acid sequence of SEQ ID NO: 22, an L-CDR2 comprising the amino acid sequence of SEQ ID NO: 23, and iii) an L-CDR3 comprising the amino acid sequence of SEQ ID NO: 24, or a variant thereof comprising up to 3, 2, or 1 amino acid substitutions in the L-CDRs;
d) the VH comprises i) an H-CDR1 comprising the amino acid sequence of SEQ ID NO: 27, ii) an H-CDR2 comprising the amino acid sequence of SEQ ID NO: 28, and iii) an H-CDR3 comprising the amino acid sequence of SEQ ID NO: 29, or a variant thereof comprising up to 3, 2, or 1 amino acid substitutions in the H-CDRs; and the VL comprises i) an L-CDR1 comprising the amino acid sequence of SEQ ID NO: 30, an L-CDR2 comprising the amino acid sequence of SEQ ID NO: 31, and iii) an L-CDR3 comprising the amino acid sequence of SEQ ID NO: 32, or a variant thereof comprising up to 3, 2, or 1 amino acid substitutions in the L-CDRs;
e) the VH comprises i) an H-CDR1 comprising the amino acid sequence of SEQ ID NO: 35, ii) an H-CDR2 comprising the amino acid sequence of SEQ ID NO: 36, and iii) an H-CDR3 comprising the amino acid sequence of SEQ ID NO: 37, or a variant thereof comprising up to 3, 2, or 1 amino acid substitutions in the H-CDRs; and the V_{L} comprises i) an L-CDR1 comprising the amino acid sequence of SEQ ID NO: 38, an L-CDR2 comprising the amino acid sequence of SEQ ID NO: 39, and iii) an L-CDR3 comprising the amino acid sequence of SEQ ID NO: 40, or a variant thereof comprising up to 3, 2, or 1 amino acid substitutions in the L-CDRs;
f) the VH comprises i) an H-CDR1 comprising the amino acid sequence of SEQ ID NO: 43, ii) an H-CDR2 comprising the amino acid sequence of SEQ ID NO: 44, and iii) an H-CDR3 comprising the amino acid sequence of SEQ ID NO: 45, or a variant thereof comprising up to 3, 2, or 1 amino acid substitutions in the H-CDRs; and the VL comprises i) an L-CDR 1 comprising the amino acid sequence of SEQ ID NO: 46, an L-CDR2 comprising the amino acid sequence of SEQ ID NO: 47, and iii) an L-CDR3 comprising the amino acid sequence of SEQ ID NO: 48, or a variant thereof comprising up to 3, 2, or 1 amino acid substitutions in the L-CDRs;
g) the VH comprises i) an H-CDR1 comprising the amino acid sequence of SEQ ID NO: 51, ii) an H-CDR2 comprising the amino acid sequence of SEQ ID NO: 52, and iii) an H-CDR3 comprising the amino acid sequence of SEQ ID NO: 53, or a variant thereof comprising up to 3, 2, or 1 amino acid substitutions in the H-CDRs; and the VL comprises i) an L-CDR1 comprising the amino acid sequence of SEQ ID NO: 54, an L-CDR2 comprising the amino acid sequence of SEQ ID NO: 55, and iii) an L-CDR3 comprising the amino acid sequence of SEQ ID NO: 56, or a variant thereof comprising up to 3, 2, or 1 amino acid substitutions in the L-CDRs;
h) the VH comprises i) an H-CDR1 comprising the amino acid sequence of SEQ ID NO: 59, ii) an H-CDR2 comprising the amino acid sequence of SEQ ID NO: 60, and iii) an H-CDR3 comprising the amino acid sequence of SEQ ID NO: 61, or a variant thereof comprising up to 3, 2, or 1 amino acid substitutions in the H-CDRs; and the VL comprises i) an L-CDR1 comprising the amino acid sequence of SEQ ID NO: 62, an L-CDR2 comprising the amino acid sequence of SEQ ID NO: 63, and iii) an L-CDR3 comprising the amino acid sequence of SEQ ID NO: 64, or a variant thereof comprising up to 3, 2, or 1 amino acid substitutions in the L-CDRs;
i) the VH comprises i) an H-CDR1 comprising the amino acid sequence of SEQ ID NO: 67, ii) an H-CDR2 comprising the amino acid sequence of SEQ ID NO: 68, and iii) an H-CDR3 comprising the amino acid sequence of SEQ ID NO: 69, or a variant thereof comprising up to 3, 2, or 1 amino acid substitutions in the H-CDRs; and the VL comprises i) an L-CDR1 comprising the amino acid sequence of SEQ ID NO: 70, an L-CDR2 comprising the amino acid sequence of SEQ ID NO: 71, and iii) an L-CDR3 comprising the amino acid sequence of SEQ ID NO: 72, or a variant thereof comprising up to 3, 2, or 1 amino acid substitutions in the L-CDRs;
j) the VH comprises i) an H-CDR1 comprising the amino acid sequence of SEQ ID NO: 75, ii) an H-CDR2 comprising the amino acid sequence of SEQ ID NO: 76, and iii) an H-CDR3 comprising the amino acid sequence of SEQ ID NO: 77, or a variant thereof comprising up to 3, 2, or 1 amino acid substitutions in the H-CDRs; and the VL comprises i) an L-CDR1 comprising the amino acid sequence of SEQ ID NO: 78, an L-CDR2 comprising the amino acid sequence of SEQ ID NO: 79, and iii) an L-CDR3 comprising the amino acid sequence of SEQ ID NO: 80, or a variant thereof comprising up to 3, 2, or 1 amino acid substitutions in the L-CDRs; or
k) the VH comprises i) an H-CDR1 comprising the amino acid sequence of SEQ ID NO: 83, ii) an H-CDR2 comprising the amino acid sequence of SEQ ID NO: 84, and iii) an H-CDR3 comprising the amino acid sequence of SEQ ID NO: 85, or a variant thereof comprising up to 3, 2, or 1 amino acid substitutions in the H-CDRs; and the VL comprises i) an L-CDR1 comprising the amino acid sequence of SEQ ID NO: 86, an L-CDR2 comprising the amino acid sequence of SEQ ID NO: 87, and iii) an L-CDR3 comprising the amino acid sequence of SEQ ID NO: 88, or a variant thereof comprising up to 3, 2, or 1 amino acid substitutions in the L-CDRs.

Embodiment 3. A pH-dependent, anti-C2 antibody, a fragment, or a fusion protein thereof comprising an VH and VL, wherein:
l) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 94, an L-CDR2 comprising the amino acid sequence of SEQ ID NO: 95, and iii) an L-CDR3 comprising the amino acid sequence of SEQ ID NO: 96;
m) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 98, an L-CDR2 comprising the amino acid sequence of SEQ ID NO: 99, and iii) an L-CDR3 comprising the amino acid sequence of SEQ ID NO: 100;
n) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 102, an L-CDR2 comprising the amino acid sequence of SEQ ID NO: 103, and iii) an L-CDR3 comprising the amino acid sequence of SEQ ID NO: 104;
o) the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 106, an L-CDR2 comprising the amino acid sequence of SEQ ID NO: 107, and iii) an L-CDR3 comprising the amino acid sequence of SEQ ID NO: 108;
p) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 110, an L-CDR2 comprising the amino acid sequence of SEQ ID NO: 111, and iii) an L-CDR3 comprising the amino acid sequence of SEQ ID NO: 112;
q) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 114, an L-CDR2 comprising the amino acid sequence of SEQ ID NO: 115, and iii) an L-CDR3 comprising the amino acid sequence of SEQ ID NO: 116;
r) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 118, an L-CDR2 comprising the amino acid sequence of SEQ ID NO: 119, and iii) an L-CDR3 comprising the amino acid sequence of SEQ ID NO: 120;
s) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 122, an L-CDR2 comprising the amino acid sequence of SEQ ID NO: 123, and iii) an L-CDR3 comprising the amino acid sequence of SEQ ID NO: 124;
t) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 126, an L-CDR2 comprising the amino acid sequence of SEQ ID NO: 127, and iii) an L-CDR3 comprising the amino acid sequence of SEQ ID NO: 128;
u) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 130, an L-CDR2 comprising the amino acid sequence of SEQ ID NO: 131, and iii) an L-CDR3 comprising the amino acid sequence of SEQ ID NO: 132;
v) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 134, ii) an L-CDR2 comprising the amino acid sequence of SEQ ID NO: 135, and iii) an L-CDR3 comprising the amino acid sequence of SEQ ID NO: 136;
w) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 138, ii) an L-CDR2 comprising the amino acid sequence of SEQ ID NO: 139, and iii) an L-CDR3 comprising the amino acid sequence of SEQ ID NO: 140;
x) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 142, ii) an L-CDR2 comprising the amino acid sequence of SEQ ID NO: 143, and iii) an L-CDR3 comprising the amino acid sequence of SEQ ID NO: 144;
y) the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 146, ii) an L-CDR2 comprising the amino acid sequence of SEQ ID NO: 147, and iii) an L-CDR3 comprising the amino acid sequence of SEQ ID NO: 148;
z) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 150, ii) an L-CDR2 comprising the amino acid sequence of SEQ ID NO: 151, and iii) an L-CDR3 comprising the amino acid sequence of SEQ ID NO: 152;
aa) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 154, ii) an L-CDR2 comprising the amino acid sequence of SEQ ID NO: 155, and iii) an L-CDR3 comprising the amino acid sequence of SEQ ID NO: 156; or ab) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 158, ii) an L-CDR2 comprising the amino acid sequence of SEQ ID NO: 159, and iii) an L-CDR3 comprising the amino acid sequence of SEQ ID NO: 160.

Embodiment 4. The anti-C2 antibody, a fragment, or a fusion protein thereof according to embodiment 2, wherein the VH comprises i) an H-CDR1 comprising the amino acid sequence of SEQ II) NO: 3, ii) an H-CDR2 comprising the amino acid sequence of SEQ II) NO: 4, and iii) an H-CDR3 comprising the amino acid sequence of SEQ ID NO: 5, or a variant thereof comprising up to 3, 2, or 1 amino acid substitutions in the H-CDRs; and the VL comprises i) an L-CDR 1 comprising the amino acid sequence of SEQ ID NO: 6, an L-CDR2 comprising the amino acid sequence of SEQ ID NO: 7, and iii) an L-CDR3 comprising the amino acid sequence of SEQ ID NO: 8, or a variant thereof comprising up to 3, 2, or 1 amino acid substitutions in the L-CDRs.

Embodiment 5. The anti-C2 antibody, a fragment, or a fusion protein thereof according to embodiment 2, wherein the VH comprises i) an H-CDR1 comprising the amino acid sequence of SEQ II) NO: 11, ii) an H-CDR2 comprising the amino acid sequence of SEQ ID NO: 12, and iii) an H-CDR3 comprising the amino acid sequence of SEQ ID NO: 13, or a variant thereof comprising up to 3, 2, or 1 amino acid substitutions in the H-CDRs; and the VL comprises i) an L-CDR1 comprising the amino acid sequence of SEQ ID NO: 14, an L-CDR2 comprising the amino acid sequence of SEQ ID NO: 15, and iii) an L-CDR3 comprising the amino acid sequence of SEQ II) NO: 16, or a variant thereof comprising up to 3, 2, or 1 amino acid substitutions in the L-CDRs.

Embodiment 6. The anti-C2 antibody, a fragment, or a fusion protein thereof according to embodiment 2, wherein the VH comprises i) an H-CDR 1 comprising the amino acid sequence of SEQ ID NO: 19, ii) an H-CDR2 comprising the amino acid sequence of SEQ ID NO: 20, and iii) an H-CDR3 comprising the amino acid sequence of SEQ ID NO: 21, or a variant thereof comprising up to 3, 2, or 1 amino acid substitutions in the H-CIDRs; and the VL comprises i) an L-CDR1 comprising the amino acid sequence of SEQ ID NO: 22, an L-CDR2 comprising the amino acid sequence of SEQ ID NO: 23, and iii) an L-CDR3 comprising the amino acid sequence of SEQ ID NO: 24, or a variant thereof comprising up to 3, 2, or 1 amino acid substitutions in the L-CDRs.

Embodiment 7. The anti-C2 antibody, a fragment, or a fusion protein thereof according to embodiment 2, wherein the VH comprises i) an H-CDR 1 comprising the amino acid sequence of SEQ ID NO: 27, ii) an H-CDR2 comprising the amino acid sequence of SEQ ID NO: 28, and iii) an H-CDR3 comprising the amino acid sequence of SEQ ID NO: 29, or a variant thereof comprising up to 3, 2, or 1 amino acid substitutions in the H-CIDRs; and the VL comprises i) an L-CDR1 comprising the amino acid sequence of SEQ ID NO: 30, an L-CDR2 comprising the amino acid sequence of SEQ ID NO: 31, and iii) an L-CDR3 comprising the amino acid sequence of SEQ ID NO: 32, or a variant thereof comprising up to 3, 2, or 1 amino acid substitutions in the L-CDRs.

Embodiment 8. The anti-C2 antibody, a fragment, or a fusion protein thereof according to embodiment 2, wherein the VH comprises i) an H-CDR 1 comprising the amino acid sequence of SEQ ID NO: 35, ii) an H-CDR2 comprising the amino acid sequence of SEQ ID NO: 36, and iii) an H-CDR3 comprising the amino acid sequence of SEQ ID NO: 37, or a variant thereof comprising up to 3, 2, or 1 amino acid substitutions in the H-CIDRs; and the VL comprises i) an L-CDR1 comprising the amino acid sequence of SEQ ID NO: 38, an L-CDR2 comprising the amino acid sequence of SEQ ID NO: 39, and iii) an L-CDR3 comprising the amino acid sequence of SEQ ID NO: 40, or a variant thereof comprising up to 3, 2, or 1 amino acid substitutions in the L-CDRs.

Embodiment 9. The anti-C2 antibody, a fragment, or a fusion protein thereof according to embodiment 2, wherein the VH comprises i) an H-CDR1 comprising the amino acid sequence of SEQ II) NO: 43, ii) an H-CDR2 comprising the amino acid sequence of SEQ ID NO: 44, and iii) an H-CDR3 comprising the amino acid sequence of SEQ ID NO: 45, or a variant thereof comprising up to 3, 2, or 1 amino acid substitutions in the H-CDRs; and the VL comprises i) an L-CDR1 comprising the amino acid sequence of SEQ ID NO: 46, an L-CDR2 comprising the amino acid sequence of SEQ ID NO: 47, and iii) an L-CDR3 comprising the amino acid sequence of SEQ ID NO: 48, or a variant thereof comprising up to 3, 2, or 1 amino acid substitutions in the L-CDRs.

Embodiment 10. The anti-C2 antibody, a fragment, or a fusion protein thereof according to embodiment 2, wherein the VH comprises i) an H-CDR1 comprising the amino acid sequence of SEQ ID NO: 51, ii) an H-CDR2 comprising the amino acid sequence of SEQ ID NO: 52, and iii) an H-CDR3 comprising the amino acid sequence of SEQ ID NO: 53, or a variant thereof comprising up to 3, 2, or 1 amino acid substitutions in the H-CDRs; and the VL comprises i) an L-CDR1 comprising the amino acid sequence of SEQ ID NO: 54, an L-CDR2 comprising the amino acid sequence of SEQ ID NO: 55, and iii) an L-CDR3 comprising the amino acid sequence of SEQ ID NO: 56, or a variant thereof comprising up to 3, 2, or 1 amino acid substitutions in the L-CDRs.

Embodiment 11. The anti-C2 antibody, a fragment, or a fusion protein thereof according to embodiment 2, wherein the VH comprises i) an H-CDR1 comprising the amino acid sequence of SEQ ID NO: 59, ii) an H-CDR2 comprising the amino acid sequence of SEQ ID NO: 60, and iii) an H-CDR3 comprising the amino acid sequence of SEQ ID NO: 61, or a variant thereof comprising up to 3, 2, or 1 amino acid substitutions in the H-CDRs; and the VL comprises i) an L-CDR1 comprising the amino acid sequence of SEQ ID NO: 62, an L-CDR2 comprising the amino acid sequence of SEQ ID NO: 63, and iii) an L-CDR3 comprising the amino acid sequence of SEQ ID NO: 64, or a variant thereof comprising up to 3, 2, or 1 amino acid substitutions in the L-CDRs.

Embodiment 12. The anti-C2 antibody, a fragment, or a fusion protein thereof according to embodiment 2, wherein the VH comprises i) an H-CDR1 comprising the amino acid sequence of SEQ ID NO: 67, ii) an H-CDR2 comprising the amino acid sequence of SEQ ID NO: 68, and iii) an H-CDR3 comprising the amino acid sequence of SEQ ID NO: 69, or a variant thereof comprising up to 3, 2, or 1 amino acid substitutions in the H-CDRs; and the VL comprises i) an L-CDR1 comprising the amino acid sequence of SEQ ID NO: 70, an L-CDR2 comprising the amino acid sequence of SEQ ID NO: 71, and iii) an L-CDR3 comprising the amino acid sequence of SEQ ID NO: 72, or a variant thereof comprising up to 3, 2, or 1 amino acid substitutions in the L-CDRs.

Embodiment 13. The anti-C2 antibody, a fragment, or a fusion protein thereof according to embodiment 2, wherein the VH comprises i) an H-CDR1 comprising the amino acid sequence of SEQ ID NO: 75, ii) an H-CDR2 comprising the amino acid sequence of SEQ ID NO: 76, and iii) an H-CDR3 comprising the amino acid sequence of SEQ ID NO: 77, or a variant thereof comprising up to 3, 2, or 1 amino acid substitutions in the H-CDRs; and the VL comprises i) an L-CDR1 comprising the amino acid sequence of SEQ ID NO: 78, an L-CDR2 comprising the amino acid sequence of SEQ ID NO: 79, and iii) an L-CDR3 comprising the amino acid sequence of SEQ ID NO: 80, or a variant thereof comprising up to 3, 2, or 1 amino acid substitutions in the L-CDRs.

Embodiment 14. The anti-C2 antibody, a fragment, or a fusion protein thereof according to embodiment 2, wherein the VH comprises i) an H-CDR1 comprising the amino acid sequence of SEQ ID NO: 83, ii) an H-CDR2 comprising the amino acid sequence of SEQ ID NO: 84, and iii) an H-CDR3 comprising the amino acid sequence of SEQ ID NO: 85, or a variant thereof comprising up to 3, 2, or 1 amino acid substitutions in the H-CDRs; and the VL comprises i) an L-CDR1 comprising the amino acid sequence of SEQ ID NO: 86, an L-CDR2 comprising the amino acid sequence of SEQ ID NO: 87, and iii) an L-CDR3 comprising the amino acid sequence of SEQ ID NO: 88, or a variant thereof comprising up to 3, 2, or 1 amino acid substitutions in the L-CDRs.

Embodiment 15. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 94, an L-CDR2 comprising the amino acid sequence of SEQ ID NO: 95, and iii) an L-CDR3 comprising the amino acid sequence of SEQ ID NO: 96.

Embodiment 16. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 98, an L-CDR2 comprising the amino acid sequence of SEQ ID NO: 99, and iii) an L-CDR3 comprising the amino acid sequence of SEQ ID NO: 100.

Embodiment 17. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 102, an L-CDR2 comprising the amino acid sequence of SEQ ID NO: 103, and iii) an L-CDR3 comprising the amino acid sequence of SEQ ID NO: 104.

Embodiment 18. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 106, an L-CDR2 comprising the amino acid sequence of SEQ ID NO: 107, and iii) an L-CDR3 comprising the amino acid sequence of SEQ ID NO: 108.

Embodiment 19. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 110, an L-CDR2 comprising the amino acid sequence of SEQ ID NO: 111, and iii) an L-CDR3 comprising the amino acid sequence of SEQ II) NO: 112.

Embodiment 20. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 114, an L-CDR2 comprising the amino acid sequence of SEQ ID NO: 115, and iii) an L-CDR3 comprising the amino acid sequence of SEQ ID NO: 116.

Embodiment 21. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 118, an L-CDR2 comprising the amino acid sequence of SEQ ID NO: 119, and iii) an L-CDR3 comprising the amino acid sequence of SEQ ID NO: 120.

Embodiment 22. The pH dependent anti-C2 antibody, a fragment, or a fusion protein thereof according to embodiment 3, wherein the VH comprises i) an H-CDR 1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 122, an L-CDR2 comprising the amino acid sequence of SEQ ID NO: 123, and iii) an L-CDR3 comprising the amino acid sequence of SEQ ID NO: 124.

Embodiment 23. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 126, an L-CDR2 comprising the amino acid sequence of SEQ ID NO: 127, and iii) an L-CDR3 comprising the amino acid sequence of SEQ ID NO: 128.

Embodiment 24. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 130, an L-CDR2 comprising the amino acid sequence of SEQ ID NO: 131, and iii) an L-CDR3 comprising the amino acid sequence of SEQ ID NO: 132.

Embodiment 25. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 134, ii) an L-CDR2 comprising the amino acid sequence of SEQ ID NO: 135, and iii) an L-CDR3 comprising the amino acid sequence of SEQ ID NO: 136.

Embodiment 26. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 138, ii) an L-CDR2 comprising the amino acid sequence of SEQ ID NO: 139, and iii) an L-CDR3 comprising the amino acid sequence of SEQ ID NO: 140.

Embodiment 27. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 142, ii) an L-CDR2 comprising the amino acid sequence of SEQ ID NO: 143, and iii) an L-CDR3 comprising the amino acid sequence of SEQ ID NO: 144.

Embodiment 28. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 146, ii) an L-CDR2 comprising the amino acid sequence of SEQ ID NO: 147, and iii) an L-CDR3 comprising the amino acid sequence of SEQ ID NO: 148.

Embodiment 29. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 150, ii) an L-CDR2 comprising the amino acid sequence of SEQ ID NO: 151, and iii) an L-CDR3 comprising the amino acid sequence of SEQ ID NO: 152.

Embodiment 30. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 154, ii) an L-CDR2 comprising the amino acid sequence of SEQ ID NO: 155, and iii) an L-CDR3 comprising the amino acid sequence of SEQ ID NO: 156.

Embodiment 31. The pH dependent anti-C2 antibody, a fragment, or a fusion protein thereof according to embodiment 3, wherein the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR 1 comprising the amino acid sequence of SEQ ID NO: 158, ii) an L-CDR2 comprising the amino acid sequence of SEQ ID NO: 159, and iii) an L-CDR3 comprising the amino acid sequence of SEQ ID NO: 160.

Embodiment 32. The anti-C2 antibody, a fragment, or a fusion protein thereof according to any one of embodiments 1-31, wherein the VH comprises an amino acid sequence of any one of SEQ ID NOs: 1, 9, 17, 25, 33, 41, 49, 57, 65, 73, 81, and 89 or a variant comprising an amino acid consequence having at least about 80% sequence identity; and/or wherein the VL comprises an amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153 and 157 or a variant comprising an amino acid consequence having at least about 80% sequence identity.

Embodiment 33. The anti-C2 antibody, a fragment, or a fusion protein thereof according to embodiment 32, wherein:
a) the V H comprises an amino acid sequence of SEQ ID NO: 1, or a variant comprising an amino acid sequence having at least about 80% sequence identity; and the VL comprises an amino acid sequence of SEQ ID NO: 2, or a variant comprising an amino acid sequence having at least about 80% sequence identity,
b) the VH comprises an amino acid sequence of SEQ ID NO: 9, or a variant comprising an amino acid sequence having at least about 80% sequence identity; and the VL comprises an amino acid sequence of SEQ ID NO: 10, or a variant comprising an amino acid sequence having at least about 80% sequence identity,
c) the VH comprises an amino acid sequence of SEQ ID NO: 17, or a variant comprising an amino acid sequence having at least about 80% sequence identity; and the VL comprises an amino acid sequence of SEQ ID NO: 18, or a variant comprising an amino acid sequence having at least about 80% sequence identity,
d) the VH comprises an amino acid sequence of SEQ ID NO: 25, or a variant comprising an amino acid sequence having at least about 80% sequence identity; and the VL comprises an amino acid sequence of SEQ ID NO: 26, or a variant comprising an amino acid sequence having at least about 80% sequence identity,
e) the VH comprises an amino acid sequence of SEQ ID NO: 33, or a variant comprising an amino acid sequence having at least about 80% sequence identity; and the VL comprises an amino acid sequence of SEQ ID NO: 34, or a variant comprising an amino acid sequence having at least about 80% sequence identity,
f) the VH comprises an amino acid sequence of SEQ ID NO: 41, or a variant comprising an amino acid sequence having at least about 80% sequence identity; and the VL comprises an amino acid sequence of SEQ ID NO: 42, or a variant comprising an amino acid sequence having at least about 80% sequence identity,
g) the VH comprises an amino acid sequence of SEQ ID NO: 49, or a variant comprising an amino acid sequence having at least about 80% sequence identity; and the VL comprises an amino acid sequence of SEQ ID NO: 50, or a variant comprising an amino acid sequence having at least about 80% sequence identity,
h) the VH comprises an amino acid sequence of SEQ ID NO: 57, or a variant comprising an amino acid sequence having at least about 80% sequence identity; and the VL comprises an amino acid sequence of SEQ ID NO: 58, or a variant comprising an amino acid sequence having at least about 80% sequence identity,
i) the VH comprises an amino acid sequence of SEQ ID NO: 65, or a variant comprising an amino acid sequence having at least about 80% sequence identity; and the VL comprises an amino acid sequence of SEQ ID NO: 66, or a variant comprising an amino acid sequence having at least about 80% sequence identity,
j) the VH comprises an amino acid sequence of SEQ ID NO: 73, or a variant comprising an amino acid sequence having at least about 80% sequence identity; and the VL comprises an amino acid sequence of SEQ ID NO: 74, or a variant comprising an amino acid sequence having at least about 80% sequence identity,
k) the VH comprises an amino acid sequence of SEQ ID NO: 81, or a variant comprising an amino acid sequence having at least about 80% sequence identity; and the VL comprises an amino acid sequence of SEQ ID NO: 82, or a variant comprising an amino acid sequence having at least about 80% sequence identity,
l) the VH comprises an amino acid sequence of SEQ ID NO: 89, or a variant comprising an amino acid sequence having at least about 80% sequence identity; and the VL comprises an amino acid sequence of SEQ ID NO: 90, or a variant comprising an amino acid sequence having at least about 80% sequence identity,
m) the VH comprises an amino acid sequence of SEQ ID NO: 89, or a variant comprising an amino acid sequence having at least about 80% sequence identity; and the VL comprises an amino acid sequence of SEQ ID NO: 97, or a variant comprising an amino acid sequence having at least about 80% sequence identity,
n) the VH comprises an amino acid sequence of SEQ ID NO: 89, or a variant comprising an amino acid sequence having at least about 80% sequence identity; and the VL comprises an amino acid sequence of SEQ ID NO: 101, or a variant comprising an amino acid sequence having at least about 80% sequence identity,
o) the VH comprises an amino acid sequence of SEQ ID NO: 89, or a variant comprising an amino acid sequence having at least about 80% sequence identity; and the VL comprises an amino acid sequence of SEQ ID NO: 105, or a variant comprising an amino acid sequence having at least about 80% sequence identity,
p) the VH comprises an amino acid sequence of SEQ ID NO: 89, or a variant comprising an amino acid sequence having at least about 80% sequence identity; and the VL comprises an amino acid sequence of SEQ ID NO: 109, or a variant comprising an amino acid sequence having at least about 80% sequence identity,
q) the VH comprises an amino acid sequence of SEQ ID NO: 89, or a variant comprising an amino acid sequence having at least about 80% sequence identity; and the VL comprises an amino acid sequence of SEQ ID NO: 113, or a variant comprising an amino acid sequence having at least about 80% sequence identity,
r) the VH comprises an amino acid sequence of SEQ ID NO: 89, or a variant comprising an amino acid sequence having at least about 80% sequence identity; and the VL comprises an amino acid sequence of SEQ ID NO: 117, or a variant comprising an amino acid sequence having at least about 80% sequence identity,
s) the VH comprises an amino acid sequence of SEQ ID NO: 89, or a variant comprising an amino acid sequence having at least about 80% sequence identity; and the VL comprises an amino acid sequence of SEQ ID NO: 121, or a variant comprising an amino acid sequence having at least about 80% sequence identity,
t) the VH comprises an amino acid sequence of SEQ ID NO: 89, or a variant comprising an amino acid sequence having at least about 80% sequence identity; and the VL comprises an amino acid sequence of SEQ ID NO: 125, or a variant comprising an amino acid sequence having at least about 80% sequence identity,
u) the VH comprises an amino acid sequence of SEQ ID NO: 89, or a variant comprising an amino acid sequence having at least about 80% sequence identity; and the VL comprises an amino acid sequence of SEQ ID NO: 129, or a variant comprising an amino acid sequence having at least about 80% sequence identity,
v) the VH comprises an amino acid sequence of SEQ ID NO: 89, or a variant comprising an amino acid sequence having at least about 80% sequence identity; and the VL comprises an amino acid sequence of SEQ ID NO: 133, or a variant comprising an amino acid sequence having at least about 80% sequence identity,
w) the VH comprises an amino acid sequence of SEQ ID NO: 89, or a variant comprising an amino acid sequence having at least about 80% sequence identity; and the VL comprises an amino acid sequence of SEQ ID NO: 137, or a variant comprising an amino acid sequence having at least about 80% sequence identity,
x) the VH comprises an amino acid sequence of SEQ ID NO: 89, or a variant comprising an amino acid sequence having at least about 80% sequence identity; and the VL comprises an amino acid sequence of SEQ ID NO: 141, or a variant comprising an amino acid sequence having at least about 80% sequence identity,
y) the VH comprises an amino acid sequence of SEQ ID NO: 89, or a variant comprising an amino acid sequence having at least about 80% sequence identity; and the VL comprises an amino acid sequence of SEQ ID NO: 145, or a variant comprising an amino acid sequence having at least about 80% sequence identity,
z) the VH comprises an amino acid sequence of SEQ ID NO: 89, or a variant comprising an amino acid sequence having at least about 80% sequence identity; and the VL comprises an amino acid sequence of SEQ ID NO: 149, or a variant comprising an amino acid sequence having at least about 80% sequence identity,
aa) the VH comprises an amino acid sequence of SEQ ID NO: 89, or a variant comprising an amino acid sequence having at least about 80% sequence identity; and the VL comprises an amino acid sequence of SEQ ID NO: 153, or a variant comprising an amino acid sequence having at least about 80% sequence identity, or
ab) the VH comprises an amino acid sequence of SEQ ID NO: 89, or a variant comprising an amino acid sequence having at least about 80% sequence identity; and the VL comprises an amino acid sequence of SEQ ID NO: 157, or a variant comprising an amino acid sequence having at least about 80% sequence identity.

Embodiment 34. An anti-C2 antibody, a fragment, or a fusion protein thereof that binds to C2 or C2a competitively with the anti-C2 antibody, a fragment, or a fusion protein thereof according to any one of embodiments 1-33.

Embodiment 35. The anti-C2 antibody, a fragment, or a fusion protein thereof according to any one of embodiments 1-34, wherein the antibody or a fragment thereof is selected from the group consisting of: a full length antibody, Fab, Fab′, F(ab)2, F(ab′)2, and scFv.

Embodiment 36. The anti-C2 antibody, a fragment, or a fusion protein thereof according to any one of embodiments 1-35, wherein the antibody or a fragment thereof further comprises an Fc region.

Embodiment 37. The anti-C2 antibody, a fragment, or a fusion protein thereof according to embodiment 36, wherein the Fc region comprises an IgG4 or an IgG1 sequence.

Embodiment 38. The anti-C2 antibody, a fragment, or a fusion protein thereof according to embodiment 37, wherein the Fc region comprises the amino acid sequence of SEQ ID NO: 183 or a variant thereof.

Embodiment 39. The anti-C2 antibody, a fragment, or a fusion protein thereof according to embodiment 37, wherein the Fc region comprises the amino acid sequence of SEQ ID NO: 210 or a variant thereof.

Embodiment 40. The anti-C2 antibody, a fragment, or a fusion protein thereof according to embodiment 38, wherein the Fc region comprises one or more mutations selected from the group consisting of S228P, M428L, N434A and N434S, wherein the mutations are relative to SEQ ID NO: 183 under the EU numbering system.

Embodiment 41. The anti-C2 antibody, a fragment, or a fusion protein thereof according to embodiment 40, wherein the Fc region comprises mutations a) S228P, M428L and N434A, or b) S228P, M428L and N434S.

Embodiment 42. The anti-C2 antibody, a fragment, or a fusion protein thereof according to embodiment 41, wherein the Fc region comprises the amino acid sequence of SEQ ID NO: 184 or 216.

Embodiment 43. The anti-C2 antibody, a fragment, or a fusion protein thereof according to embodiment 39, wherein the Fc region comprises one or more mutations selected from the group consisting of L234A, L235A, M428L, N434A and N434S, wherein the mutations are relative to SEQ ID NO: 210 under the EU numbering system.

Embodiment 44. The anti-C2 antibody, a fragment, or a fusion protein thereof according to embodiment 43, wherein the Fc region comprises mutations a) L234A, L235A, M428L and N434A, or b) S228P, M428L and N434S.

Embodiment 45. The anti-C2 antibody, a fragment, or a fusion protein thereof according to any one of embodiments 44, wherein the Fc region comprises the amino acid sequence of SEQ ID NO: 214 or 215.

Embodiment 46. The anti-C2 antibody, a fragment, or a fusion protein thereof according to any one of embodiments 1-45, wherein the antibody or a fragment thereof is an isolated anti-C2 antibody or a fragment thereof.

Embodiment 47. A fusion protein comprising the anti-C2 antibody according to any one of embodiments 1-46.

Embodiment 48. The anti-C2 antibody, a fragment thereof or a fusion protein thereof according to any one of embodiments 1-47, wherein the antibody or a fragment thereof is a fully human antibody or a fragment thereof.

Embodiment 49. An anti-C2 antibody fusion protein comprising a heavy chain-factor H fusion polypeptide and a light chain, wherein the heavy chain-factor H fusion comprises an amino acid sequence of any one of SEQ ID NOs: 202-204, 206, 211-213, and 220-228, and the light chain comprises an amino acid sequence of any one of SEQ ID NOs: 205, 207, and 229-245.

Embodiment 50. The anti-C2 antibody, a fragment, or a fusion protein thereof according to any one of embodiments 15-49, wherein the low-pH dissociation factor of the antibody or a fragment thereof dissociating from human C2 is between about 40% to about 95%.

Embodiment 51. The anti-C2 antibody, a fragment, or a fusion protein thereof according to any one of embodiments 15-50, wherein the neutral-pH dissociation factor of the antibody or a fragment thereof dissociating from human C2 is between about 0% to about 15%.

Embodiment 52. The anti-C2 antibody, a fragment, or a fusion protein thereof according to any one of embodiments 15-51, wherein the ratio of low-pH dissociation to neutral-pH dissociation from human C2 is 5 or more.

Embodiment 53. The anti-C2 antibody, a fragment, or a fusion protein thereof according to any one of embodiments 15-52, wherein the low-pH dissociation factor of the antibody or a fragment thereof dissociating from cynomolgus C2 is between about 40% to about 80%.

Embodiment 54. The anti-C2 antibody, a fragment, or a fusion protein thereof according to any one of embodiments 15-53, wherein the neutral-pH dissociation factor of the antibody or a fragment thereof dissociating from cynomolgus C2 is between about 0% to about 10%.

Embodiment 55. The anti-C2 antibody, a fragment, or a fusion protein thereof according to any one of embodiments 15-54, wherein the ratio of low-pH dissociation to neutral-pH dissociation from cynomolgus C2 is 7 or more.

Embodiment 56. The anti-C2 antibody, a fragment, or a fusion protein thereof according to any one of embodiments 1-55, wherein the antibody or a fragment thereof inhibits the cleavage of human C2 into fragments C2a and C2b.

Embodiment 57. The anti-C2 antibody, a fragment, or a fusion protein thereof according to any one of embodiments 1-56, wherein the antibody or a fragment thereof binds to human C2a.

Embodiment 58. The anti-C2 antibody, a fragment, or a fusion protein thereof according to any one of embodiments 1-57, wherein the antibody or a fragment thereof cross-reacts with a cynomolgus monkey C2.

Embodiment 59. The anti-C2 antibody, a fragment, or a fusion protein thereof according to any one of embodiments 1-58 or a fragment thereof, wherein the antibody or a fragment thereof cross-reacts with a cynomolgus monkey C2a.

Embodiment 60. The anti-C2 antibody, a fragment, or a fusion protein thereof according to any one of embodiments 1-59 or a fragment thereof, wherein the antibody or a fragment thereof has a serum half-life in humans that is at least about 2 days.

Embodiment 61. The anti-C2 antibody, a fragment, or a fusion protein thereof according to any one of embodiments 1-60, wherein the antibody or a fragment thereof is manufactured in CHO cells.

Embodiment 62. A nucleic acid encoding the antibody, a fragment, or fusion protein thereof according to any one of embodiments 1-61.

Embodiment 63. A vector comprising the nucleic acid of embodiment 62.

Embodiment 64. A host cell comprising the vector of embodiment 63.

Embodiment 65. A method of producing the antibody, a fragment, or fusion protein thereof according to any one of embodiments 1-61 by allowing expression of the antibody or a fragment thereof by cell under a sufficient condition.

Embodiment 66. A pharmaceutical composition comprising the antibody, a fragment, or fusion protein thereof according to any one of embodiments 1-61 and a pharmaceutically acceptable carrier.

Embodiment 67. A method of treating an individual having a complement-associated disease or condition, comprising administering to the individual an effective amount of the pharmaceutical composition of embodiment 66.

Embodiment 68. The method of embodiment 67, wherein the disease or disorder is at least selected from the group consisting of: macular degeneration (MD), age-related macular degeneration (AMD), ischemia reperfusion injury, arthritis, rheumatoid arthritis, lupus, ulcerative colitis, stroke, post-surgery systemic inflammatory syndrome, asthma, allergic asthma, chronic obstructive pulmonary disease (COPD), paroxysmal nocturnal hemoglobinuria (PNH) syndrome, autoimmune hemolytic anemia (AIHA), Gaucher disease, myasthenia gravis, neuromyelitis optica, (NMO), multiple sclerosis, delayed graft function, antibody-mediated rejection, atypical hemolytic uremic syndrome (aHUS), central retinal vein occlusion (CRVO), central retinal artery occlusion (CRAO), epidermolysis bullosa, Hidradenitis Suppurativa, bullous pemphigoid, Myelin oligodendrocyte glycoprotein antibody-associated disease (MOGAD), sepsis, septic shock, organ transplantation, inflammation (including, but not limited to, inflammation associated with cardiopulmonary bypass surgery and kidney dialysis), C3 glomerulopathy, membranous nephropathy, IgA nephropathy, glomerulonephritis (including, but not limited to, anti-neutrophil cytoplasmic antibody (ANCA)-mediated glomerulonephritis, lupus nephritis, and combinations thereof), ANCA-mediated vasculitis, Shiga toxin induced HUS, and antiphospholipid antibody-induced pregnancy loss, graft versus host disease (GVHD) or any combinations thereof.

Embodiment 69. A method of reducing the activity of a complement system in an individual, comprising administering to the individual an effective amount of the pharmaceutical composition of embodiment 66.

Embodiment 70. The anti-C2 antibody fusion protein according to any one of embodiments 46-61, wherein the anti-C2 antibody fusion protein is capable of overcoming the C2 bypass phenomenon.

Embodiment 71. The anti-C2 antibody fusion protein according to any one of embodiments 46-61 and 70, wherein the anti-C2 antibody fusion protein comprises a VL sequence selected from any one of SEQ ID NOs: 229-245, and a heavy-chain FH fusion sequence selected from any one of SEQ ID NOs: 223-228.

### Examples

The examples below are intended to be purely exemplary of the invention and should therefore not be considered to limit the invention in any way. The following examples and detailed description are offered by way of illustration and not by way of limitation.

### Example 1: Antigen Used for Anti-C2 Antibody Generation

The human complement component 2 (C2) is a single chain polypeptide precursor of 752 residues, which can be in vivo cleaved by proteases such as complement component 1s (C1s) and MASP2 into two fragments: C2a (C-terminal 244-752) and C2b (N-terminal 21-243). C2 provides the catalytic subunit C2a to the classical/lectin pathway C3- and C5-convertases C4bC2a and C4bC2aC3b, respectively. Antigens were selected to develop naturally optimized human antibodies targeting the human complement C2, which were subsequently screened for development of immunotherapeutics.

The antigen for immunization was the recombinant human complement C2 (NP_000054.2), C-terminal 6His-tag, (HEK-293 cell expression), obtained from Sino Biological (Catalog Number: 10154-H08H). A total amount of 3-5 mg was used with a purity of 95% or above and endotoxin less than 1.0 EU/μg protein (as determined by the LAL method).

For screening, characterization and cross-species reactivity assays, recombinant human complement C2 (NP_000054.2), human C2a, cynomolgus (cyno) complement C2 (XP_005553508.1), and cyno C2a, all with C-terminal 6His-tag, made in house (CHO cell expression), were used as reference antigens for hybridoma screen and for determination of antibody binding activity and cross species reactivity. Rabbit C2 and mouse C2 were also prepared for further analysis.

The cDNA sequences encoding human complement C2, human C2a, cyno complement C2, and cyno C2a proteins with a C-terminal 6 histidine tag were synthesized and inserted to a mammalian cell expressing vector with CMV promotor (pCDNA3.4, for all recombinant complement fragments). The C2 and C2a proteins were expressed in CHO cells in non-serum suspension culture condition and purified from culture medium with Ni-TED Sefinose affinity chromatography. The C2 proteins were further purified with SP Sefinose cation-exchange chromatography. The purity of each protein preparation was determined by SDS-PAGE.

### Example 2: Identification of Construction and Selection of the Anti-C2 Antibodies

### 1. Animal Immunization and Generation of Human Antibodies

The OmniRat hybridoma platform was used to develop human monoclonal antibodies targeting human complement component 2 (C2). The rats were immunized with human C2 (GenBank accession number: NP_000054.2), expressed and purified from the conditional medium of HEK293 cells, with adjuvant. The serum titer was measured by ELISA against human C2. The lymphocytes isolated from lymph nodes and spleen were used for cell fusion with Sp2/0 myeloma cells. Hybridoma supernatants were screened in ELISA binding assays against human C2 protein. Positive cells were selected for subcloning. The subclones were screened by ELISA against both human and cynomolgus monkey (Macaca fascicularis) C2 (GenBank accession number: XP_005553508.1), generated using the same method as the human C2 protein described above. The binding positive clones were further screened in sheep RBC lysis assay.

### 2. Hybridoma Screen for Leads and Anti-C2 Antibody Identification

### 2.1 Cell Fusion and Screening

B cells isolated from lymph nodes and spleen of immunized rats were mixed with Sp2/0 myeloma cells and fused in electric fusion solution by an electro-fusion apparatus (BTX, ECM2001). The fused cells were resuspended in DMEM medium (GIBCO, 31053-028) supplemented with 20% FBS (ExCell Bio, FND500) and 1×HAT (Sigma, H0262), and then were transferred into 96-well plates with 1.5×10⁴ cells per well. The fused cells were kept in a 37° C., 5% CO₂ incubator for 10˜14 days.

Following cell fusion, hybridoma supernatant was screened for binding to human C2 followed by binding to cyno C2 in ELISA. 7520 hybridoma supernatants were screened against human C2. 240 hybridomas that positively bind to human C2 were expanded in 24 well plates. The supernatants of the 24 well plates were confirmed for binding to both human C2 and cyno C2 in ELISA, as well as functional activity in sheep RBC lysis assay. Combining the functional and the binding activities, 20 hybridoma were selected for subcloning.

Visible single colony per well were picked and supernatants of the clone lines of the clones were collected for confirmative screening. Based on the binding and the functional activity data, 11 clones were selected for sequencing. Recombinant antibody from each hybridoma clonal line were expressed, purified and used for further characterization.

### 2.2 Recombinant Antibody Generation

A total of 11 clones were selected from the screen procedures for recombinant antibody generation. VH- and VL-genes of each hybridoma clone were PCR amplified and sequenced. The V region cDNA were cloned into IgG4 PLA expression vector. Antibodies were produced using Expi-CHO-S expression system and purified by protein-A affinity chromatography, and buffered in PBS (pH7.4). One clone (Ab ID: 78.46) was not further developed because of relatively low antigen binding activities. The antigen-binding activities and functional activities were validated as described below.

TABLE 2
VH and VL of selected clones

| AbID | Amino acid sequences |
|---|---|
| 34.5 | QVQLQESGPGLVKPSQTLSLTCTVSGGSISRGGHQWTWIRQHPGKGLEWIGYIHHSGST |
| VH | YYNPSLNDRLTILVDTPKNQFSLKLSSVTAADTAVYYCARWNLGSYGDYWGQGTLVTVS |
| | S (SEQ ID NO: 1) |
| 34.5 | AIVMTQSPDSLAVSLGERATINCKSSQSVLYSPUNKNYLAWYLQKPGQPPKLLIYWAST |
| VL | RESGVPDRFSGSGSGTDFTLTISSLQAEDVAVYYCQQYYTTPLTFGGGTKVEIK |
| | (SEQ ID NO: 2) |
| 199.1 | QVQLQESGPGLVKPSQTLSLTCTVSGGSISRGGHQWSWIRQNPEKGLEWIGYIHHSGST |
| VH | YYNPSLNNRLTILVDTPKNQFSLKLSSVTAADTAVYYCARWNLGSYGDYWGQGTLVTVS |
| | S (SEQ ID NO: 9) |
| 199.1 | DIVMTQSPDSLAVSLGERATINCKSSQSVLYSPNNQNFLAWYLQKPGQPPKLLIYWAST |
| VL | RESGVPDRFSGSGSGTDFTLTISSLQAEDVAVYYCQQYYTTPLTFGGGTKVEIK |
| | (SEQ ID NO: 10) |
| 3.2 | EVQLLESGGGLVQPGGSLRLSCAASGFTFRHYAMSWVRQAPGKGLEWVSLISGSGASTF |
| VH | YADSVKGRFTISRDNSENMLYLQMNSLRAEDTAVYYCAKDSLAVAGSEYFQHWGQGTLV |
| | TVSS (SEQ ID NO: 17) |
| 3.2 | SYVLTQAPSVSVAPGQTARITCGGNYFGGKSVHWYQQRPGQAPVLVVYDDSDRPSGIPE |
| VL | RFSGSKSGNTATLAISRVEAGDEAAYYCQVYDTSSDHWVFGGGTKLTVL |
| | (SEQ ID NO: 18) |
| 39.4 | EVQLLESGGGLVQPGGSLSLSCTASGFTFSHYAMSWVRQAPGKGLEWVSVISGSGASTF |
| VH | YADSVKGRFTISRDNSENTLYLQMNSLRAEDTAVYYCAKDSIAVAGSEYFQHWGQGTLV |
| | TVSS (SEQ ID NO: 25) |
| 39.4 | SYVLTQAPSVSVAPGQTARITCGGNNIGGKTVHWYQQKPGQAPMLVVYDDSDRPSGIPE |
| VL | RFSGSKSGNMAILTISRVEAGDEADYYCQLYDIYSDHWVFGGGTKLTVL |
| | (SEQ ID NO: 26) |
| 48.14 | EVQLLESGGGLVQPGGSLRLSCAASGFKFSYYAMSWVRQAPGKGLEWVSLISGSGYSTY |
| VH | FADSVKGRFTISRDNSKNTLYLQMNSLRAEDTAVYYCAKDSIAVAGSEYFQHWGQGTLV |
| | TVSS (SEQ ID NO: 33) |
| 48.14 | SYVLTQPPSVSVAPGQTARITCGGDNIGGKSVHWYQQKPGQAPVLVVYDDSDRPSGIPE |
| VL | RFSGSDSGNTATLTISWVEAGDEADYYCQVWDDSSDHWVFGGGTKLTVL |
| | (SEQ ID NO: 34) |
| 59.45 | QLQLQESGPGLVKTSETLSLTCTVSGGSITSGVYYWGWIRQPPGKGLEWIGSIYHGGNT |
| VH | YNNPSLKSRVTIFIDTSKNQFSLKLSSVTAADTAVYYCAEGVSSGWSYYFDYWGQGTLV |
| | TVSS (SEQ ID NO: 41) |
| 59.45 | SSELTQDPAVSVALGQTVRITCQGDSLRRFYASWYQQKPGQAPVLVGYGKNNRPSGIPD |
| VL | RFSGSTSGNAASLTITGSQAEDEADYYCNSRDSSGDHLIFGGGTKLTVL |
| | (SEQ ID NO: 42) |
| 69.44 | QVQLVQSGAEVKKPGASVKVSCKASGYSFTTYYMYWVRQAPGQGLEWMGIITPSGGSTT |
| VH | YAQKFQDRVTMTRDASTTTVYMELSSLRSEDTAVYYCARGYFGSYGFDHWGQGTLVTVS |
| | S (SEQ ID NO: 49) |
| 69.44 | SYELTQPPSVSLSPGQTASITCSGDKLGNKYTCWYQQKPGQSPVLVIYQDNKRPSGIPE |
| VL | RFSGSNSGNTATLTISGTQAMDEADYYCQTWDSSTAVFGGGTKLTVL |
| | (SEQ ID NO: 50) |
| 83.16 | QLQLQESGPGLVKSSETLSLTCTVSGGPISSSSYYWGWIRQPPGKQLEWIGTIYYSGRT |
| VH | YYNPSLKSRGTISVGTSKNQFSLKLSSVTAADTAVFYCAGGVGVTWDYWGQGTLVTVSS |
| | (SEQ ID NO: 57) |
| 83.16 | SYDLSQPLSVSVALGQTARITCGGNDIESKNVHWYQQRPGQAPVLVIYRNSNRPSGIPE |
| VL | RFSGSNSGNTATLTISRGQGGDEADYYCQVWDSSTVIFGGGTKLTVL |
| | (SEQ ID NO: 58) |
| 139.14 | QVQLRESGPGLMKPSETLSLTCTVSGGSVSSGVYYWTWIRQPPGKGLEWIGSMFYSGNA |
| VH | DYNPSLKSRVTISVDTSKNQFSLRLNSVSAADMAVYYCARGYASGWSYYFDYWGQATLV |
| | TVSS (SEQ ID NO: 65) |
| 139.14 | SSELTQDPTVSVALGQTVRITCQGDSLRTYYASWYQQKPGQAPVLVLYGKNNRPSGIPD |
| VL | RFSGSSSGNTASLTITGAQAEDEADYYCNSRYSSGTHVVFGGGTKLTVL |
| | (SEQ ID NO: 66) |
| 149.46 | QVQLQQSGPGLVKPSQPLSLTCAISGDSVSSNSATWSWIRQSPSRGLEWLGRTYYRSKW |
| VH | YHDYAVFVKSRMTINPDTSKNQFSLQLNSVTPEDTAVYYCARGGMPASDIPHFDNWGQG |
| | ILVTVSS (SEQ ID NO: 73) |
| 149.46 | SYELTQPPSLSVSPGQTASITCSGHKLGDKYACWYQQKPGQSPVLVIYQDNKRPSGIPE |
| VL | RFSGSNSGNTATLTISGTQALDEADYYCQAWDSITYVFGTGTKVTVL |
| | (SEQ ID NO: 74) |
| 78.46 | EVQLVESGGGLVKPRGSLRLSCGASGFTFTNAWMTWVRQAPGKGLEWVGRIKSKTDGGT |
| VH | TDYAAPVKGRFTISRDDSKNTLYLQMNSLKTEDTAVYYCNRERELGRGPWDYFYYGMDV |
| | WGQGTTVTVSS (SEQ ID NO: 81) |
| 78.46 | SYVLTQPPSVSVAPGQTARITCGGNDIGSKSVHWYQQKPGQAPVLVVYDDNDRSSGIPE |
| VL | RFSGSNSGNTAALTISRVEAGDEADYYCQVWDSSSDHWVFGGGTKLTVL |
| | (SEQ ID NO: 82) |

### Example 3: Binding and Functional Characterization of Antibodies and FH Fusion Proteins

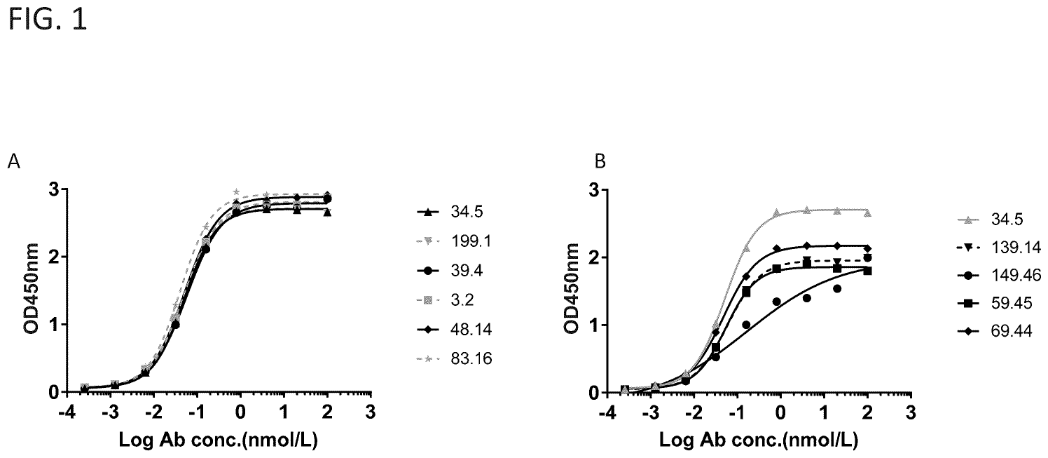

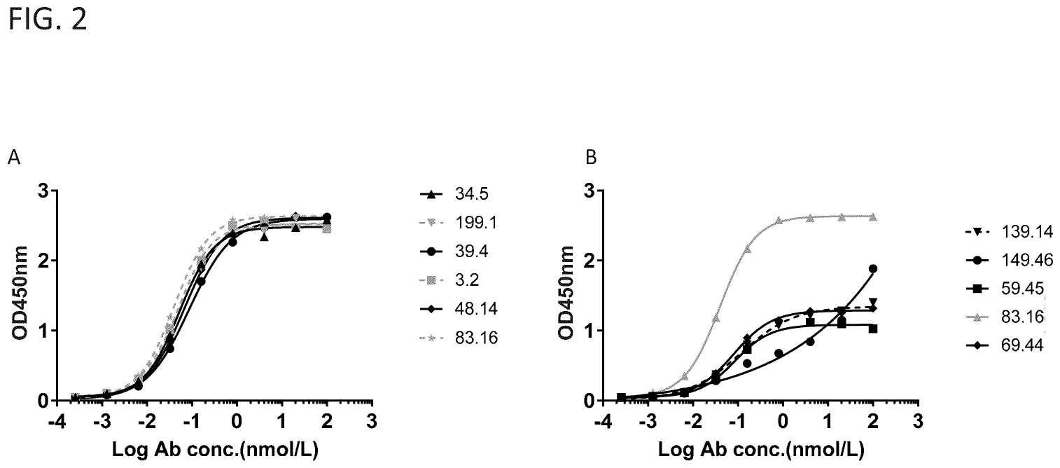

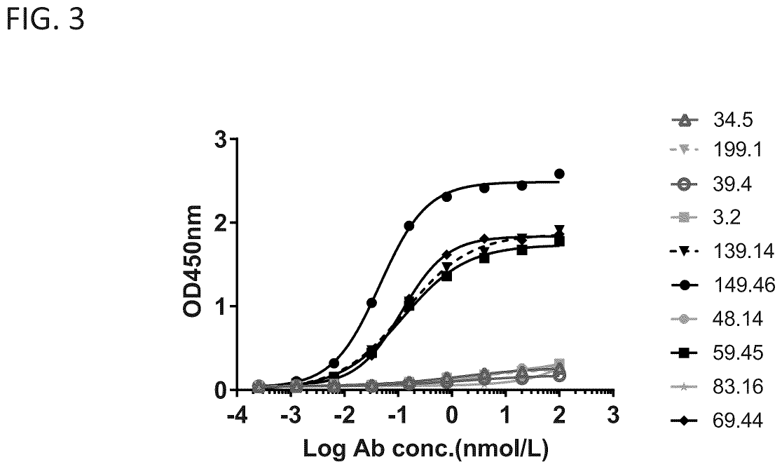

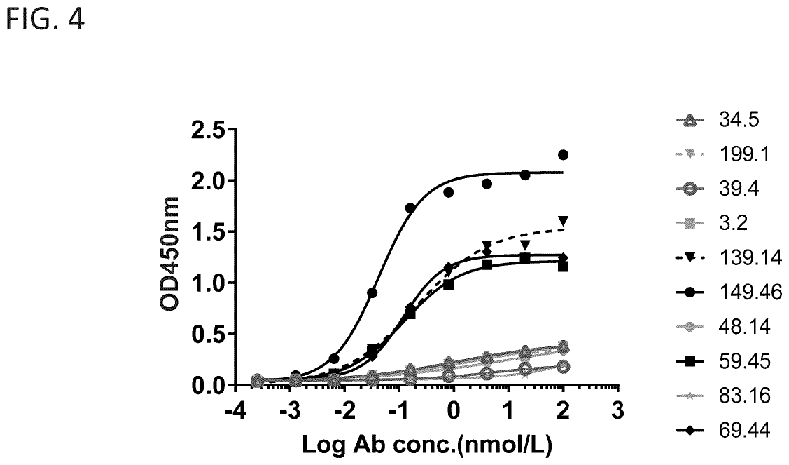

Human and cyno C2 binding activities were characterized by ELISA. All recombinant antibodies show human C2 and cyno C2 binding activities (FIGS. 1 and 2). Among the 10 antibodies, 34.5, 199.1, 39.4, 3.2, 48.14, and 83.16 are more potent than 139.14, 149.46, 59.45, and 69.44 in binding to human C2 and cyno C2; 139.14, 149.46, 59.45, and 69.44 bind to both human C2a and cyno C2a, while others have no C2a binding activities (FIGS. 3 and 4). Table 3 summarizes the EC50 of each antibody binding to human and cyno C2/C2a.

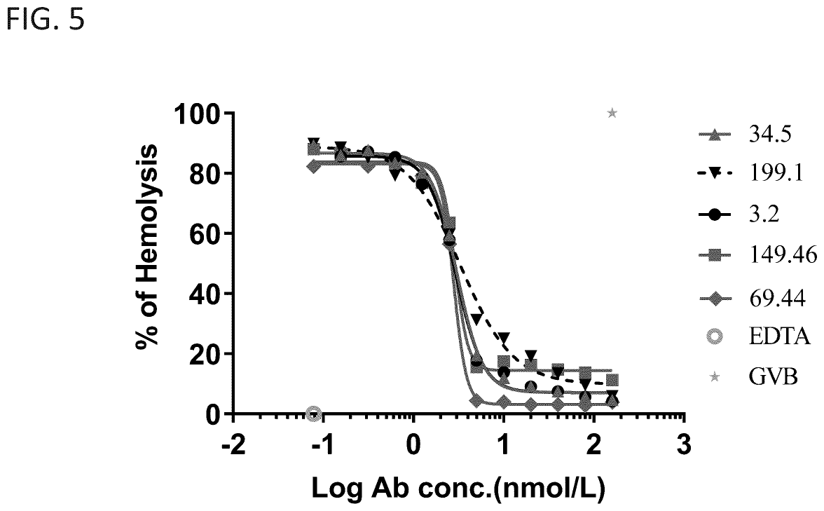

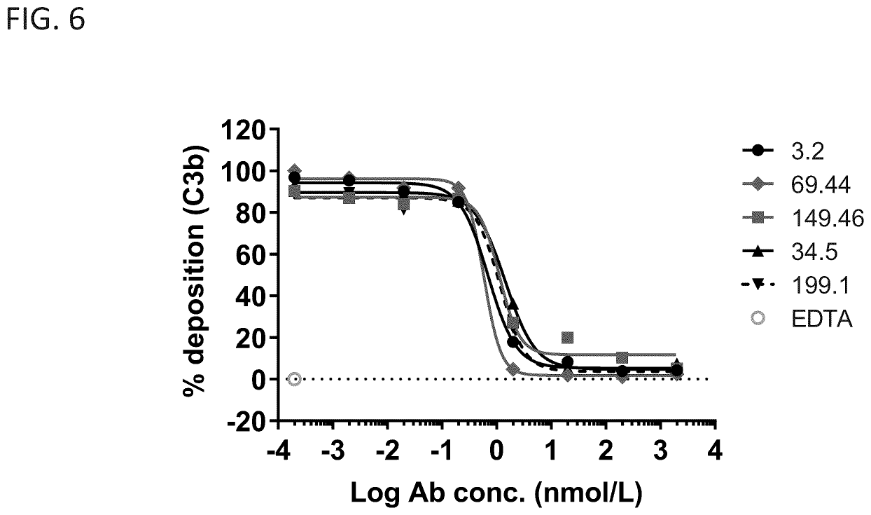

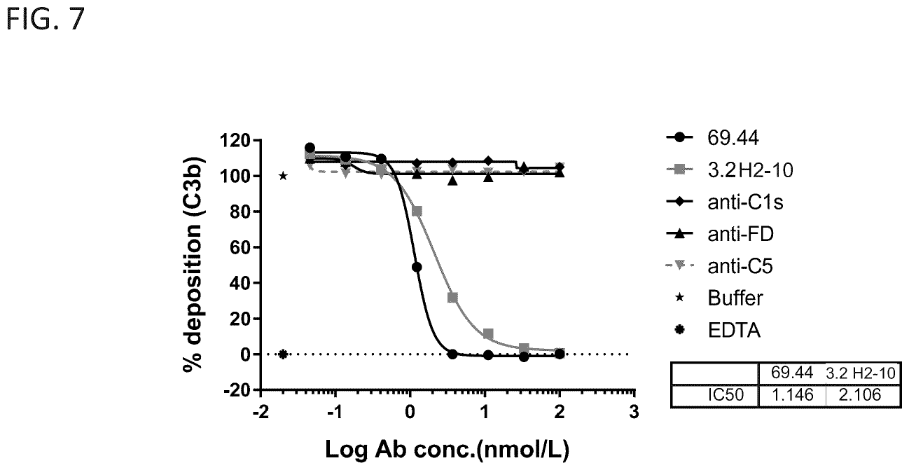

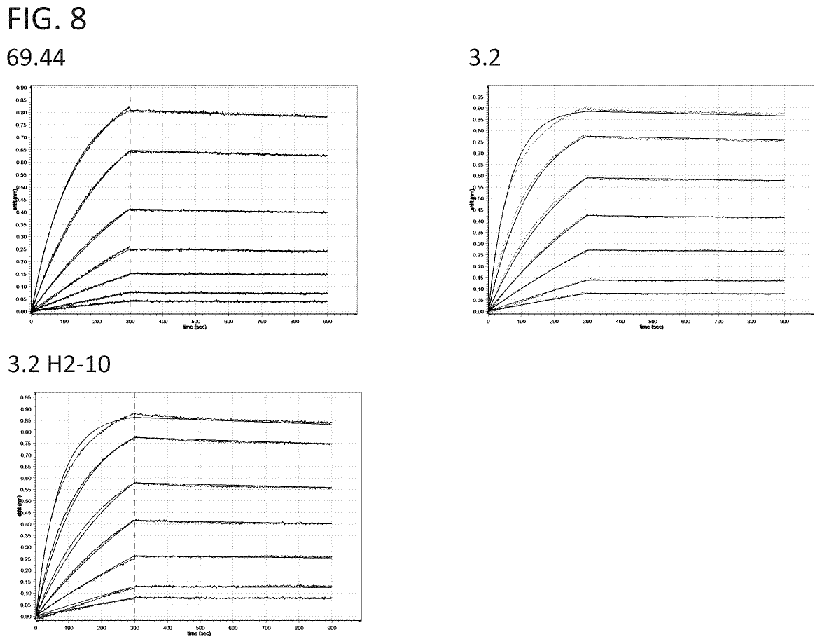

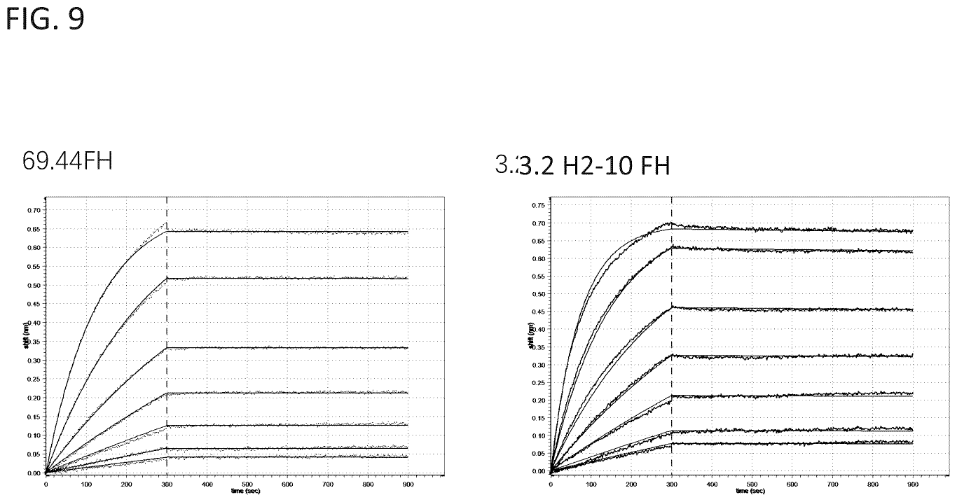

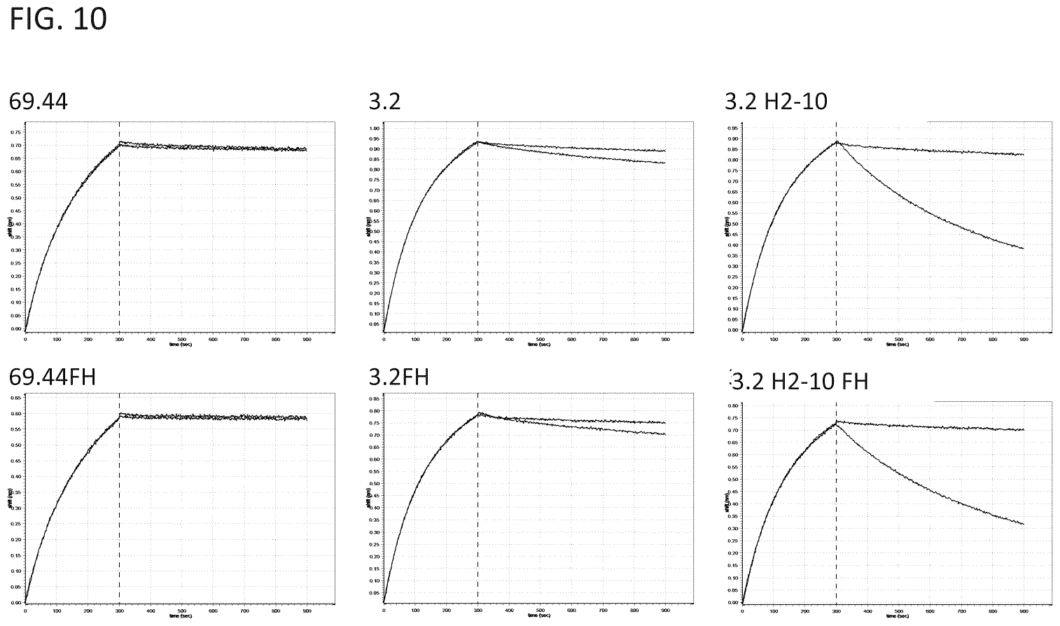

Five of the 10 best binders, 34.5, 199.1, 3.2, 149.46, and 69.44 were further characterized in biological functional assays. In the sheep RBC lysis assay, 34.5, 199.1, 3.2, 149.46, and 69.44 show potent inhibitory activities (FIG. 5). In IgM-C3b ELISA, these antibodies reduced C3b level following IgM stimulation of human and cyno serum complement activity (FIG. 6) as well as in the mannan-mediated C3b deposition assay (FIG. 7).

TABLE 3
Human and cyno monkey C2 binding activity assessed by ELISA

EC50 (nmol/L)

| Ab ID | Human C2 | Human C2a | Cyno C2 | Cyno C2a |
|---|---|---|---|---|
| 34.5 | 0.0512 | 1.292 | 0.0521 | 1.107 |
| 199.1 | 0.0479 | 2.841 | 0.05 | 0.9502 |
| 39.4 | 0.0576 | 1.698 | 0.0869 | 4.965 |
| 3.2 | 0.0504 | NA | 0.0478 | 2.462 |
| 139.14 | 0.0599 | 0.1311 | 0.1149 | 0.2077 |
| 149.46 | 0.1652 | 0.0457 | NA | 0.042 |
| 48.14 | 0.0514 | 5.412 | 0.0682 | 11.4 |
| 59.45 | 0.054 | 0.1238 | 0.263 | 0.1228 |
| 83.16 | 0.0408 | NA | 0.0397 | NA |
| 69.44 | 0.0474 | 0.1174 | 0.0794 | 0.1186 |

### Example 4. Antibody pH-Dependent Binding Engineering

1. Construction of the pH-Dependent Binding Anti-C2 Antibodies

To selectively increase antigen dissociation at pH 5.8 while maintaining high antigen binding affinity at pH 7.4, histidine mutation(s) in the targeted VH and/or VL CDR region of anti-C2 construct 3.2 were introduced by QuikChange® mutagenesis.

Constructs with a single point histidine mutation in the H-CDRs and L-CDRs were screened for their pH dependent binding to human C2 at pH 7.4 and 5.8. Among the constructs tested, SEQ ID NOs: 89 and 90 (3.2 H2-10 parental) demonstrated highest percentage dissociation at pH 5.8 while retaining its binding to human C2 at pH 7.4.

3.2 H2-10 parental was selected for further engineering by introducing histidine mutations in the L-CDRs. The resulting histidine mutants harboring two mutations, one in VH-CDR of H2-10 parental and the other in VL-CDR, were screened for the combined pH dependent binding effect resulted from of the light chain histidine mutation and heavy chain histidine mutation in H2-10 parental.

A total of 17 combined VH-VL histidine mutation constructs were made including the H2-10 parental; these constructs are collected denoted by 3.2 H2-10. The sequences of the pH-dependent anti-C2 antibodies are summarized below in Table 4.

TABLE 4
VH and VL sequences of the pH-dependent anti-C2 antibodies

| Ab ID | Sequences |
|---|---|
| 3.2 H2-10 | EVQLLESGGGLVQPGGSLRLSCAASGFTFRHYAMSWVRQAPGKGLEWVSLISGSG |
| parental | ASTHYADSVKGRFTISRDNSENMLYLQMNSLRAEDTAVYYCAKDSLAVAGSEYFQ |
| VH | HWGQGTLVTVSS (SEQ ID NO: 89) |
| 3.2 H2-10 | SYVLTQAPSVSVAPGQTARITCGGNYFGGKSVHWYQQRPGQAPVLVVYDDSDRPS |
| parental | GIPERFSGSKSGNTATLAISRVEAGDEAAYYCQVYDTSSDHWVFGGGTKLTVL |
| VL | (SEQ ID NO: 90) |
| 3.2 H2- | SYVLTQAPSVSVAPGQTARITCGHNYFGGKSVHWYQQRPGQAPVLVVYDDSDRPS |
| 10 × L1-2 | GIPERFSGSKSGNTATLAISRVEAGDEAAYYCQVYDTSSDHWVFGGGTKLTVL |
| VL | (SEQ ID NO: 97) |
| 3.2 H2- | SYVLTQAPSVSVAPGQTARITCGGNHFGGKSVHWYQQRPGQAPVLVVYDDSDRPS |
| 10 × L1-4 | GIPERFSGSKSGNTATLAISRVEAGDEAAYYCQVYDTSSDHWVFGGGTKLTVL |
| VL | (SEQ ID NO: 101) |
| 3.2 H2- | SYVLTQAPSVSVAPGQTARITCGGNYHGGKSVHWYQQRPGQAPVLVVYDDSDRPS |
| 10 × L1-5 | GIPERFSGSKSGNTATLAISRVEAGDEAAYYCQVYDTSSDHWVFGGGTKLTVL |
| VL | (SEQ ID NO: 105) |
| 3.2 H2- | SYVLTQAPSVSVAPGQTARITCGGNYFGHKSVHWYQQRPGQAPVLVVYDDSDRPS |
| 10 × L1-7 | GIPERFSGSKSGNTATLAISRVEAGDEAAYYCQVYDTSSDHWVFGGGTKLTVL |
| VL | (SEQ ID NO: 109) |
| 3.2 H2- | SYVLTQAPSVSVAPGQTARITCGGNYFGGHSVHWYQQRPGQAPVLVVYDDSDRPS |
| 10 × L1-8 | GIPERFSGSKSGNTATLAISRVEAGDEAAYYCQVYDTSSDHWVFGGGTKLTVL |
| VL | (SEQ ID NO: 113) |
| 3.2 H2- | SYVLTQAPSVSVAPGQTARITCGGNYFGGKSHHWYQQRPGQAPVLVVYDDSDRPS |
| 10 × L1-10 | GIPERFSGSKSGNTATLAISRVEAGDEAAYYCQVYDTSSDHWVFGGGTKLTVL |
| VL | (SEQ ID NO: 117) |
| 3.2 H2- | SYVLTQAPSVSVAPGQTARITCGGNYFGGKSVHWYQQRPGQAPVLVVYHDSDRPS |
| 10 × L2-1 | GIPERFSGSKSGNTATLAISRVEAGDEAAYYCQVYDTSSDHWVFGGGTKLTVL |
| VL | (SEQ ID NO: 121) |
| 3.2 H2- | SYVLTQAPSVSVAPGQTARITCGGNYFGGKSVHWYQQRPGQAPVLVVYDDHDRPS |
| 10 × L2-3 | GIPERFSGSKSGNTATLAISRVEAGDEAAYYCQVYDTSSDHWVFGGGTKLTVL |
| VL | (SEQ ID NO: 125) |
| 3.2 H2- | SYVLTQAPSVSVAPGQTARITCGGNYFGGKSVHWYQQRPGQAPVLVVYDDSDHPS |
| 10 × L2-5 | GIPERFSGSKSGNTATLAISRVEAGDEAAYYCQVYDTSSDHWVFGGGTKLTVL |
| VL | (SEQ ID NO: 129) |
| 3.2 H2- | SYVLTQAPSVSVAPGQTARITCGGNYFGGKSVHWYQQRPGQAPVLVVYDDSDRHS |
| 10 × L2-6 | GIPERFSGSKSGNTATLAISRVEAGDEAAYYCQVYDTSSDHWVFGGGTKLTVL |
| VL | (SEQ ID NO: 133) |
| 3.2 H2- | SYVLTQAPSVSVAPGQTARITCGGNYFGGKSVHWYQQRPGQAPVLVVYDDSDRPH |
| 10 × L2-7 | GIPERFSGSKSGNTATLAISRVEAGDEAAYYCQVYDTSSDHWVFGGGTKLTVL |
| VL | (SEQ ID NO: 137) |
| 3.2 H2- | SYVLTQAPSVSVAPGQTARITCGGNYFGGKSVHWYQQRPGQAPVLVVYDDSDRPS |
| 10 × L3-4 | GIPERFSGSKSGNTATLAISRVEAGDEAAYYCQVYHTSSDHWVFGGGTKLTVL |
| VL | (SEQ ID NO: 141) |
| 3.2 H2- | SYVLTQAPSVSVAPGQTARITCGGNYFGGKSVHWYQQRPGQAPVLVVYDDSDRPS |
| 10 × 13-5 | GIPERFSGSKSGNTATLAISRVEAGDEAAYYCQVYDHSSDHWVFGGGTKLTVL |
| VL | (SEQ ID NO: 145) |
| 3.2 H2- | SYVLTQAPSVSVAPGQTARITCGGNYFGGKSVHWYQQRPGQAPVLVVYDDSDRPS |
| 10 × L3-6 | GIPERFSGSKSGNTATLAISRVEAGDEAAYYCQVYDTHSDHWVFGGGTKLTVL |
| VL | (SEQ ID NO: 149) |
| 3.2 H2- | SYVLTQAPSVSVAPGQTARITCGGNYFGGKSVHWYQQRPGQAPVLVVYDDSDRPS |
| 10 × L3-7 | GIPERFSGSKSGNTATLAISRVEAGDEAAYYCQVYDTSHDHWVFGGGTKLTVL |
| VI | (SEQ ID NO: 153) |
| 3.2 H2- | SYVLTQAPSVSVAPGQTARITCGGNYFGGKSVHWYQQRPGQAPVLVVYDDSDRPS |
| 10 × L3-11 | GIPERFSGSKSGNTATLAISRVEAGDEAAYYCQVYDTSSDHWHFGGGTKLTVL |
| VL | (SEQ ID NO: 157 |

2. pH-Dependent Dissociation from Human C2

The histidine mutant constructs in Table 4 were tested for their pH-dependent dissociation from human C2. Probe-immobilized anti-C2 antibodies were incubated with C2 at pH 7.4 for 600s, followed by a 600s dissociation period in K Buffer at pH 7.4 or pH 5.8. The data were processed and analyzed by the Gator evaluation software.

All histidine mutant constructs showed significantly higher percentage of dissociation from human C2 at pH 5.8 (low-pH dissociation factor) than at pH 7.4 (neutral-pH dissociation factor), ranging from 45% to over 90%, comparing to H2-10 parental. The histidine mutant constructs maintained bound to C2 at pH 7.4, with the percentage dissociation (neutral-pH dissociation factor) under 10%. The wild type anti-C2 construct 3.2 before pH-dependent engineering showed less than 10% dissociation from C2, under both pH 7.4 and pH 5.8 conditions.

TABLE 5
pH-dependent dissociation of the histidine
mutant constructs from human C2

| | Dissociation (%) | | Fold dissociation |
|---|---|---|---|
| Antibody | pH 7.4 | pH 5.8 | pH 5.8/pH 7.4 |
| 3.2 H2-10 parental | 7.74 | 51.26 | 6.6 |
| 3.2 H2-10 × L1-2 | 5.10 | 56.52 | 11.1 |
| 3.2 H2-10 × L1-4 | 5.90 | 55.79 | 9.5 |
| 3.2 H2-10 × L1-5 | 6.42 | 66.67 | 10.4 |
| 3.2 H2-10 × L1-7 | 4.05 | 52.00 | 12.8 |
| 3.2 H2-10 × L1-8 | 5.00 | 51.78 | 10.4 |
| 3.2 H2-10 × L1-10 | 5.45 | 56.56 | 10.4 |
| 3.2 H2-10 × L2-1 | 13.94 | 81.30 | 5.8 |
| 3.2 H2-10 × L2-3 | 5.69 | 46.22 | 8.1 |
| 3.2 H2-10 × L2-5 | 5.33 | 51.65 | 9.7 |
| 3.2 H2-10 × L2-6 | 5.97 | 52.60 | 8.8 |
| 3.2 H2-10 × L2-7 | 5.39 | 52.67 | 9.8 |
| 3.2 H2-10 × L3-4 | 13.33 | 82.80 | 6.2 |
| 3.2 H2-10 × L3-5 | 12.63 | 90.67 | 7.2 |
| 3.2 H2-10 × L3-6 | 7.61 | 68.71 | 9.0 |
| 3.2 H2-10 × L3-7 | 7.00 | 59.97 | 8.6 |
| 3.2 H2-10 × L3-11 | 7.16 | 62.57 | 8.7 |
| “WT” 3.2 | 3.59 | 8.20 | 2.3 |

### Example 4: Construction of the Anti-C2 Antibody Factor H Fusion Proteins

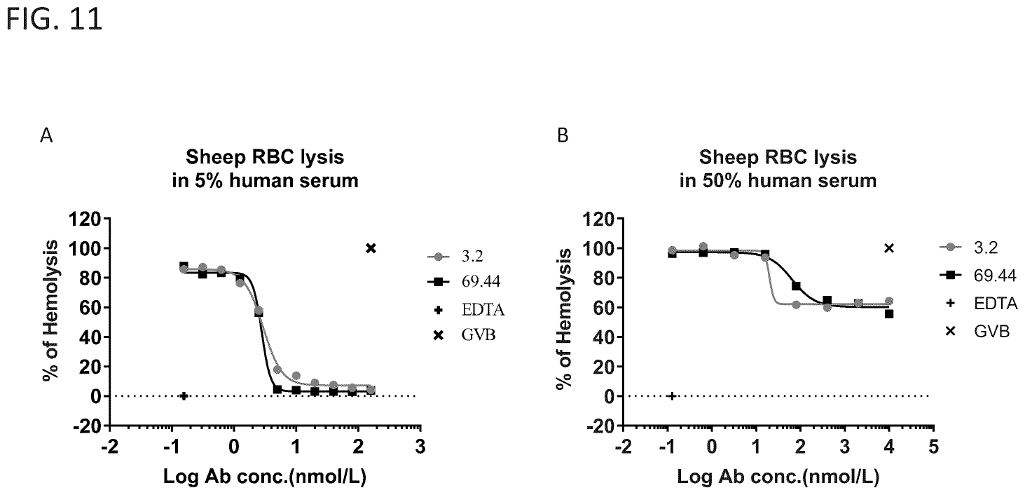

At high (50%) serum concentration, C2 activities in sheep RBC lysis and IgM mediated C3b and C5b-9 assays could not be completely inhibited by C2 antibodies. For example, anti-C2 mAb showed approximately only 40% sheep RBC lysis inhibition if 50% of serum is used in the assay (FIG. 11). This phenomenon might be explained by a so-called “C2 bypass” mechanism, in which complement alternative pathway components such as Bb be involved, forming C4bBb complex and activating C3 without C2a. We therefore designed a fusion protein in which the activity domains of complement FH protein. FH1-5, was fused to C2 antibody heavy chain C-terminus. The fusion protein has bi-functional anti-C2 and FH1-5 domains able to inhibit the classical and the lectin pathways via C2 targeting, as well as alternative pathway by FH1-5 domains. Factor H 1-5 fusion with 69.44, 3.2, and a representative pH dependent 3.2 comprising a histidine mutation in the heavy chain CDR2 (denoted by 3.2 H2-10) were constructed for further characterization (denoted by 69.44FH, 3.2FH, and 3.2H2-10 FH respectively). The amino acid sequences of FH1-5 and Fc that may be used in the fusion constructs, and exemplary factor H fusion constructs are shown in Table 6.

TABLE 6
Sequences of components in the factor H fusion constructs
and exemplary factor H fusion constructs

| FH1-5 peptide sequence | SEQ ID NO: | 3.2 H2-10 × L1-4 FH fusion | SEQ ID NO: |
|---|---|---|---|
| | 201 | VL | 231 |
| IgG4 WT Fc | SEQ ID NO: | 3.2 H2-10 × L1-5 FH fusion | SEQ ID NO: |
| | 183 | VL | 232 |
| IgG4 Fc PLA | SEQ ID NO: | 3.2 H2-10 × L1-7 FH fusion | SEQ ID NO: |
| | 184 | VL | 233 |
| IgG1 Fc | SEQ ID NO: | 3.2 H2-10 × L1-8 FH fusion | SEQ ID NO: |
| | 210 | VL | 234 |
| IgG1 Fc LALA-LS | SEQ ID NO: | 3.2 H2-10 × L1-10 FH fusion | SEQ ID NO: |
| | 214 | VL | 235 |
| IgG1 Fc LALA-LA | SEQ ID NO: | 3.2 H2-10 × L2-1 VL | SEQ ID NO: |
| | 215 | | 236 |
| IgG4 Fc PLS | SEQ ID NO: | 3.2 H2-10 × L2-3 FH fusion | SEQ ID NO: |
| | 216 | VL | 237 |
| 3.2 H2-10 IgG1 Fc FH fusion | SEQ ID NO: | 3.2 H2-10 × L2-5 FH fusion | SEQ ID NO: |
| | 223 | VL | 238 |
| 3.2 H2-10 IgG1 LALA-LS Fc FH fusion | SEQ ID NO: | 3.2 H2-10 × L2-6 FH fusion | SEQ ID NO: |
| | 224 | VL | 239 |
| 3.2 H2-10 IgG1 LALA-LA Fc FH fusion | SEQ ID NO: | 3.2 H2-10 × L2-7 FH fusion | SEQ ID NO: |
| | 225 | VL | 240 |
| 3.2 H2-10 IgG4 Fc FH fusion | SEQ ID NO: | 3.2 H2-10 × L3-4 FH fusion | SEQ ID NO: |
| | 226 | VL | 241 |
| 3.2 H2-10 IgG4 PLA Fc FH fusion | SEQ ID NO: | 3.2 H2-10 × L3-5 FH fusion | SEQ ID NO: |
| | 227 | VL | 242 |
| 3.2 H2-10 IgG4 PLS Fc FH fusion | SEQ ID NO: | 3.2 H2-10 × L3-6 FH fusion | SEQ ID NO: |
| | 228 | VL | 243 |
| 3.2 H2-10 parental FH fusion VL | SEQ ID NO: | 3.2 H2-10 × L3-7 FH fusion | SEQ ID NO: |
| | 229 | VL | 244 |
| 3.2 H2-10 × L1-2 FH fusion VL | SEQ ID NO: | 3.2 H2-10 × L3-11 FH fusion | SEQ ID NO: |
| | 230 | VL | 245 |

### Example 5: Binding Constants of the Anti-C2 Constructs to Human C2 and Cyno C2

### Binding to Human C2

Selected anti-C2 constructs and factor H fusion proteins were tested for their binding behaviors to human C2 under neutral pH using the method demonstrated in Example 3. The results are summarized in Table 7.

TABLE 7
Binding constants of the anti-C2 constructs to human C2

| Antibody | Antigen | koff(1/s) | kon(1/Ms) | KD(M) |
|---|---|---|---|---|
| WT | Human C2 | 6.12E−05 | 3.08E+05 | 1.98E−10 |
| H2-10 × L1-2 | Human C2 | 9.39E−05 | 2.84E+05 | 3.30E−10 |
| H2-10 × L1-5 | Human C2 | 1.12E−04 | 2.72E+05 | 4.10E−10 |
| H2-10 × L1-10 | Human C2 | 8.72E−05 | 2.65E+05 | 3.29E−10 |
| H2-10 × L3-6 | Human C2 | 1.19E−04 | 2.83E+05 | 4.20E−10 |
| H2-10 × L1-4 | Human C2 | 8.18E−05 | 2.93E+05 | 2.80E−10 |
| 69.44FH | Human C2 | 1.17E−06 | 1.95E+05 | 6.02E−12 |
| 3.2 H2-10 FH | Human C2 | 1.85E−05 | 3.43E+05 | 5.39E−11 |
| 69.44 | Human C2 | 5.85E−05 | 1.97E+05 | 2.96E−10 |
| 3.2 | Human C2 | 3.73E−05 | 3.79E+05 | 9.84E−11 |
| 3.2 H2-10 | Human C2 | 6.05E−05 | 3.53E+05 | 1.72E−10 |

Table 7 shows that compared to the “WT” anti-C2 construct 3.2, histidine mutant constructs retained high affinities to human C2. The histidine mutations did not affect the on-rate between the antibodies and human C2. Factor H fusion constructs possessed higher affinity compared to non-fusion constructs to human C2.

### Binding to Cyno 52

69.44, 3.2, a representative pH-dependent construct of 3.2, and selected factor H fusion proteins were further tested for binding to cynomolgus C2. The results are summarized in Table 8.

TABLE 8
Binding constants of the anti-C2 constructs to cyno C2

| Antibody | Antigen | koff(1/s) | kon(1/Ms) | KD(M) |
|---|---|---|---|---|
| 69.44 | Cyno C2 | 5.89E−05 | 1.94E+05 | 3.04E−10 |
| 3.2 | Cyno C2 | 5.82E−05 | 4.61E+05 | 1.26E−10 |
| 3.2 H2-10 | Cyno C2 | 9.84E−05 | 4.42E+05 | 2.22E−10 |
| 69.44FH | cyno C2 | 3.86E−05 | 2.00E+05 | 1.92E−10 |
| 3.2 H2-10 FH | cyno C2 | 5.63E−05 | 4.36E+05 | 1.29E−10 |

Table 8 shows that all five constructs tested had high affinities to cyno C2 in the subnanomolar range.

Binding to human and cyno C2a

69.44 and factor H fusion protein thereof were further tested for its binding affinities and constants to human and cyno C2a. The results are summarized in Table 9. The results show that 69.44 and 69.44-FH bound to human and cyno C2a with sub-nM binding affinity.

TABLE 9
Binding constants of 69.44 to human and cyno C2a

| Antibody | Antigen | koff(1/s) | kon(1/Ms) | KD(M) |
|---|---|---|---|---|
| 69.44 | human C2a | 6.68E−05 | 1.93E+05 | 3.47E−10 |
| 69.44 | cyno C2a | 8.79E−05 | 2.34E+05 | 3.76E−10 |
| 69.44FH | human C2a | 4.01E−05 | 1.82E+05 | 2.21E−10 |
| 69.44FH | cyno C2a | 7.30E−05 | 2.11E+05 | 3.46E−10 |

### Example 6: PH-Dependent Binding and Dissociation of Factor H Fusion Constructs from C2

The factor H constructs (69.44FH, 3.2FH and 3.2 H2-10 FH) were tested for their pH binding dependency following the same experimental procedure in Example 2. Non-fusion mAb were used for comparison. Results are summarized in Table 10.

TABLE 10
pH-dependent dissociation of factor H fusion constructs from C2

| | | Dissociation (%) | | Fold dissociation |
|---|---|---|---|---|
| Antibody | Antigen | pH 7.4 | pH 5.8 | pH 5.8/pH 7.4 |
| 69.44 | human C2 | 3.15 | 2.45 | 0.78 |
| 69.44FH | human C2 | 1.71 | 0.93 | 0.54 |
| 3.2 | human C2 | 5.27 | 10.39 | 1.97 |
| 3.2FH | human C2 | 4.91 | 10.47 | 2.13 |
| 3.2 H2-10 | human C2 | 6.91 | 56.67 | 8.20 |
| 3.2 H2-10 FH | human C2 | 5.11 | 56.08 | 10.97 |
| 69.44 | cyno C2 | 4.81 | 7.22 | 1.50 |
| 69.44FH | cyno C2 | 1.40 | 6.05 | 4.32 |
| 3.2 | cyno C2 | 5.37 | 14.51 | 2.70 |
| 3.2FH | cyno C2 | 4.93 | 16.03 | 3.25 |
| 3.2 H2-10 | cyno C2 | 8.35 | 62.86 | 7.53 |
| 3.2 H2-10 FH | cyno C2 | 7.19 | 61.36 | 8.53 |

Table 10 shows that 3.2 H2-10 FH fusion protein had comparable dissociation from human and cynomolgus C2 at pH 5.8 to their pH dependent binding antibody parental, at approximately 55%-65%, while binding dissociation at pH 7.4 was below 10%. The ratios between the low-pH dissociation factor and the neutral-pH dissociation factor for 3.2 H2-10 FH was approximately 10-fold.

### Example 7: In Vitro Functional Assays Characterization of Ant-C2-Antibody and the mAbFH1-5 Fusion Proteins

### 1. Sheep RBC Lysis Assay

A classical pathway complement-mediated sheep red blood cell lysis assay was used to assess the C2 inhibitory effect of the anti-C2 antibodies 3.2, 69.44, and factor H fusion constructs 3.2 H2-10 FH and 69.44FH.

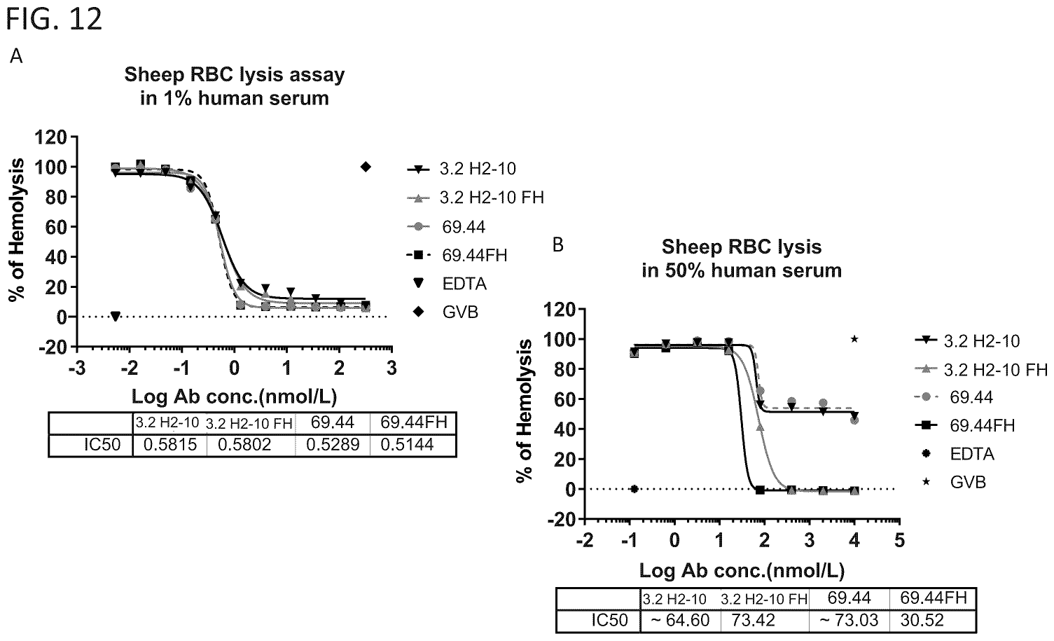

Antibody-sensitized sheep RBCs (1×10⁷ cells/assay in PBS, Complement Technology Inc) were incubated at 37° C. for 20 min with 5% normal human serum (NHS, from Complement Technology Inc) in gelatin veronal buffer (GVB2+, Sigma; total assay volume: 100 μl). NHS was pre-incubated with anti-C5 mAbs for 1 hour at 4° C. before addition into the sheep RBCs. Lysis reaction was stopped by addition of 40 mM EDTA in ice-cold PBS. The incubation mixtures were centrifuged for 5 min at 1500 rpm. The supernatant from each mixture was collected and measured for OD405 nm. A sample with only the EDTA buffer added was used as the negative lysis control, and a sample of sheep RBCs lysed completely with GVB added was used as the positive control (100% lysis) against which % lysis in other samples was normalized. Results are summarized in FIGS. 11 and 12.

FIG. 11 shows the results of inhibition of sheep RBC lysis by 3.2 and 69.44 at different antibody concentration levels. 3.2 and 69.44 had IC₅₀ values of 2.9 nM and 2.8 nM, respectively when assayed using low (5%) serum. However, the inhibition was reduced to 40% when assayed using 50% serum.

FIG. 12 shows that 3.2 H2-10 FH and 69.44FH completely inhibited sheep RBC lysis with an IC₅₀ value of 0.58 nM and 0.51 nM, respectively, in 1% human serum. The IC₅₀ value of inhibition by 3.2 H2-10 FH and 69.44FH in 50% human serum were 73.42 nM and 30.52 nM, respectively.

### 2. IgM-C3b ELISA

An IgM-C3b ELISA was used to assess the anti-C2 antibodies 3.2, 69.44, and factor H fusion constructs 3.2 H2-10 FH and 69.44FH in inhibition of the complement pathways.

For IgM-C3b ELISA, serum samples were preincubated with serially diluted anti-C2 antibodies or fusion constructs at 4° C. for 1 h, then added to ELISA 96-well plate coated with IgM (100 ng/well) and incubated at 37° C. for 1 h. The CP complement activation was stopped by adding 100 μl/well pre-cold 40 mM EDTA. After washing, the plate was incubated with 100 μl/well of anti-C3 antibody-HRP (1:4,000) at room temperature for 1 h, followed by washing, adding 100 μl/well of TMB to develop color, and then adding 50 μl/well of 2N H₂SO₄ to stop the reaction. The absorbance at 450 nm was read on a densitometric plate reader. A sample with only the EDTA buffer added was used as the negative control (NC, 0% deposition), and a sample with only GVB added was used as the positive control (PC, 100% deposition). The % deposition of C3b of each sample was calculated with the following formula: % deposition (C3b)=(ODₛₐₘₚₗₑ−OD_{NC})/(OD_{PC}−OD_{NC})×100%.

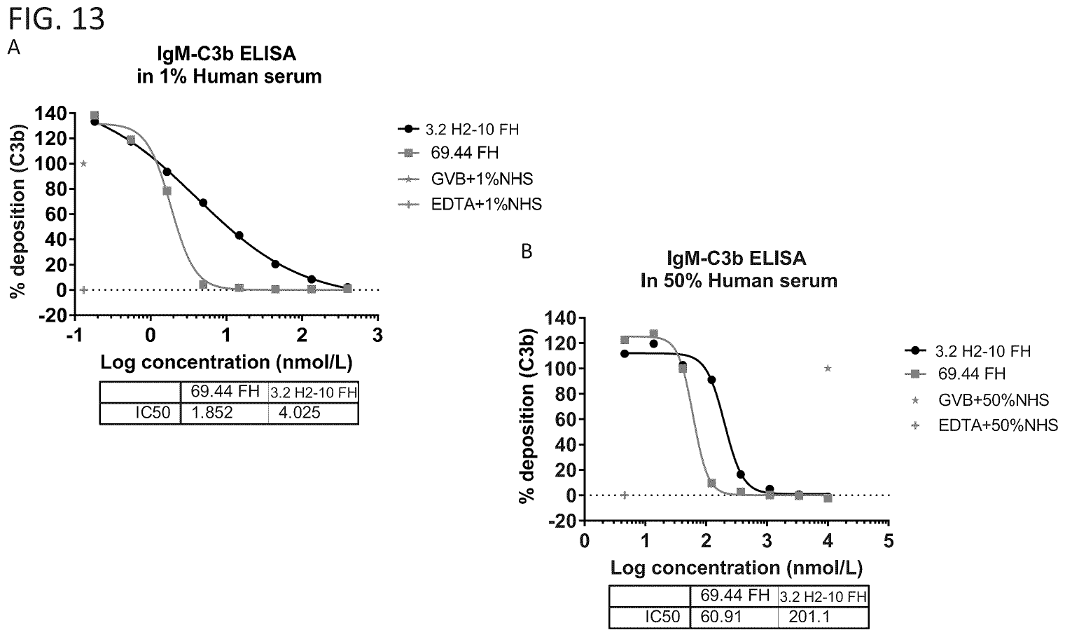

FIG. 13 shows that 3.2 H2-10 FH and 69.44FH potently inhibited the classical pathway with an IC₅₀ value of 4.01 nM and 1.85 nM, respectively, in 1% human serum. The IC₅₀ value of inhibition by 3.2 H2-10 FH and 69.44FH in 50% human serum were 209.1 nM and 60.91 nM, respectively.

### 3. Mannan-C3b ELISA

The mannan-binding lectin (MBL) pathway of complement activation was used to assess the C2 inhibitory effect of the anti-C2 antibodies 3.2, 69.44, and factor H fusion constructs 3.2 H2-10 FH and 69.44FH.

For Mannan-C3b ELISA, serum samples were preincubated with serially diluted anti-C2 antibodies or fusion constructs at 4° C. for 30 min, then added to ELISA 96-well plate coated with mannan (100 ng/well) and incubated at 37° C. for 1 h. The LP complement activation was stopped by adding 100 μl/well pre-cold 40 mM EDTA. After washing, the plate was incubated with 100 μl/well of anti-C3 antibody-HRP (1:4,000) at room temperature for 1 h, followed by washing, adding 100 μl/well of TMB to develop color, and then adding 50 μl/well of 2N H₂SO₄ to stop the reaction. The absorbance at 450 nm was read on a densitometric plate reader. A sample with only the EDTA buffer added was used as the negative control (NC, 0% deposition), and a sample with only assay buffer added was used as the positive control (PC, 100% deposition). The % deposition of C3b of each sample was calculated with the following formula: % deposition (C3b)=(ODₛₐₘₚₗₑ−OD_{NC})/(OD_{PC}−OD_{NC})×100%. MBL is a carbohydrate-binding serum protein, which circulates in complex with serine proteases known as mannan-binding lectin associated serine proteases (MASPs). When bound to microorganisms, the MBL complex activates the complement components C4 and C2, thereby generating the C3 convertase and leading to opsonisation by the deposition of C4b and C3b fragments.

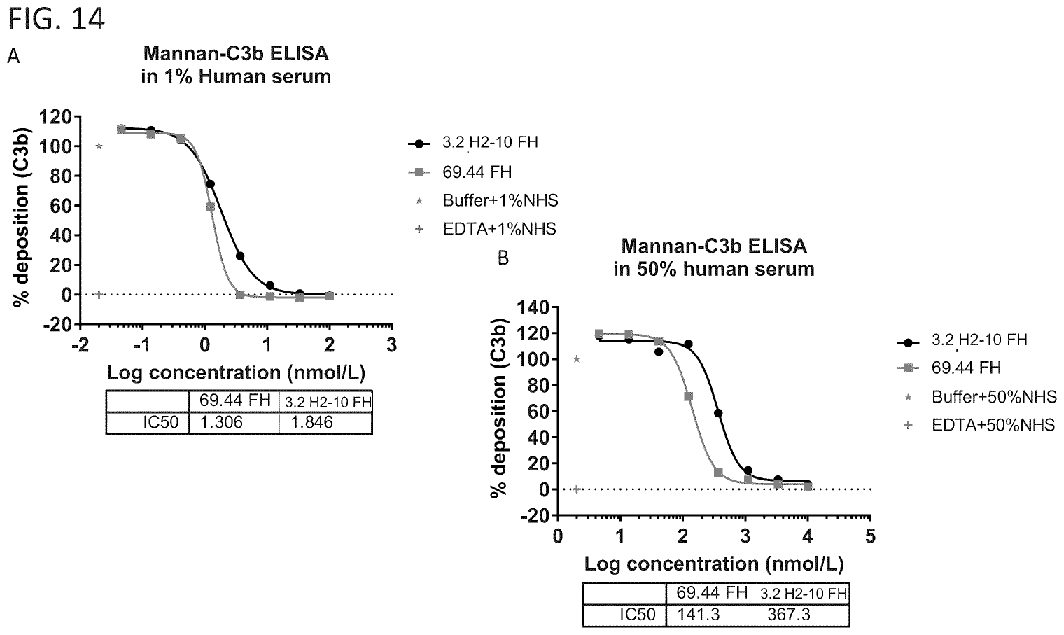

The results are shown in FIG. 14. 3.2 H2-10 FH and 69.44FH potently inhibited the lectin pathway with an IC₅₀ value of 1.85 nM and 1.31 nM, respectively, in 1% human serum. The IC₅₀ value of inhibition by 3.2 H2-10 FH and 69.44FH in 50% human serum were 367.3 nM and 141.3 nM, respectively.

### 4. LPS-C3b ELISA

An LPS-C3b ELISA was used to assess the anti-C2 factor H fusion constructs 3.2 H2-10 FH and 69.44FH in inhibition of the alternative pathway.

For LPS-C3b ELISA, serum samples were preincubated with serially diluted anti-C2 antibodies or fusion constructs at 4° C. for 1.5 h, then added to ELISA 96-well plate coated with LPS (500ng/well) and incubated at 37° C. for 1 h. The AP complement activation was stopped by adding 100 μl/well pre-cold 40 mM EDTA. After washing, the plate was incubated with 100 μl/well of anti-C3 antibody-HRP (1:4,000) at room temperature for 1 h, followed by washing, adding 100 μl/well of TMB to develop color, and then adding 50 μl/well of 2N H2SO₄ to stop the reaction. The absorbance at 450 nm was read on a densitometric plate reader. A sample with only the EDTA buffer added was used as the negative control (NC, 0% deposition), and a sample with only AP buffer (APB) added was used as the positive control (PC, 100% deposition). The % deposition of C3b of each sample was calculated with the following formula: % deposition (C3b)=(ODₛₐₘₚₗₑ−OD_{NC})/(OD_{PC}−OD_{NC})×100%.

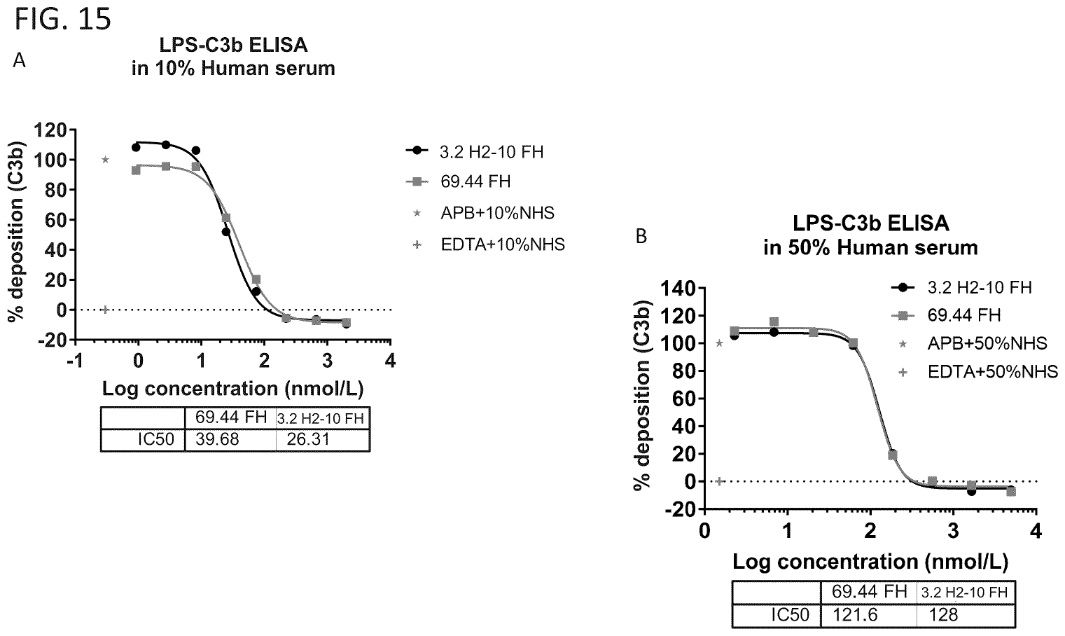

FIG. 15 shows that 3.2 H2-10 FH and 69.44FH-inhibited the alternative pathway with an IC₅₀ value of 26.32 nM and 39.68 nM, respectively, in 10% human serum. The IC₅₀ value of inhibition by 3.2 H2-10 FH1 and 69.44F1-H in 50% human serum was 128 nM and 121.6 nM, respectively.

### 5. IgM-C5b-9 ELISA

An IgM-C5b-9 ELISA was used to assess the anti-C2 factor H fusion constructs 3.2 H2-10 FH and 69.44F1-H in inhibition of the classical pathway-dependent C5 activation.

For IgM-C5b-9 ELISA, serum samples were preincubated with serially diluted anti-C2 antibodies or fusion constructs at 4° C. for 1 h, then added to ELISA 96-well plate coated with IgM (100ng/well) and incubated at 37° C. for 1 h. The CP complement activation was stopped by adding 100 μl/well pre-cold 40 mM EDTA. After washing, the plate was incubated with 100 μl/well of anti-human C5b-9 antibody (1:100) at room temperature for 1 h, followed by washing, adding 100 μl/well of anti-mouse IgG antibody-HRP (1:40,000) at room temperature for 1 h. After washing, 100 μl/well of TMB was added to develop color, and then the reaction was stopped by adding 50 μl/well of 2N H₂SO₄. The absorbance at 450 nm was read on a densitometric plate reader. A sample with only the EDTA buffer added was used as the negative control (NC, 0% deposition), and a sample with only GVB added was used as the positive control (PC, 100% deposition). The % deposition of C5b-9 of each sample was calculated with the following formula: % deposition (C5b-9)=(ODₛₐₘₚₗₑ−OD_{NC})/(OD_{PC}−OD_{NC})×100%.

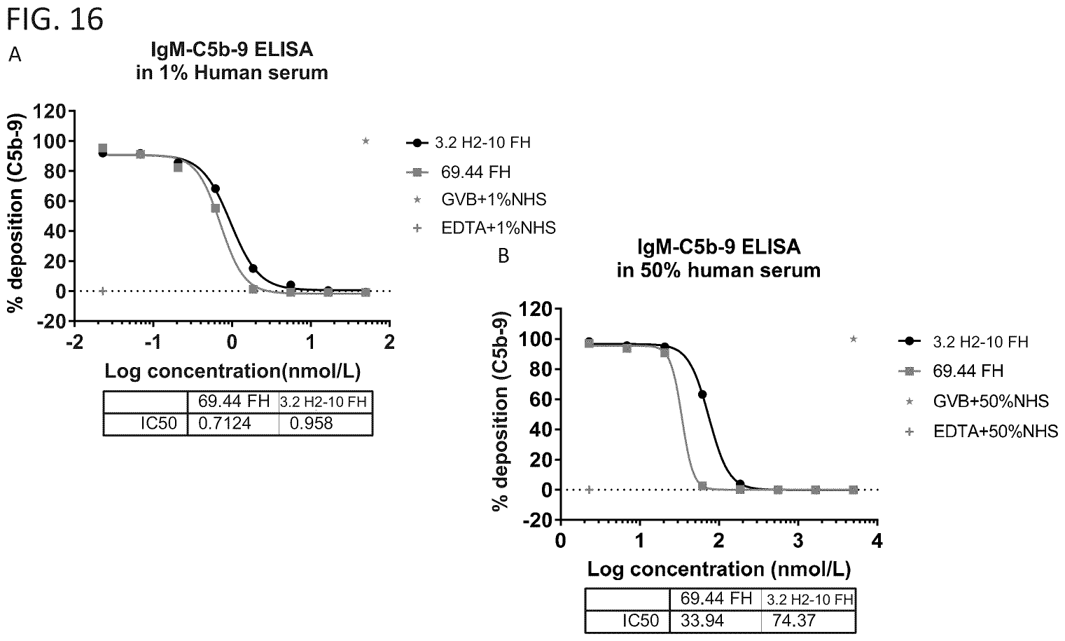

FIG. 16 shows that 3.2 H2-10 FH and 69.44 FH inhibited the classical/terminal pathway with an IC₅₀ value of 0.958 nM and 0.7124 nM, respectively, in 1% human serum. The IC₅₀ value of inhibition by 3.2 H2-10 FH and 69.44FH in 50% human serum was 74.37 nM and 33.94 nM, respectively.

### 6. LPS-C5b-9 ELISA

An LPS-C5b-9 ELISA was used to assess the anti-C2 factor H fusion constructs 3.2 H2-10 FH and 69.44FH in inhibition of the alternative pathway-dependent C5 activation.

For LPS-C5b-9 ELISA, serum samples were preincubated with serially diluted anti-C2 antibodies or fusion constructs at 4° C. for 1.5 h, then added to ELISA 96-well plate coated with LPS (500ng/well) and incubated at 37° C. for 1 h. The CP complement activation was stopped by adding 100 μl/well pre-cold 40 mM EDTA. After washing, the plate was incubated with 100 μl/well of anti-human C5b-9 antibody (1:100) at room temperature for 1 h, followed by washing, adding 100 μl/well of anti-mouse IgG antibody-HRP (1:40,000) at room temperature for 1 h. After washing, 100 μl/well of TMB was added to develop color, and then the reaction was stopped by adding 50 μl/well of 2N H₂SO₄. The absorbance at 450 nm was read on a densitometric plate reader. A sample with only the EDTA buffer added was used as the negative control (NC, 0% deposition), and a sample with only APB added was used as the positive control (PC, 100% deposition). The % deposition of C5b-9 of each sample was calculated with the following formula: % deposition (C5b-9)=(ODₛₐₘₚₗₑ−OD_{NC})/(OD_{PC}-OD_{NC})×100%.

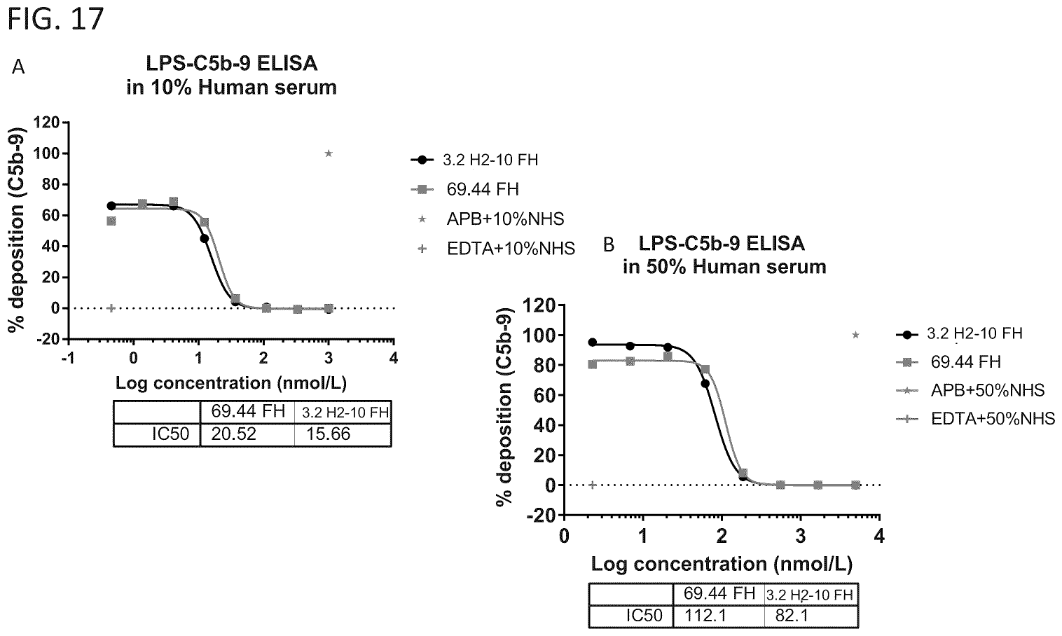

FIG. 17 shows that 3.2 H2-10 FH and 69.44FH inhibited the alternative/terminal pathway with an IC50 value of 15.66 nM and 20.52 nM, respectively, in 10% human serum. The IC50 value of inhibition by 3.2 H2-10 FH and 69.44FH in 50% human serum was 82.1 nM and 112.1 nM, respectively.

### Example 8: Pharmacokinetic (PK) and Pharmacodynamics Studies in Monkey

Single dose of 20 mg/kg or 60 mg/kg of FH fusion protein, or 20 mg/kg anti-C2 IgG1 antibodies were injected intravenously, each in 1 female and 1 male Cynomolgus monkey (Macaca fascicularis). Monkey blood was collected at designated time points pre- and post-dosing. Serum complement pathway activities (for both monoclonal antibodies and antibody-FH 1-5 fusions) were determined by sheep RBC hemolysis assay and rabbit RBC hemolysis assay, respectively. Briefly, the monkey serum samples collected at different time points were incubated with the sensitized sheep RBCs at 37° C. for 30 min, or incubated with the rabbit RBCs at 37° C. for 40 min, the reaction was then stopped by adding EDTA solution, after centrifugation, the supernatant was transfer to 96-well plates, and the absorbance at 405 nm was measured on BioTek microplate reader.

Drug concentration in monkey serum collected at different time points was measured by two different methods respectively for anti-C2 FH fusion proteins and anti-C2 IgG1 proteins. For anti-C2 fH fusion protein PK assay, Maxisorp plate was coated with mouse anti-human IgG (CH2 domain) at 4° C. overnight. After washing and blocking, serum samples were added to the plate and incubated at RT for 1 hr. After washing, mouse anti-human factor H (OX-24)-biotin was added to the plate to detect captured drug. After washing, HRP-streptavidin was added and incubated at RT for 30 min. The color was developed by adding TMB substrate and the absorbance was measured with a BioTek microplate reader. Calibration standards and quality controls were performed in each ELISA plate.

For anti-C2 IgG1 PK assay, a bridging assay using biotin-labeled and sulfo-TAG-labeled mouse anti-human IgG (CH2 domain) was developed and qualified. Briefly, serum samples were mixed with biotin-anti-CH2 and sulfo-anti-CH2, then the mixture was incubated at RT for 1 hr. Block the MSD High Bind plate with 3% BSA for 1 hr at RT. After washing, the above mixture was added to the plate and incubated for 1 hr at RT. After washing, 150 μL/well Read Buffer was added to the plate and the electrochemiluminescence signal was measured with a MSD SQ120 instrument. As for the anti-C2 MH fusion protein PK assay, calibration standards and quality controls for anti-C2 IgG1 were performed in each ELISA plate.

TABLE 11
Pharmacokinetics parameters and pharmacodynamics of anti-C2
mAb and FH fusion following single doses of anti-C2 in NHP

| | | PK | | | | | PD | | |
|---|---|---|---|---|---|---|---|---|---|
| Doses | | AUC0-t | AUC0-inf | Cmax | T½ | CL | % of | Days of | |
| (mg/kg) | Antibody | (μg · h/ml) | (μg · h/ml) | (μg/ml) | (d) | (L/d/kg) | inhibition | inhibition | |
| 20 | 69.44-FH- | 46747.3 | 49573.8 | 666.3 | 2.44 | 9.68E−03 | ~90 | 5 | (120 h) |
| | IgG4 | | | | | | | | |
| 60 | 69.44-FH- | 129971.6 | 148307.2 | 1403.8 | 3.46 | 9.71E−03 | ~90 | 10 | (240 h) |
| | IgG4 | | | | | | | | |
| 20 | 3.2 H2-10 | 45047.4 | 45668.6 | 601.9 | 2.07 | 1.05E−02 | ~90 | 5 | (120 h) |
| | FH-IgG4 | | | | | | | | |
| 60 | 3.2 H2-10 | 212278.1 | 319164.8 | 1749.8 | 7.30 | 4.51E−03 | ~90 | 12 | (288 h) |
| | FH IgG4 | | | | | | | | |
| 20 | 69.44-IgG1 | 102221.5 | 136658.2 | 732 | 7.31 | 3.51E−03 | 50-70 | 14 | (336 h) |
| 20 | 3.2 H2-10- | 64867.9 | 87493.8 | 725.6 | 3.89 | 5.49E−03 | ~60 | 8 | (192 h) |

IgG1

The PKPD results show T1/2 of 3.2 H2-10 FH and 69.44-FH were 7.3 and 3.46 days, respectively, when dosed at 60 mg/kg, higher than the T1/2 of 2.44- and 2.04-days respectively, when dosed at 20 mg/kg. pH dependent binding of C2 may contribute to the extended serum T1/2 for 3.2 H2-10 FH. Correlated to the PK, 3.2 H2-10 FH showed maximum inhibition of rabbit RBC lysis for 12 days while 69.44-FH showed 10 days of full inhibition of rabbit RBC lysis. IgG1 Fe appears to increase serum T1/2 anti-C2 to the antibodies. 3.2 H2-10 IgG1 and 69.44-IgG1 showed T1/2 of 3.89- and 7.31-days respectively, which were longer than the T1/2 their IgG4 counterpart under the same dose condition.

### Example 9: Pharmacokinetic (PK) and Pharmacodynamics (PD) Studies for 3.2(WT), 69.44, and 3.2 pH in Mice

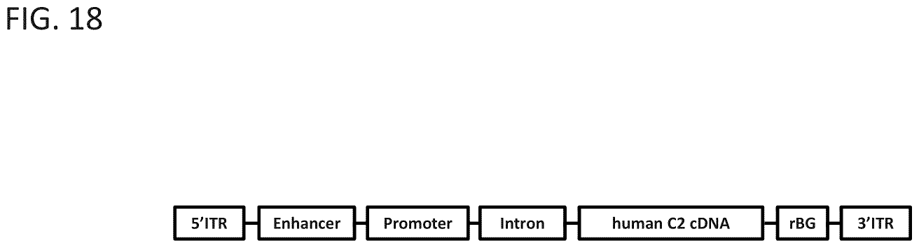

Mice expressing human C2 were generated to test the pharmacokinetic (PK) and pharmacodynamic (PD) properties of anti-C2 antibodies. Mice injected with an AAV carrying human C2 cDNA (FIG. 18) were measured for human C2 plasma concentration two weeks post-injection using an ELISA. While SCID/FnRn (Jackson Laboratory, strain #: 018441) mice given a single injection of 1×10¹⁰ or 1×10¹¹ particles of the human C2 AAV yielded low concentrations of human C2 (3.5-11.8 μg/ml), a single injection of 1×10¹² particles resulted in a high concentration of human C2 (67.2-87.78 μg/ml). C57BL/6J (B6) wild type mice showed a viral quantity-dependent increase of human C2 concentration in the plasma. A single injection of 1×10¹¹ particles of the human C2 AAV into B6 mice resulted in detection of 35.37-61.86 μg/ml human C2, whereas a single injection of 3×10¹¹ particles of the human C2 AAV into C57BL/6J wild type mice resulted in human C2 concentrations of 63.47-80.77 μg/ml. A single injection of 1×10¹² particles led to human C2 concentrations ranging from 67.18-151.16 μg/ml. Based on these preliminary studies, 3×10¹¹ AAV particles was chosen for injection into SCID/FnRn mice. Subsequent studies showed this dosage produced human C2 plasma concentrations averaging about 42 μg/ml across ten mice.

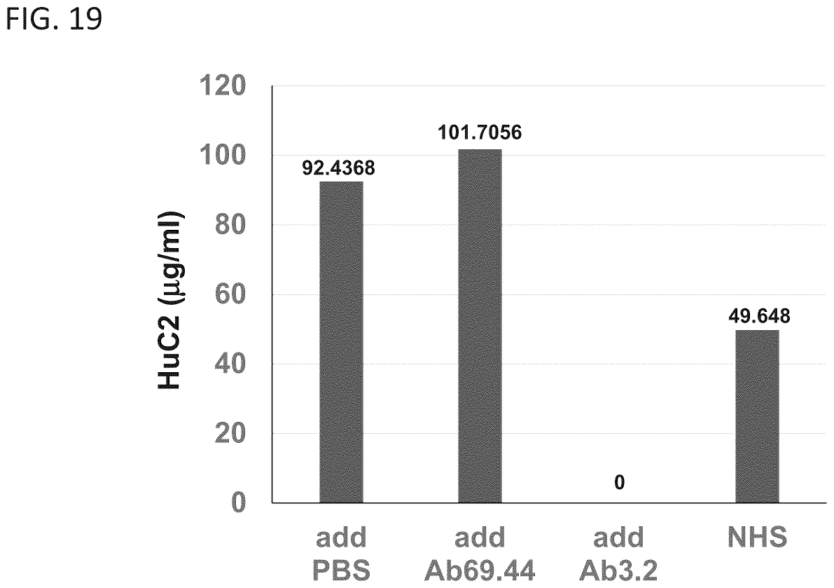

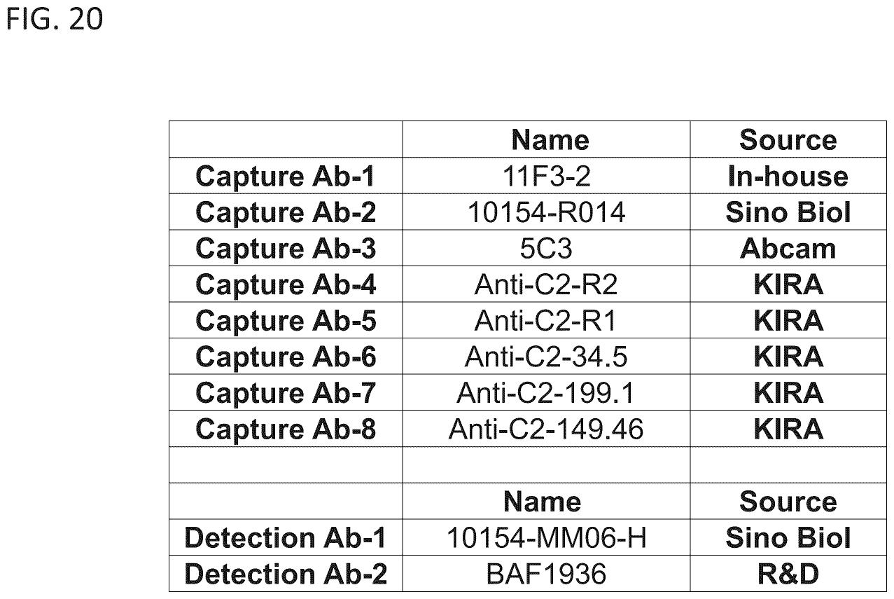

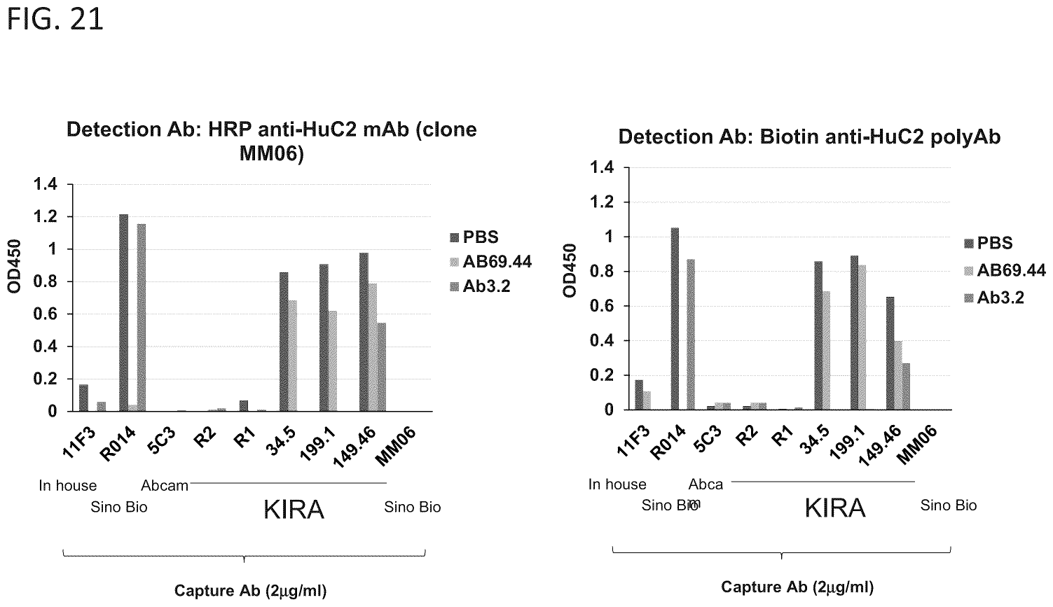

Optimization of the ELISA assay for human C2 was performed using different combinations of capture and detection antibodies. Plasma from a SCID/FnRn mouse injected with 3×10¹¹ human C2 AAV particles was mixed 1:1 with either PBS, 400 μg/ml 69.44, or 400 μg/ml 3.2. The mixture was incubated on ice for 30 min and measured for human C2 using an ELISA. As shown in FIG. 19, when the capture antibody was the anti-human C2 monoclonal antibody (mAb) clone 11F3 and the detection antibody was HRP anti-human C2 clone MM06 (Sino Biologicals), 69.44 was detected, but 3.2 did not show any signal. FIG. 20 shows the various capture and detection antibodies used to optimize the human C2 ELISA assay so both 3.2 and 69.44 could be detected. As shown in FIG. 21, use of an HRP anti-human C2 mAb (10154-MM06-H, Sino Biologicals) for the detection antibody resulted in detection of only 3.2 when 10154-R014 and 149.46 were used as the capture antibodies, detection of only 69.44 when 34.5 and 199.1 were used as the capture antibodies, and detection of both 3.2 and 69.44 when 149.46 was used as the capture antibody. When a biotin anti-human C2 polyclonal antibody (polyAb) was used as the detection antibody, only 3.2 was detected with 10154-R014 as the capture antibody, only 69.44 was detected with 34.5 and 199.1 as the capture antibodies, and both 3.2 and 69.44 were detected with 149.46 as the capture antibody, but to lower levels than when MM06 was used as the detection antibody. Based on these results, in order to perform total human C2 ELISAs to test for C2 accumulation, testing of 69.44 used 11F3 for the capture antibody and MM06-HRP for the detection antibody while testing of 3.2 used 10154-R014 (Sino Biologicals) for the capture antibody and MM06-HRP for the detection antibody.

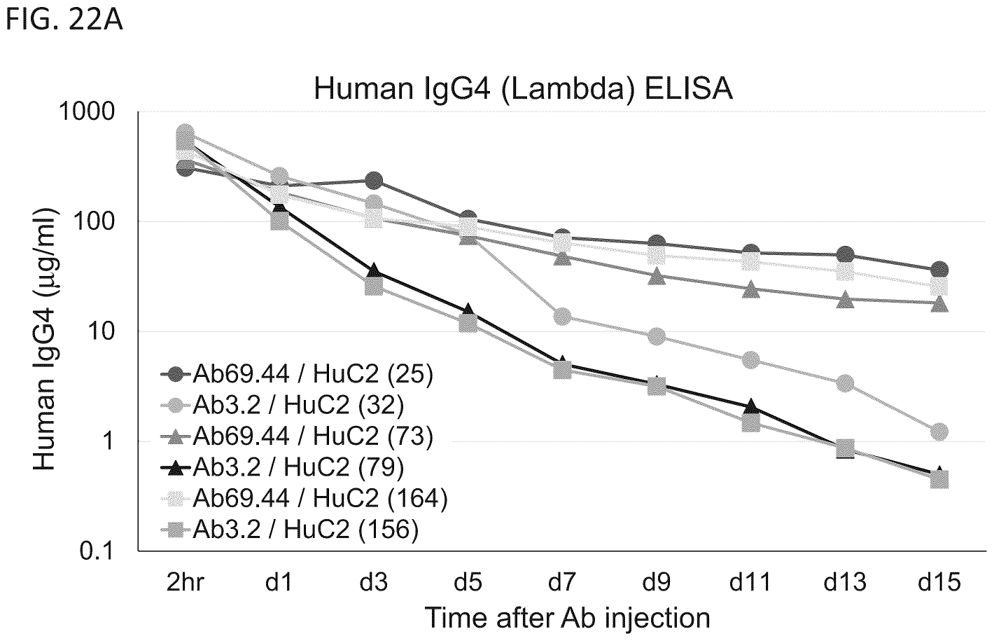

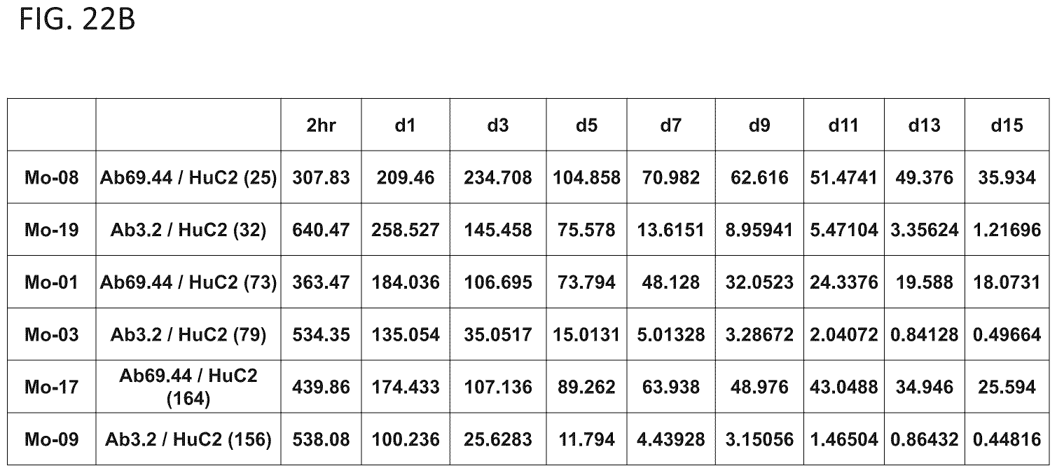

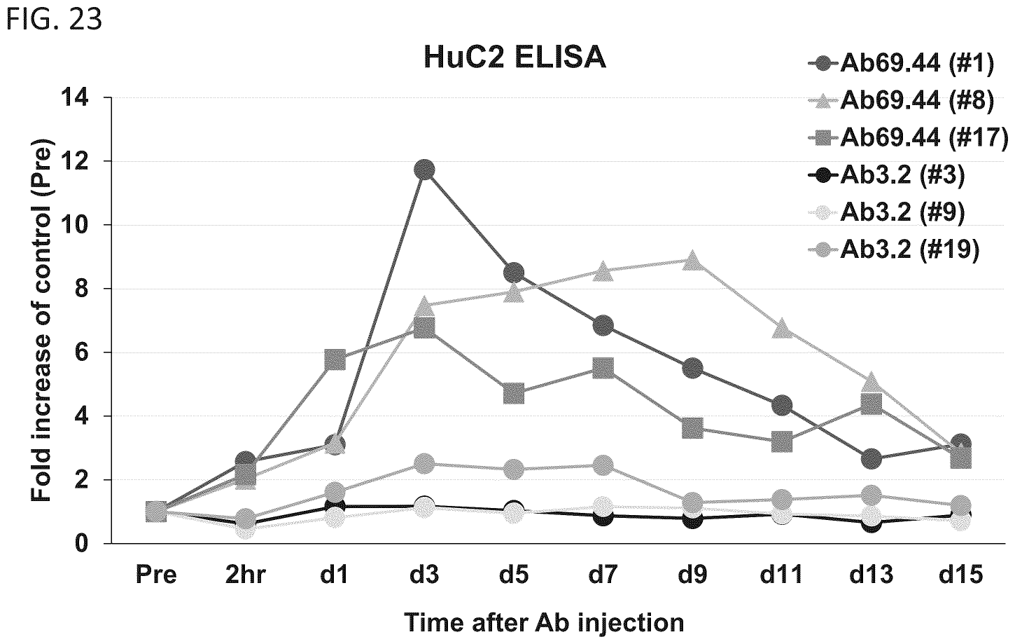

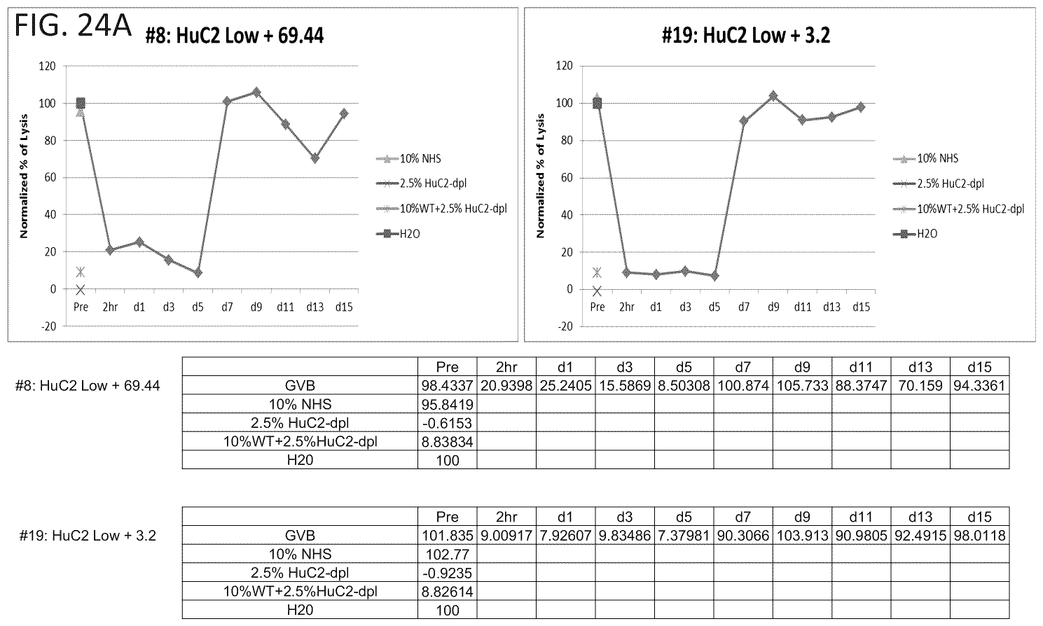

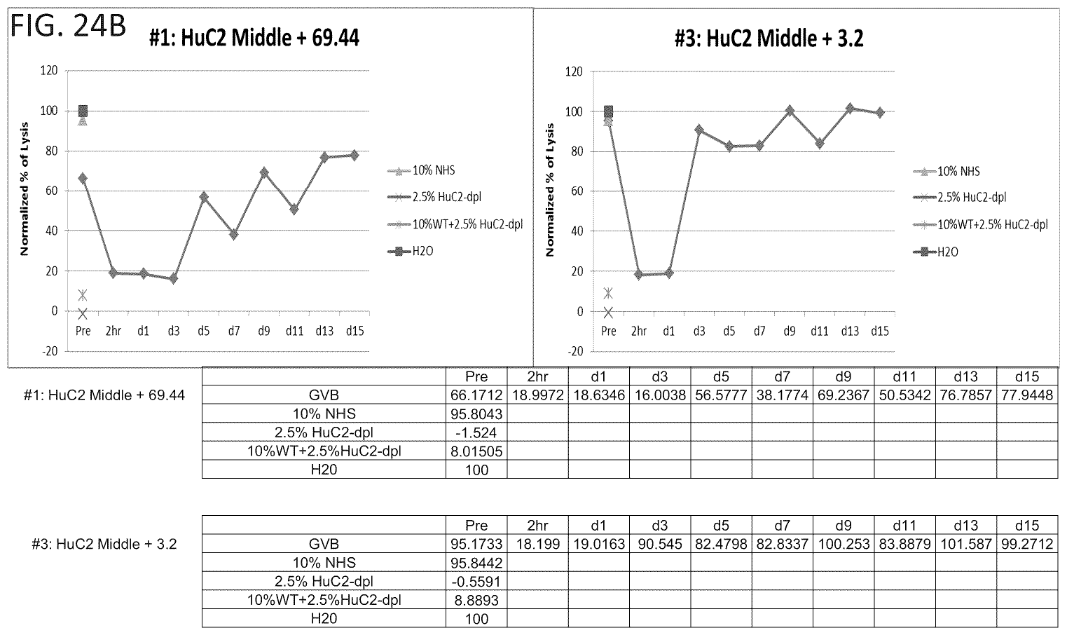

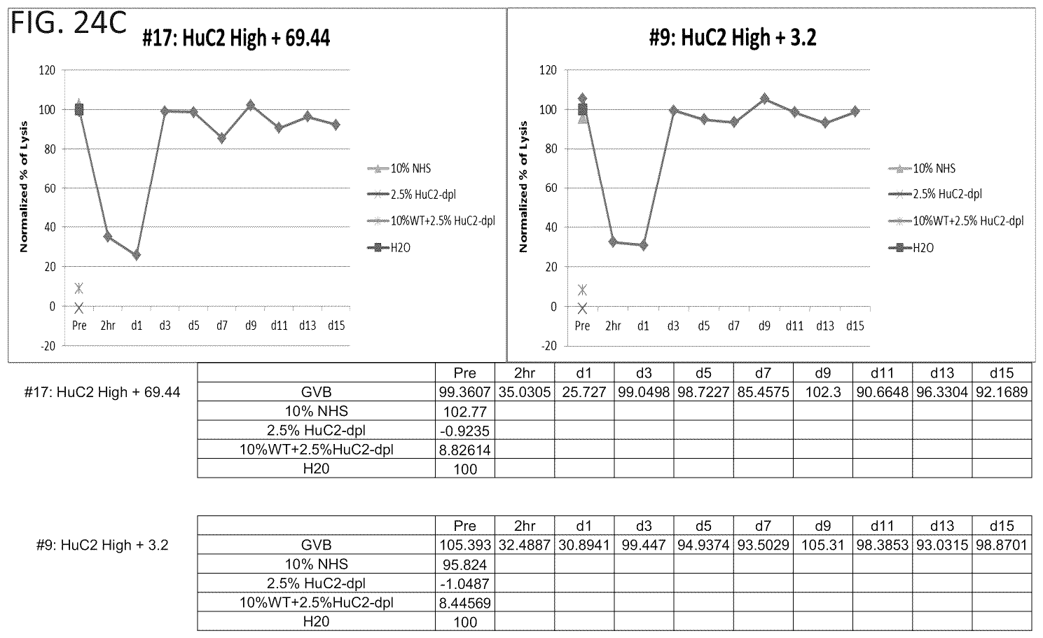

To determine whether anti-human C2 clone 3.2 or 69.44 had better binding, 40 mg/kg antibody was injected intravenously (i.v.) and tested for binding in SCID/FcRn mice that expressed low (HuC2 low, HuC2 (25), or HuC2 (32)), middle (HuC2 middle, HuC2 (73), or HuC2 (79)), or high (HuC2 high, HuC2 (164), or HuC2 (156)) levels of human C2. Plasma was collected at different time points (pre-injection, 2 hrs, day 1 (D1), D3, D5, D7, D9, D11 and D15). A human IgG4 (lambda) ELISA was used for PK analysis, a sheep RBC lysis assay was used for PD analysis, and a human C2 ELISA was used for C2 accumulation analysis. As shown in FIGS. 22A and 22B, the 69.44 clone had better binding than the 3.2 clone regardless of the expression levels of human C2 in mice. However, the 69.44 clone showed 10-fold higher C2 accumulation than the 3.2 clone regardless of the expression levels of human C2 in mice (FIG. 23). To determine the C2 blocking activity of the anti-human C2 mAbs in C2-humanized mice, a PD assay measuring the degree of sheep red blood cell (RBC) lysis was performed in 10% humanized mouse serum+2.5% C2-depleted normal human serum (NHS). Water and 10% NHS were used as positive controls for 100% lysis. 2.5% NHS depleted of C2 (“2.5% HuC2-dp1”) as well as 10% normal mouse serum together with 2.5% NHS depleted of C2 (“10% WT+2.5% HuC2-dp1”) were negative controls that showed no lysis. As shown in FIGS. 24A-24C, prior to dosing the mice (at timepoint “pre”) with anti-C2 mAbs, 100% sheep RBC were lysed as expected, except for 69.44 injected into a HuC2 middle mouse (FIG. 24B, left). In HuC2 low mice, both 69.44 and 3.2 inhibited lysis by 80-90% from 2 hrs up to 5 days (FIG. 24A). While injection of 69.44 into a HuC2 middle mouse led to 80% lysis inhibition from 2 hrs to 3 days, injection of 3.2 into a HuC2 middle mouse led to the same amount of lysis inhibition for up to 1 day (FIG. 24B). 69.44 and 3.2 introduced into HuC2 high mice resulted in 25-35% lysis inhibition for up to 1 day (FIG. 24C). Full lysis activity was restored when the anti-human C2 mAbs were metabolized.

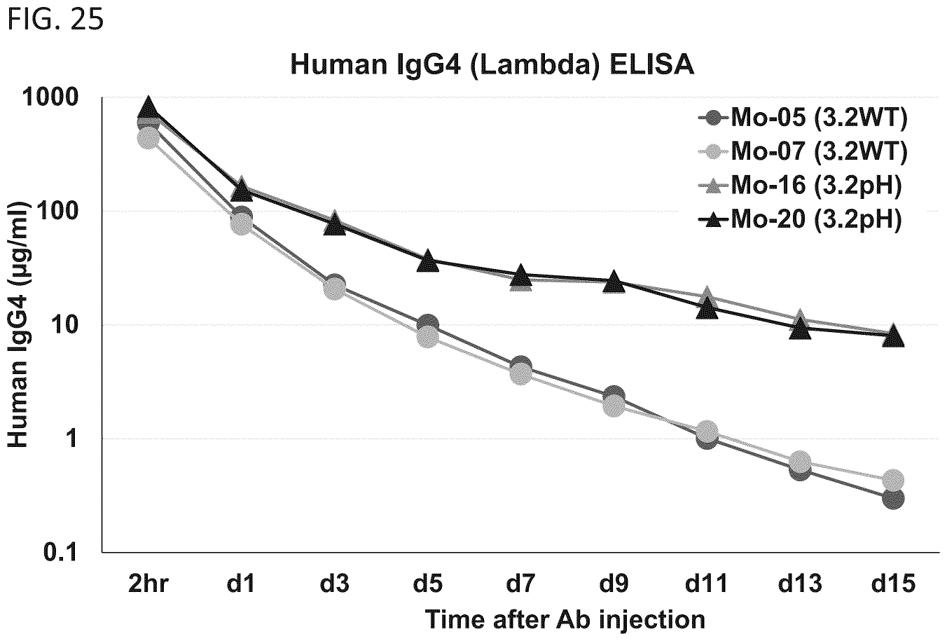

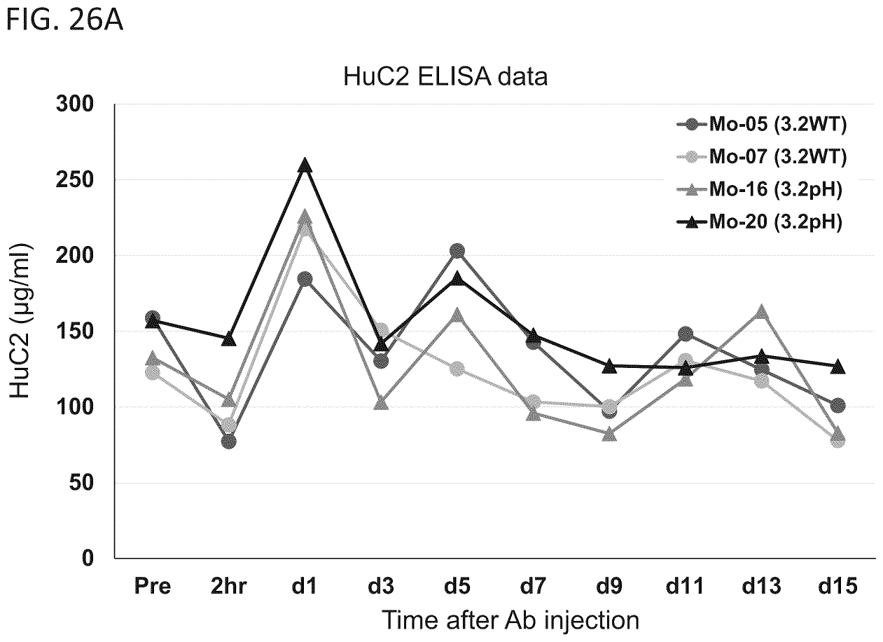

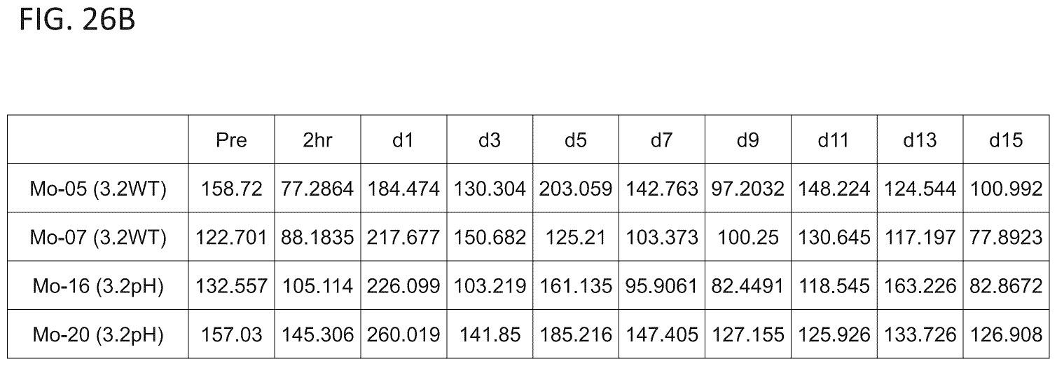

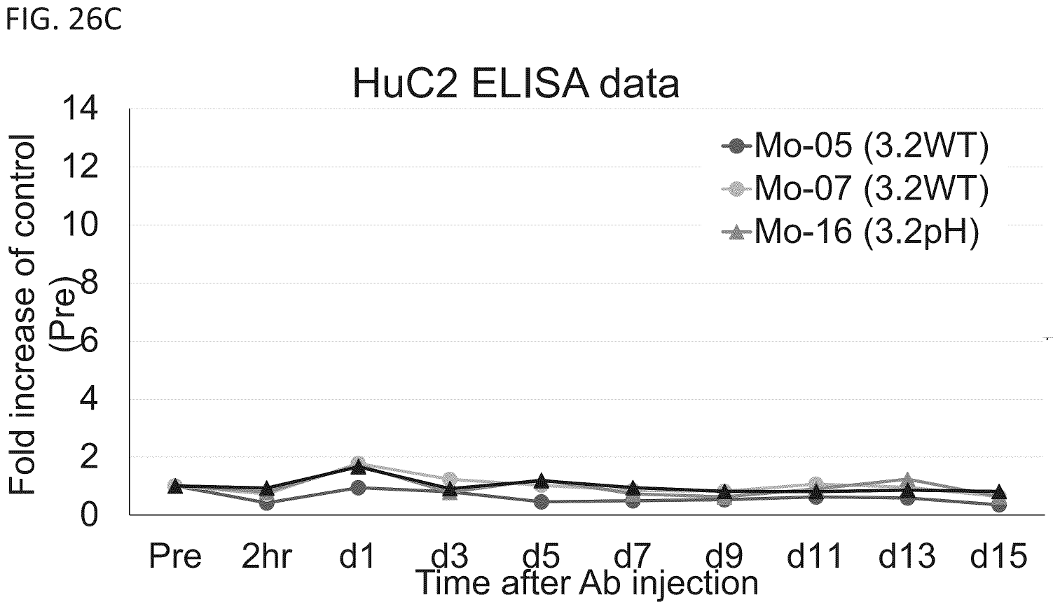

Clone 3.2 was ideal because it did not lead to C2 accumulation. Thus, to improve the PK of the antibody, a pH-dependent clone (3.2 pH) was generated. 40 mg/kg antibody was injected (i.v.) and tested for binding in SCID/FcRn mice that expressed high levels of human C2, plasma was collected at different time points, and a human IgG4 (lambda) ELISA was used for PK analysis while a human C2 ELISA was used for C2 accumulation analysis. As shown in FIG. 25, 3.2 pH exhibited better binding than 3.2WT. Additionally, the C2 accumulation for 3.2 pH was comparable to that of 3.2WT (FIGS. 26A and 26B) and showed no fold increase compared to pre-injection control (FIG. 26C).

TABLE 12
Pharmacokinetics parameter (T½) of anti-C2
mAb following single doses of anti-C2 in mice

| | | Average |
|---|---|---|
| Dose (mg/kg) | Antibody | T½ (day) |
| 40 | 149.46 | 2.14 |
| 40 | 69.44 | 6.88 |
| 40 | 199.1 | 1.51 |
| 40 | 34.5 | 1.66 |
| 40 | 3.2 | 2.27 |
| 40 | 3.2 pH | 4.51 |
| 40 | 69.44-IgG1 | 10.9 |
| 40 | 69.44-IgG4 | 4.3 |
| 40 | 3.2 pH-IgG1 | 4.3 |
| 40 | 3.2 pH-IgG4 | 2.8 |
| 40 | 3.2 pH-IgG1 LALA-LS-FH | 3.94 |
| 40 | 3.2 pHGL-IgG1 LALA-LS-FH | 2.9 |
| 40 | 3.2 pH-IgG4-PLA-FH | 2.16 |
| 40 | 69-IgG4-PLA-FH | 2.92 |

Of the anti-human C2 clones (3.2, 34.5, 199.1, 69.44, 149.46), 69.44 had the longest average T1/2 (6.88 days) while 34.5 and 199.1 had the shortest average T1/2 (1.66 and 1.51 days, respectively). Between 3.2 and the pH-dependent 3.2 clone (3.2 pH), 3.2pH had a twice longer half-life of 4.51 days. Between 69.44 and 3.2 pH, which had the two longest half-lives, the T1/2 was better with the IgG1 construct in both cases, compared to those with an IgG4 isotype. Additionally, fusion to factor H led to shortened half-lives for both 3.2 and 69.44, compared to only the IgG1 and IgG4 constructs. The germline version of 3.2 pH-IgG1 LALA-LS-FH (“3.2pHGL-IgG1 LALA-LS-FIH”) had a shorter T1/2 at 2.9 days, compared to the non-germline version, which had a half-life of 3.94 days.

### Example 10: Generation of Germline-Derived Anti-C2 mAbs of the 3.2 Clone

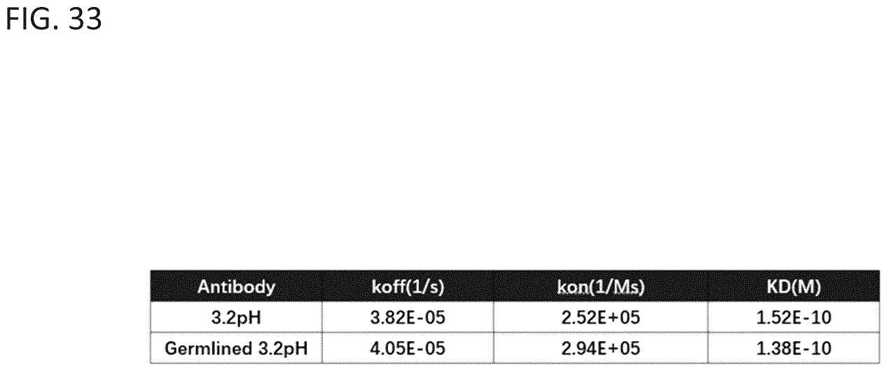

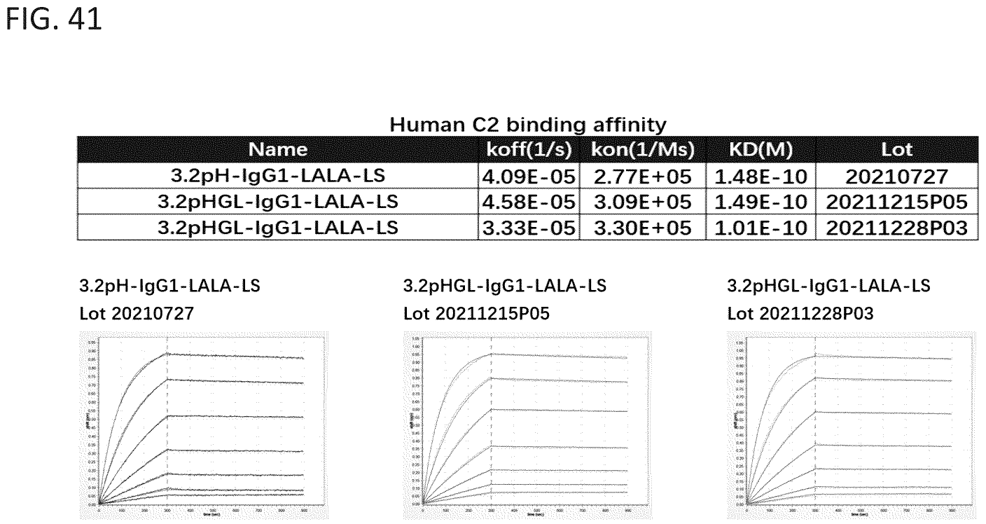

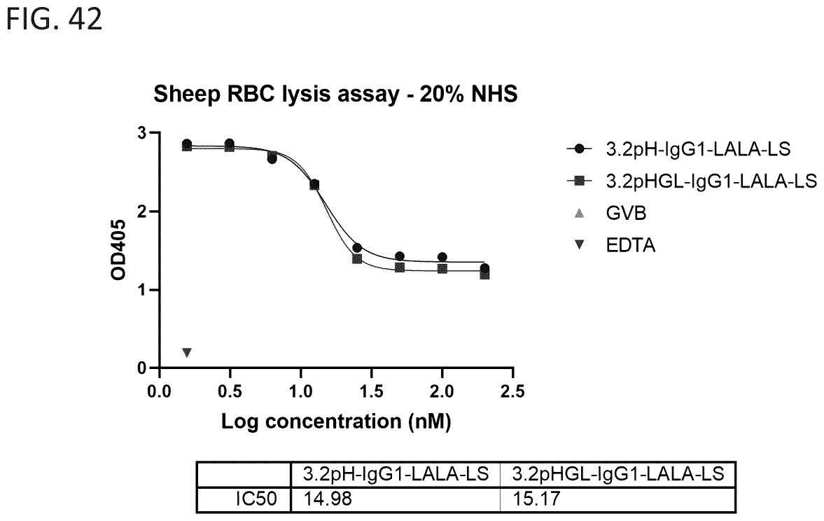

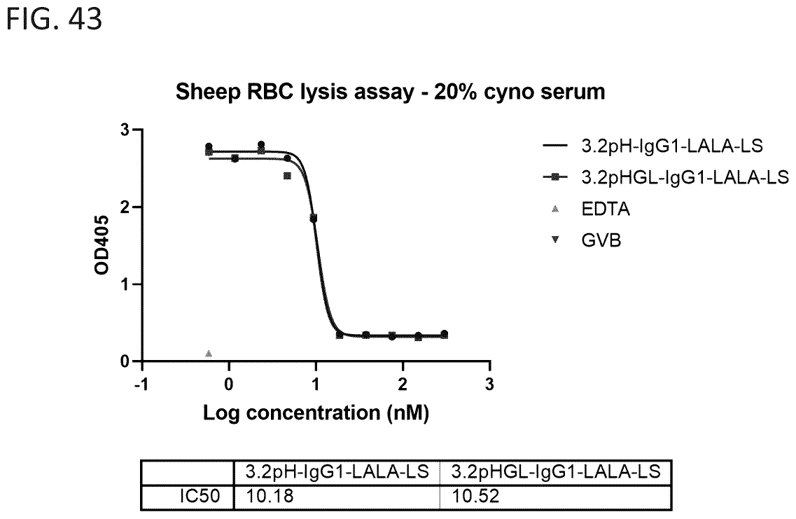

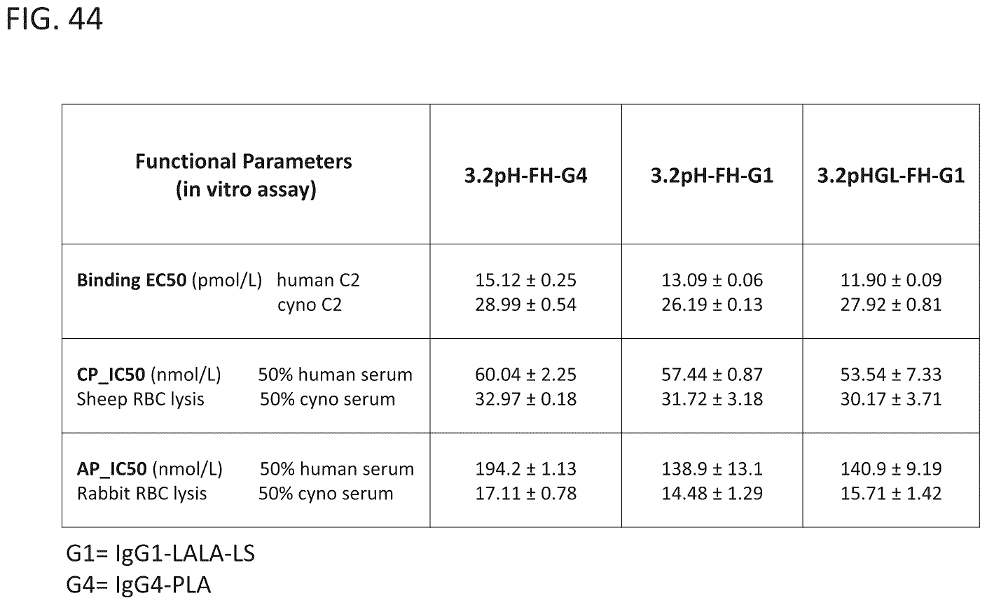

A 3.2 pH construct (3.2pHGL) derived from a human germline framework sequence was generated. 3.2pHGL presented similar association and dissociation rates compared to the original 3.2 pH construct (FIG. 33). 3.2 pH-IgG1-LALA-LS derived from germline (“3.2pHGL-IgG1-LALA-LS”) demonstrated nearly identical binding affinity and biological activity as measured by a sheep RBC lysis assay, compared to 3.2 pH-IgG1-LALA-LS that was derived from the OmniRat hybridoma platform (FIGS. 41-43). Furthermore, the germline fusion protein (3.2pHGL-FH-IgG1-LALA-LS) presented identical biological activities to hybridoma-derived 3.2 pH-FH-IgG4-PLA and 3.2 pH-FH-IgG1-LALA-LS when compared to the corresponding human or cynomolgus in vitro assays (FIG. 44).

### Example 11: Pharmacokinetic (PK) and Pharmacodynamics (PD) Studies for 69.44-FH, 3.2WT, 3.2pHGL, and 3.2pHGL-FH in Mice

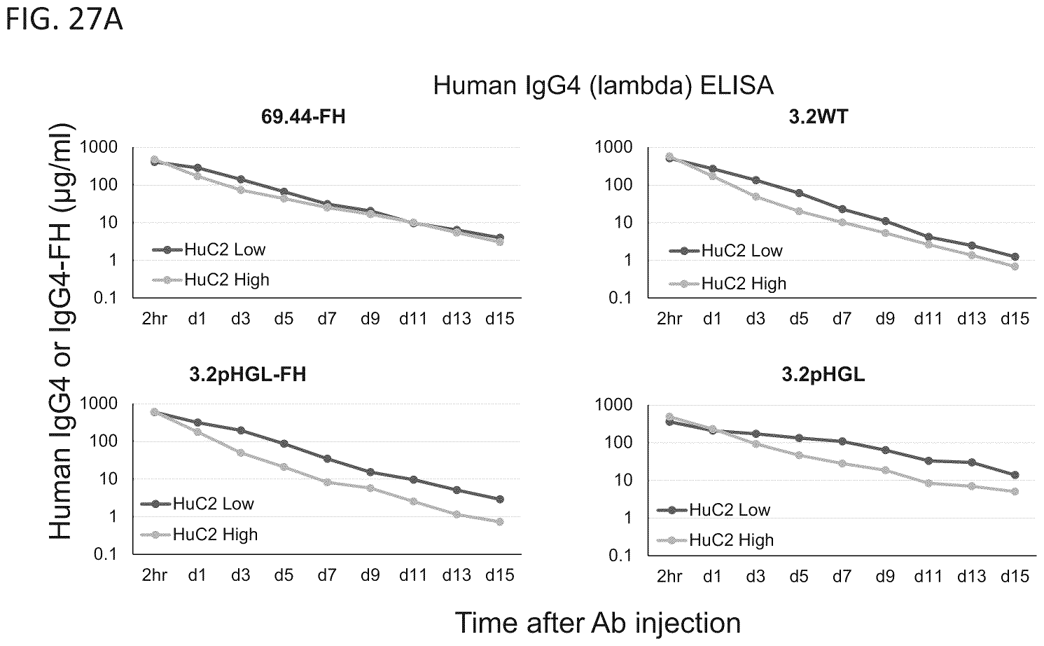

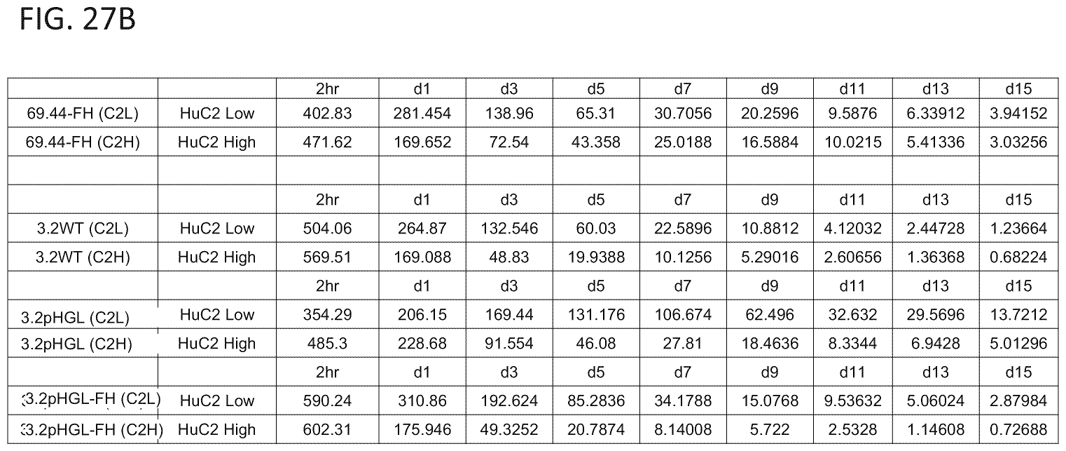

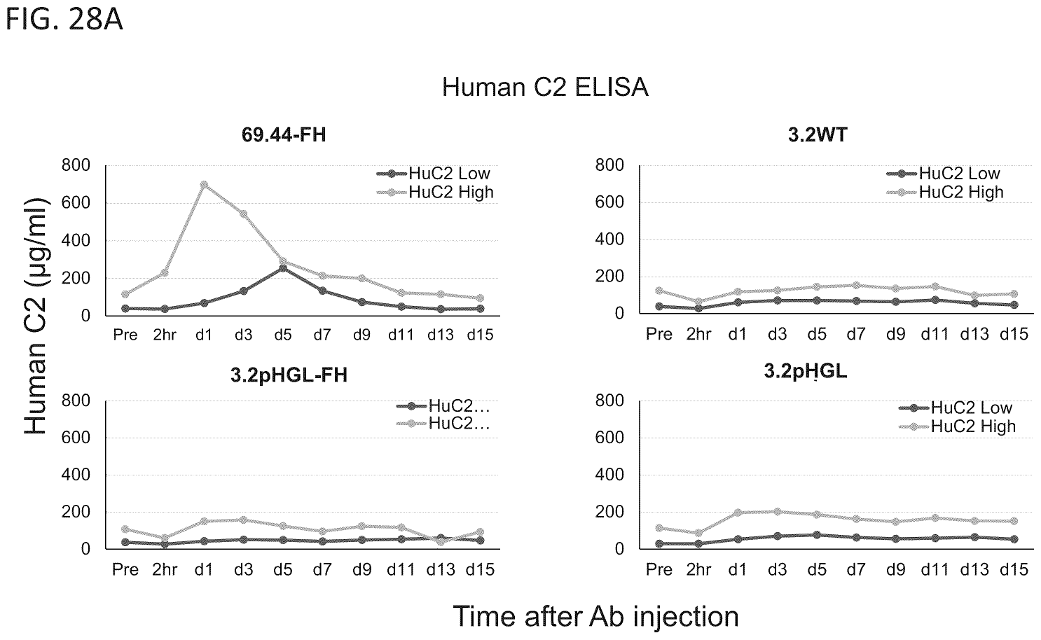

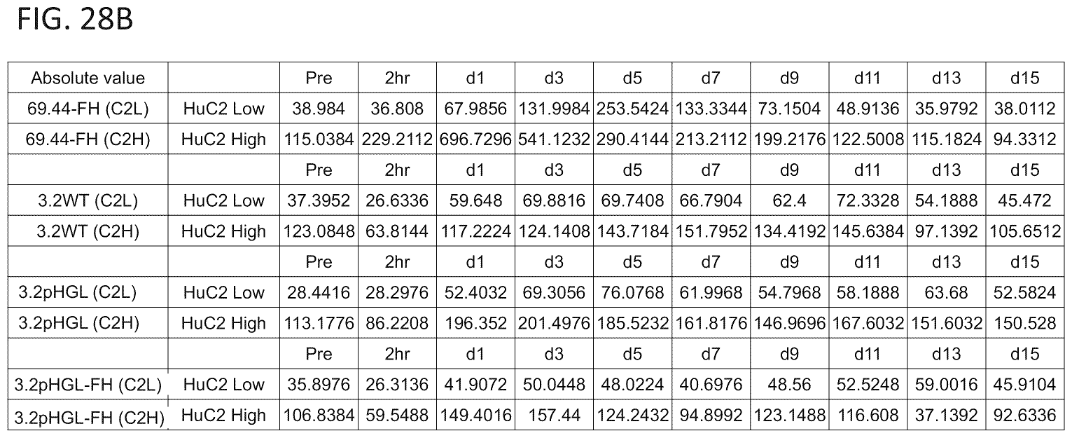

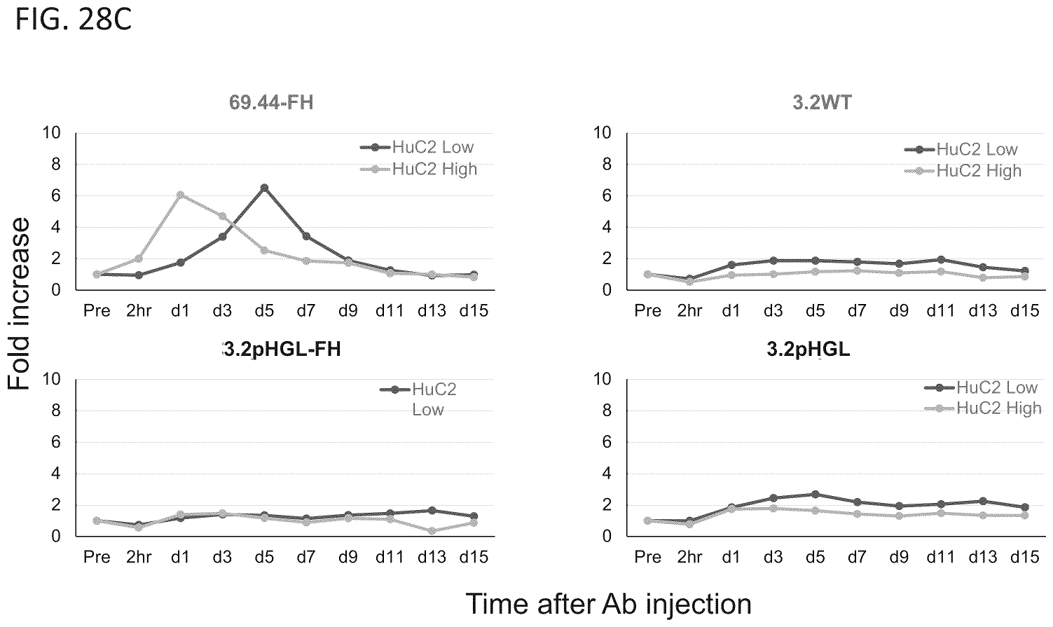

SCID/FnRn mice expressing low or high levels of human C2 were injected (i.v.) with 40 mg/kg 69.44-FH, 3.2WT, 3.2pHGL, or 3.2pHGL-FH. Plasma was collected at different time points (pre-injection, 2 hrs, D1, D3, D5, D7, D9, D11 and D15). A human IgG4 (lambda) ELISA was used for PK analysis and a human C2 ELISA was used for C2 accumulation analysis. As shown in FIGS. 27A and 27B, 69.44-FH had comparable binding regardless of human C2 levels in mice. 3.2WT, 3.2pHGL, and 3.2pHGL-FH had better binding in mice expressing low human C2 levels. However, 69.44-FH had high C2 accumulation while the other three constructs did not result in C2 accumulation regardless of human C2 levels in mice (FIGS. 28A-28C).

### Example 12: Pharmacokinetic (PK) and Pharmacodynamics (PD) Studies for 64.99, 149, 199, and 34.5 in Mice

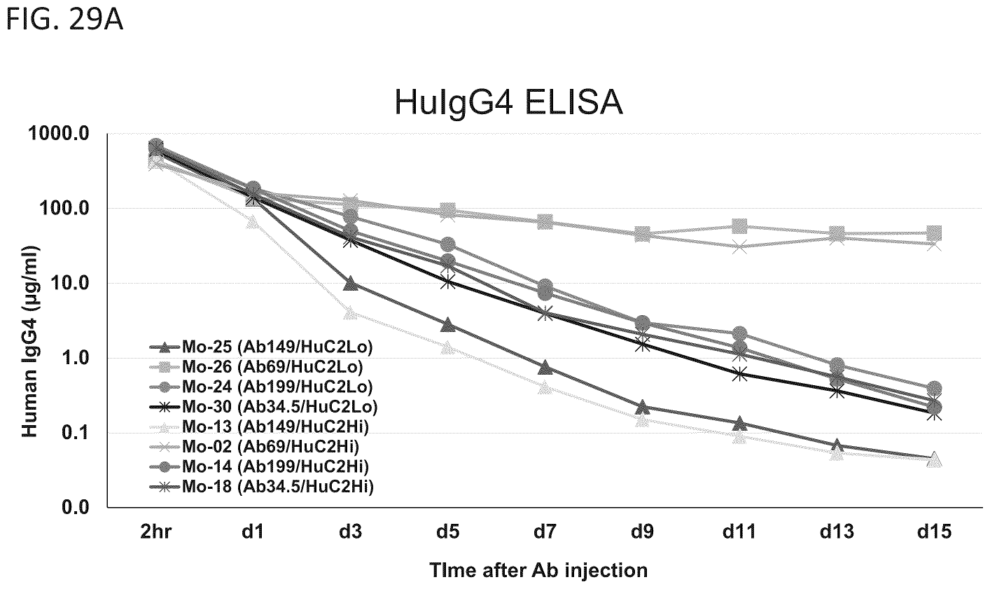

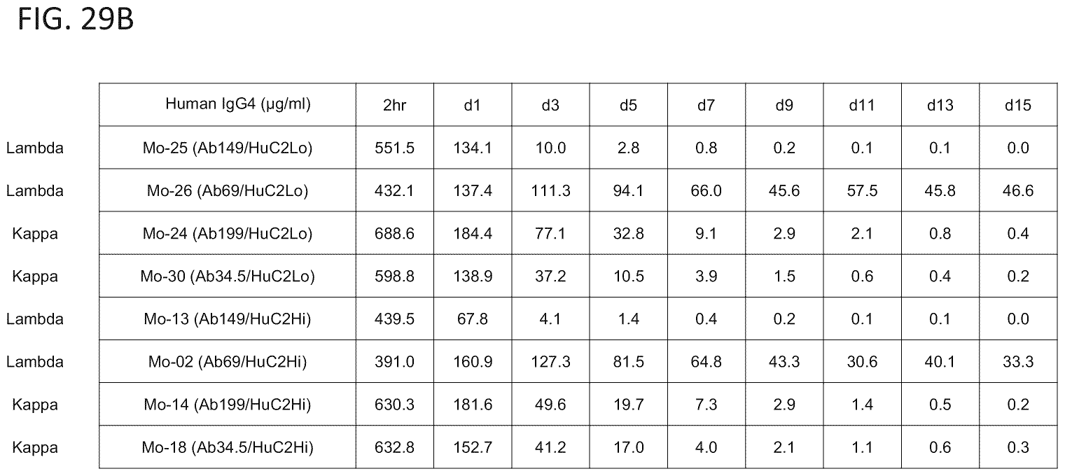

SCID/FnRn mice expressing low or high levels of human C2 were injected (i.v.) with 40 mg/kg 69.44, 149, 199, or 34.5. Plasma was collected at different time points (pre-injection, 2 hrs, D1, D3, D5, D7, D9, D11 and D15). To determine the PK and PD of four different anti-human C2 mAbs, a human IgG4 (lambda) ELISA was used for PK analysis and a human C2 ELISA was used for C2 accumulation analysis. As shown in FIGS. 29A and 29B, 69.44 exhibited the best binding regardless of human C2 expression levels in mice. The binding of 34.5 and 199 was between those of 69.44 and 149.

### Example 13: Pharmacokinetic (PK) and Pharmacodynamics (PD) Studies for 69.44-IgG1, 69.44-IgG4, 3.2 pH-IgG1, and 3.2 pH-IgG4 in Mice

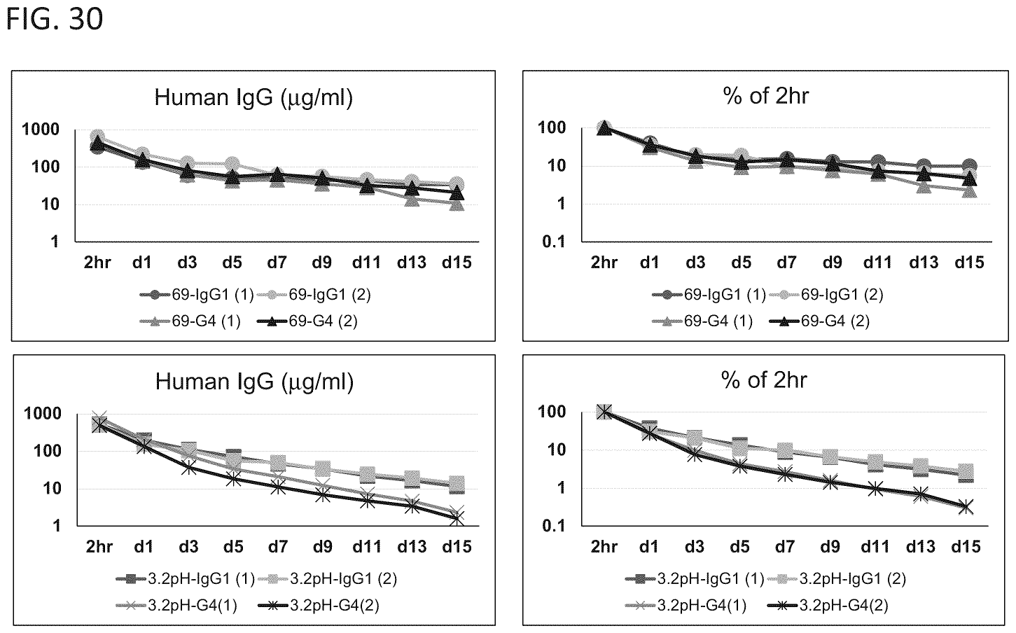

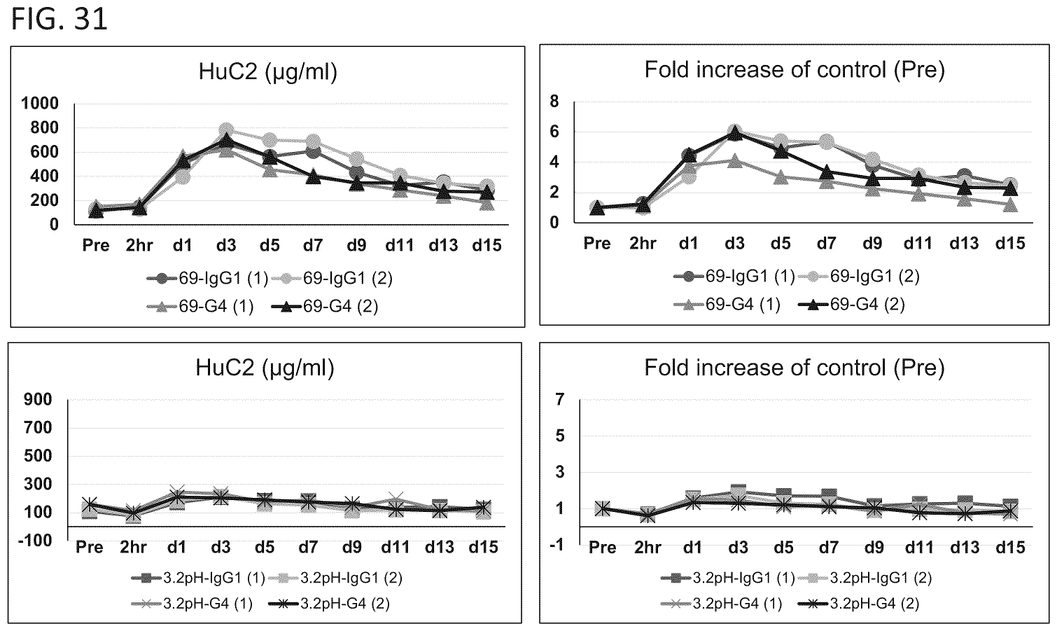

SCID/FnRn mice expressing low or high levels of human C2 were injected with 40 mg/kg 69.44-IgG1, 69.44-IgG4, 3.2pH-IgG1, or 3.2 pH-IgG4 (n=2/group). Plasma was collected at different time points (pre-injection, 2 hrs, D1, D3, D5, D7, D9, D11 and D15). To determine whether IgG1 constructs have improved PK over IgG4 constructs, a human IgG4 (lambda) ELISA was used for PK analysis. To determine whether 69.44-IgG1 accumulates C2 to the same degree as 69.44-IgG4, a human C2 ELISA was used for C2 accumulation analysis. While binding was similar between 69.44-IgG1 and 69.44-IgG4, binding for 3.2pH-IgG1 was better than that of 3.2 pH-IgG4 (FIG. 30). This suggested that IgG1 had improved PK over IgG4 constructs for 3.2 pH clones. In addition to no PK improvement for the 69.44 constructs with different IgGs, both 69.44-IgG1 and 69.44-IgG4 had high C2 accumulation, with up to 6-fold increase in C2 accumulation on day 3 (FIG. 31, top). Conversely, both 3.2 pH-IgG1 and 3.2 pH-IgG4 led to minimal C2 accumulation with highest accumulation on day 3 at a nearly 2-fold increase compared to pre-injection control sample, but this accumulation was not present by day 9 (FIG. 31, bottom).

### Example 14: Pharmacokinetic (PK) and Pharmacodynamics (PD) Studies for 69.44-IgG4-FH and 3.2 pH-IgG4-FH in Mice

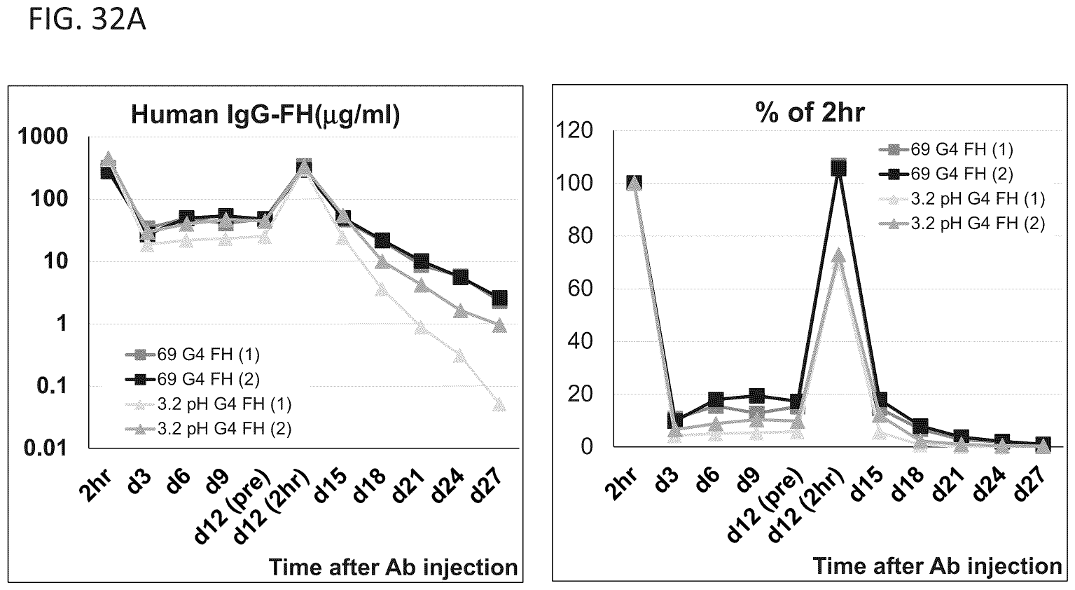

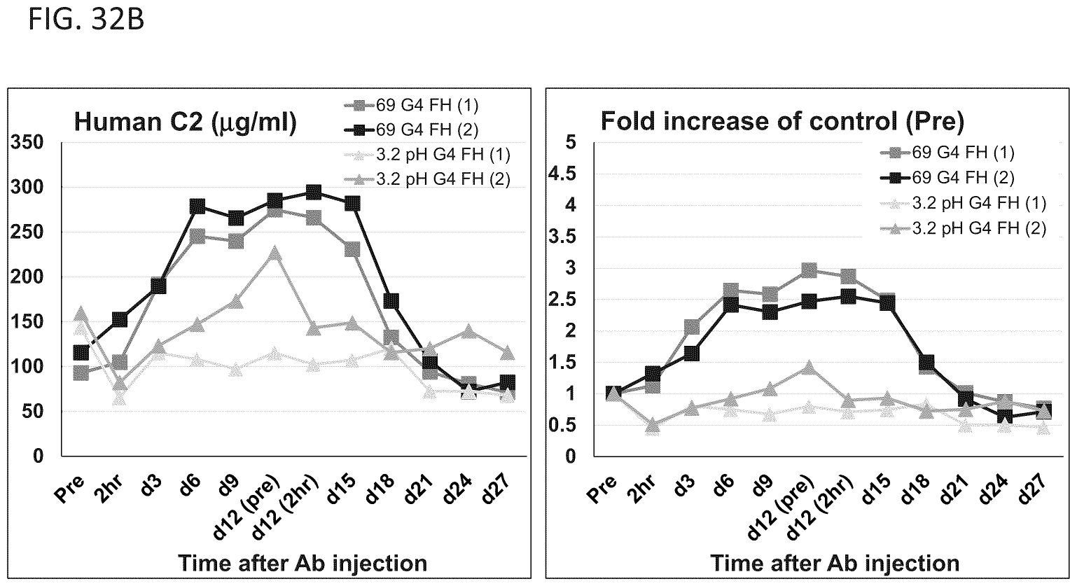

SCID/FnRn mice expressing low or high levels of human C2 were repeatedly injected (five times every three days at D0, D3, D6, D9, and D12) with 30 mg/kg 69.44-IgG4-FH and 3.2 pH-IgG4-FH (n=2/group). Plasma was collected at different time points (D0, D3, D6, D9, and D12). Plasma collected on days 3, 6, and 9 were collected prior to dosing. Plasma was collected prior to and after dosing on day 12. To compare fusion proteins containing IgG1 or IgG4, a human IgG1 or IgG4 ELISA was used for PK analysis and a human C2 ELISA was used for C2 accumulation analysis. 69.44-IgG4-FH exhibited the best binding, and the replicates were comparable (FIG. 32A). The 3.2 pH-IgG4-FH construct had similar binding compared to 69.44-IgG4-FH up to day 15. Although the 69.44 fusion constructs resulted in high C2 accumulation (up to 3-fold increase on D12, pre-dosing), 3.2pH-IgG4-FH exhibited either no C2 accumulation or only a 1.5-fold increase of C2 at day 12 (pre-dosing), compared to the pre-dosing control time point (FIG. 32B).

### Example 15: Pharmacokinetic (PK) and Pharmacodynamics (PD) Studies for 3.2 pH IgG4 PLA, 3.2 pH IgG4 PLS, 3.2 pH IgG1 LALALA, and 3.2 pH IgG1 LALALS in Mice

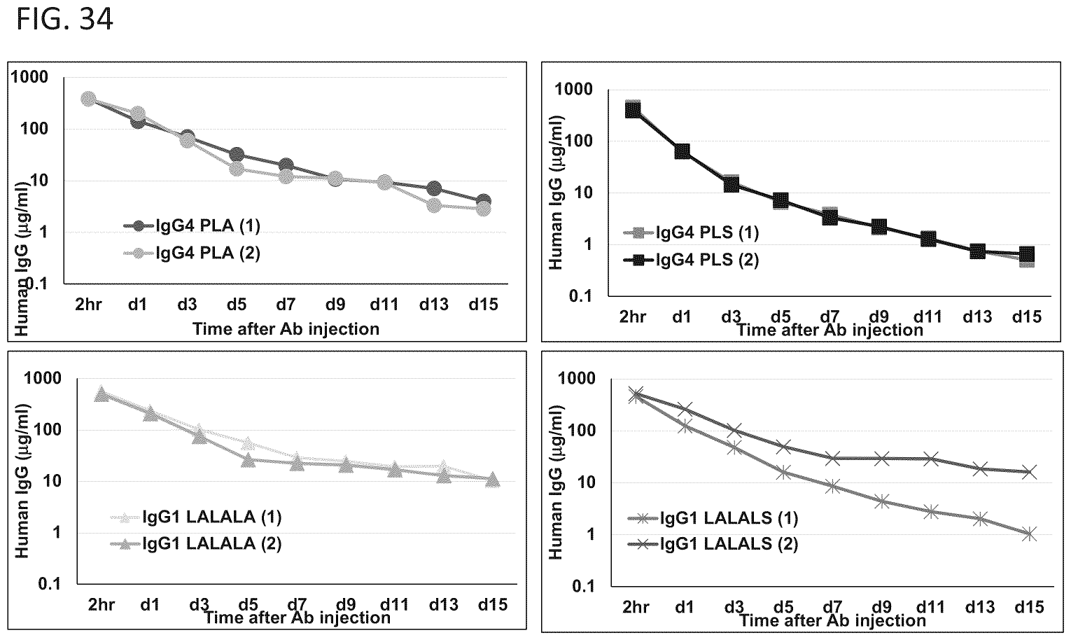

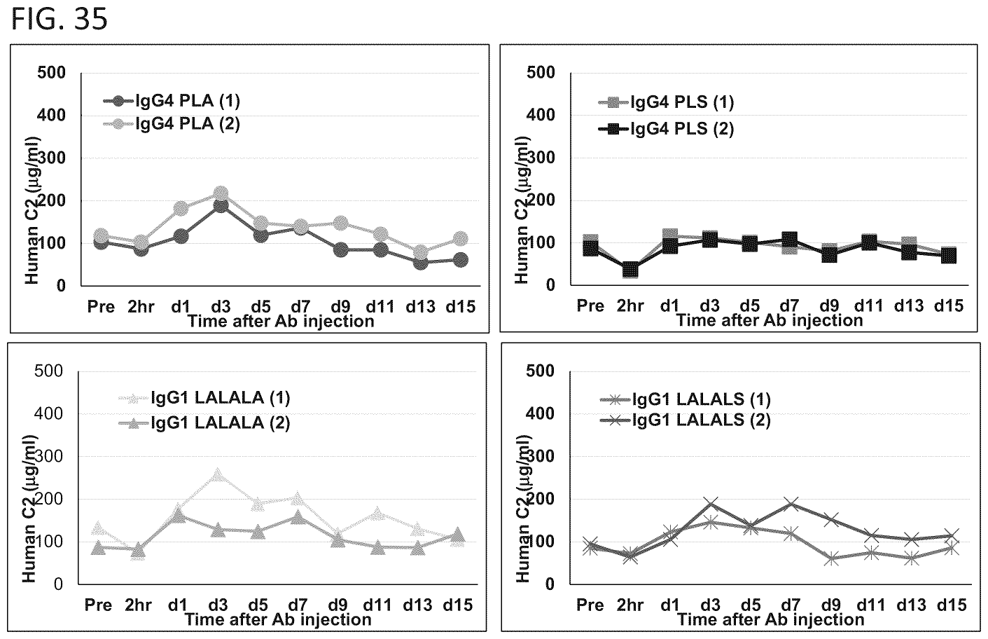

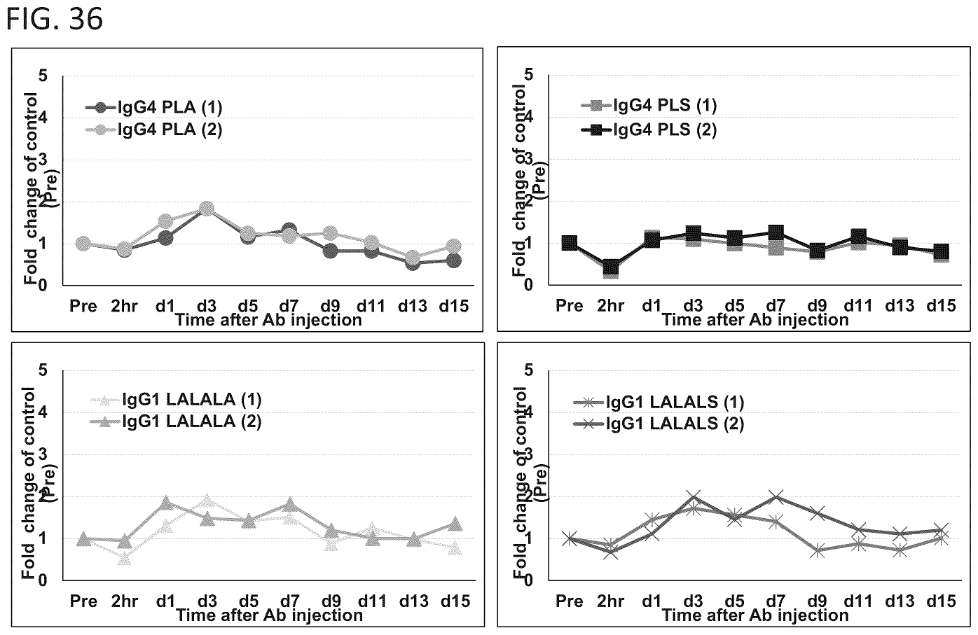

SCID/FnRn mice expressing low or high levels of human C2 were injected with 40 mg/kg 3.2 pH IgG4 PLA, 3.2 pH IgG4 PLS, 3.2 pH IgG1 LALALA, or 3.2 pH IgG1 LALALS (n=2/group). Plasma was collected at different time points (pre-injection, 2 hrs, D1, D3, D5, D7, D9, D11 and D15). A human IgG1 or IgG4 ELISA for PK analysis and a human C2 ELISA for C2 accumulation analysis were used to determine whether mutations in the constant domain of IgG1 or IgG4 for the 3.2 pH construct improved PK and C2 accumulation, respectively. Clone R10Z8E9 (MCA5748G, Bio-Rad) was used as the capture antibody and HRP anti-human IgG (9040-05, Southern Biotech) was used as the detection antibody in the human IgG1 and IgG4 sandwich ELISAs. The injected antibody for each mouse served as the standard for human IgG1 and IgG4 quantification. For the human C2 sandwich ELISA, clone 10154-R014 (Sino Biologicals) was used as the capture antibody and HRP anti-human C2 (10154-MM06-H, Sino Biologicals) was used as the detection antibody. Human C2 (Complement Tech) was used for the standard, samples were diluted 1:1600 to 1:102400, and developed for 90 seconds. 3.2 pH IgG4 PLA showed higher binding from days 1-15, compared to 3.2 pH IgG4 PLS (FIG. 34, top). Although the results for the 3.2 pH IgG1 LALALS replicates were inconsistent, the second replicate (3.2 pH IgG1 LALALS (2)) showed better binding than 3.2 pH IgG1 LALALA from days 9-15 (FIG. 34, bottom). C2 accumulation for 3.2 pH IgG4 PLA was 2-fold greater at day 3 while there was consistently no accumulation for 3.2 pH IgG4 PLS (FIGS. 35, top and 36, top). Both the 3.2 pH IgG1 LALALA and 3.2 pH IgG1 LALALS constructs resulted in nearly 2-fold accumulation of C2 from days 1-7 (FIGS. 35, bottom and 36, bottom).

### Example 16: Pharmacokinetic (PK) and Pharmacodynamics (PD) Studies for 69 IgG4 PLA-FH, 3.2 pH IgG4 PLA-FH, 3.2pHGL-IgG1 LALA-LS-FH (Germline), and 3.2 pH-IgG1 LALA-LS-FH in Mice

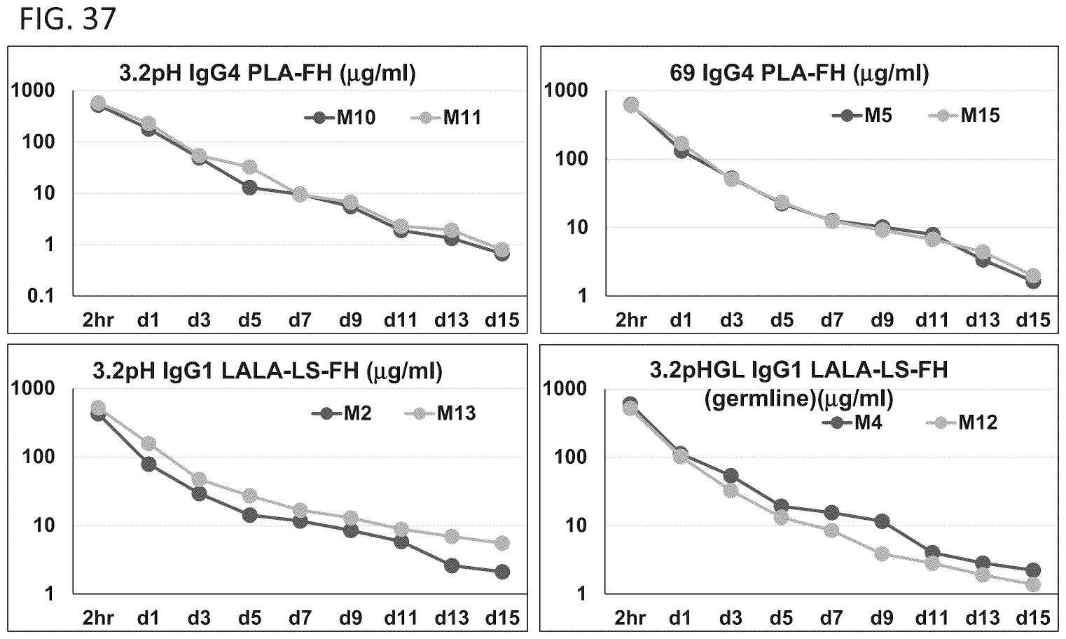

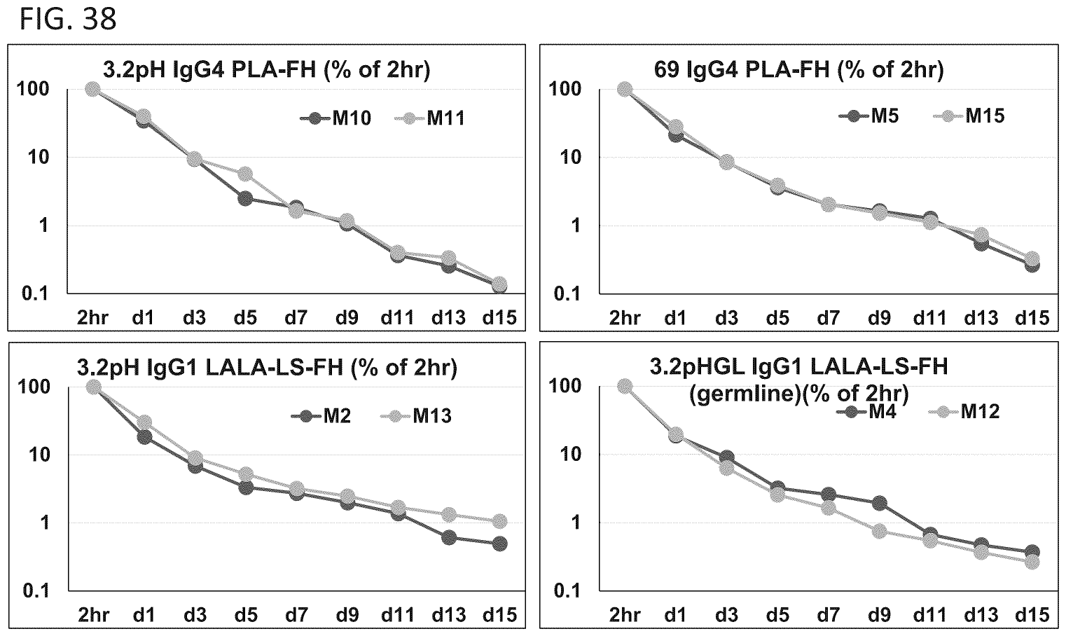

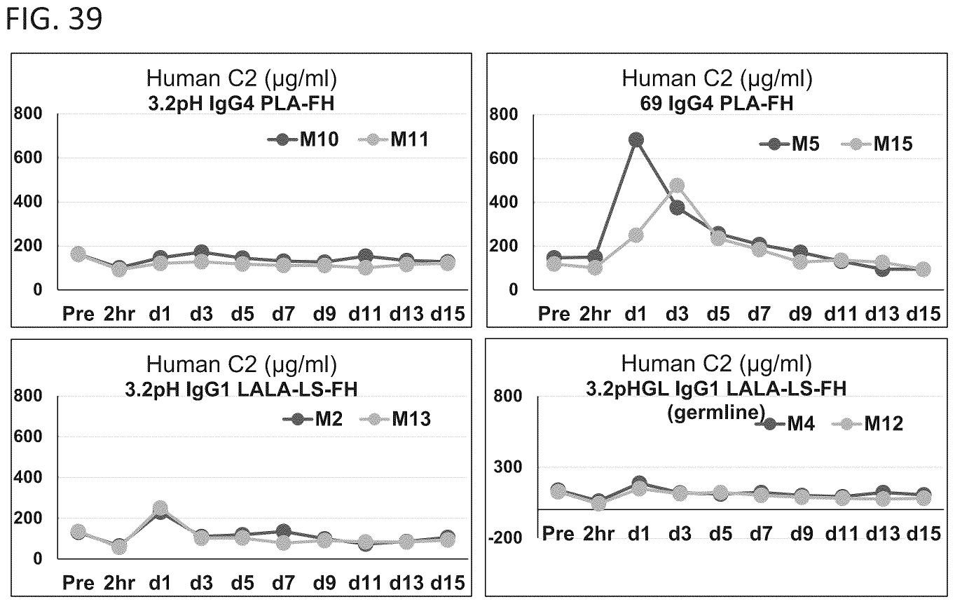

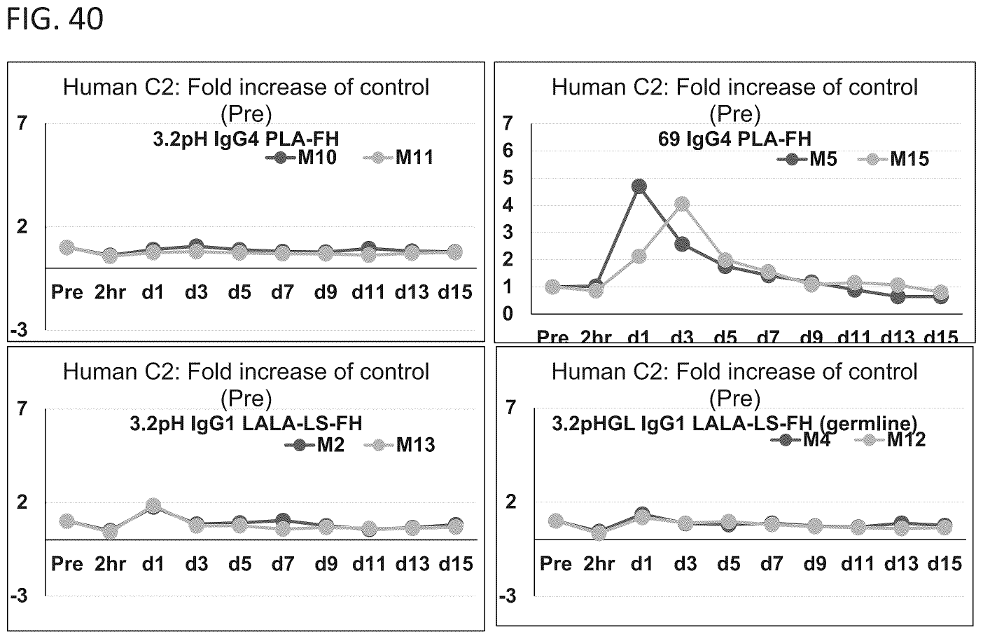

SCID/FnRn mice expressing low or high levels of human C2 were injected with 40 mg/kg 69 IgG4 PLA-FH, 3.2 pH IgG4 PLA-FH, 3.2pHGL-IgG1 LALA-LS-FH, or 3.2 pH-IgG1 LALA-LS-FH (n=2/group). Plasma was collected at different time points (pre-injection, 2 hrs, D1, D3, D5, D7, D9, D11 and D15). A human IgG1 or IgG4 E LISA for PK analysis and a human C2 ELISA for C2 accumulation analysis were used to determine whether mutations in the constant domain of IgG1 or IgG4 for the 69 and 3.2 pH fusion constructs improved PK and C2 accumulation, respectively. Clone R10Z8E9 (MCA5748G, Bio-Rad) was used as the capture antibody and HRP anti-human IgG (9040-05, Southern Biotech) was used as the detection antibody in the human IgG1 and IgG4 sandwich ELISAs. The injected antibody for each mouse served as the standard for human IgG1 and IgG4 quantification. For the human C2 sandwich ELISA, clone 10154-R014 (Sino Biologicals) was used as the capture antibody and HRP anti-human C2 (10154-MM06-H, Sino Biologicals) was used as the detection antibody. Human C2 (Complement Tech) was used for the standard, samples were diluted 1:1600 to 1:102400, and developed for 90 seconds. Beginning on day 3, 69 IgG4 PLA-FH exhibited higher binding than 3.2 pH IgG4 PLA-FH (FIGS. 37, top and 38, top). 3.2pHGL-IgG1 LALA-LS-FH showed better binding than 3.2 pH-IgG1 LALA-LS-FH from days 1-9 (FIGS. 37, top and 38, bottom). While the 69 IgG4 PLA-FH construct resulted in 4- to 5-fold increase in C2 accumulation on days 1 and 3 (FIGS. 39, top right and 40, top right), the 3.2 pH IgG4 PLA-FH construct showed no C2 accumulation over time (FIGS. 39, top left and 40, top left). Both 3.2 pH-IgG1 LALA-LS-FH and 3.2pHGL-IgG1 LALA-LS-FH similarly showed no C2 accumulation except at day 1, when there was 2-fold and 1.5-fold increase in C2 accumulation increase for 3.2 pH-IgG1 LALA-LS-FH and 3.2pHGL-IgG1 LALA-LS-FH respectively (FIGS. 39, bottom and 40, bottom).

SEQUENCE LISTING

| SEQ | | |
|---|---|---|
| ID NO: | Notes | Sequences |
| 1. | 34.5 VH | QVQLQESGPGLVKPSQTLSLTCTVSGGSISRGGHQWTWIRQHPGKGLEWI |
| | | GYIHHSGSTYYNPSLNDRLTILVDTPKNQFSLKLSSVTAADTAVYYCARW |
| | | NLGSYGDYWGQGTLVTVSS |
| 2. | 34.5 VL | AIVMTQSPDSLAVSLGERATINCKSSQSVLYSPNNKNYLAWYLQKPGQPP |
| | | KLLIYWASTRESGVPDRFSGSGSGTDFTLTISSLQAEDVAVYYCQQYYTTP |
| | | LTFGGGTKVEIK |
| 3. | 34.5 H-CDR1 | RGGHQWT |
| | (Kabat) | |
| 4. | 34.5 H-CDR2 | YIHHSGSTYYNPSLND |
| | (Kabat) | |
| 5. | 34.5 H-CDR3 | WNLGSYGDY |
| | (Kabat) | |
| 6. | 34.5 L-CDR1 | KSSQSVLYSPNNKNYLA |
| | (Kabat) | |
| 7. | 34.5 L-CDR2 | WASTRES |
| | (Kabat) | |
| 8. | 34.5 L-CDR3 | QQYYTTPLT |
| | (Kabat) | |
| 9. | 199.1 VH | QVQLQESGPGLVKPSQTLSLTCTVSGGSISRGGHQWSWIRQNPEKGLEWI |
| | | GYIHHSGSTYYNPSLNNRLTILVDTPKNQFSLKLSSVTAADTAVYYCARW |
| | | NLGSYGDYWGQGTLVTVSS |
| 10. | 199.1 VL | DIVMTQSPDSLAVSLGERATINCKSSQSVLYSPNNQNFLAWYLQKPGQPP |
| | | KLLIYWASTRESGVPDRFSGSGSGTDFTLTISSLQAEDVAVYYCQQYYTTP |
| | | LTFGGGTKVEIK |
| 11. | 199.1 H- | RGGHQWS |
| | CDR1 (Kabat) | |
| 12. | 199.1 H- | YIHHSGSTYYNPSLNN |
| | CDR2 (Kabat) | |
| 13. | 199.1 H- | WNLGSYGDY |
| | CDR3 (Kabat) | |
| 14. | 199.1 L-CDR1 | KSSQSVLYSPNNQNFLA |
| | (Kabat) | |
| 15. | 199.1 L-CDR2 | WASTRES |
| | (Kabat) | |
| 16. | 199.1 L-CDR3 | QQYYTTPLT |
| | (Kabat) | |
| 17. | 3.2 VH | EVQLLESGGGLVQPGGSLRLSCAASGFTFRHYAMSWVRQAPGKGLEWVS |
| | | LISGSGASTFYADSVKGRFTISRDNSENMLYLQMNSLRAEDTAVYYCAKD |
| | | SLAVAGSEYFQHWGQGTLVTVSS |
| 18. | 3.2 VL | SYVLTQAPSVSVAPGQTARITCGGNYFGGKSVHWYQQRPGQAPVLVVYD |
| | | DSDRPSGIPERFSGSKSGNTATLAISRVEAGDEAAYYCQVYDTSSDHWVF |
| | | GGGTKLTVL |
| 19. | 3.2 H-CDR1 | HYAMS |
| | (Kabat) | |
| 20. | 3.2 H-CDR2 | LISGSGASTFYADSVKG |
| | (Kabat) | |
| 21. | 3.2 H-CDR3 | DSLAVAGSEYFQH |
| | (Kabat) | |
| 22. | 3.2 L-CDR1 | GGNYFGGKSVH |
| | (Kabat) | |
| 23. | 3.2 L-CDR2 | DDSDRPS |
| | (Kabat) | |
| 24. | 3.2 L-CDR3 | QVYDTSSDHWV |
| | (Kabat) | |
| 25. | 39.4 VH | EVQLLESGGGLVQPGGSLSLSCTASGFTFSHYAMSWVRQAPGKGLEWVS |
| | | VISGSGASTFYADSVKGRFTISRDNSENTLYLQMNSLRAEDTAVYYCAKD |
| | | SIAVAGSEYFQHWGQGTLVTVSS |
| 26. | 39.4 VL | SYVLTQAPSVSVAPGQTARITCGGNNIGGKTVHWYQQKPGQAPMLVVY |
| | | DDSDRPSGIPERFSGSKSGNMAILTISRVEAGDEADYYCQLYDIYSDHWVF |
| | | GGGTKLTVL |
| 27. | 39.4 H-CDR1 | HYAMS |
| | (Kabat) | |
| 28. | 39.4 H-CDR2 | VISGSGASTFYADSVKG |
| | (Kabat) | |
| 29. | 39.4 H-CDR3 | DSIAVAGSEYFQH |
| | (Kabat) | |
| 30. | 39.4 L-CDR1 | GGNNIGGKTVH |
| | (Kabat) | |
| 31. | 39.4 L-CDR2 | DDSDRPS |
| | (Kabat) | |
| 32. | 39.4 L-CDR3 | QLYDIYSDHWV |
| | (Kabat) | |
| 33. | 48.14 VH | EVQLLESGGGLVQPGGSLRLSCAASGFKFSYYAMSWVRQAPGKGLEWVS |
| | | LISGSGYSTYFADSVKGRFTISRDNSKNTLYLQMNSLRAEDTAVYYCAKD |
| | | SIAVAGSEYFQHWGQGTLVTVSS |
| 34. | 48.14 VL | SYVLTQPPSVSVAPGQTARITCGGDNIGGKSVHWYQQKPGQAPVLVVYD |
| | | DSDRPSGIPERFSGSDSGNTATLTISWVEAGDEADYYCQVWDDSSDHWV |
| | | FGGGTKLTVL |
| 35. | 48.14 H- | YYAMS |
| | CDR1 (Kabat) | |
| 36. | 48.14 H- | LISGSGYSTYFADSVKG |
| | CDR2 (Kabat) | |
| 37. | 48.14 H- | DSIAVAGSEYFQH |
| | CDR3 (Kabat) | |
| 38. | 48.14 L-CDR1 | GGDNIGGKSVH |
| | (Kabat) | |
| 39. | 48.14 L-CDR2 | DDSDRPS |
| | (Kabat) | |
| 40. | 48.14 L-CDR3 | QVWDDSSDHWV |
| | (Kabat) | |
| 41. | 59.45 VH | QLQLQESGPGLVKTSETLSLTCTVSGGSITSGVYYWGWIRQPPQKGLEWI |
| | | GSIYHGGNTYNNPSLKSRVTIFIDTSKNQFSLKLSSVTAADTAVYYCAEGV |
| | | SSGWSYYFDYWGQGTLVTVSS |
| 42. | 59.45 VL | SSELTQDPAVSVALGQTVRITCQGDSLRRFYASWYQQKPGQAPVLVGYG |
| | | KNNRPSGIPDRFSGSTSGNAASLTITGSQAEDEADYYCNSRDSSGDHLIFG |
| | | GGTKLTVL |
| 43. | 59.45 H- | SGVYYWG |
| | CDR1 (Kabat) | |
| 44. | 59.45 H- | SIYHGGNTYNNPSLKS |
| | CDR2 (Kabat) | |
| 45. | 59.45 H- | GVSSGWSYYFDY |
| | CDR3 (Kabat) | |
| 46. | 59.45 L-CDR1 | QGDSLRRFYAS |
| | (Kabat) | |
| 47. | 59.45 L-CDR2 | GKNNRPS |
| | (Kabat) | |
| 48. | 59.45 L-CDR3 | NSRDSSGDHLI |
| | (Kabat) | |
| 49. | 69.44 VH | QVQLVQSGAEVKKPGASVKVSCKASGYSFTTYYMYWVRQAPGQGLEW |
| | | MGIITPSGGSTTYAQKFQDRVTMTRDASTTTVYMELSSLRSEDTAVYYCA |
| | | RGYEGSYGFDHWGQGTLVTVSS |
| 50. | 69.44 VL | SYELTQPPSVSLSPGQTASITCSGDKLQNKYTCWYQQKPGQSPVLVIYQD |
| | | NKRPSGIPERFSGSNSGNTATLTISGTQAMDEADYYCQTWDSSTAVFGGG |
| | | TKLTVL |
| 51. | 69.44 H- | TYYMY |
| | CDR1 (Kabat) | |
| 52. | 69.44 H- | IITPSGGSTTYAQKFQD |
| | CDR2 (Kabat) | |
| 53. | 69.44 H- | GYEGSYGFDH |
| | CDR3 (Kabat) | |
| 54. | 69.44 L-CDR1 | SGDKLGNKYTC |
| | (Kabat) | |
| 55. | 69.44 L-CDR2 | QDNKRPS |
| | (Kabat) | |
| 56. | 69.44 L-CDR3 | QTWDSSTAV |
| | (Kabat) | |
| 57. | 83.16 VH | QLQLQESGPGLVKSSETLSLTCTVSGGPISSSSYYWGWIRQPPGKGLEWIG |
| | | TIYYSGRTYYNPSLKSRGTISVGTSKNQFSLKLSSVTAADTAVFYCAGGV |
| | | GVTWDYWGQGTLVTVSS |
| 58. | 83.16 VL | SYDLSQPLSVSVALGQTARITCGGNDIESKNVHWYQQRPGQAPVLVIYRN |
| | | SNRPSGIPERFSGSNSGNTATLTISRGQGGDEADYYCQVWDSSTVIFGGGT |
| | | KLTVL |
| 59. | 83.16 H- | SSSYYWG |
| | CDR1 (Kabat) | |
| 60. | 83.16 H- | TIYYSGRTYYNPSLKS |
| | CDR2 (Kabat) | |
| 61. | 83.16 H- | GVGVTWDY |
| | CDR3 (Kabat) | |
| 62. | 83.16 L-CDR1 | GGNDIESKNVH |
| | (Kabat) | |
| 63. | 83.16 L-CDR2 | RNSNRPS |
| | (Kabat) | |
| 64. | 83.16 L-CDR3 | QVWDSSTVI |
| | (Kabat) | |
| 65. | 139.14 VH | QVQLRESGPGLMKPSETLSLTCTVSGGSVSSGVYYWTWIRQPPGKGLEWI |
| | | GSMFYSGNADYNPSLKSRVTISVDTSKNQFSLRLNSVSAADMAVYYCAR |
| | | GYASGWSYYFDYWGQATLVTVSS |
| 66. | 139.14 VL | SSELTQDPTVSVALGQTVRITCQGDSLRTYYASWYQQKPGQAPVLVLYG |
| | | KNNRPSGIPDRFSGSSSGNTASLTITGAQAEDEADYYCNSRYSSGTHVVFG |
| | | GGTKLTVL |
| 67. | 139.14 H- | SGVYYWT |
| | CDR1 (Kabat) | |
| 68. | 139.14 H- | SMFYSGNADYNPSLK |
| | CDR2 (Kabat) | |
| 69. | 139.14 H- | GYASGWSYYFDY |
| | CDR3 (Kabat) | |
| 70. | 139.14 L- | QGDSLRTYYAS |
| | CDR1 (Kabat) | |
| 71. | 139.14 L- | GKNNRPS |
| | CDR2 (Kabat) | |
| 72. | 139.14 L- | NSRYSSGTHVV |
| | CDR3 (Kabat) | |
| 73. | 149.46 VH | QVQLQQSGPGLVKPSQPLSLTCAISGDSVSSNSATWSWIRQSPSRGLEWL |
| | | GRTYYRSKWYHDYAVFVKSRMTINPDTSKNQFSLQLNSVTPEDTAVYYC |
| | | ARGGMPASDIPHFDNWGQGILVTVSS |
| 74. | 149.46 VL | SYELTQPPSLSVSPGQTASITCSGHKLGDKYACWYQQKPGQSPVLVIYQD |
| | | NKRPSGIPERFSGSNSGNTATLTISGTQALDEADYYCQAWDSITYVFGTGT |
| | | KVTVL |
| 75. | 149.46 H- | SNSATWS |
| | CDR1 (Kabat) | |
| 76. | 149.46 H- | RTYYRSKWYHDYAVFVKS |
| | CDR2 (Kabat) | |
| 77. | 149.46 H- | GGMPASDIPHFDN |
| | CDR3 (Kabat) | |
| 78. | 149.46 L- | SGHKLGDKYAC |
| | CDR1 (Kabat) | |
| 79. | 149.46 L- | QDNKRPS |
| | CDR2 (Kabat) | |
| 80. | 149.46 L- | QAWDSITYV |
| | CDR3 (Kabat) | |
| 81. | 78.46 VH | EVQLVESGGGLVKPRGSLRLSCGASGFTFTNAWMTWVRQAPGKGLEWV |
| | | GRIKSKTDGGTTDYAAPVKGRFTISRDDSKNTLYLQMNSLKTEDTAVYY |
| | | CNRERELGRGPWDYFYYGMDVWGQGTTVTVSS |
| 82. | 78.46 VL | SYVLTQPPSVSVAPGQTARITCGGNDIGSKSVHWYQQKPGQAPVLVVYD |
| | | DNDRSSGIPERFSGSNSGNTAALTISRVEAGDEADYYCQVWDSSSDHWVF |
| | | GGGTKLTVL |
| 83. | 78.46 H- | NAWMT |
| | CDR.1 (Kabat) | |
| 84. | 78.46 H- | RIKSKTDGGTTDYAAPVKG |
| | CDR2 (Kabat) | |
| 85. | 78.46 H- | ERELGRGPWDYFYYGMDV |
| | CDR3 (Kabat) | |
| 86. | 78.46 L-CDR1 | GGNDIGSKSVH |
| | (Kabat) | |
| 87. | 78.46 L-CDR2 | DDNDRSS |
| | (Kabat) | |
| 88. | 78.46 L-CDR3 | QVWDSSSDHWV |
| | (Kabat) | |
| 89. | 3.2 H2-10 | EVQLLESGGGLVQPGGSLRLSCAASGFTFRHYAMSWVRQAPGKGLEWVS |
| | parental VH | LISGSGASTHYADSVKGRFTISRDNSENMLYLQMNSLRAEDTAVYYCAK |
| | | DSLAVAGSEYFQHWGQGTLVTVSS |
| 90. | 3.2 H2-10 | SYVLTQAPSVSVAPGQTARITCGGNYFGGKSVHWYQQRPGQAPVLVVYD |
| | parental VL | DSDRPSGIPERFSGSKSGNTATLAISRVEAGDEAAYYCQVYDTSSDHWVF |
| | | GGGTKLTVL |
| 91. | 3.2 H2-10 | HYAMS |
| | parental H- | |
| | CDR1 (Kabat) | |
| 92. | 3.2 H2-10 | LISGSGASTHYADSVKG |
| | parental H- | |
| | CDR2 (Kabat) | |
| 93. | 3.2 H2-10 | DSLAVAGSEYFQH |
| | parental H- | |
| | CDR3 (Kabat) | |
| 94. | 3.2 H2-10 | GGNYFGGKSVH |
| | parental L- | |
| | CDR1 (Kabat) | |
| 95. | 3.2 H2-10 | DDSDRPS |
| | parental L- | |
| | CDR2 (Kabat) | |
| 96. | 3.2 H2-10 | QVYDTSSDHWV |
| | parental L- | |
| | CDR3 (Kabat) | |
| 97. | 3.2 H2-10 × L1- | SYVLTQAPSVSVAPGQTARITCGHNYFGGKSVHWYQQRPGQAPVLVVY |
| | 2 VL | DDSDRPSGIPERFSGSKSGNTATLAISRVEAGDEAAYYCQVYDTSSDHWV |
| | | FGGGTKLTVL |
| 98. | 3.2 H2-10 × L1- | GHNYFGGKSVH |
| | 2 L-CDR1 | |
| | (Kabat) | |
| 99. | 3.2 H2-10 × L1- | DDSDRPS |
| | 2 L-CDR2 | |
| | (Kabat) | |
| 100. | 3.2 H2-10 × L1- | QVYDTSSDHWV |
| | 2 L-CDR3 | |
| | (Kabat) | |
| 101. | 3.2 H2-10 × L1- | SYVLTQAPSVSVAPGQTARITCGGNHFGGKSVHWYQQRPGQAPVLVVY |
| | 4 VL | DDSDRPSGIPERFSGSKSGNTATLAISRVEAGDEAAYYCQVYDTSSDHWV |
| | | FGGGTKLTVL |
| 102. | 3.2 H2-10 × L1- | GGNHFGGKSVH |
| | 4 L-CDR1 | |
| | (Kabat) | |
| 103. | 3.2 H2-10 × L1- | DDSDRPS |
| | 4 L-CDR2 | |
| | (Kabat) | |
| 104. | 3.2 H2-10 × L1- | QVYDTSSDHWV |
| | 4 L-CDR3 | |
| | (Kabat) | |
| 105. | 3.2 H2-10 × L1- | SYVLTQAPSVSVAPGQTARITCGGNYHGGKSVHWYQQRPGQAPVLVVY |
| | 5 VL | DDSDRPSGIPERFSGSKSGNTATLAISRVEAGDEAAYYCQVYDTSSDHWV |
| | | FGGGTKLTVL |
| 106. | 3.2 H2-10 × L1- | GGNYHGGKSVH |
| | 5 L-CDR1 | |
| | (Kabat) | |
| 107. | 3.2 H2-10 × L1- | DDSDRPS |
| | 5 L-CDR2 | |
| | (Kabat) | |
| 108. | 3.2 H2-10 × L1- | QVYDTSSDHWV |
| | 5 L-CDR3 | |
| | (Kabat) | |
| 109. | 3.2 H2-10 × L1- | SYVLTQAPSVSVAPGQTARITCGGNYFGHKSVHWYQQRPGQAPVLVVY |
| | 7 VL | DDSDRPSGIPERFSGSKSGNTATLAISRVEAGDEAAYYCQVYDTSSDHWV |
| | | FGGGTKLTVL |
| 110. | 3.2 H2-10 × L1- | GGNYFGHKSVH |
| | 7 L-CDR1 | |
| | (Kabat) | |
| 111. | 3.2 H2-10 × L1- | DDSDRPS |
| | 7 L-CDR2 | |
| | (Kabat) | |
| 112. | 3.2 H2-10 × L1- | QVYDTSSDHWV |
| | 7 L-CDR3 | |
| | (Kabat) | |
| 113. | 3.2 H2-10 × L1- | SYVLTQAPSVSVAPGQTARITCGGNYFGGHSVHWYQQRPGQAPVLVVY |
| | 8 VL | DDSDRPSGIPERFSGSKSGNTATLAISRVEAGDEAAYYCQVYDTSSDHWV |
| | | FGGGTKLTVL |
| 114. | 3.2 H2-10 × L1- | GGNYFGGHSVH |
| | 8 L-CDR1 | |
| | (Kabat) | |
| 115. | 3.2 H2-10 × L1- | DDSDRPS |
| | 8 L-CDR2 | |
| | (Kabat) | |
| 116. | 3.2 H2-10 × L1- | QVYDTSSDHWV |
| | 8 L-CDR3 | |
| | (Kabat) | |
| 117. | 3.2 H2-10 × L1- | SYVLTQAPSVSVAPGQTARITCGGNYFGGKSHHWYQQRPGQAPVLVVY |
| | 10 VL | DDSDRPSGIPERFSGSKSGNTATLAISRVEAGDEAAYYCQVYDTSSDHWV |
| | | FGGGTKLTVL |
| 118. | 3.2 H2-10 × L1- | GGNYFGGKSHH |
| | 10 L-CDR1 | |
| | (Kabat) | |
| 119. | 3.2 H2-10 × L1- | DDSDRPS |
| | 10 L-CDR2 | |
| | (Kabat) | |
| 120. | 3.2 H2-10 × L1- | QVYDTSSDHWV |
| | 10 L-CDR3 | |
| | (Kabat) | |
| 121. | 3.2 H2-10 × L2- | SYVLTQAPSVSVAPGQTARITCGGNYFGGKSVHWYQQRPGQAPVLVVY |
| | 1 VL | HDSDRPSGIPERFSGSKSGNTATLAISRVEAGDEAAYYCQVYDTSSDHWV |
| | | FGGGTKLTVL |
| 122. | 3.2 H2-10 × L2- | GGNYFGGKSVH |
| | 1 L-CDR1 | |
| | (Kabat) | |
| 123. | 3.2 H2-10 × L2- | HDSDRPS |
| | 1 L-CDR2 | |
| | (Kabat) | |
| 124. | 3.2 H2-10 × L2- | QVYDTSSDHWV |
| | 1 L-CDR3 | |
| | (Kabat) | |
| 125. | 3.2 H2-10 × L2- | SYVLTQAPSVSVAPGQTARITCGGNYFGGKSVHWYQQRPGQAPVLVVYD |
| | 3 VL | DHDRPSGIPERFSGSKSGNTATLAISRVEAGDEAAYYCQVYDTSSDHWVF |
| | | GGGTKLTVL |
| 126. | 3.2 H2-10 × L2- | GGNYFGGKSVH |
| | 3 L-CDR1 | |
| | (Kabat) | |
| 127. | 3.2 H2-10 × L2- | DDHDRPS |
| | 3 L-CDR2 | |
| | (Kabat) | |
| 128. | 3.2 H2-10 × L2- | QVYDTSSDHWV |
| | 3 L-CDR3 | |
| | (Kabat) | |
| 129. | 3.2 H2-10 × L2- | SYVLTQAPSVSVAPGQTARITCGGNYFGGKSVHWYQQRPGQAPVLVVYD |
| | 5 VL | DSDHPSGIPERFSGSKSGNTATLAISRVEAGDEAAYYCQVYDTSSDHWVF |
| | | GGGTKLTVL |
| 130. | 3.2 H2-10 × L2- | GQNYFGGKSVH |
| | 5 L-CDR1 | |
| | (Kabat) | |
| 131. | 3.2 H2-10 × L2- | DDSDHPS |
| | 5 L-CDR2 | |
| | (Kabat) | |
| 132. | 3.2 H2-10 × L2- | QVYDTSSDHWV |
| | 5 L-CDR3 | |
| | (Kabat) | |
| 133. | 3.2 H2-10 × L2- | SYVLTQAPSVSVAPGQTARITCGGNYFGGKSVHWYQQRPGQAPVLVVYD |
| | 6 VL | DSDRHSGIPERFSGSKSGNTATLAISRVEAGDEAAYYCQVYDTSSDHWVF |
| | | GGGTKLTVL |
| 134. | 3.2 H2-10 × 12- | GGNYFGGKSVH |
| | 6L-CDR1 | |
| | (Kabat) | |
| 135. | 3.2 H2-10 × L2- | DDSDRHS |
| | 6 L-CDR2 | |
| | (Kabat) | |
| 136. | 3.2 H2-10 × L2- | QVYDTSSDHWV |
| | 6 L-CDR3 | |
| | (Kabat) | |
| 137. | 3.2 H2-10 × L2- | SYVLTQAPSVSVAPGQTARITCGGNYFGGKSVHWYQQRPGQAPVLVVYD |
| | 7 VL | DSDRPHGIPERFSGSKSGNTATLAISRVEAGDEAAYYCQVYDTSSDHWVF |
| | | GGGTKLTVL |
| 138. | 3.2 H2-10 × L2- | GGNYFGGKSVH |
| | 7L-CDR1 (Kabat) | |
| 139. | 3.2 H2-10 × L2- | DDSDRPH |
| | 7 L-CDR2 | |
| | (Kabat) | |
| 140. | 3.2 H2-10 × L2- | QVYDTSSDHWV |
| | 7 L-CDR3 | |
| | (Kabat) | |
| 141. | 3.2 H2-10 × L3- | SYVLTQAPSVSVAPGQTARITCGGNYFGGKSVHWYQQRPGQAPVLVVYD |
| | 4 VL | DSDRPSGIPERFSGSKSGNTATLAISRVEAGDEAAYYCQVYHTSSDHWVF |
| | | GGGTKLTVL |
| 142. | 3.2 H2-10 × L3- | GGNYFGGKSVH |
| | 4 L-CDR1 | |
| | (Kabat) | |
| 143. | 3.2 H2-10 × L3- | DDSDRPS |
| | 4 L-CDR2 | |
| | (Kabat) | |
| 144. | 3.2 H2-10 × L3- | QVYHTSSDHWV |
| | 4 L-CDR3 | |
| | (Kabat) | |
| 145. | 3.2 H2-10 × L3- | SYVLTQAPSVSVAPGQTARITCGGNYFGGKSVHWYQQRPGQAPVLVVYD |
| | 5 VL | DSDRPSGIPERFSGSKSGNTATLAISRVEAGDEAAYYCQVYDHSSDHWVF |
| | | GGGTKLTVL |
| 146. | 3.2 H2-10 × L3- | GGNYFGGKSVH |
| | 5 L-CDR1 | |
| | (Kabat) | |
| 147. | 3.2 H2-10 × L3- | DDSDRPS |
| | 5 L-CDR2 | |
| | (Kabat) | |
| 148. | 3.2 H2-10 × L3- | QVYDHSSDHWV |
| | 5 L-CDR3 | |
| | (Kabat) | |
| 149. | 3.2 H2-10 × L3- | SYVLTQAPSVSVAPGQTARITCGGNYFGGKSVHWYQQRPGQAPVLVVYD |
| | 6 VL | DSDRPSGIPERFSGSKSGNTATLAISRVEAGDEAAYYCQVYDTHSDHWVF |
| | | GGGTKLTVL |
| 150. | 3.2 H2-10 × L3- | GGNYFGGKSVH |
| | 6 L-CDR1 | |
| | (Kabat) | |
| 151. | 3.2 H2-10 × L3- | DDSDRPS |
| | 6L-CDR2 (Kabat) | |
| 152. | 3.2 H2-10 × L3- | QVYDTHSDHWV |
| | 6 L-CDR3 | |
| | (Kabat) | |
| 153. | 3.2 H2-10 × L3- | SYVLTQAPSVSVAPGQTARITCGGNYFGGKSVHWYQQRPGQAPVLVVYD |
| | 7 VL | DSDRPSGIPERFSGSKSGNTATLAISRVEAGDEAAYYCQVYDTSHDHWVF |
| | | GGGTKLTVL |
| 154. | 3.2 H2-10 × L3- | GGNYFGGKSVH |
| | 7 L-CDR1 | |
| | (Kabat) | |
| 155. | 3.2 H2-10 × L3- | DDSDRPS |
| | 7 L-CDR2 | |
| | (Kabat) | |
| 156. | 3.2 H2-10 × L3- | QVYDTSHDHWV |
| | 7L-CDR3 | |
| | (Kabat) | |
| 157. | 3.2 H2-10 × L3- | SYVLTQAPSVSVAPGQTARITCGGNYFGGKSVHWYQQRPGQAPVLVVYD |
| | 11 VL | DSDRPSGIPERFSGSKSGNTATLAISRVEAGDEAAYYCQVYDTSSDHWHF |
| | | GGGTKLTVL |
| 158. | 3.2 H2-10 × L3- | GGNYFGGKSVH |
| | 11 L-CDR1 | |
| | (Kabat) | |
| 159. | 3.2 H2-10 × L3- | DDSDRPS |
| | 11 L-CDR2 | |
| | (Kabat) | |
| 160. | 3.2 H2-10 × L3- | QVYDTSSDHWH |
| | 11L-CDR3 | |
| | (Kabat) | |
| 161. | 34.5 VH | CAGGTGCAGCTGCAGGAGTCGGGCCCAGGACTGGTGAAGCCGTCACA |
| | nucleotide | GACCCTGTCCCTCACCTGCACTGTCTCTGGTGGCTCCATCAGCAGGGG |
| | | TGGTCACCAGTGGACTTGGATCCGCCAGCACCCAGGGAAGGGCCTGG |
| | | AGTGGATTGGGTACATCCACCACAGTGGGAGCACCTACTACAACCCGT |
| | | CCCTCAACGACCGACTTACCATATTGGTAGACACGCCTAAGAACCAGT |
| | | TTTCCCTGAAGCTGAGCTCTGTGACTGCCGCGGACACGGCCGTGTATT |
| | | ACTGTGCGAGATGGAATTTGGGGAGTTATGGGGACTACTGGGGCCAG |
| | | GGAACCCTGGTCACCGTCTCCTCA |
| 162. | 34.5 VL | GCCATCGTGATGACCCAGTCTCCAGACTCCCTGGCTGTGTCTCTGGGC |
| | nucleotide | GAGAGGGCCACCATCAACTGTAAGTCCAGCCAGAGTGTTTTATACAGC |
| | | CCCAACAATAAGAACTACTTGGCTTGGTACCTGCAGAAACCAGGGCA |
| | | GCCTCCTAAGCTGCTCATTTACTGGGCATCTACCCGGGAATCCGGGGT |
| | | CCCTGACCGATTCAGTGGCAGCGGGTCTGGGACAGATTTCACTCTCAC |
| | | CATCAGCAGCCTGCAGGCTGAAGATGTGGCAGTTTATTACTGTCAGCA |
| | | ATATTATACTACTCCTCTCACTTTCGGCGGAGGGACCAAGGTGGAGAT |
| | | CAAA |
| 163. | 199.1 VH | CAGGTGCAGCTGCAGGAGTCGGGCCCAGGACTGGTGAAGCCGTCCCA |
| | nucleotide | GACCCTGTCCCTCACTTGCACTGTCTCTGGTGGCTCCATCAGCAGGGGT |
| | | GGTCACCAGTGGAGTTGGATCCGCCAGAACCCAGAGAAGGGCCTGGA |
| | | GTGGATTGGGTACATCCACCACAGTGGGAGCACCTACTACAACCCGTC |
| | | CCTCAACAATCGACTTACCATTTTGGTAGACACGCCTAAGAACCAGTT |
| | | TTCCCTGAAGCTGAGCTCTGTGACTGCCGCGGACACGGCCGTGTATTA |
| | | CTGTGCGAGATGGAATTTGGGGAGTTATGGGGACTACTGGGGCCAGG |
| | | GAACCCTGGTCACCGTCTCCTCA |
| 164. | 199.1 VL | GACATCGTGATGACCCAGTCTCCAGACTCCCTGGCTGTGTCTCTGGGC |
| | nucleotide | GAGAGGGCCACCATCAACTGCAAGTCCAGCCAGAGTGTTTTATACAGC |
| | | CCCAACAATCAGAACTTCTTAGCTTGGTACCTGCAGAAACCAGGTCAG |
| | | CCTCCTAAGCTGCTCATTTACTGGGCATCTACCCGGGAATCCGGGGTC |
| | | CCTGACCGATTCAGTGGCAGCGGGTCTGGGACAGATTTCACTCTCACC |
| | | ATCAGCAGCCTGCAGGCTGAAGATGTGGCAGTTTATTACTGTCAGCAA |
| | | TATTATACTACTCCTCTCACTTTCGGCGGAGGGACCAAGGTGGAGATC |
| | | AAA |
| 165. | 3.2 VH | GAGGTGCAGCTGTTGGAGTCTGGGGGAGGCCTGGTACAGCCTGGGGG |
| | nucleotide | GTCCCTGAGACTCTCCTGTGCAGCCTCTGGATTCACCTTTAGGCACTAT |
| | | GCCATGAGCTGGGTCCGCCAGGCTCCAGGGAAGGGGCTGGAGTGGGT |
| | | CTCACTTATCAGTGGTAGTGGTGCCAGCACATTCTACGCAGACTCCGT |
| | | GAAGGGCCGGTTCACCATCTCCAGAGACAATTCCGAGAACATGCTGTA |
| | | TCTGCAAATGAACAGCCTGAGAGCCGAGGACACGGCCGTCTATTACTG |
| | | TGCGAAAGATAGTTTAGCAGTAGCTGGTTCTGAATACTTCCAGCACTG |
| | | GGGCCAGGGCACCCTGGTCACCGTCTCCTCA |
| 166. | 3.2 VL | TCCTATGTGCTGACTCAGGCGCCCTCGGTGTCAGTGGCCCCAGGACAG |
| | nucleotide | ACGGCCAGGATTACCTGTGGGGGAAACTATTTTGGAGGTAAAAGTGTA |
| | | CACTGGTACCAGCAGAGGCCAGGCCAGGCCCCTGTCCTGGTCGTCTAT |
| | | GATGATAGCGACCGGCCCTCAGGGATCCCTGAGCGATTCTCTGGCTCC |
| | | AAGTCTGGGAACACGGCCACCCTGGCCATCAGCAGGGTCGAAGCCGG |
| | | GGATGAGGCCGCCTATTACTGTCAGGTGTATGATACTAGTAGTGATCA |
| | | TTGGGTGTTCGGCGGAGGGACCAAGCTGACCGTCCTA |
| 167. | 39.4 VH | GAGGTGCAGCTGTTGGAGTCTGGGGGAGGCTTGGTACAGCCTGGGGG |
| | nucleotide | GTCCCTGAGTCTCTCCTGTACAGCCTCTGGATTCACCTTTAGCCACTAT |
| | | GCCATGAGCTGGGTCCGCCAGGCTCCAGGGAAGGGGCTGGAGTGGGT |
| | | CTCAGTTATCAGTGGTAGTGGTGCCAGCACATTCTACGCAGACTCCGT |
| | | GAAGGGCCGGTTCACCATCTCCAGAGACAATTCCGAGAACACGCTGTA |
| | | TCTGCAAATGAACAGCCTGAGAGCCGAGGACACGGCCGTATATTACTG |
| | | TGCGAAAGATAGTATAGCAGTAGCTGGTTCTGAATACTTCCAGCACTG |
| | | GGGCCAGGGCACCCTGGTCACCGTCTCCTCA |
| 168. | 39.4 VL | TCCTATGTGCTGACTCAGGCGCCCTCGGTGTCAGTGGCCCCAGGACAG |
| | nucleotide | ACGGCCAGGATTACCTGTGGGGGAAACAATATTGGAGGTAAAACTGT |
| | | ACACTGGTACCAGCAGAAGCCAGGCCAGGCCCCTATGCTGGTCGTCTA |
| | | TGATGATAGCGACCGGCCCTCAGGGATCCCTGAGCGATTCTCTGGCTC |
| | | CAAGTCTGGGAATATGGCCATCCTGACCATCAGTAGGGTCGAAGCCGG |
| | | GGATGAGGCCGACTATTACTGTCAACTGTATGATATTTATAGTGATCA |
| | | TTGGGTGTTCGGCGGAGGGACCAAGCTGACCGTCCTA |
| 169. | 48.14 VH | GAGGTGCAGCTGTTGGAGTCTGGGGGAGGTTTGGTACAGCCTGGGGG |
| | nucleotide | GTCCCTGAGACTCTCCTGTGCAGCCTCTGGATTCAAATTTAGCTACTAT |
| | | GCCATGAGCTGGGTCCGCCAGGCTCCAGGGAAGGGGCTGGAGTGGGT |
| | | CTCACTTATTAGTGGTAGTGGTTATAGCACATACTTCGCAGACTCCGTG |
| | | AAGGGCCGGTTCACCATCTCCAGAGACAATTCCAAGAACACGCTGTAT |
| | | CTTCAAATGAACAGCCTGAGAGCCGAGGACACGGCCGTATATTACTGT |
| | | GCGAAAGATTCGATAGCAGTGGCTGGTAGTGAATACTTCCAGCACTGG |
| | | GGCCAGGGCACCCTGGTCACCGTCTCCTCA |
| 170. | 48.14 VL | TCCTATGTGCTGACTCAGCCACCCTCGGTGTCAGTGGCCCCAGGACAG |
| | nucleotide | ACGGCCAGGATTACCTGTGGGGGAGACAACATTGGAGGTAAAAGTGT |
| | | GCACTGGTACCAACAGAAGCCAGGCCAGGCCCCTGTGCTGGTCGTCTA |
| | | TGATGATAGCGACCGGCCCTCAGGTATCCCTGAGCGCTTCTCTGGCTC |
| | | CGACTCTGGGAACACGGCCACCCTGACCATCAGTTGGGTCGAAGCCGG |
| | | GGATGAGGCCGACTATTACTGTCAGGTGTGGGATGATAGTAGTGATCA |
| | | TTGGGTGTTCGGCGGAGGGACCAAGCTGACCGTCCTA |
| 171. | 59.45 VH | CAGCTGCAGTTGCAGGAGTCGGGCCCAGGACTGGTGAAGACTTCGGA |
| | nucleotide | GACCCTGTCCCTCACCTGCACTGTCTCTGGTGGTTCCATCACCAGTGGT |
| | | GTTTACTACTGGGGCTGGATCCGCCAGCCCCCAGGGAAGGGGCTGGA |
| | | GTGGATTGGGAGTATCTATCATGGTGGGAACACCTACAACAACCCGTC |
| | | CCTCAAGAGTCGAGTCACCATATTCATAGACACGTCCAAGAACCAGTT |
| | | CTCCCTGAAGCTGAGTTCTGTGACCGCCGCTGACACGGCTGTGTATTA |
| | | CTGTGCGGAGGGAGTTAGCAGTGGCTGGTCCTACTACTTTGACTATTG |
| | | GGGCCAGGGAACCCTGGTCACCGTCTCCTCA |
| 172. | 59.45 VL | TCTTCTGAACTGACTCAGGACCCTGCTGTGTCTGTGGCCTTGGGACAG |
| | nucleotide | ACAGTCAGGATCACATGCCAAGGAGACAGCCTCAGACGCTTTTATGCT |
| | | AGCTGGTACCAGCAGAAGCCAGGACAGGCCCCTGTTCTTGTCGGCTAT |
| | | GGTAAAAATAACCGGCCCTCAGGGATCCCCGACCGATTCTCCGGCTCC |
| | | ACCTCGGGAAACGCAGCTTCTTTGACCATCACTGGGTCTCAGGCGGAA |
| | | GATGAGGCTGACTATTACTGTAACTCCCGGGACAGCAGTGGTGACCAT |
| | | CTGATATTCGGCGGAGGTACCAAGTTGACCGTCCTA |
| 173. | 69.44 VH | CAGGTGCAGCTGGTGCAGTCTGGGGCTGAGGTGAAGAAGCCTGGGGC |
| | nucleotide | CTCAGTGAAGGTTTCCTGTAAGGCATCTGGATACAGTTTCACCACTTA |
| | | CTATATGTACTGGGTGCGACAGGCCCCTGGACAAGGGCTTGAGTGGAT |
| | | GGGAATCATCACCCCTAGCGGTGGTAGTACAACCTACGCACAGAAGTT |
| | | CCAGGACAGAGTCACCATGACAAGGGACGCGTCCACGACTACAGTCT |
| | | ACATGGAGCTGAGCAGCCTGAGATCTGAGGACACGGCCGTGTATTACT |
| | | GTGCGAGAGGGTATGAGGGGAGCTACGGGTTTGACCACTGGGGCCAG |
| | | GGAACCCTGGTCACCGTCTCCTCA |
| 174. | 69.44 VL | TCCTATGAACTGACTCAGCCACCCTCAGTGTCCCTGTCCCCAGGACAG |
| | nucleotide | ACAGCCAGCATCACCTGCTCTGGAGATAAATTGGGAAATAAATATACT |
| | | TGCTGGTATCAGCAGAAGCCAGGCCAGTCCCCTGTCCTGGTCATCTAT |
| | | CAAGATAACAAGCGGCCCTCGGGGATCCCTGAGCGATTCTCTGGCTCC |
| | | AACTCTGGGAACACAGCCACTCTGACCATCAGCGGGACCCAGGCTATG |
| | | GATGAGGCTGACTATTACTGTCAGACGTGGGACAGCAGCACTGCGGTG |
| | | TTCGGCGGAGGGACCAAGCTGACCGTCCTA |
| 175. | 83.16 VH | CAGCTGCAGCTGCAGGAGTCGGGCCCAGGATTAGTGAAGTCTTCGGA |
| | nucleotide | GACCCTGTCCCTCACCTGCACTGTCTCTGGTGGCCCCATCAGCAGTAGT |
| | | AGTTACTACTGGGGCTGGATCCGCCAGCCCCCAGGGAAGGGGCTGGA |
| | | GTGGATTGGGACTATTTATTATAGTGGGAGGACCTACTACAATCCGTC |
| | | CCTCAAGAGTCGAGGCACCATATCCGTAGGAACGTCCAAGAACCAGTT |
| | | CTCCCTGAAGCTGAGCTCTGTGACCGCCGCAGACACGGCAGTGTTTTA |
| | | CTGCGCGGGGGGAGTGGGAGTTACTTGGGACTACTGGGGCCAGGGAA |
| | | CCCTGGTCACCGTCTCCTCA |
| 176. | 83.16 VL | TCCTATGACCTGTCTCAGCCACTCTCAGTGTCAGTGGCCCTGGGACAG |
| | nucleotide | ACGGCCAGGATTACCTGTGGGGGAAACGACATTGAAAGTAAAAATGT |
| | | GCACTGGTACCAGCAGAGGCCAGGCCAGGCCCCTGTGCTGGTCATCTA |
| | | TAGGAATAGCAACCGGCCCTCTGGGATCCCTGAGCGATTCTCTGGCTC |
| | | CAACTCGGGGAACACGGCCACCCTGACCATCAGCAGAGGCCAAGGCG |
| | | GGGATGAGGCTGACTATTACTGTCAGGTGTGGGACAGCAGCACTGTGA |
| | | TTTTCGGCGGAGGGACCAAGCTGACCGTCCTA |
| 177. | 139.14 VH | CAGGTGCAGCTGCGGGAGTCGGGCCCAGGACTGATGAAGCCTTCGGA |
| | nucleotide | GACCCTGTCCCTCACCTGCACTGTCTCTGGTGGCTCCGTCAGCAGTGGC |
| | | GTTTACTACTGGACCTGGATCCGGCAGCCCCCAGGGAAGGGACTGGA |
| | | ATGGATTGGGTCTATGTTTTACAGTGGGAACGCCGACTACAACCCCTC |
| | | CCTCAAGAGTCGAGTCACCATATCAGTAGACACGTCCAAGAACCAGTT |
| | | CTCCCTGAGGCTGAACTCTGTGTCCGCTGCGGACATGGCCGTTTATTAC |
| | | TGTGCGAGAGGATATGCCAGTGGGTGGTCCTACTACTTTGACTACTGG |
| | | GGCCAGGCAACCCTGGTCACCGTCTCCTCA |
| 178. | 139.14 VL | TCTTCTGAGCTGACTCAGGACCCTACTGTGTCTGTGGCCTTGGGACAG |
| | nucleotide | ACAGTCAGGATCACATGCCAAGGAGACAGCCTCAGAACCTATTATGC |
| | | AAGCTGGTACCAGCAGAAGCCAGGACAGGCCCCTGTACTTGTCCTCTA |
| | | TGGTAAAAACAACCGGCCCTCAGGGATCCCAGACCGCTTCTCTGGCTC |
| | | CAGCTCAGGAAACACAGCTTCCTTGACCATCACTGGGGCTCAGGCGGA |
| | | AGATGAGGCTGACTATTACTGTAACTCCCGATACAGCAGTGGTACCCA |
| | | TGTGGTATTCGGCGGAGGGACCAAGCTGACCGTCCTA |
| 179. | 149.46 VH | CAGGTACAGCTGCAGCAGTCAGGTCCAGGACTGGTGAAGCCCTCGCA |
| | nucleotide | GCCCCTCTCACTCACCTGTGCCATCTCCGGGGACAGTGTCTCTAGCAA |
| | | CAGTGCTACTTGGAGCTGGATCAGGCAGTCCCCATCGAGAGGCCTTGA |
| | | GTGGCTGGGAAGGACATACTACAGGTCCAAGTGGTATCATGATTATGC |
| | | AGTATTTGTGAAGAGTCGAATGACCATCAACCCAGACACATCCAAGA |
| | | ACCAGTTCTCCCTGCAGCTGAACTCTGTGACTCCCGAGGACACGGCTG |
| | | TGTATTACTGTGCAAGAGGGGGTATGCCAGCCAGTGATATACCCCACT |
| | | TTGACAACTGGGGCCAGGGTATTCTGGTCACCGTCTCTTCA |
| 180. | 149.46 VL | TCCTATGAGCTGACCCAGCCACCCTCTTTGTCCGTGTCCCCAGGACAG |
| | nucleotide | ACAGCCAGCATCACCTGCTCTGGACATAAGTTGGGGGATAAATATGCT |
| | | TGTTGGTATCAGCAGAAGCCAGGCCAGTCCCCTGTGTTGGTCATCTAT |
| | | CAAGATAACAAGCGGCCCTCAGGGATCCCTGAGCGATTCTCTGGCTCC |
| | | AACTCTGGGAACACAGCCACTCTGACCATCAGCGGGACCCAGGCTTTG |
| | | GATGAGGCTGACTATTACTGTCAGGCGTGGGACAGCATCACTTATGTC |
| | | TTCGGAACTGGGACCAAGGTCACCGTCCTA |
| 181. | 78.46 VH | GAGGTGCAGCTGGTGGAGTCTGGGGGAGGCTTGGTAAAGCCTAGGGG |
| | nucleotide | GTCCCTTAGACTCTCCTGTGGAGCCTCTGGATTCACTTTCACTAATGCC |
| | | TGGATGACCTGGGTCCGCCAGGCTCCAGGGAAGGGGCTGGAGTGGGT |
| | | TGGCCGTATTAAAAGCAAAACTGATGGTGGGACAACAGACTACGCTG |
| | | CACCCGTGAAAGGCAGATTCACCATCTCAAGAGATGACTCAAAAAAC |
| | | ACGCTGTATCTGCAAATGAACAGCCTGAAAACCGAGGACACTGCCGT |
| | | GTATTACTGTAATAGAGAGAGGGAACTGGGGAGGGGCCCGTGGGACT |
| | | ACTTCTACTACGGTATGGACGTCTGGGGCCAAGGGACCACGGTCACCG |
| | | TCTCCTCA |
| 182. | 78.46 VL | TCCTATGTGCTGACTCAGCCACCCTCGGTGTCAGTGGCCCCAGGACAG |
| | nucleotide | ACGGCCAGGATTACCTGTGGGGGAAACGACATTGGAAGTAAAAGTGT |
| | | GCACTGGTACCAGCAGAAGCCAGGCCAGGCCCCTGTGCTGGTCGTCTA |
| | | TGATGATAACGACCGGTCCTCAGGGATCCCTGAGCGATTTTCTGGCTC |
| | | CAACTCTGGGAACACGGCCGCCCTGACCATCAGCAGGGTCGAAGCCG |
| | | GGGATGAGGCCGACTATTACTGTCAGGTGTGGGATAGTAGTAGTGATC |
| | | ATTGGGTGTTCGGCGGAGGGACCAAGCTGACCGTCCTA |
| 183. | IgG4 Fc WT | ASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKC |
| | | KVSNKGLPSSIEKTISKAKGQPREPQVYTLPPSQEEMTKNQVSLTCLVKGF |
| | | YPSDIAVEWESNGQPENNYKTTPPVLDSDGSFFLYSRLTVDKSRWQEGNV |
| | | FSCSVMHEALHNHYTQKSLSLSLGK |
| 184. | IgG4 Fc PLA | ASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVSQEDPE |
| | | VQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEG |
| | | NVFSCSVLHEALHAHYTQKSLSLSLGK |
| 185. | Human C2 | MGPLMVLFCLLFLYPGLADSAPSCPQNVNISGGTFTLSHGWAPGSLLTYS |
| | UniProt | CPQGLYPSPASRLCKSSGQWQTPGATRSLSKAVCKPVRCPAPVSFENGIYT |
| | P06681) | PRLGSYPVGGNVSFECEDGFILRGSPVRQCRPNGMWDGETAVCDNGAGH |
| | | CPNPGISLGAVRTGFRFGHGDKVRYRCSSNLVLTGSSERECQGNGVWSGT |
| | | EPICRQPYSYDFPEDVAPALGTSFSHMLGATNPTQKTKESLGRKIQIQRSG |
| | | HLNLYLLLDCSQSVSENDFLIFKESASLMVDRIFSFEINVSVAIITFAS |
| | | EPKVLMSVLNDNSRDMTEVISSLENANYKDHENGTGTNTYAALNSVYLM |
| | | MNNQMRLLGMETMAWQEIRHAIILLTDGKSNMGGSPKTAVDHIREILNIN |
| | | QKRNDYLDIYAIGVGKLDVDWRELNELGSKKDGERHAFILQDTKALHQV |
| | | FEHMLDVSKLTDTICGVGNMSANASDQERTPWHVTIKPKSQETCRGALIS |
| | | DQWVLTAAHCFRDGNDHSLWRVNVGDPKSQWGKEFLIEKAVISPGFDVF |
| | | AKKNQGILEFYGDDIALLKLAQKVKMSTHARPICLPCTMEANLALRRPQG |
| | | STCRDHENELLNKQSVPAHFVALNGSKLNINLKMGVEWTSCAEVVSQEK |
| | | TMFPNLTDVREVVTDQFLCSGTQEDESPCKGESGGAVFLERRFRFFQVGL |
| | | VSWGLYNPCLGSADKNSRKRAPRSKVPPPRDFHINLFRMQPWLRQHLGD |
| | | VLNFLPL |
| 186. | Human C2a | KIQIQRSGHLNLYLLLDCSQSVSENDFLIFKESASLMVDRIFSFEINVSVAII |
| | | TFASEPKVLMSVLNDNSRDMTEVISSLENANYKDHENGTGTNTYAALNS |
| | | VYLMMNNQMRLLGMETMAWQEIRHAIILLTDGKSNMGGSPKTAVDHIR |
| | | EILNINQKRNDYLDIYAIGVGKLDVDWRELNELGSKKDGERHAFILQDTK |
| | | ALHQVFEHMLDVSKLTDTICGVGNMSANASDQERTPWHVTIKPKSQETC |
| | | RGALISDQWVLTAAHCFRDGNDHSLWRVNVGDPKSQWGKEFLIEKAVIS |
| | | PGFDVFAKKNQGILEFYGDDIALLKLAQKVKMSTHARPICLPCTMEANLA |
| | | LRRPQGSTCRDHENELLNKQSVPAHFVALNGSKLNINLKMGVEWTSCAE |
| | | VVSQEKTMFPNLTDVREVVTDQFLCSGTQEDESPCKGESGGAVFLERRFR |
| | | FFQVGLVSWGLYNPCLGSADKNSRKRAPRSKVPPPRDFHINLFRMQPWL |
| | | RQHLGDVLNFLPL |
| 187. | Cyno C2 | MDPLMVLFCLLFLYPGPADSATSCPQNVNISGGTFTLSHGWAPGSLLIYSC |
| | (UniProt | PQGLYPSPASRLCKSSGQWQTPRATRSLTKAVCKPVRCPAPVSFENGIYTP |
| | G8F3W0) | RLGSYPVGGNVSFECEDGFILRGSPVRQCRPNGMWDGETAVCDNGAGHC |
| | | PNPGISLGAVRTGSRFGHGDKVRYRCSSNLVLTGSAERECQGNGVWSGT |
| | | EPICRQPYSYDFPEDVAPALGTSFSHMLGATNPTQRTKESLGRKIQIQRSG |
| | | HLNLYLLLDSSQSVSENDFLIFKESASLMVDRIFSFEINVSVAIITFASEPKV |
| | | LMSVLNVNSRDITEVISSLENAKYKDHENGTGTNTYAALNSVYLMMNNQ |
| | | MQLLGMKTMAWQEIRHAIILLTDGKSNMGGSPKTAVDQIREILNINQKRN |
| | | DYLDIYAIGVGKLDVDWRELNELGSKKDGERHAFILQDTKALHQVFEHM |
| | | LDVSKLTDTICGVGNMSANASDQERTPWHVTIKPKSQETCRGALISDQW |
| | | VLTAAHCFRDGNDHSLWRVNVGDPKSQWGKEFLIEKAVISPGFDVFAKK |
| | | NQGILEFYGDDIALLKLAQKVKMSTHARPICLPCTMEANLALRRPQGSTC |
| | | RDHENELLNKQSVPAHFVALNGSKLNINLKMGVEWTSCAEVVSQEKTMF |
| | | PNLTDVREVVTDQFLCSGTQEDESPCKGESGGAVFLERRFRFFQVGLVSW |
| | | GLYNPCLGSADKNSRKRAPRSKVPPPRDFHINLFRMQPWLRQHLGDVLN |
| | | FLPL |
| 188. | Cyno C2a | KIQIQRSGHLNLYLLLDSSQSVSENDFLIFKESASLMVDRIFSFEINVSVAIIT |
| | | FASEPKVLMSVLNVNSRDITEVISSLENAKYKDHENGTGTNTYAALNSVY |
| | | LMMNNQMQLLGMKTMAWQEIRHAIILLTDGKSNMGGSPKTAVDQIREIL |
| | | NINQKRNDYLDIYAIGVGKLDVDWRELNELGSKKDGERHAFILQDTKAL |
| | | HQVFEHMLDVSKLTDTICGVGNMSANASDQERTPWHVTIKPKSQETCRG |
| | | ALISDQWVLTAAHCFRDGNDHSLWRVNVGDPKSQWGKEFLIEKAVISPG |
| | | FDVFAKKNQGILEFYGDDIALLKLAQKVKMSTHARPICLPCTMEANLALR |
| | | RPQGSTCRDHENELLNKQSVPAHFVALNGSKLNINLKMGVEWTSCAEVV |
| | | SQEKTMFPNLTDVREVVTDQFLCSGTQEDESPCKGESGGAVFLERRFRFF |
| | | QVGLVSWGLYNPCLGSADKNSRKRAPRSKVPPPRDFHINLFRMQPWLRQ |
| | | HLGDVLNFLPL |
| 189. | 3.2 H2-10 | GAGGTGCAGCTGCTGGAATCCGGGGGGGGACTCGTCCAACCCGGCGG |
| | parental VH | ATCTCTGCGGCTGTCCTGTGCTGCTTCTGGCTTCACCTTCAGACACTAC |
| | DNA | GCCATGTCCTGGGTGCGGCAGGCCCCTGGCAAAGGCCTGGAATGGGTC |
| | | AGCCTGATCTCCGGATCTGGCGCCTCTACCCATTATGCCGATTCCGTGA |
| | | AGGGCAGATTTACCATCTCTCGCGACAACAGCGAGAACATGCTGTACC |
| | | TGCAGATGAACTCCCTGAGAGCCGAGGACACCGCCGTGTACTACTGCG |
| | | CCAAGGACTCCCTGGCTGTGGCTGGCTCCGAGTACTTCCAGCACTGGG |
| | | GCCAGGGCACACTGGTGACCGTGTCCAGC |
| 190. | 3.2 H2-10 × L1- | AGCTATGTGCTGACCCAAGCCCCTTCTGTGTCTGTGGCCCCTGGACAG |
| | 2 VL DNA | ACAGCTAGAATCACCTGTGGACACAACTACTTTGGAGGCAAGAGCGT |
| | | GCACTGGTATCAGCAGAGACCTGGCCAAGCCCCTGTGCTGGTGGTGTA |
| | | TGATGACTCTGACAGACCTTCTGGCATCCCTGAGAGATTCTCTGGCAG |
| | | CAAGTCTGGCAACACAGCCACCCTGGCCATCAGCAGAGTGGAGGCTG |
| | | GAGATGAGGCTGCCTACTACTGCCAAGTGTATGACACAAGCTCTGACC |
| | | ACTGGGTGTTTGGAGGCGGTACCAAGCTGACCGTGCTG |
| 191. | 3.2 H2-10 × L1- | AGCTATGTGCTGACCCAAGCCCCTTCTGTGTCTGTGGCCCCTGGACAG |
| | 5 VL DNA | ACAGCTAGAATCACCTGTGGAGGCAACTACCATGGAGGCAAGAGCGT |
| | | GCACTGGTATCAGCAGAGACCTGGCCAAGCCCCTGTGCTGGTGGTGTA |
| | | TGATGACTCTGACAGACCTTCTGGCATCCCTGAGAGATTCTCTGGCAG |
| | | CAAGTCTGGCAACACAGCCACCCTGGCCATCAGCAGAGTGGAGGCTG |
| | | GAGATGAGGCTGCCTACTACTGCCAAGTGTATGACACAAGCTCTGACC |
| | | ACTGGGTGTTTGGAGGCGGTACCAAGCTGACCGTGCTG |
| 192. | 3.2 H2-10 × L1- | AGCTATGTGCTGACCCAAGCCCCTTCTGTGTCTGTGGCCCCTGGACAG |
| | 10 VL DNA | ACAGCTAGAATCACCTGTGGAGGCAACTACTTTGGAGGCAAGAGCCA |
| | | CCACTGGTATCAGCAGAGACCTGGCCAAGCCCCTGTGCTGGTGGTGTA |
| | | TGATGACTCTGACAGACCTTCTGGCATCCCTGAGAGATTCTCTGGCAG |
| | | CAAGTCTGGCAACACAGCCACCCTGGCCATCAGCAGAGTGGAGGCTG |
| | | GAGATGAGGCTGCCTACTACTGCCAAGTGTATGACACAAGCTCTGACC |
| | | ACTGGGTGTTTGGAGGCGGTACCAAGCTGACCGTGCTG |
| 193. | 3.2 H2-10 × L2- | AGCTATGTGCTGACCCAAGCCCCTTCTGTGTCTGTGGCCCCTGGACAG |
| | 1 VL DNA | ACAGCTAGAATCACCTGTGGAGGCAACTACTTTGGAGGCAAGAGCGT |
| | | GCACTGGTATCAGCAGAGACCTGGCCAAGCCCCTGTGCTGGTGGTGTA |
| | | TCATGACTCTGACAGACCTTCTGGCATCCCTGAGAGATTCTCTGGCAG |
| | | CAAGTCTGGCAACACAGCCACCCTGGCCATCAGCAGAGTGGAGGCTG |
| | | GAGATGAGGCTGCCTACTACTGCCAAGTGTATGACACAAGCTCTGACC |
| | | ACTGGGTGTTTGGAGGCGGTACCAAGCTGACCGTGCTG |
| 194. | 3.2 H2-10 × L3- | AGCTATGTGCTGACCCAAGCCCCTTCTGTGTCTGTGGCCCCTGGACAG |
| | 4 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTACCACACAAGCTCTGACCA |
| | | CTGGGTGTTTGGAGGCGGTACCAAGCTGACCGTGCTG |
| 195 | 3.2 H2-10 × L3- | AGCTATGTGCTGACCCAAGCCCCTTCTGTGTCTGTGGCCCCTGGACAG |
| | 5 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACCACAGCTCTGACCA |
| | | CTGGGTGTTTGGAGGCGGTACCAAGCTGACCGTGCTG |
| 196 | 3.2 H2-10 × L3- | AGCTATGTGCTGACCCAAGCCCCTTCTGTGTCTGTGGCCCCTGGACAG |
| | 6 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACACACACTCTGACCA |
| | | CTGGGTGTTTGGAGGCGGTACCAAGCTGACCGTGCTG |
| 197. | 3.2 H2-10 × L3- | AGCTATGTGCTGACCCAAGCCCCTTCTGTGTCTGTGGCCCCTGGACAG |
| | 7 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACACAAGCCATGACCA |
| | | CTGGGTGTTTGGAGGCGGTACCAAGCTGACCGTGCTG |
| 198 | 3.2 H2-10 × L3- | AGCTATGTGCTGACCCAAGCCCCTTCTGTGTCTGTGGCCCCTGGACAG |
| | 10 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACACAAGCTCTGACCA |
| | | CCATGTGTTTGGAGGCGGTACCAAGCTGACCGTGCTG |
| 199. | 3.2 H2-10 × L3- | AGCTATGTGCTGACCCAAGCCCCTTCTGTGTCTGTGGCCCCTGGACAG |
| | 11 VL DNA | ACAGCTAGAATCACCTGTGGAGGCAACTACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACACAAGCTCTGACCA |
| | | CTGGCACTTTGGAGGCGGTACCAAGCTGACCGTGCTG |
| 200. | 3.2 H2-10 × L1- | AGCTATGTGCTGACCCAAGCCCCTTCTGTGTGTGTGGCCCCTGGACAG |
| | 4 VL DNA | ACAGCTAGAATCACCTGTGGAGGCAACCACTTTGGAGGCAAGTCTGTG |
| | | CACTGGTATCAGCAGAGACCTGGCCAAGCCCCTGTGCTGGTGGTGTAT |
| | | GATGACTCTGACAGACCTTCTGGCATCCCTGAGAGATTCTCTGGCAGC |
| | | AAGTCTGGCAACACAGCCACCCTGGCCATCAGCAGAGTGGAGGCTGG |
| | | AGATGAGGCTGCCTACTACTGCCAAGTGTATGACACAAGCTCTGACCA |
| | | CTGGGTGTTTGGAGGCGGTACCAAGCTGACCGTGCTG |
| 201. | Factor H | EDCNELPPRRNTEILTGSWSDQTYPEGTQAIYKCRPGYRSLGNIIMVCRKG |
| | domain 1-5 | EWVALNPLRKCQKRPCGHPGDTPFGTFTLTGGNVFEYGVKAVYTCNEGY |
| | | QLLGEINYRECDTDGWTNDIPICEVVKCLPVTAPENGKIVSSAMEPDREY |
| | | HFGQAVRFVCNSGYKIEGDEEMHCSDDGFWSKEKPKCVEISCKSPDVING |
| | | SPISQKIIYKENERFQYKCNMGYEYSERGDAVCTESGWRPLPSCEEKSCDN |
| | | PYIPNGDYSPLRIKHRTGDEITYQCRNGFYPATRGNTAKCTSTGWIPAPRC |
| | | TLKP |
| 202. | IgG4 Fc WT | ASTKGPSVFPLAPCSRSTSESTAALGCLVKDYFPEPVTVSWNSGALTSGVH |
| | factor H fusion | TFPAVLQSSGLYSLSSVVTVPSSSLGTKTYTCNVDHKPSNTKVDKRVESK |
| | | YGPPCPSCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKC |
| | | KVSNKGLPSSIEKTISKAKGQPREPQVYTLPPSQEEMTKNQVSLTCLVKGF |
| | | YPSDIAVEWESNGQPENNYKTTPPVLDSDGSFFLYSRLTVDKSRWQEGNV |
| | | FSCSVMHEALHNHYTQKSLSLSLGKEDCNELPPRRNTEILTGSWSDQTYP |
| | | EGTQAIYKCRPGYRSLGNIIMVCRKGEWVALNPLRKCQKRPCGHPGDTPF |
| | | GTFTLTGGNVFEYGVKAVYTCNEGYQLLGEINYRECDTDGWTNDIPICEV |
| | | VKCLPVTAPENGKIVSSAMEPDREYHFGQAVRFVCNSGYKIEGDEEMHCS |
| | | DDGFWSKEKPKCVEISCKSPDVINGSPISQKIIYKENERFQYKCNMGYEYS |
| | | ERGDAVCTESGWRPLPSCEEKSCDNPYIPNGDYSPLRIKHRTGDEITYQCR |
| | | NGFYPATRGNTAKCTSTGWIPAPRCTLKP |
| 203. | IgG4 Fc PLA | ASTKGPSVFPLAPCSRSTSESTAALGCLVKDYFPEPVTVSWNSGALTSGVH |
| | factor H fusion | TFPAVLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVSQEDPE |
| | | VQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEG |
| | | NVFSCSVLHEALHAHYTQKSLSLSLGKEDCNELPPRRNTEILTGSWSDQT |
| | | YPEGTQAIYKCRPGYRSLGNIIMVCRKGEWVALNPLRKCQKRPCGHPGD |
| | | TPFGTFTLTGGNVFEYGVKAVYTCNEGYQLLGEINYRECDTDGWTNDIPI |
| | | CEVVKCLPVTAPENGKIVSSAMEPDREYHFGQAVRFVCNSGYKIEGDEE |
| | | MHCSDDGFWSKEKPKCVEISCKSPDVINGSPISQKIIYKENERFQYKCNMG |
| | | YEYSERGDAVCTESGWRPLPSCEEKSCDNPYIPNGDYSPLRIKHRTGDEIT |
| | | YQCRNGFYPATRGNTAKCTSTGWIPAPRCTLKP |
| 204. | 69.44HC- | QVQLVQSGAEVKKPGASVKVSCKASGYSFTTYYMYWVRQAPGQGLEW |
| | FH1-5 IgG4 | MGIITPSGGSTTYAQKFQDRVTMTRDASTTTVYMELSSLRSEDTAVYYCA |
| | PLApeptide | RGYEGSYGFDHWGQGTLVTVSSASTKGPSVFPLAPCSRSTSESTAALGCL |
| | sequence | VKDYFPEPVTVSWNSGALTSGVHTFPAVLQSSGLYSLSSVVTVPSSSLGTK |
| | | TYTCNVDHKPSNTKVDKRVESKYGPPCPPCPAPEFLGGPSVFLFPPKPKDT |
| | | LMISRTPEVTCVVVDVSQEDPEVQFNWYVDGVEVHNAKTKPREEQFNST |
| | | YRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVL |
| | | DSDGSFFLYSRLTVDKSRWQEGNVFSCSVLHEALHAHYTQKSLSLSLGKE |
| | | DCNELPPRRNTEILTGSWSDQTYPEGTQAIYKCRPGYRSLGNIIMVCRKGE |
| | | WVALNPLRKCQKRPCGHPGDTPFGTFTLTGGNVFEYQVKAVYTCNEGY |
| | | QLLGEINYRECDTDGWTNDIPICEVVKCLPVTAPENGKIVSSAMEPDREY |
| | | HFGQAVRFVCNSGYKIEGDEEMHCSDDGFWSKEKPKCVEISCKSPDVING |
| | | SPISQKIIYKENERFQYKCNMGYEYSERGDAVCTESGWRPLPSCEEKSCDN |
| | | PYIPNGDYSPLRIKHRTGDEITYQCRNGFYPATRGNTAKCTSTGWIPAPRC |
| | | TLKP |
| 205. | 69.44LC | SYELTQPPSVSLSPGQTASITCSGDKLGNKYTCWYQQKPGQSPVLVIYQD |
| | peptide | NKRPSGIPERFSGSNSGNTATLTISGTQAMDEADYYCQTWDSSTAVFGGG |
| | sequence | TKLTVLGQPKAAPSVTLFPPSSEELQANKATLVCLISDFYPGAVTVAWKA |
| | | DSSPVKAGVETTTPSKQSNNKYAASSYLSLTPEQWKSHRSYSCQVTHEGS |
| | | TVEKTVAPTECS |
| 206. | 3.2HC-FH1-5 | EVQLLESGGGLVQPGGSLRLSCAASGFTFRHYAMSWVRQAPGKGLEWVS |
| | IgG4 PLA | LISGSGASTFYADSVKGRFTISRDNSENMLYLQMNSLRAEDTAVYYCAKD |
| | peptide | SLAVAGSEYFQHWGQGTLVTVSSASTKGPSVFPLAPCSRSTSESTAALGC |
| | sequence | LVKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPKD |
| | | TLMISRTPEVTCVVVDVSQEDPEVQFNWYVDGVEVHNAKTKPREEQFNS |
| | | TYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVL |
| | | DSDGSFFLYSRLTVDKSRWQEGNVFSCSVLHEALHAHYTQKSLSLSLGKE |
| | | DCNELPPRRNTEILTGSWSDQTYPEGTQAIYKCRPGYRSLGNIIMVCRKGE |
| | | WVALNPLRKCQKRPCGHPGDTPFGTFTLTGGNVFEYGVKAVYTCNEGY |
| | | QLLGEINYRECDTDGWTNDIPICEVVKCLPVTAPENGKIVSSAMEPDREY |
| | | HFGQAVRFVCNSGYKIEGDEEMHCSDDGFWSKEKPKCVEISCKSPDVING |
| | | SPISQKIIYKENERFQYKCNMGYEYSERGDAVCTESGWRPLPSCEEKSCDN |
| | | PYIPNGDYSPLRIKHRTGDEITYQCRNGFYPATRGNTAKCTSTGWIPAPRC |
| | | TLKP |
| 207. | 3.2LC peptide | SYVLTQAPSVSVAPGQTARITCGGNYFGGKSVHWYQQRPGQAPVLVVYD |
| | sequence | DSDRPSGIPERFSGSKSGNTATLAISRVEAGDEAAYYCQVYDTSSDHWVF |
| | | GGGTKLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVT |
| | | HEGSTVEKTVAPTECS |
| 208. | 69.44HC- | CAGGTGCAGCTGGTGCAGTCTGGGGCTGAGGTGAAGAAGCCTGGGGC |
| | FH1-5 | CTCAGTGAAGGTTTCCTGTAAGGCATCTGGATACAGTTTCACCACTTA |
| | nucleotide | CTATATGTACTGGGTGCGACAGGCCCCTGGACAAGGGCTTGAGTGGAT |
| | sequence | GGGAATCATCACCCCTAGCGGTGGTAGTACAACCTACGCACAGAAGTT |
| | | CCAGGACAGAGTCACCATGACAAGGGACGCGTCCACGACTACAGTCT |
| | | ACATGGAGCTGAGCAGCCTGAGATCTGAGGACACGGCCGTGTATTACT |
| | | GTGCGAGAGGGTATGAGGGGAGCTACGGGTTTGACCACTGGGGCCAG |
| | | GGAACCCTGGTCACCGTCTCCTCAGCTAGCACCAAGGGCCCATCGGTC |
| | | TTCCCCCTGGCGCCCTGCTCCAGGAGCACCTCCGAGAGCACAGCCGCC |
| | | CTGGGCTGCCTGGTCAAGGACTACTTCCCCGAACCGGTGACGGTGTCG |
| | | TGGAACTCAGGCGCCCTGACCAGCGGCGTGCACACCTTCCCGGCTGTC |
| | | CTACAGTCCTCAGGACTCTACTCCCTCAGCAGCGTGGTGACCGTGCCC |
| | | TCCAGCAGCTTGGGCACGAAGACCTACACCTGCAACGTAGATCACAA |
| | | GCCCAGCAACACCAAGGTGGACAAGAGAGTTGAGTCCAAATATGGTC |
| | | CCCCATGCCCACCATGCCCAGCACCTGAGTTCCTGGGGGGACCATCAG |
| | | TCTTCCTGTTCCCCCCAAAACCCAAGGACACTCTCATGATCTCCCGGAC |
| | | CCCTGAGGTCACGTGCGTGGTGGTGGACGTGAGCCAGGAAGACCCCG |
| | | AGGTCCAGTTCAACTGGTACGTGGATGGCGTGGAGGTGCATAATGCCA |
| | | AGACAAAGCCGCGGGAGGAGCAGTTCAACAGCACGTACCGTGTGGTC |
| | | AGCGTCCTCACCGTCCTGCACCAGGACTGGCTGAACGGCAAGGAGTAC |
| | | AAGTGCAAGGTCTCCAACAAAGGCCTCCCGTCCTCCATCGAGAAAACC |
| | | ATCTCCAAAGCCAAAGGGCAGCCCCGAGAGCCACAGGTGTACACCCT |
| | | GCCCCCATCCCAGGAGGAGATGACCAAGAACCAGGTCAGQCTGACCT |
| | | GCCTGGTCAAAGGCTTCTACCCCAGCGACATCGCCGTGGAGTGGGAGA |
| | | GCAATGGGCAGCCGGAGAACAACTACAAGACCACGCCTCCCGTGCTG |
| | | GACTCCGACGGCTCCTTCTTCCTCTACAGCAGGCTAACCGTGGACAAG |
| | | AGCAGGTGGCAGGAGGGGAATGTCTTCTCATGCTCCGTGCTGCATGAG |
| | | GCTCTGCACGCCCACTACACACAGAAGAGCCTCTCCCTGTCTCTGGGT |
| | | AAAGAGGATTGTAACGAGCTGCCACCCAGAAGAAACACAGAGATCCT |
| | | GACCGGAAGCTGGAGCGATCAGACATACCCCGAGGGCACCCAGGCCA |
| | | TTTACAAGTGCAGACCCGGATATAGAAGCCTGGGCAACATTATCATGG |
| | | TGTGCAGAAAAGGAGAGTGGGTGGCCCTGAACCCTCTGAGAAAGTGC |
| | | CAGAAAAGACCATGCGGCCACCCAGGCGACACCCCCTTCGGAACCTTC |
| | | ACACTGACAGGCGGCAACGTGTTTGAGTACGGCGTGAAAGCTGTGTAT |
| | | ACCTGCAACGAGGGCTACCAGCTGCTGGGAGAAATCAACTACAGAGA |
| | | GTGCGACACCGACGGCTGGACCAACGACATCCCTATTTGCGAGGTGGT |
| | | GAAGTGCCTGCCCGTGACTGCCCCCGAGAACGGAAAGATCGTGAGCA |
| | | GCGCCATGGAGCCCGACAGAGAGTACCACTTTGGACAGGCCGTGAGA |
| | | TTTGTGTGCAACAGCGGCTATAAGATCGAGGGCGACGAGGAAATGCA |
| | | CTGCAGCGACGACGGCTTTTGGTCAAAGGAGAAGCCCAAATGCGTGG |
| | | AGATCAGCTGCAAATCTCCCGACGTGATCAACGGCAGCCCTATCAGCC |
| | | AGAAGATCATTTACAAGGAGAACGAGAGATTCCAGTACAAGTGCAAC |
| | | ATGGGATACGAGTACTCCGAGAGAGGCGACGCCGTGTGCACCGAGAG |
| | | CGGCTGGAGACCACTGCCCAGCTGCGAGGAGAAGAGCTGCGACAACC |
| | | CCTACATTCCAAACGGCGACTACAGCCCACTGAGGATCAAGCACAGA |
| | | ACAGGAGATGAGATTACATACCAGTGTAGAAACGGCTTCTACCCCGCC |
| | | ACCAGAGGAAACACCGCCAAATGCACCAGCACCGGCTGGATTCCTGC |
| | | CCCCCGGTGCACCCTGAAACCATGA |
| 209. | 69.44LC | TCCTATGAACTGACTCAGCCACCCTCAGTGTCCCTGTCCCCAGGACAG |
| | nucleotide | ACAGCCAGCATCACCTGCTCTGGAGATAAATTGGGAAATAAATATACT |
| | sequence | TGCTGGTATCAGCAGAAGCCAGGCCAGTCCCCTGTCCTGGTCATCTAT |
| | | CAAGATAACAAGCGGCCCTCGGGGATCCCTGAGCGATTCTCTGGCTCC |
| | | AACTCTGGGAACACAGCCACTCTGACCATCAGCGGGACCCAGGCTATG |
| | | GATGAGGCTGACTATTACTGTCAGACGTGGGACAGCAGCACTGCGGTG |
| | | TTCGGCGGAGGGACCAAGCTGACCGTCCTAGGTCAGCCCAAGGCTGCC |
| | | CCCTCGGTCACTCTGTTCCCGCCCTCCTCTGAGGAGCTTCAAGCCAACA |
| | | AGGCCACACTGGTGTGTCTCATAAGTGACTTCTACCCGGGAGCCGTGA |
| | | CAGTGGCCTGGAAGGCAGATAGCAGCCCCGTCAAGGCGGGAGTGGAG |
| | | ACCACCACACCCTCCAAACAAAGCAACAACAAGTACGCGGCCAGCAG |
| | | CTATCTGAGCCTGACGCCTGAGCAGTGGAAGTCCCACAGAAGCTACAG |
| | | CTGCCAGGTCACGCATGAAGGGAGCACCGTGGAGAAGACAGTGGCCC |
| | | CTACAGAATGTTCATAG |
| 210. | IgG1 Fc WT | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| 211. | IgG1 Fc WT | ASTKGPSVFPLAPSSKSTSGGTAALGCLVKDYFPEPVTVSWNSGALTSGV |
| | factor H fusion | HTFPAVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGKEDCNELPPRRNTEILTGSWS |
| | | DQTYPEGTQAIYKCRPGYRSLGNIIMVCRKGEWVALNPLRKCQKRPCGH |
| | | PGDTPFGTFTLTGGNVFEYGVKAVYTCNEGYQLLGEINYRECDTDGWTN |
| | | DIPICEVVKCLPVTAPENGKIVSSAMEPDREYHFGQAVRFVCNSGYKIEGD |
| | | EEMHCSDDGFWSKEKPKCVEISCKSPDVINGSPISQKIIYKENERFQYKCN |
| | | MGYEYSERGDAVCTESGWRPLPSCEEKSCDNPYIPNGDYSPLRIKHRTGD |
| | | EITYQCRNGFYPATRGNTAKCTSTGWIPAPRCTLKP |
| 212. | 69.44HC- | QVQLVQSGAEVKKPGASVKVSCKASGYSFTTYYMYWVRQAPGQGLEW |
| | FH1-5 (IgG1) | MGIITPSGGSTTYAQKFQDRVTMTRDASTTTVYMELSSLRSEDTAVYYCA |
| | peptide | RGYEGSYGFDHWGQGTLVTVSSASTKGPSVFPLAPSSKSTSGGTAALGCL |
| | sequence | VKDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSRDELTKNQVSLTCLVKGFYPSDIAVEWESNGQPENNYKTTPP |
| | | VLDSDGSFFLYSKLTVDKSRWQQGNVFSCSVMHEALHNHYTQKSLSLSP |
| | | GKEDCNELPPRRNTEILTGSWSDQTYPEGTQAIYKCRPGYRSLGNIIMVCR |
| | | KGEWVALNPLRKCQKRPCGHPGDTPFGTFTLTGGNVFEYGVKAVYTCNE |
| | | GYQLLGEINYRECDTDGWTNDIPICEVVKCLPVTAPENGKIVSSAMEPDR |
| | | EYHFGQAVRFVCNSGYKIEGDEEMHCSDDGFWSKEKPKCVEISCKSPDVI |
| | | NGSPISQKIIYKENERFQYKCNMGYEYSERGDAVCTESGWRPLPSCEEKSC |
| | | DNPYIPNGDYSPLRIKHRTGDEITYQCRNGFYPATRGNTAKCTSTGWIPAP |
| | | RCTLKP |
| 213. | 3.2HC-FH1-5 | EVQLLESGGGLVQPGGSLRLSCAASGFTFRHYAMSWVRQAPGKGLEWVS |
| | (IgG1) peptide | LISGSGASTFYADSVKGRFTISRDNSENMLYLQMNSLRAEDTAVYYCAKD |
| | sequence | SLAVAGSEYFQHWGQGTLVTVSSASTKGPSVFPLAPSSKSTSGGTAALGC |
| | | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGKEDCNELPPRRNTEILTGSWSDQTYPEGTQAIYKCRPGYRSLGNIIMV |
| | | CRKGEWVALNPLRKCQKRPCGHPGDTPFGTFTLTGGNVFEYGVKAVYTC |
| | | NEGYQLLGEINYRECDTDGWTNDIPICEVVKCLPVTAPENGKIVSSAMEP |
| | | DREYHFGQAVRFVCNSGYKIEGDEEMHCSDDGFWSKEKPKCVEISCKSP |
| | | DVINGSPISQKIIYKENERFQYKCNMGYEYSERGDAVCTESGWRPLPSCEE |
| | | KSCDNPYIPNGDYSPLRIKHRTGDEITYQCRNGFYPATRGNTAKCTSTGWI |
| | | PAPRCTLKP |
| 214. | IgG1 Fc | ASTKGPSVFPLAPSSKSTSGGTAALGCLVKDYFPEPVTVSWNSGALTSGV |
| | LALA-LS | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVLHEALHSHYTQKSLSLSPGK |
| 215. | IgG1 Fc | ASTKGPSVFPLAPSSKSTSGGTAALGCLVKDYFPEPVTVSWNSGALTSGV |
| | LALA-LA | HTFPAVLQSSGLYSLSSVVTVPSSSLGTQTYICNVNHKPSNTKVDKKVEPK |
| | | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVLHEALHAHYTQKSLSLSPGK |
| 216. | IgG4 Fc PLS | ASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESK |
| | IgG1 Fc | YGPPCPPCPAPEFLGGPSVFLFPPKPKDTLMISRTPEVTCVVVDVSQEDPE |
| | LALA-LS | VQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEG |
| | | NVFSCSVLHEALHSHYTQKSLSLSLGK |
| 217. | IgG1 Fc | GCTAGCACCAAGGGCCCATCGGTCTTCCCCCTGGCACCCTCCTCCAAG |
| | | AGCACCTCTGGGGGCACAGCGGCCCTGGGCTGCCTGGTCAAGGACTAC |
| | | TTCCCCGAACCGGTGACGGTGTCGTGGAACTCAGGCGCCCTGACCAGC |
| | | GGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCT |
| | | ACATCTGCAACGTGAATCACAAGCCCAGCAACACCAAGGTGGACAAG |
| | | AAAGTTGAGCCCAAATCTTGTGACAAAACTCACACATGCCCACCGTGC |
| | | CCAGCACCTGAAGCAGCAGGGGGACCGTCAGTCTTCCTCTTCCCCCCA |
| | | AAACCCAAGGACACCCTCATGATCTCCCGGACCCCTGAGGTCACATGC |
| | | GTGGTGGTGGACGTGAGCCACGAAGACCCTGAGGTCAAGTTCAACTG |
| | | GTACGTGGACGGCGTGGAGGTGCATAATGCCAAGACAAAGCCGCGGG |
| | | AGGAGCAGTACAACAGCACGTACCGTGTGGTCAGCGTCCTCACCGTCC |
| | | TGCACCAGGACTGGCTGAATGGCAAGGAGTACAAGTGCAAGGTCTCC |
| | | AACAAAGCCCTCCCAGCCCCCATCGAGAAAACCATCTCCAAAGCCAA |
| | | AGGGCAGCCCCGAGAACCACAGGTGTACACCCTGCCCCCATCCCGGG |
| | | ATGAGCTGACCAAGAACCAGGTCAGCCTGACCTGCCTGGTCAAAGGCT |
| | | TCTATCCCAGCGACATCGCCGTGGAGTGGGAGAGCAATGGGCAGCCG |
| | | GAGAACAACTACAAGACCACGCCTCCCGTGCTGGACTCCGACGGATCC |
| | | TTCTTCCTCTACAGCAAGCTCACCGTGGACAAGAGCAGGTGGCAGCAG |
| | | GGGAACGTCTTCTCATGCTCCGTGCTGCATGAGGCTCTGCACTCCCACT |
| | | ACACGCAGAAGAGCCTCTCCCTGTCTCCGGGTAAATAG |
| 218. | LALA-LA | GCTAGCACCAAGGGCCCATCGGTCTTCCCCCTQGCACCCTCCTCCAAG |
| | | AGCACCTCTGGGGGCACAGCGGCCCTGGGCTGCCTGGTCAAGGACTAC |
| | | TTCCCCGAACCGGTGACGGTGTCGTGGAACTCAGGCGCCCTGACCAGC |
| | | GGCGTGCACACCTTCCCGGCTGTCCTACAGTCCTCAGGACTCTACTCCC |
| | | TCAGCAGCGTGGTGACCGTGCCCTCCAGCAGCTTGGGCACCCAGACCT |
| | | ACATCTGCAACGTGAATCACAAGCCCAGCAACACCAAGGTGGACAAG |
| | | AAAGTTGAGCCCAAATCTTGTGACAAAACTCACACATGCCCACCGTGC |
| | | CCAGCACCTGAAGCAGCAGGGGGACCGTCAGTCTTCCTCTTCCCCCCA |
| | | AAACCCAAGGACACCCTCATGATCTCCCGGACCCCTGAGGTCACATGC |
| | | GTGGTGGTGGACGTGAGCCACGAAGACCCTGAGGTCAAGTTCAACTG |
| | | GTACGTGGACGGCGTGGAGGTGCATAATGCCAAGACAAAGCCGCGGG |
| | | AGGAGCAGTACAACAGCACGTACCGTGTGGTCAGCGTCCTCACCGTCC |
| | | TGCACCAGGACTGGCTGAATGGCAAGGAGTACAAGTGCAAGGTCTCC |
| | | AACAAAGCCCTCCCAGCCCCCATCGAGAAAACCATCTCCAAAGCCAA |
| | | AGGGCAGCCCCGAGAACCACAGGTGTACACCCTGCCCCCATCCCGGG |
| | | ATGAGCTGACCAAGAACCAGGTCAGCCTGACCTGCCTGGTCAAAGGCT |
| | | TCTATCCCAGCGACATCGCCGTGGAGTGGGAGAGCAATGGGCAGCCG |
| | | GAGAACAACTACAAGACCACGCCTCCCGTGCTGGACTCCGACGGATCC |
| | | TTCTTCCTCTACAGCAAGCTCACCGTGGACAAGAGCAGGTGGCAGCAG |
| | | GGGAACGTCTTCTCATGCTCCGTGCTGCATGAGGCTCTGCACGCCCAC |
| | | TACACGCAGAAGAGCCTCTCCCTGTCTCCGGGTAAATAG |
| 219. | IgG4 Fc PLS | GCTAGCACCAAGGGCCCATCGGTCTTCCCCCTGGCGCCCTGCTCCAGG |
| | | AGCACCTCCGAGAGCACAGCCGCCCTGGGCTGCCTGGTCAAGGACTAC |
| | | TTCCCCGAACCGGTGACGGTGTCGTGGAACTCAGGCGCCCTGACCAGC |
| | | GGCGTGCACACCTTCCCGGCTGTCCTACAGTCCTCAGGACTCTACTCCC |
| | | TCAGCAGCGTGGTGACCGTGCCCTCCAGCAGCTTGGGCACGAAGACCT |
| | | ACACCTGCAACGTAGATCACAAGCCCAGCAACACCAAGGTGGACAAG |
| | | AGAGTTGAGTCCAAATATGGTCCCCCATGCCCACCATGCCCAGCACCT |
| | | GAGTTCCTGGGGGGACCATCAGTCTTCCTGTTCCCCCCAAAACCCAAG |
| | | GACACTCTCATGATCTCCCGGACCCCTGAGGTCACGTGCGTGGTGGTG |
| | | GACGTGAGCCAGGAAGACCCCGAGGTCCAGTTCAACTGGTACGTGGA |
| | | TGGCGTGGAGGTGCATAATGCCAAGACAAAGCCGCGGGAGGAGCAGT |
| | | TCAACAGCACGTACCGTGTGGTCAGCGTCCTCACCGTCCTGCACCAGG |
| | | ACTGGCTGAACGGCAAGGAGTACAAGTGCAAGGTCTCCAACAAAGGC |
| | | CTCCCGTCCTCCATCGAGAAAACCATCTCCAAAGCCAAAGGGCAGCCC |
| | | CGAGAGCCACAGGTGTACACCCTGCCCCCATCCCAGGAGGAGATGAC |
| | | CAAGAACCAGGTCAGCCTGACCTGCCTGGTCAAAGGCTTCTACCCCAG |
| | | CGACATCGCCGTGGAGTGGGAGAGCAATGGGCAGCCGGAGAACAACT |
| | | ACAAGACCACGCCTCCCGTGCTGGACTCCGACGGCTCCTTCTTCCTCTA |
| | | CAGCAGGCTAACCGTGGACAAGAGCAGGTGGCAGGAGGGGAATGTCT |
| | | TCTCATGCTCCGTGCTGCATGAGGCTCTGCACTCCCACTACACACAGA |
| | | AGAGCCTCTCCCTGTCTCTGGGTAAATAG |
| 220. | IgG1 Fc | ASTKGPSVFPLAPSSKSTSGGTAALGCLVKDYFPEPVTVSWNSGALTSGV |
| | LALA-LS | HTFPAVLQSSGLYSLSSVVTVPSSSLGTQTYICNVNHKPSNTKVDKKVEPK |
| | factor H fusion | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVLHEALHSHYTQKSLSLSPGKEDCNELPPRRNTEILTGSWS |
| | | DQTYPEGTQAIYKCRPGYRSLGNIIMVCRKGEWVALNPLRKCQKRPCGH |
| | | PGDTPFGTFTLTGGNVFEYGVKAVYTCNEGYQLLGEINYRECDTDGWTN |
| | | DIPICEVVKCLPVTAPENGKIVSSAMEPDREYHFGQAVRFVCNSGYKIEGD |
| | | EEMHCSDDGFWSKEKPKCVEISCKSPDVINGSPISQKIIYKENERFQYKCN |
| | | MGYEYSERGDAVCTESGWRPLPSCEEKSCDNPYIPNGDYSPLRIKHRTGD |
| | | EITYQCRNGFYPATRGNTAKCTSTGWIPAPRCTLKP |
| 221. | IgG1 Fc | ASTKGPSVFPLAPSSKSTSGGTAALGCLVKDYFPEPVTVSWNSGALTSGV |
| | LALA-LA | HTFPAVLQSSGLYSLSSVVTVPSSSLGTQTYICNVNHKPSNTKVDKKVEPK |
| | factor H fusion | SCDKTHTCPPCPAPEAAGGPSVFLFPPKPKDTLMISRTPEVTCVVVDVSHE |
| | | DPEVKFNWYVDGVEVHNAKTKPREEQYNSTYRVVSVLTVLHQDWING |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVLHEALHAHYTQKSLSLSPGKEDCNELPPRRNTEILTGSWS |
| | | DQTYPEGTQAIYKCRPGYRSLGNIIMVCRKGEWVALNPLRKCQKRPCGH |
| | | PGDTPFGTFTLTGGNVFEYGVKAVYTCNEGYQLLGEINYRECDTDGWTN |
| | | DIPICEVVKCLPVTAPENGKIVSSAMEPDREYHFGQAVRFVCNSGYKIEGD |
| | | EEMHCSDDGFWSKEKPKCVEISCKSPDVINGSPISQKIIYKENERFQYKCN |
| | | MGYEYSERGDAVCTESGWRPLPSCEEKSCDNPYIPNGDYSPLRIKHRTGD |
| | | EITYQCRNGFYPATRGNTAKCTSTGWIPAPRCTLKP |
| 222. | hand gG4 Fc PLS | ASTKGPSVFPLAPCSRSTSESTAALGCLVKDYFPEPVTVSWNSGALTSGVH |
| | factor H fusion | TFPAVLQSSGLYSLSSVVTVPSSSLGTKTYTCNVDHKPSNTKVDKRVESK |
| | | YGPPCPPCPAPEFLGGPSVFLFPPKPKDTLMISRTPEVTCVVVDVSQEDPE |
| | | VQFNWYVDGVEVHNAKTKPREEQFNSTYRVVSVLTVLHQDWLNGKEY |
| | | KCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEG |
| | | NVFSCSVLHEALHSHYTQKSLSLSLGKEDCNELPPRRNTEILTGSWSDQTY |
| | | PEGTQAIYKCRPGYRSLGNIIMVCRKGEWVALNPLRKCQKRPCGHPGDTP |
| | | FGTFTLTGGNVFEYGVKAVYTCNEGYQLLGEINYRECDTDGWTNDIPICE |
| | | VVKCLPVTAPENGKIVSSAMEPDREYHFGQAVRFVCNSGYKIEGDEEMH |
| | | CSDDGFWSKEKPKCVEISCKSPDVINGSPISQKIIYKENERFQYKCNMGYE |
| | | YSERGDAVCTESGWRPLPSCEEKSCDNPYIPNGDYSPLRIKHRTGDEITYQ |
| | | CRNGFYPATRGNTAKCTSTGWIPAPRCTLKP |
| 223. | 3.2 H2-10 | EVQLLESGGGLVQPGGSLRLSCAASGFTFRHYAMSWVRQAPGKGLEWVS |
| | IgG1 Fc FH | LISGSGASTHYADSVKGRFTISRDNSENMLYLQMNSLRAEDTAVYYCAK |
| | fusion | DSLAVAGSEYFQHWGQGTLVTVSASTKGPSVFPLAPSSKSTSGGTAALGC |
| | | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPPK |
| | | PKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGKEDCNELPPRRNTEILTGSWSDQTYPEGTQAIYKCRPGYRSLGNIIMV |
| | | CRKGEWVALNPLRKCQKRPCGHPGDTPFGTFTLTGGNVFEYGVKAVYTC |
| | | NEGYQLLGEINYRECDTDGWTNDIPICEVVKCLPVTAPENGKIVSSAMEP |
| | | DREYHFGQAVRFVCNSGYKIEGDEEMHCSDDGFWSKEKPKCVEISCKSP |
| | | DVINGSPISQKHIYKENERFQYKCNMGYEYSERGDAVCTESGWRPLPSCEE |
| | | KSCDNPYIPNGDYSPLRIKHRTGDEITYQCRNGFYPATRGNTAKCTSTGWI |
| | | PAPRCTLKP |
| 224. | 3.2 H2-10 | EVQLLESGGGLVQPGGSLRLSCAASGFTFRHYAMSWVRQAPGKGLEWVS |
| | IgG1 LALA- | LISGSGASTHYADSVKGRFTISRDNSENMLYLQMNSLRAEDTAVYYCAK |
| | LS Fc FH | DSLAVAGSEYFQHWGQGTLVTVSASTKGPSVFPLAPSSKSTSGGTAALGC |
| | fusion | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPEAAGGPSVFLFPP |
| | | KPKDTLMISRTPEVTCVVVDVSHEDPEVKENWYVDGVEVHNAKTKPREE |
| | | QYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQKSLSL |
| | | SPGKEDCNELPPRRNTEILTGSWSDQTYPEGTQAIYKCRPGYRSLGNIIMV |
| | | CRKGEWVALNPLRKCQKRPCGHPGDTPFGTFTLTGGNVFEYGVKAVYTC |
| | | NEGYQLLGEINYRECDTDGWTNDIPICEVVKCLPVTAPENGKIVSSAMEP |
| | | DREYHFGQAVRFVCNSGYKIEGDEEMHCSDDGFWSKEKPKCVEISCKSP |
| | | DVINGSPISQKIIYKENERFQYKCNMGYEYSERGDAVCTESGWRPLPSCEE |
| | | KSCDNPYIPNGDYSPLRIKHRTGDEITYQCRNGFYPATRGNTAKCTSTGWI |
| | | PAPRCTLKP |
| 225. | 3.2 H2-10 | EVQLLESGGGLVQPGGSLRLSCAASGFTFRHYAMSWVRQAPGKGLEWVS |
| | IgGI LALA- | LISGSGASTHYADSVKGRFTISRDNSENMLYLQMNSLRAEDTAVYYCAK |
| | LAFc FH | DSLAVAGSEYFQHWGQGTLVTVSASTKGPSVFPLAPSSKSTSGGTAALGC |
| | fusion | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKT |
| | | TPPVLDSDGSFFLYSKLTVDKSRWQQGNVFSCSVLHEALHAHYTQKSLSL |
| | | SPGKEDCNELPPRRNTEILTGSWSDQTYPEGTQAIYKCRPGYRSLGNIIMV |
| | | CRKGEWVALNPLRKCQKRPCGHPGDTPFGTFTLTGGNVFEYGVKAVYTC |
| | | NEGYQLLGEINYRECDTDGWTNDIPICEVVKCLPVTAPENGKIVSSAMEP |
| | | DREYHFGQAVRFVCNSGYKIEGDEEMHCSDDGFWSKEKPKCVEISCKSP |
| | | DVINGSPISQKIIYKENERFQYKCNMGYEYSERGDAVCTESGWRPLPSCEE |
| | | KSCDNPYIPNGDYSPLRIKHRTGDEITYQCRNGFYPATRGNTAKCTSTGWI |
| | | PAPRCTLKP |
| 226. | 3.2 H2-10 | EVQLLESGGGLVQPGGSLRLSCAASGFTFRHYAMSWVRQAPGKGLEWVS |
| | IgG4 Fc FH | LISGSGASTHYADSVKGRFTISRDNSENMLYLQMNSLRAEDTAVYYCAK |
| | fusion | DSLAVAGSEYFQHWGQGTLVTVS |
| | | ASTKGPSVFPLAPCSRSTSESTAALGCLVKDYFPEPVTVSWNSGALTSGVH |
| | | TFPAVLQSSGLYSLSSVVTVPSSSLGTKTYTCNVDHKPSNTKVDKRVESK |
| | | YGPPCPSCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKC |
| | | KVSNKGLPSSIEKTISKAKGQPREPQVYTLPPSQEEMTKNQVSLTCLVKGF |
| | | YPSDIAVEWESNGQPENNYKTTPPVLDSDGSFFLYSRLTVDKSRWQEGNV |
| | | FSCSVMHEALHNHYTQKSLSLSLGKEDCNELPPRRNTEILTGSWSDQTYP |
| | | EGTQAIYKCRPGYRSLGNIIMVCRKGEWVALNPLRKCQKRPCGHPGDTPF |
| | | GTFTLTGGNVFEYGVKAVYTCNEGYQLLGEINYRECDTDGWTNDIPICEV |
| | | VKCLPVTAPENGKIVSSAMEPDREYHFGQAVRFVCNSGYKIEGDEEMHCS |
| | | DDGFWSKEKPKCVEISCKSPDVINGSPISQKIIYKENERFQYKCNMGYEYS |
| | | ERGDAVCTESGWRPLPSCEEKSCDNPYIPNGDYSPLRIKHRTGDEITYQCR |
| | | NGFYPATRGNTAKCTSTGWIPAPRCTLKP |
| 227. | 3.2 H2-10 | EVQLLESGGGLVQPGGSLRLSCAASGFTFRHYAMSWVRQAPGKGLEWVS |
| | IgG4 PLAFc | LISGSGASTHYADSVKGRFTISRDNSENMLYLQMNSLRAEDTAVYYCAK |
| | FH fusion | DSLAVAGSEYFQHWGQGTLVTVSASTKGPSVFPLAPCSRSTSESTAALGC |
| | | LVKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVLHEALHAHYTQKSLSLSLGK |
| | | EDCNELPPRRNTEILTGSWSDQTYPEGTQAIYKCRPGYRSLGNIIMVCRKG |
| | | EWVALNPLRKCQKRPCGHPGDTPFGTFTLTGGNVFEYGVKAVYTCNEGY |
| | | QLLGEINYRECDTDGWTNDIPICEVVKCLPVTAPENGKIVSSAMEPDREY |
| | | HFGQAVRFVCNSGYKIEGDEEMHCSDDGFWSKEKPKCVEISCKSPDVING |
| | | SPISQKIIYKENERFQYKCNMGYEYSERGDAVCTESGWRPLPSCEEKSCDN |
| | | PYIPNGDYSPLRIKHRTGDEITYQCRNGFYPATRGNTAKCTSTGWIPAPRC |
| | | TLKP |
| 228. | 3.2 H2-10 | EVQLLESGGGLVQPGGSLRLSCAASGFTFRHYAMSWVRQAPGKGLEWVS |
| | IgG4 PLS Fc | LISGSGASTHYADSVKGRFTISRDNSENMLYLQMNSLRAEDTAVYYCAK |
| | FH fusion | DSLAVAGSEYFQHWGQGTLVTVSASTKGPSVFPLAPCSRSTSESTAALGC |
| | | LVKDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKPK |
| | | DTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQ |
| | | VYTLPPSQEEMTKNQVSLTCLVKGFYPSDIAVEWESNGQPENNYKTTPPV |
| | | LDSDGSFFLYSRLTVDKSRWQEGNVFSCSVLHEALHSHYTQKSLSLSLGK |
| | | EDCNELPPRRNTEILTGSWSDQTYPEGTQAIYKCRPGYRSLGNIIMVCRKG |
| | | EWVALNPLRKCQKRPCGHPGDTPFGTFTLTGGNVFEYGVKAVYTCNEGY |
| | | QLLGEINYRECDTDGWTNDIPICEVVKCLPVTAPENGKIVSSAMEPDREY |
| | | HFGQAVRFVCNSGYKIEGDEEMHCSDDGFWSKEKPKCVEISCKSPDVING |
| | | SPISQKIIYKENERFQYKCNMGYEYSERGDAVCTESGWRPLPSCEEKSCDN |
| | | PYIPNGDYSPLRIKHRTGDEITYQCRNGFYPATRGNTAKCTSTGWIPAPRC |
| | | TLKP |
| 229. | 3.2 H2-10 | SYVLTQAPSVSVAPGQTARITCGGNYFGGKSVHWYQQRPGQAPVLVVYD |
| | parental FH | DSDRPSGIPERFSGSKSGNTATLAISRVEAGDEAAYYCQVYDTSSDHWVF |
| | fusion VL | GGGTKLTVL |
| 230. | 3.2 H2-10 × L1- | SYVLTQAPSVSVAPGQTARITCGHNYFGGKSVHWYQQRPGQAPVLVVY |
| | 2 FH fusion | DDSDRPSGIPERFSGSKSGNTATLAISRVEAGDEAAYYCQVYDTSSDHWV |
| | VL | FGGGTKLTVL |
| 231. | 3.2 H2-10 × L1- | SYVLTQAPSVSVAPGQTARITCGGNHFGGKSVHWYQQRPGQAPVLVVY |
| | 4 FH fusion | DDSDRPSGIPERFSGSKSGNTATLAISRVEAGDEAAYYCQVYDTSSDHWV |
| | VL | FGGGTKLTVL |
| 232. | 3.2 H2-10 × L1- | SYVLTQAPSVSVAPGQTARITCGGNYHGGKSVHWYQQRPGQAPVLVVY |
| | 5 FH fusion | DDSDRPSGIPERFSGSKSGNTATLAISRVEAGDEAAYYCQVYDTSSDHWV |
| | VL | FGGGTKLTVL |
| 233. | 3.2 H2-10 × L1- | SYVLTQAPSVSVAPGQTARITCGGNYEGHIKSVHWYQQRPGQAPVLVVY |
| | 7 FH fusion | DDSDRPSGIPERFSGSKSGNTATLAISRVEAGDEAAYYCQVYDTSSDHWV |
| | VL | FGGGTKLTVL |
| 234. | 3.2 H2-10 × L1- | SYVLTQAPSVSVAPGQTARITCGGNYFGGHSVHWYQQRPGQAPVLVVY |
| | 8 FH fusion | DDSDRPSGIPERFSGSKSGNTATLAISRVEAGDEAAYYCQVYDTSSDHWV |
| | VL | FGGGTKLTVL |
| 235. | 3.2 H2-10 × L1- | SYVLTQAPSVSVAPGQTARITCGGNYFGGKSHHWYQQRPGQAPVLVVY |
| | 10 FH fusion | DDSDRPSGIPERFSGSKSGNTATLAISRVEAGDEAAYYCQVYDTSSDHWV |
| | VL | FGGGTKLTVL |
| 236. | 3.2 H2-10 × L2- | SYVLTQAPSVSVAPGQTARITCGGNYFGGKSVHWYQQRPGQAPVLVVY |
| | 1 VL | HDSDRPSGIPERFSGSKSGNTATLAISRVEAGDEAAYYCQVYDTSSDHWV |
| | | FGGGTKLTVL |
| 237. | 3.2 H2-10 × L2- | SYVLTQAPSVSVAPGQTARITCGGNYFGGKSVHWYQQRPGQAPVLVVYD |
| | 3 FH fusion | DHDRPSGIPERFSGSKSGNTATLAISRVEAGDEAAYYCQVYDTSSDHWVF |
| | VL | GGGTKLTVL |
| 238. | 3.2 H2-10 × L2- | SYVLTQAPSVSVAPGQTARITCGGNYFGGKSVHWYQQRPGQAPVLVVYD |
| | 5 FH fusion | DSDHPSGIPERFSGSKSGNTATLAISRVEAGDEAAYYCQVYDTSSDHWVF |
| | VL | GGGTKLTVL |
| 239. | 3.2 H2-10 × L2- | SYVLTQAPSVSVAPGQTARITCGGNYFGGKSVHWYQQRPGQAPVLVVYD |
| | 6 FH fusion | DSDRHSGIPERFSGSKSGNTATLAISRVEAGDEAAYYCQVYDTSSDHWVF |
| | VL | GGGTKLTVL |
| 240 | 3.2 H2-10 × L2- | SYVLTQAPSVSVAPGQTARITCGGNYFGGKSVHWYQQRPGQAPVLVVYD |
| | 7 FH fusion | DSDRPHGIPERFSGSKSGNTATLAISRVEAGDEAAYYCQVYDTSSDHWVF |
| | VL | GGGTKLTVL |
| 241. | 3.2 H2-10 × L3- | SYVLTQAPSVSVAPGQTARITCGGNYFGGKSVHWYQQRPGQAPVLVVYD |
| | 4 FH fusion | DSDRPSGIPERFSGSKSGNTATLAISRVEAGDEAAYYCQVYHTSSDHWVF |
| | VL | GGGTKLTVL |
| 242 | 3.2 H2-10 × L3- | SYVLTQAPSVSVAPGQTARITCGGNYFGGKSVHWYQQRPGQAPVLVVYD |
| | 5 FH fusion | DSDRPSGIPERFSGSKSGNTATLAISRVEAGDEAAYYCQVYDHSSDHWVF |
| | VL | GGGTKLTVL |
| 243. | 3.2 H2-10 × L3- | SYVLTQAPSVSVAPGQTARITCGGNYFGGKSVHWYQQRPGQAPVLVVYD |
| | 6 FH fusion | DSDRPSGIPERFSGSKSGNTATLAISRVEAGDEAAYYCQVYDTHSDHWVF |
| | VL | GGGTKLTVL |
| 244. | 3.2 H2-10 × L3- | SYVLTQAPSVSVAPGQTARITCGGNYFGGKSVHWYQQRPGQAPVLVVYD |
| | 7 FH fusion | DSDRPSGIPERFSGSKSGNTATLAISRVEAGDEAAYYCQVYDTSHDHWVF |
| | VL | GGGTKLTVL |
| 245. | 3.2 H2-10 × L3- | SYVLTQAPSVSVAPGQTARITCGGNYFGGKSVHWYQQRPGQAPVLVVYD |
| | 11 FH fusion | DSDRPSGIPERFSGSKSGNTATLAISRVEAGDEAAYYCQVYDTSSDHWHF |
| | VI | GGGTKLTVL |
| 246. | 3.2pH-GL VH | EVQLLESGGGLVQPGGSLRLSCAASGFTFRHYAMSWVRQAPGKGLEWVS |
| | | LISGSGASTHYADSVKGRFTISRDNSKNTLYLQMNSLRAEDTAVYYCAK |
| | | DSLAVAGSEYFQHWGQGTLVTVSS |
| 247. | 3.2pH-GL VL | SYVLTQPPSVSVAPGQTARITCGHNYFGGKSVHWYQQKPGQAPVLVVYD |
| | | DSDRPSGIPERFSGSNSGNTATLTISRVEAGDEADYYCQVYDTSSDHWVF |
| | | GGGTKLTVL |
| 248. | 3.2HC-GL- | EVQLLESGGGLVQPGGSLRLSCAASGFTFRHYAMSWVRQAPGKGLEWVS |
| | FH1-5-IgG1- | LISGSGASTHYADSVKGRFTISRDNSKNTLYLQMNSLRAEDTAVYYCAKD |
| | LALA-LS | SLAVAGSEYFQHWGQGTLVTVSSASTKGPSVFPLAPSSKSTSGGTAALGC |
| | | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPEAAGGPSVFLFPP |
| | | KPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKT |
| | | TPPVLDSDGSFFLYSKLTVDKSRWQQGNVFSCSVLHEALHSHYTQKSLSL |
| | | SPGKEDCNELPPRRNTEILTGSWSDQTYPEGTQAIYKCRPGYRSLGNIIMV |
| | | CRKGEWVALNPLRKCQKRPCGHPGDTPFGTFTLTGGNVFEYGVKAVYTC |
| | | NEGYQLLGEINYRECDTDGWTNDIPICEVVKCLPVTAPENGKIVSSAMEP |
| | | DREYHFGQAVRFVCNSGYKIEGDEEMHCSDDGFWSKEKPKCVEISCKSP |
| | | DVINGSPISQKIIYKENERFQYKCNMGYEYSERGDAVCTESGWRPLPSCEE |
| | | KSCDNPYIPNGDYSPLRIKHRTGDEITYQCRNGFYPATRGNTAKCTSTGWI |
| | | PAPRCTLKP |
| 249. | 3.2LC-GL | SYVLTQPPSVSVAPGQTARITCGHNYFGGKSVHWYQQKPGQAPVLVVYD |
| | | DSDRPSGIPERFSGSNSGNTATLTISRVEAGDEADYYCQVYDTSSDHWVF |
| | | GGGTKLTVLGQPKAAPSVTLFPPSSEELQANKATLVCLISDFYPGAVTVA |
| | | WKADSSPVKAGVETTTPSKQSNNKYAASSYLSLTPEQWKSHRSYSCQVT |
| | | HEGSTVEKTVAPTECS |
| 250. | 3.2pH-GL H- | DSLAVAGSEYFQHW |
| | CDR3 | |

1. An anti-C2 antibody or a fragment thereof comprising a heavy chain variable region (VH) and a light chain variable region (VL), wherein:
a) the VH comprises i) an H-CDR1 comprising the amino acid sequence of SEQ ID NO: 3, ii) an H-CDR2 comprising the amino acid sequence of SEQ ID NO: 4, and iii) an H-CDR3 comprising the amino acid sequence of SEQ ID NO: 5; and the VL comprises i) an L-CDR1 comprising the amino acid sequence of SEQ ID NO: 6, an L-CDR2 comprising the amino acid sequence of SEQ ID NO: 7, and iii) an L-CDR3 comprising the amino acid sequence of SEQ ID NO: 8;
b) the VH comprises i) an H-CDR1 comprising the amino acid sequence of SEQ ID NO: 11, ii) an H-CDR2 comprising the amino acid sequence of SEQ ID NO: 12, and iii) an H-CDR3 comprising the amino acid sequence of SEQ ID NO: 13; and the VL comprises i) an L-CDR1 comprising the amino acid sequence of SEQ ID NO: 14, an L-CDR2 comprising the amino acid sequence of SEQ ID NO: 15, and iii) an L-CDR3 comprising the amino acid sequence of SEQ ID NO: 16;
c) the VH comprises i) an H-CDR1 comprising the amino acid sequence of SEQ ID NO: 19, ii) an H-CDR2 comprising the amino acid sequence of SEQ ID NO: 20, and iii) an H-CDR3 comprising the amino acid sequence of SEQ ID NO: 21; and the VL comprises i) an L-CDR1 comprising the amino acid sequence of SEQ ID NO: 22, an L-CDR2 comprising the amino acid sequence of SEQ ID NO: 23, and iii) an L-CDR3 comprising the amino acid sequence of SEQ ID NO: 24;
d) the VH comprises i) an H-CDR1 comprising the amino acid sequence of SEQ ID NO: 27, ii) an H-CDR2 comprising the amino acid sequence of SEQ ID NO: 28, and iii) an H-CDR3 comprising the amino acid sequence of SEQ ID NO: 29; and the VL comprises i) an L-CDR1 comprising the amino acid sequence of SEQ ID NO: 30, an L-CDR2 comprising the amino acid sequence of SEQ ID NO: 31, and iii) an L-CDR3 comprising the amino acid sequence of SEQ ID NO: 32;
e) the VH comprises i) an H-CDR1 comprising the amino acid sequence of SEQ ID NO: 35, ii) an H-CDR2 comprising the amino acid sequence of SEQ ID NO: 36, and iii) an H-CDR3 comprising the amino acid sequence of SEQ ID NO: 37; and the VL comprises i) an L-CDR1 comprising the amino acid sequence of SEQ ID NO: 38, an L-CDR2 comprising the amino acid sequence of SEQ ID NO: 39, and iii) an L-CDR3 comprising the amino acid sequence of SEQ ID NO: 40;
f) the VH comprises i) an H-CDR1 comprising the amino acid sequence of SEQ ID NO: 43, ii) an H-CDR2 comprising the amino acid sequence of SEQ ID NO: 44, and iii) an H-CDR3 comprising the amino acid sequence of SEQ ID NO: 45; and the VL comprises i) an L-CDR1 comprising the amino acid sequence of SEQ ID NO: 46, an L-CDR2 comprising the amino acid sequence of SEQ ID NO: 47, and iii) an L-CDR3 comprising the amino acid sequence of SEQ ID NO: 48;
g) the VH comprises i) an H-CDR1 comprising the amino acid sequence of SEQ ID NO: 51, ii) an H-CDR2 comprising the amino acid sequence of SEQ ID NO: 52, and iii) an H-CDR3 comprising the amino acid sequence of SEQ ID NO: 53; and the VL comprises i) an L-CDR1 comprising the amino acid sequence of SEQ ID NO: 54, an L-CDR2 comprising the amino acid sequence of SEQ ID NO: 55, and iii) an L-CDR3 comprising the amino acid sequence of SEQ ID NO: 56;
h) the VH comprises i) an H-CDR1 comprising the amino acid sequence of SEQ ID NO: 59, ii) an H-CDR2 comprising the amino acid sequence of SEQ ID NO: 60, and iii) an H-CDR3 comprising the amino acid sequence of SEQ ID NO: 61; and the VL comprises i) an L-CDR1 comprising the amino acid sequence of SEQ ID NO: 62, an L-CDR2 comprising the amino acid sequence of SEQ ID NO: 63, and iii) an L-CDR3 comprising the amino acid sequence of SEQ ID NO: 64;
i) the VH comprises i) an H-CDR1 comprising the amino acid sequence of SEQ ID NO: 67, ii) an H-CDR2 comprising the amino acid sequence of SEQ ID NO: 68, and iii) an H-CDR3 comprising the amino acid sequence of SEQ ID NO: 69; and the VL comprises i) an L-CDR1 comprising the amino acid sequence of SEQ ID NO: 70, an L-CDR2 comprising the amino acid sequence of SEQ ID NO: 71, and iii) an L-CDR3 comprising the amino acid sequence of SEQ ID NO: 72;
j) the VH comprises i) an H-CDR1 comprising the amino acid sequence of SEQ ID NO: 75, ii) an H-CDR2 comprising the amino acid sequence of SEQ ID NO: 76, and iii) an H-CDR3 comprising the amino acid sequence of SEQ ID NO: 77; and the VL comprises i) an L-CDR1 comprising the amino acid sequence of SEQ ID NO: 78, an L-CDR2 comprising the amino acid sequence of SEQ ID NO: 79, and iii) an L-CDR3 comprising the amino acid sequence of SEQ ID NO: 80;
k) the VH comprises i) an H-CDR1 comprising the amino acid sequence of SEQ ID NO: 83, ii) an H-CDR2 comprising the amino acid sequence of SEQ ID NO: 84, and iii) an H-CDR3 comprising the amino acid sequence of SEQ ID NO: 85; and the VL comprises i) an L-CDR1 comprising the amino acid sequence of SEQ ID NO: 86, an L-CDR2 comprising the amino acid sequence of SEQ ID NO: 87, and iii) an L-CDR3 comprising the amino acid sequence of SEQ ID NO: 88;
l) the VH comprises i) an H-CDR1 comprising the amino acid sequence of SEQ ID NO: 19, ii) an H-CDR2 comprising the amino acid sequence of SEQ ID NO: 92, and iii) an H-CDR3 comprising the amino acid sequence of SEQ ID NO: 250; and the VL comprises i) an L-CDR1 comprising the amino acid sequence of SEQ ID NO: 98, an L-CDR2 comprising the amino acid sequence of SEQ ID NO: 23, and iii) an L-CDR3 comprising the amino acid sequence of SEQ ID NO: 24;
m) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 94, an L-CDR2 comprising the amino acid sequence of SEQ ID NO: 95, and iii) an L-CDR3 comprising the amino acid sequence of SEQ ID NO: 96;
n) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 98, an L-CDR2 comprising the amino acid sequence of SEQ ID NO: 99, and iii) an L-CDR3 comprising the amino acid sequence of SEQ ID NO: 100;
o) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 102, an L-CDR2 comprising the amino acid sequence of SEQ ID NO: 103, and iii) an L-CDR3 comprising the amino acid sequence of SEQ ID NO: 104;
p) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 106, an L-CDR2 comprising the amino acid sequence of SEQ ID NO: 107, and iii) an L-CDR3 comprising the amino acid sequence of SEQ ID NO: 108;
q) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 110, an L-CDR2 comprising the amino acid sequence of SEQ ID NO: 111, and iii) an L-CDR3 comprising the amino acid sequence of SEQ ID NO: 112;
r) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 114, an L-CDR2 comprising the amino acid sequence of SEQ ID NO: 115, and iii) an L-CDR3 comprising the amino acid sequence of SEQ ID NO: 116;
s) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 118, an L-CDR2 comprising the amino acid sequence of SEQ ID NO: 119, and iii) an L-CDR3 comprising the amino acid sequence of SEQ ID NO: 120;
t) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 122, an L-CDR2 comprising the amino acid sequence of SEQ ID NO: 123, and iii) an L-CDR3 comprising the amino acid sequence of SEQ ID NO: 124;
u) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 126, an L-CDR2 comprising the amino acid sequence of SEQ ID NO: 127, and iii) an L-CDR3 comprising the amino acid sequence of SEQ ID NO: 128;
v) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 130, an L-CDR2 comprising the amino acid sequence of SEQ ID NO: 131, and iii) an L-CDR3 comprising the amino acid sequence of SEQ ID NO: 132;
w) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 134, ii) an L-CDR2 comprising the amino acid sequence of SEQ ID NO: 135, and iii) an L-CDR3 comprising the amino acid sequence of SEQ ID NO: 136;
x) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 138, ii) an L-CDR2 comprising the amino acid sequence of SEQ ID NO: 139, and iii) an L-CDR3 comprising the amino acid sequence of SEQ ID NO: 140;
y) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 142, ii) an L-CDR2 comprising the amino acid sequence of SEQ ID NO: 143, and iii) an L-CDR3 comprising the amino acid sequence of SEQ ID NO: 144;
z) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 146, ii) an L-CDR2 comprising the amino acid sequence of SEQ ID NO: 147, and iii) an L-CDR3 comprising the amino acid sequence of SEQ ID NO: 148;
aa) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 150, ii) an L-CDR2 comprising the amino acid sequence of SEQ ID NO: 151, and iii) an L-CDR3 comprising the amino acid sequence of SEQ ID NO: 152;
ab) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 154, ii) an L-CDR2 comprising the amino acid sequence of SEQ ID NO: 155, and iii) an L-CDR3 comprising the amino acid sequence of SEQ ID NO: 156;
ac) the VH comprises i) an H-CDR1 comprising the amino acid sequence of SEQ ID NO: 91, ii) an H-CDR2 comprising the amino acid sequence of SEQ ID NO: 92, and iii) an H-CDR3 comprising the amino acid sequence of SEQ ID NO: 93; and the VL comprises i) an L-CDR1 comprising the amino acid sequence of SEQ ID NO: 158, ii) an L-CDR2 comprising the amino acid sequence of SEQ ID NO: 159, and iii) an L-CDR3 comprising the amino acid sequence of SEQ ID NO: 160; or
ad) the VH comprises i) an H-CDR1 comprising the amino acid sequence of SEQ ID NO: 19, ii) an H-CDR2 comprising the amino acid sequence of SEQ ID NO: 92, and iii) an H-CDR3 comprising the amino acid sequence of SEQ ID NO: 250; and the VL comprises i) an L-CDR1 comprising the amino acid sequence of SEQ ID NO: 98, ii) an L-CDR2 comprising the amino acid sequence of SEQ ID NO: 23, and iii) an L-CDR3 comprising the amino acid sequence of SEQ ID NO: 24.

2. (canceled)

3. The anti-C2 antibody or the fragment thereof according to claim 1 comprising an VH and VL, wherein;
a) the VH comprises an amino acid sequence of any one of SEQ ID NOs: 1, 9, 17, 25, 33, 41, 49, 57, 65, 73, 81, 89, and 246, or a variant comprising an amino acid consequence having at least about 80% sequence identity;
b) the VL comprises an amino acid sequence of any one of SEQ ID NOs: 2, 10, 18, 26, 34, 42, 50, 58, 66, 74, 82, 90, 97, 101, 105, 109, 113, 117, 121, 125, 129, 133, 137, 141, 145, 149, 153, 157, and 247 or a variant comprising an amino acid consequence having at least about 80% sequence identity: or
c) any combination of a) and b)

4. (canceled)

5. The anti-C2 antibody or the fragment thereof according to claim 1, wherein;
a) the VH comprises an amino acid sequence of SEQ ID NO: 1; and the VL comprises an amino acid sequence of SEQ ID NO: 2;
b) the VH comprises an amino acid sequence of SEQ ID NO: 9; and the VL comprises an amino acid sequence of SEQ ID NO: 10;
c) the VH comprises an amino acid sequence of SEQ ID NO: 17; and the VL comprises an amino acid sequence of SEQ ID NO: 18;
d) the VH comprises an amino acid sequence of SEQ ID NO: 25; and the VL comprises an amino acid sequence of SEQ ID NO: 26;
e) the VH comprises an amino acid sequence of SEQ ID NO: 33; and the VL comprises an amino acid sequence of SEQ ID NO: 34;
f) the VH comprises an amino acid sequence of SEQ ID NO: 41; and the VL comprises an amino acid sequence of SEQ ID NO: 42;
g) the VH comprises an amino acid sequence of SEQ ID NO: 49; and the VL comprises an amino acid sequence of SEQ ID NO: 50;
h) the VH comprises an amino acid sequence of SEQ ID NO: 57; and the VL comprises an amino acid sequence of SEQ ID NO: 58;
i) the VH comprises an amino acid sequence of SEQ ID NO: 65; and the VL comprises an amino acid sequence of SEQ ID NO: 66;
j) the VH comprises an amino acid sequence of SEQ ID NO: 73; and the VL comprises an amino acid sequence of SEQ ID NO: 74;
k) the VH comprises an amino acid sequence of SEQ ID NO: 81; and the VL comprises an amino acid sequence of SEQ ID NO: 82;
l) the VH comprises an amino acid sequence of SEQ ID NO: 89; and the VL comprises an amino acid sequence of SEQ ID NO: 90;
m) the VH comprises an amino acid sequence of SEQ ID NO: 89; and the VL comprises an amino acid sequence of SEQ ID NO: 97;
n) the VH comprises an amino acid sequence of SEQ ID NO: 89; and the VL comprises an amino acid sequence of SEQ ID NO: 101;
o) the VH comprises an amino acid sequence of SEQ ID NO: 89; and the VL comprises an amino acid sequence of SEQ ID NO: 105;
p) the VH comprises an amino acid sequence of SEQ ID NO: 89; and the VL comprises an amino acid sequence of SEQ ID NO: 109;
q) the VH comprises an amino acid sequence of SEQ ID NO: 89; and the VL comprises an amino acid sequence of SEQ ID NO: 113;
r) the VH comprises an amino acid sequence of SEQ ID NO: 89; and the VL comprises an amino acid sequence of SEQ ID NO: 117;
s) the VH comprises an amino acid sequence of SEQ ID NO: 89; and the VL comprises an amino acid sequence of SEQ ID NO: 121;
t) the VH comprises an amino acid sequence of SEQ ID NO: 89; and the VL comprises an amino acid sequence of SEQ ID NO: 125;
u) the VH comprises an amino acid sequence of SEQ ID NO: 89; and the VL comprises an amino acid sequence of SEQ ID NO: 129;
v) the VH comprises an amino acid sequence of SEQ ID NO: 89; and the VL comprises an amino acid sequence of SEQ ID NO: 133;
w) the VH comprises an amino acid sequence of SEQ ID NO: 89; and the VL comprises an amino acid sequence of SEQ ID NO: 137;
x) the VH comprises an amino acid sequence of SEQ ID NO: 89; and the VL comprises an amino acid sequence of SEQ ID NO: 141;
y) the VH comprises an amino acid sequence of SEQ ID NO: 89; and the VL comprises an amino acid sequence of SEQ ID NO: 145;
z) the VH comprises an amino acid sequence of SEQ ID NO: 89; and the VL comprises an amino acid sequence of SEQ ID NO: 149;
aa) the VH comprises an amino acid sequence of SEQ ID NO: 89; and the VL comprises an amino acid sequence of SEQ ID NO: 153;
ab) the VH comprises an amino acid sequence of SEQ ID NO: 89; and the VL comprises an amino acid sequence of SEQ ID NO: 157; or
ac) the VH comprises an amino acid sequence of SEQ ID NO: 246; and the VL comprises an amino acid sequence of SEQ ID NO: 247.

6. An anti-C2 antibody or a fragment thereof that binds to C2 or C2a competitively with the anti-C2 antibody or the fragment thereof according to claim 1.

7. The anti-C2 antibody or the fragment thereof according to claim 1, wherein the antibody or the fragment thereof is a full-length antibody, Fab, Fab′, F(ab)₂, F(ab′)₂, scFv, or any combination thereof.

8. The anti-C2 antibody or the fragment thereof according to claim 7, wherein the full-length antibody comprises an Fc region.

9. The anti-C2 antibody or the fragment thereof according to claim 8, wherein the Fc region comprises
a) an IgG4 or an IgG1 sequence;
b) the amino acid sequence of SEQ ID NO: 183 or 210, or a variant thereof;
c) one or more mutations selected from the group consisting of S228P, M428L, N434A, and N434S, wherein the mutations are relative to SEQ ID NO: 183 under the EU numbering system;
d) mutations a) S228P, M428L and N434A or b) S228P, M428L and N434S, wherein the mutations are relative to SEQ ID NO: 183 under the EU numbering system;
e) the amino acid sequence of SEQ ID NO: 184 or 216;
f) one or more mutations selected from the group consisting of L234A, L235A, M428L, N434A and N434S, wherein the mutations are relative to SEQ ID NO: 210 under the EU numbering system;
g) mutations a) L234A, L235A, M428L and N434A, or b) S228P, M428L and N434S, wherein the mutations are relative to SEQ ID NO: 210 under the EU numbering system;
h) the amino acid sequence of SEQ ID NO: 214 or 215; or
i) any combination of a) through h).

10-16. (canceled)

17. The anti-C2 antibody or the fragment thereof according to claim 1,
wherein
a) the antibody or the fragment thereof is an isolated anti-C2 antibody or a fragment thereof;
b) the antibody or the fragment thereof is a fully human antibody or a fragment thereof;
c) the antibody or a fragment thereof inhibits the cleavage of human C2 into fragments C2a and C2b;
d) the antibody or a fragment thereof binds to human C2a;
e) the antibody or a fragment thereof cross-reacts with a cynomolgus monkey C2;
f) the antibody or a fragment thereof cross-reacts with a cynomolgus monkey C2a;
g) the antibody or a fragment thereof has a serum half-life in humans that is at least about 2 days;
h) the antibody or a fragment thereof is manufactured in CHO cells; or
i) any combination of a) through h).

18. A fusion protein comprising
a) the anti-C2 antibody or the fragment thereof according to claim 1;
b) a heavy chain-factor H fusion polypeptide and a light chain, wherein
i) the heavy chain-factor H fusion comprises an amino acid sequence of any of SEQ ID NOs: 202-204, 206, 211-213, 220-228, and 248, or a variant comprising an amino acid consequence having at least about 80% sequence identity;
ii) the light chain comprises an amino acid sequence of any of SEQ ID NOs: 205, 207, 229-245, and 249 or a variant comprising an amino acid consequence having at least about 80% sequence identity; or
iii) any combination of i) and ii);
c) the fusion protein comprises an amino acid sequence of SEQ ID NO: 201, or a variant comprising an amino acid consequence having at least about 80% sequence identity;
d) the fusion protein is capable of overcoming the C2 bypass phenomenon; or
e) any combination of a) through d).

19. The fusion protein according to claim 18, wherein
a)
i) the heavy chain-factor H fusion comprises an amino acid sequence of any of SEQ ID NOs: 223-228 and 248, or a variant comprising an amino acid consequence having at least about 80% sequence identity;
ii) the light chain comprises an amino acid sequence of any of SEQ ID NOs: 229-245 and 249 or a variant comprising an amino acid consequence having at least about 80% sequence identity; or
iii) any combination of i) and ii);
b) the heavy chain-factor H fusion comprises an amino acid sequence of any of SEQ ID NOs: 202-204, 206, 211-213, and 220-228, and the light chain comprises an amino acid sequence of any of SEQ ID NOs: 205, 207, and 229-245; or
c) any combination of a) and b).

20. (canceled)

21. The anti-C2 antibody or the fragment thereof according to claim 1, wherein
a) the low-pH dissociation factor of the antibody or the fragment thereof dissociating from human C2 is between about 40% to about 95%;
b) the neutral-pH dissociation factor of the antibody or the fragment thereof dissociating from human C2 is between about 0% to about 15%;
c) the ratio of low-pH dissociation to neutral-pH dissociation from human C2 is 5 or more;
d) the low-pH dissociation factor of the antibody or a fragment thereof dissociating from cynomolgus C2 is between about 40% to about 80%;
e) the neutral-pH dissociation factor of the antibody or a fragment thereof dissociating from cynomolgus C2 is between about 0% to about 10%;
f) the ratio of low-pH dissociation to neutral-pH dissociation from cynomolgus C2 is 7 or more; or
g) any combination of a) through f).

22-32. (canceled)

33. An isolated nucleic acid encoding the antibody or the fragment thereof according to claim 1.

34. A vector comprising the isolated nucleic acid according to claim 33.

35. A host cell comprising the isolated nucleic acid according to claim 33.

36. A method of producing a fusion protein comprising:
a) culturing a host cell according to claim 35 under a condition suitable for expression of the fusion protein; and
b) obtaining the expressed fusion protein from said cell.

37. A pharmaceutical composition comprising the antibody or the fragment thereof according to claim 1 and a pharmaceutically acceptable carrier.

38. A method of treating an individual having a complement-associated disease or condition, comprising administering to the individual an effective amount of the antibody or the fragment thereof according to claim 1 or a composition thereof.

39. (canceled)

40. A method of reducing the activity of a complement system in an individual, comprising administering to the individual an effective amount of the antibody or the fragment thereof according to claim 1 or a composition thereof.

41-42. (canceled)

43. The anti-C2 antibody or the fragment thereof according to claim 6, wherein the C2 or C2a comprises an amino acid sequence of any one of SEQ ID NOs: 185-188, or a variant comprising an amino acid consequence having at least about 80% sequence identity.

44. The vector according to claim 34, wherein the vector is a viral vector selected from the group consisting of a retrovirus vector, an adenovirus vector, an adeno-associated virus vector, an herpes virus vector, a lentivirus vector, a murine stem cell virus vector, a moloney murine leukemia virus vector, and an human immunodeficiency virus vector.
